(19) United States
(12) Patent Application Publication
(10) Pub. No.: US 2022/0096149 A1
(43) Pub. Date: Mar. 31, 2022

(54) Visualization-Enabled Ablation Catheters, Systems, and Methods

(71) Applicant: Santa Anna Tech LLC, Santa Ana, CA (US)

(72) Inventors: Virender K. Sharma, Paradise Valley, AZ (US); George W. Bourne, Boston, MA (US)

(21) Appl. No.: 17/450,273

(22) Filed: Oct. 7, 2021

Related U.S. Application Data
(63) Continuation-in-part of application No. 17/005,982, filed on Aug. 28, 2020.
(60) Provisional application No. 63/210,523, filed on Jun. 15, 2021.
(60) Provisional application No. 63/089,450, filed on Oct. 8, 2020.
(60) Provisional application No. 63/025,867, filed on May 15, 2020.
(60) Provisional application No. 62/953,116, filed on Dec. 23, 2019.
(60) Provisional application No. 62/893,062, filed on Aug. 28, 2019.

(51) Int. Cl. A61B 18/14; A61B 34/20; A61B 1/04; A61B 1/06; A61B 1/00; A61B 1/015

(52) U.S. Cl. CPC A61B 18/1492; A61B 18/1477; A61B 34/20; A61B 2018/00547; A61B 1/06; A61B 1/00135; A61B 1/015; A61B 1/04

(57) ABSTRACT

Ablation catheters and systems include catheter tips with at least one hollow needle that is extendable at an angle from the catheter body to ablate a target prostate tissue while avoiding structures in regions near the prostate tissue, including the urethra, the ejaculatory duct, and the rectum wall. The vapor ablation system has a pump, a catheter that includes a connection port positioned on a proximal end of the catheter, a lumen in fluid communication with the connection port and configured to receive, via the connection port, saline from the pump, at least one electrode positioned within the lumen, and at least two positioning elements, having a lumen with vapor ports between them and configured to be coupled to the distal tip of the catheter. Embodiments of catheter handle mechanisms are described that provide ergonomic methods for handling and using the catheter.

### CROSS-REFERENCE

The present application relies on, for priority, U.S. Patent Provisional Application No. 63/210,523, entitled “Systems and Methods for Ablating Tissue” and filed on Jun. 15, 2021 and U.S. Patent Provisional Application No. 63/089,450, entitled “Systems and Methods for Ablating Tissue” and filed on Oct. 8, 2020.

The present application is a continuation-in-part application of U.S. patent application Ser. No. 17/005,982, entitled “Systems and Methods for Ablating Prostate Tissue” and filed on Aug. 28, 2020, which relies on, for priority, U.S. Patent Provisional Application No. 63/025,867, entitled “Systems and Methods for Genitourinary Ablation” and filed on May 15, 2020, U.S. Patent Provisional Application No. 62/953,116, entitled “Systems and Methods for Prostate and Endometrial Ablation” and filed on Dec. 23, 2019, and U.S. Patent Provisional Application No. 62/893,062, of the same title and filed on Aug. 28, 2019.

The present application is related to U.S. patent application Ser. No. 15/600,670, entitled “Ablation Catheter with Integrated Cooling” and filed on May 19, 2017, which relies on U.S. Provisional Patent Application No. 62/425,144, entitled “Methods and Systems for Ablation” and filed on Nov. 22, 2016, and U.S. Provisional Patent Application No. 62/338,871, entitled “Cooled Coaxial Ablation Catheter” and filed on May 19, 2016, for priority.

U.S. patent application Ser. No. 15/600,670 is also a continuation-in-part application of U.S. patent application Ser. No. 15/144,768, entitled “Induction-Based Micro-Volume Heating System”, filed on May 2, 2016, and issued as U.S. Pat. No. 10,064,697 on Sep. 4, 2018, which is a continuation-in-part application of U.S. patent application Ser. No. 14/594,444, entitled “Method and Apparatus for Tissue Ablation”, filed on Jan. 12, 2015, and issued as U.S. Pat. No. 9,561,068 on Feb. 7, 2017, which is a continuation-in-part application of U.S. patent application Ser. No. 14/158,687, of the same title, filed on Jan. 17, 2014, and issued as U.S. Pat. No. 9,561,067 on Feb. 7, 2017, which, in turn, relies on U.S. Provisional Patent Application No. 61/753,831, of the same title and filed on Jan. 17, 2013, for priority.

U.S. patent application Ser. No. 14/158,687 is also a continuation-in-part application of U.S. patent application Ser. No. 13/486,980, entitled “Method and Apparatus for Tissue Ablation”, filed on Jun. 1, 2012, and issued as U.S. Pat. No. 9,561,066 on Feb. 7, 2017, which, in turn, relies on U.S. Provisional Patent Application No. 61/493,344, of the same title and filed on Jun. 3, 2011, for priority.

U.S. patent application Ser. No. 13/486,980 is also a continuation-in-part application of U.S. patent application Ser. No. 12/573,939, entitled “Method and Apparatus for Tissue Ablation” and filed on Oct. 6, 2009, which, in turn, relies on U.S. Provisional Patent Application No. 61/102,885, of the same title and filed on Oct. 6, 2008, for priority.

All of the above referenced applications are herein incorporated by reference in their entirety.

### FIELD

The present specification relates to systems and methods configured to generate and deliver vapor for ablation therapy. More particularly, the present specification relates to systems and methods comprising a handle mechanism for vapor ablation catheter.

### BACKGROUND

Benign Prostatic Hyperplasia (BPH) refers to enlargement of the prostate gland. The enlargement may be non-cancerous and is common in men as they grow older. However, the enlargement of the prostate gland, due to BPH, may result in compressing of the urethra, thereby impeding the flow of urine from the bladder through the urethra. Anatomically, the median and lateral lobes are usually enlarged, due to their highly glandular composition. The anterior lobe has little in the way of glandular tissue and is seldom enlarged. Carcinoma of the prostate typically occurs in the posterior lobe—hence the ability to discern an irregular outline per rectal examination.

The earliest microscopic signs of BPH usually begin between the age of 30 and 50 years old men in the pen-urethral zone (PuZ), which is posterior to the proximal urethra. In BPH, most of the growth occurs in the transition zone (TZ) of the prostate. In addition to these two classic areas, the peripheral zone (PZ) is also involved to a lesser extent. Prostatic cancer typically occurs in the PZ. However, BPH nodules, usually from the TZ, are often biopsied anyway to rule out cancer in the TZ. BPH is nodular hyperplasia and not diffuse hyperplasia, affecting the TZ and PuZs of the prostate. Adenoma from the TZ form the lateral lobes while adenoma from the PuZ form the middle lobe in clinical diseases.

Transurethral needle ablation (TUNA) is a procedure used to treat the symptoms caused by BPH. The ablation procedure is used to treat the extra prostate tissue that causes the symptoms of BPH.

Prostate cancer is diagnosed in approximately 8% of men between the ages of 50 and 70 and tends to occur in men as they grow older. Men experiencing symptoms with prostate cancer often exhibit symptoms similar to those encountered with BPH and can also suffer from sexual problems caused by the disease. Typically, men diagnosed with prostate cancer when the cancer is at an early stage have a very good prognosis. Therapy ranges from active surveillance to surgery and radiation and chemotherapy depending on the severity of the disease and the age of the patient.

Dysfunctional uterine bleeding (DUB), or menorrhagia, affects 30% of women in reproductive age. The associated symptoms have considerable impact on a woman's health and quality of life. The condition is typically treated with endometrial ablation or a hysterectomy. The rates of surgical intervention in these women are high. Almost 30% of women in the US will undergo hysterectomy by the age of 60, with menorrhagia or DUB being the cause for surgery in 50-70% of these women. Endometrial ablation techniques have been FDA approved for women with abnormal uterine bleeding and with intramural fibroids less than 2 cm in size. The presence of submucosal uterine fibroids and a large uterus size have been shown to decrease the efficacy of standard endometrial ablation. Of the five FDA approved global ablation devices (namely, Thermachoice, hydrothermal ablation, Novasure, Her Option, and microwave ablation (MEA)) only microwave ablation has been approved for use where the submucosal fibroids are less than 3 cm in size and are not occluding the endometrial cavity and, additionally, for large uteri up to 14 cm in width.

Bladder cancer is a rare form of cancer occurring as a result of an abnormal growth of cells within the bladder. The abnormal cells form a tumor. FIG. 22A illustrates multiple stages of cancer of a bladder 2200, as known in the medical field. Referring to the figure, at a first stage (Tis), a bladder tumor 2202 is above a mucosa 2204 layer within the bladder 2200. At a second stage (Ta), a tumor 2206 spreads to the mucosa 2204. At a third stage (T1), a tumor 2208 spreads to a submucosa 2210 layer beneath the mucosa 2204. At a fourth stage (T2), a tumor 2212 spreads to a superficial muscle 2214 beneath the submucosa 2210. At a fifth stage (T3a), a tumor 2216 spreads to a deep muscle 2218 beneath the superficial muscle 2214. At a sixth stage (T3b), a tumor 2220 spreads to a perivesical fat layer 2222 beyond the deep muscle 2218. At a seventh stage (T4b), a tumor 2224 spreads to areas outside the perivesical fat layer 2222. At an eighth stage (T4a), a tumor 2226 spreads to extravesical structures 2228 outside the bladder 2200. Ablation techniques may be used to treat cancer of stages from first to fourth, which are non-muscle invasive or superficial bladder cancers. Further, ablation techniques may be used to palliate cancer of stages from fifth onwards, which are invasive bladder cancers.

A bladder's function is to hold urine that is made in the kidneys and travels down to the bladder through tubes called ureters. Urine exits the bladder into the urethra which in turn ports the urine out of the body. Some individuals suffer from an over-active bladder (OAB) that leads to an urge to pass urine several times in a day, even when the bladder is not full. Ablation techniques may be used to treat patients with OAB.

As the bladder is meant to hold urine, vapors from ablation methods may not be effective in the presence of urine over the tissue that needs to be ablated. It is therefore desirable to provide a way to ablate bladder tissues after complete removal of fluids, water, and/or urine away from the target tissue.

Ablation, as it pertains to the present specification, relates to the removal or destruction of a body tissue, via the introduction of a destructive agent, such as radiofrequency energy, laser energy, ultrasonic energy, cyroagents, or steam. Ablation is commonly used to eliminate diseased or unwanted tissues, such as, but not limited to cysts, polyps, tumors, hemorrhoids, and other similar lesions. Ablation techniques may be used in combination with chemotherapy, radiation, surgery, and Bacillus Calmette-Guérin (BCG) vaccine therapy, among others.

Steam-based ablation systems, such as the ones disclosed in U.S. Pat. Nos. 9,615,875, 9,433,457, 9,376,497, 9,561,068, 9,561,067, and 9,561,066, disclose ablation systems that controllably deliver steam through one or more lumens toward a tissue target. One problem that all such steam-based ablation systems have is the potential overheating or burning of healthy tissue. Steam passing through a channel within a body cavity heats up surfaces of the channel and may cause exterior surfaces of the medical tool, other than the operational tool end itself, to become excessively hot. As a result, physicians may unintentionally burn healthy tissue when external portions of the device, other than the distal operational end of the tool, accidentally contacts healthy tissue. U.S. Pat. Nos. 9,561,068, 9,561,067, and 9,561,066 are hereby incorporated herein by reference.

It is desirable to have steam-based ablation devices that integrate into the device itself safety mechanisms which prevent unwanted ablation during use. It is further desirable to have a catheter handle that allows the user to ergonomically hold the device during a vapor ablation treatment. Finally, it is desirable to provide device holding and handling mechanisms for use with a single hand.

### SUMMARY

The present specification discloses an ablation catheter configured to deliver an ablation fluid to at least one of a volume of prostate tissue or a volume of fibroid tissue, comprising: a sheath having at least one lumen, wherein the lumen is configured to receive a volume of fluid; at least one needle positioned within a distal tip of the catheter and configured to be deployed from a surface of the distal tip; at least one port positioned in the at least one needle; at least one heating component positioned within the lumen and proximate the distal tip, wherein the at least one heating component is configured to receive the volume of fluid; a handle coupled to a proximal end of the sheath; a camera positioned proximate the distal tip and configured to visually capture a movement and location of the at least one needle when the at least one needle is extended out from the surface of the distal tip; a light source positioned proximate the camera, wherein the camera and the light source are physically coupled into the sheath; and optical data transmission circuitry coupled to the camera, wherein a value defining a maximum diameter of the catheter is equal to, or less than, 8 mm.

Optionally, the at least one heating component is a flat electrode.

Optionally, the at least one needle is configured to be deployed at an angle relative to a longitudinal axis defining a direction of the distal tip. The angle may be in a range of 10 degrees to 90 degrees relative to the longitudinal axis defining the direction of the distal tip.

Optionally, the camera and the light source are not positioned in a scope physically separate from the sheath.

Optionally, the maximum diameter of the catheter is in a range of 4 mm to 6 mm.

Optionally, the at least one heating component comprises an electrode wherein the electrode is tapered such that a distal tip of the electrode is thinner than a proximal portion of the electrode.

Optionally, the fluid is saline.

Optionally, the sheath comprises a second lumen running parallel to the at least one lumen wherein the optical data transmission circuitry is positioned within the second lumen.

The present specification also discloses an ablation system configured to deliver an ablation fluid to at least one of a volume of prostate tissue or a volume of fibroid tissue, comprising: a catheter having: a sheath having at least one lumen, wherein the lumen is configured to receive a volume of fluid; at least one needle positioned within a distal tip of the catheter and configured to be deployed from a surface of the distal tip; at least one port positioned in the at least one needle; at least one heating component positioned within the lumen and proximate the distal tip, wherein the at least one heating component is configured to receive the volume of fluid; a handle coupled to a proximal end of the sheath; a camera positioned proximate the distal tip and configured to visually capture a movement and location of the at least one needle when the at least one needle is extended out from the surface of the distal tip; a light source positioned proximate the camera, wherein the camera and the light source are physically coupled to the sheath; and optical data transmission circuitry coupled to the camera, wherein a value defining a maximum diameter of the catheter is equal to, or less than, 8 mm; a fluid reservoir configured to contain the volume of fluid and coupled to the at least one lumen; a pump in pressure communication with the fluid reservoir; and a controller coupled to the pump, wherein the controller is in electrical communication with the at least one heating component and programmed to deliver an electrical current to the at least one heating component and to cause the volume of fluid to pass into the lumen from the fluid reservoir when activated.

Optionally, the ablation system further comprises a power source positioned in the controller and coupled to the light source and the camera.

Optionally, the at least one heating component is a flat electrode.

Optionally, the at least one needle is configured to be deployed at an angle relative to a longitudinal axis defining a direction of the distal tip. The angle may be in a range of 10 degrees to 90 degrees relative to the longitudinal axis defining the direction of the distal tip.

Optionally, the camera and the light source are not positioned in a scope physically separate from the sheath.

Optionally, the maximum diameter of the catheter is in a range of 4 mm to 6 mm.

Optionally, the at least one heating component is an electrode wherein the electrode is tapered such that a distal tip of the electrode is thinner than a proximal portion of the electrode.

Optionally, the controller is programmed to deliver the electrical current to the at least one heating component for a continuous period of time that is equal to, or less than, one minute.

Optionally, the sheath comprises a second lumen running parallel to the at least one lumen wherein the optical data transmission circuitry is positioned within the second lumen. Optionally, the second lumen has a diameter that is equal to or less than 4 mm and the at least one lumen has a diameter that is equal to or less than 4 mm.

The present specification also discloses a handle mechanism for a catheter device used for ablating tissue of a patient, comprising: an elongate tubular structure comprising a proximal end and a distal end; a catheter shaft extending through the tubular structure and extending from the distal end of the tubular structure; at least two positioning elements positioned inside the catheter shaft, wherein the positioning elements are configured to be coupled to a distal tip of the catheter shaft, comprising: a proximal positioning element positioned at a distal tip of the catheter shaft; and a distal positioning element positioned at a distal side of the proximal positioning element; at least one hole configured between the at least two positioning elements, wherein the at least one hole is configured to provide an exit for steam for ablating the patient tissue; a button comprising a safety feature, wherein the button when enabled, activates generation of steam for ablation through the at least one hole; a first control attached to the catheter shaft, configured to perform at least one of linear advancing and retracting of the proximal positioning element; a second control attached to the catheter shaft, configured to perform at least one of linear advancing and retracting of the distal positioning element; a third control attached to the catheter shaft, configured to fix positions of the proximal positioning element and the distal positioning element; and an indicator to show an extent of separation between the proximal positioning element and the distal positioning element.

Optionally, the catheter shaft comprises an inner catheter that is coaxially positioned inside the catheter shaft. Optionally, the at least two positioning elements are configured towards a distal portion of the inner catheter. Optionally, the at least one hole is configured on the inner catheter.

Optionally, the handle mechanism is operable with a single hand.

Optionally, the button, the first control, the second control, the third control, and the indicator, are provided on a first lateral side of the handle mechanism.

Optionally, the button is at least one of a press button, a slide button, or a rotating wheel.

Optionally, the first control and the second control are parallel to each other.

Optionally, the second control surrounds the first control.

Optionally, the indicator comprises a scale with markings visible through a window wherein the markings align with at least one arrow outside the window to show the extent of separation between the proximal positioning element and the distal positioning element.

Optionally, each of the first control and the second control comprise at least one of a slider, a lever that is squeezed, a trigger arm, a toggle button, and a combination of directional press buttons where each button indicates a direction of linear movement of the at least two positioning elements.

Optionally, the third control comprises at least one of a rotating lever, a rotating knob, a toggle button, a press button.

Optionally, the handle mechanism further comprises a strain relief positioned at the distal end over a portion of the catheter shaft.

Optionally, the handle mechanism further comprises a fluid line extending from the proximal end and in fluid communication with the catheter shaft. Optionally, the handle mechanism further comprises a power line extending from the proximal end and at least one electrode in a proximal portion of the catheter shaft, wherein the power line is in electrical communication with the at least one electrode and is configured to provide an electrical current to the electrode. Optionally, the handle mechanism further comprises a strain relief positioned at the proximal end over a portion of the fluid line and power line.

The present specification also discloses a handle mechanism for a catheter device used for ablating tissue of a patient, comprising: a structure comprising two portions, wherein a first portion is rotatable to bring the first portion at an angle relative to a second portion, and comprising a proximal end and a distal end; a catheter shaft extending through the structure and extending from the distal end of the structure; at least two positioning elements positioned inside the catheter shaft, wherein the positioning elements are configured to be coupled to a distal tip of the catheter shaft, comprising: a proximal positioning element positioned at a distal tip of the catheter shaft; and a distal positioning element positioned at a distal side of the proximal positioning element; at least one hole configured between the at least two positioning elements, wherein the at least one hole is configured to provide an exit for steam for ablating the patient tissue; a button comprising a safety feature, wherein the button when enabled, activates generation of steam for ablation through the at least one hole; a first control attached to the catheter shaft, configured to perform at least one of linear advancing and retracting of the proximal positioning element; a second control attached to the catheter shaft, configured to perform at least one of linear advancing and retracting of the distal positioning element; a third control attached to the catheter shaft, configured to fix positions of the proximal positioning element and the distal positioning element; and an indicator to show an extent of separation between the proximal positioning element and the distal positioning element.

Optionally, the angle is in a range from 0 and 180 degrees.

Optionally, the handle mechanism further comprises a button to enable and disable rotation of the first portion relative to the second portion.

The present specification also discloses a handle mechanism for a catheter device used for ablating tissue of a patient, comprising: an elongate planar structure comprising a proximal end and a distal end; a catheter shaft extending through the structure and extending from the distal end of the structure; at least two positioning elements positioned inside the catheter shaft, wherein the positioning elements are configured to be coupled to a distal tip of the catheter shaft, comprising: a proximal positioning element positioned at a distal tip of the catheter shaft; and a distal positioning element positioned at a distal side of the proximal positioning element; at least one hole configured between the at least two positioning elements, wherein the at least one hole is configured to provide an exit for steam for ablating the patient tissue; a button comprising a safety feature, wherein the button when enabled, activates generation of steam for ablation through the at least one hole; a first control attached to the catheter shaft, configured to incrementally perform at least one of linear advancing and retracting of the proximal positioning element, wherein a position of the proximal positioning element is fixed when the first control is at a rest; a second control attached to the catheter shaft, configured to incrementally perform at least one of linear advancing and retracting of the distal positioning element, wherein a position of the distal positioning element is fixed when the second control is at a rest; and a scale with markings adjacent to each of the first control and the second control to indicate an extent of advancing and retracting of the proximal positioning element and the distal positioning element.

The present specification discloses a handle mechanism for a catheter device used for ablating tissue of a patient, comprising: an elongate tubular structure comprising a proximal end and a distal end; a catheter shaft extending through the tubular structure and extending from the distal end of the tubular structure; at least one thermally conductive, elongate element positioned inside the catheter shaft, wherein the thermally conductive element is configured to be coupled to a distal tip of the catheter shaft; a button comprising a safety feature, wherein the button when enabled, activates generation of steam for ablation through the at least one thermally conductive element; a first control attached to the catheter shaft, configured to perform at least one of linear advancing and retracting of the at least one thermally conductive element when actuated; and a second control attached to the catheter shaft and the at least one thermally conductive element, configured to cause a rotation of the thermally conductive element when actuated.

Optionally, the at least one thermally conductive, elongate element is a needle.

The handle mechanism may be operable with a single hand.

Optionally, the button is at least one of a press button, a slide button, or a rotating wheel.

Optionally, the first control comprises an incremental control for advancing the at least one thermally conductive element.

Optionally, the first control is configured to instantly retract the at least one thermally conductive element when actuated.

Optionally, the first control comprises at least one of a slider, a lever that is squeezed, a trigger arm, a toggle button, and a combination of directional press buttons where each button indicates a direction of linear movement of the at least one thermally conductive element.

Optionally, the second control comprises at least one of a rotating wheel, a rotating knob, or a combination of directional press buttons where each button indicates a direction of rotation of the at least one thermally conductive element.

Optionally, the handle mechanism further comprises grooves along the elongated tubular structure for an ergonomic grip.

Optionally, the handle mechanism further comprises first markings that show units of distance travelled by the at least one thermally conductive element when using the first control. The markings may comprise haptic feedback.

Optionally, the handle mechanism further comprises second markings that show units of angle rotated by the at least one thermally conductive element when using the second control.

Optionally, the handle mechanism further comprises a strain relief positioned at the distal end over a portion of the catheter shaft.

Optionally, the handle mechanism further comprises a fluid line extending from the proximal end and in fluid communication with the catheter shaft. Optionally, the handle mechanism further comprises a power line extending from the proximal end and at least one electrode in a proximal portion of the catheter shaft, wherein the power line is in electrical communication with the at least one electrode and is configured to provide an electrical current to the electrode. Optionally, the handle mechanism further comprises a strain relief positioned at the proximal end over a portion of the fluid line and power line.

The present specification also discloses a handle mechanism for a catheter device used for ablating tissue of a patient, comprising: an angled structure comprising two portions, wherein a first portion is at an angle relative to a second portion, and comprising a proximal end and a distal end; a catheter shaft extending through the angular structure and extending from the distal end; at least one thermally conductive, elongated element positioned inside the catheter shaft, wherein the thermally conductive element is configured to be coupled to a distal tip of the catheter shaft; a button comprising a safety feature, wherein the button when enabled, activates generation of steam for ablation through the at least one thermally conductive element; a first control attached to the catheter shaft, configured to perform at least one of advancing and retracting of the at least one thermally conductive element when actuated; and a second control attached to the catheter shaft and the at least one thermally conductive element, configured to cause rotation of the thermally conductive element when actuated.

Optionally, the angle is in a range from 0 and 180 degrees.

The present specification also discloses a handle mechanism for a catheter device used for ablating tissue of a patient, comprising: a first elongate portion; a second elongate portion connected to said first elongate portion by a pivot and at a rotatable angle relative the second portion; a catheter shaft extending through the handle mechanism and extending from a distal end of the second portion; at least one thermally conductive, elongated element positioned inside the catheter shaft, wherein the thermally conductive element is configured to be coupled to a distal tip of the catheter shaft; a button comprising a safety feature, wherein the button when enabled, activates generation of steam for ablation through the at least one thermally conductive element; a first control attached to the catheter shaft, configured to perform at least one of advancing and retracting of the at least one thermally conductive element when actuated; and a second control attached to the catheter shaft and the at least one thermally conductive element, configured to cause rotation of the thermally conductive element.

Optionally, the angle is in a range from 0 to 180 degrees.

The present specification also discloses a vapor ablation system for ablating prostate tissue of a patient, wherein the system comprises: at least one pump; a catheter having a length extending between a proximal end and a distal tip, wherein the catheter comprises: a connection port positioned on the proximal end of the catheter, wherein, through the connection port, the catheter is in fluid communication with the at least one pump; a first lumen in fluid communication with the connection port and configured to receive, via the connection port, saline from the at least one pump; at least one electrode positioned within the first lumen; and at least one thermally conductive, elongated element having a lumen and configured to be coupled to the distal tip of the catheter such that a proximal end of the at least one thermally conductive, elongated element is positioned at least 0.1 mm and no more than 60 mm from a distal most electrode of the at least one electrode and such that the lumen of the at least one thermally conductive, elongated element is in fluid communication with the first lumen; and a controller having at least one processor in data communication with the at least one pump, wherein, upon activating, the controller is configured to: control a delivery of saline into the first lumen; and control a delivery of an electrical current to the at least one electrode positioned within the first lumen.

Optionally, the at least one thermally conductive, elongated element comprises a needle and a needle attachment component. The needle may have a tapered distal tip. The needle and the needle attachment component may be made of the same material and the same material may be stainless steel. A proximal portion of the needle may be configured to be threaded onto a distal end of the needle attachment component

Optionally, the vapor ablation system further comprises a needle chamber coupled to the distal tip of the catheter and configured to be retractable along a length of the catheter. The needle chamber may have an exterior surface and an internal lumen that defines an internal surface, wherein the exterior surface comprises a first material, wherein the internal surface comprises a second material, and wherein the first material is different from the second material. The first material may be a polymer and the second material may be metal. The needle chamber may have an internal lumen that defines an internal surface, wherein the internal lumen is curved to receive a curved needle. The at least one thermally conductive, elongated element may comprise a needle, wherein, in a pre-deployment state, the needle chamber is configured to be positioned over the needle and wherein, in a post-deployment state, the needle chamber is configured to be retracted toward a proximal end of the catheter and the needle is positioned outside the needle chamber. Optionally, the needle is further adapted to have a pre-needle chamber state, wherein, in the pre-needle chamber state, the needle has a first degree of curvature, wherein, in the pre-deployment state, the needle has a second degree of curvature, wherein, in the post-deployment state, the needle has a third degree of curvature, wherein the first degree of curvature is different from both the second degree of curvature and third degree of curvature, and wherein the second degree of curvature is different from the third degree of curvature. Optionally, the needle is further adapted to have a pre-needle chamber state, wherein, in the pre-needle chamber state, the needle has a first degree of curvature, wherein, in the pre-deployment state, the needle has a second degree of curvature, wherein, in the post-deployment state, the needle has a third degree of curvature, wherein the first degree of curvature is greater than both the second degree of curvature and third degree of curvature, and wherein the third degree of curvature is greater than the second degree of curvature. Optionally, in a post-deployment state, the needle is configured to extend outward at an angle between 30° and 90° from an external surface of the catheter.

Optionally, the at least one thermally conductive, elongated element comprises a needle and a needle attachment component wherein the needle comprises an internal channel in fluid communication with the first lumen and a port to allow a passage of vapor to an external environment from the internal channel.

Optionally, the at least one thermally conductive, elongated element comprises more than one needle.

Optionally, the at least one thermally conductive, elongated element comprises a needle having a length extending from a proximal end to a tapered, distal end and further comprises insulation positioned over the length of needle. The insulation may be adapted to cover at least 5% of the length of the needle, beginning from the proximal end wherein the insulation is adapted to no more than 90% of the length of the needle, beginning from the proximal end.

Optionally, the controller is adapted to control the delivery of saline into the first lumen and control the delivery of the electrical current to the at least one electrode such that greater than 0% and less than 75% of a contiguous circumference of a prostatic urethra of the patient is ablated.

Optionally, the controller is adapted to control the delivery of saline into the first lumen and control the delivery of the electrical current to the at least one electrode such that greater than 0% and less than 75% of a contiguous circumference of an ejaculatory duct of the patient is ablated.

Optionally, the controller is adapted to control the delivery of saline into the first lumen and control the delivery of the electrical current to the at least one electrode such that greater than 0% and less than 75% of a thickness of the rectal wall is ablated.

Optionally, the controller is adapted to control the delivery of saline into the first lumen and control the delivery of the electrical current to the at least one electrode such that greater than 0% and less than 75% of one of a contiguous circumference of an ejaculatory duct and a central zone of the prostate is ablated.

Optionally, the controller is adapted to control the delivery of saline into the first lumen and control the delivery of the electrical current to the at least one electrode such that a transitional zone of a prostate of the patient is ablated and greater than 0% and less than 75% of an anterior fibromuscular strauma of the patient is ablated.

The present specification also discloses a vapor ablation system for treating diseases, wherein the system comprises: at least one pump; a catheter in fluid communication through a catheter connection port with the at least one pump, wherein a proximal end of the catheter is connected to the catheter connection port to place the catheter in fluid communication with the at least one pump, wherein the catheter comprises: at least one lumen to transport saline delivered from the at least one pump; at least one electrode positioned within the at least one lumen; a plurality of openings proximate to a distal end of the catheter; a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to: control the delivery of saline into the at least one lumen in the catheter; control the delivery of an electrical current to the at least one electrode positioned within the at least one lumen of the first catheter; and control vapor generated from the saline.

Optionally, the plurality of thermally conductive elements are needles.

Optionally, the plurality of thermally conductive elements are extended at an angle between 30° and 90° from the catheter.

Optionally, the system is used for ablating prostate tissue of a patient through the urethra of the patient, wherein greater than 0% and less than 75% of a contiguous circumference of a prostatic urethra of the patient is ablated.

Optionally, the system is used for ablating prostate tissue of a patient through the urethra of the patient, wherein greater than 0% and less than 75% of a contiguous circumference of an ejaculatory duct of the patient is ablated.

Optionally, the system is used for ablating prostate tissue of a patient through the rectal wall of the patient, wherein greater than 0% and less than 75% of a thickness of the rectal wall is ablated.

Optionally, the system is configured to ablate at least one of a central zone or a transitional zone of a prostate while ablating greater than 0% and less than 75% of a contiguous circumference of a prostatic urethra.

Optionally, the system is configured to ablate at least one of a central zone or a transitional zone of a prostate while ablating greater than 0% and less than 75% of a contiguous circumference of an ejaculatory duct.

Optionally, the system is configured to ablate a median lobe of a prostate while ablating greater than 0% and less than 75% of one of a contiguous circumference of an ejaculatory duct and a central zone of the prostate.

Optionally, the system is configured to ablate a transitional zone of a prostate while ablating greater than 0% and less than 75% of an Anterior Fibromuscular Strauma (AFS).

The present specification also discloses a method of ablating a prostatic tissue of a patient, comprising: providing an ablation system comprising: at least one pump; a catheter in fluid communication with the at least one pump, wherein a proximal end of the catheter is connected to the catheter connection port to place the catheter in fluid communication with the at least one pump, wherein the catheter comprises: at least one lumen configured to transport saline delivered from the at least one pump; at least one electrode positioned within the at least one lumen; a plurality of openings proximate to a distal end of the catheter; and a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to control the delivery of saline into the at least one lumen in the catheter, wherein the electrode is configured to receive an electrical current and convert said saline into vapor for ablation; inserting said catheter into a urethra of said patient; extending said thermally conductive elements through said plurality of openings and into said prostatic tissue; and programming said controller to control a delivery of said vapor such that greater than 0% and less than 75% of a circumference of a prostatic tissue or proximate tissue is ablated.

Optionally, the thermally conductive elements comprise needles.

Optionally, said prostatic tissue or proximate tissue is a prostatic urethra.

Optionally, said prostatic tissue or proximate tissue is an ejaculatory duct.

Optionally, said prostatic tissue or proximate tissue is a rectal wall.

The present specification also discloses a vapor ablation system for treating diseases, wherein the system comprises: at least one pump; a coaxial catheter for inserting into a vagina of a patient towards the cervix, the coaxial catheter comprising: an outer catheter for advancing to the internal os of the cervix of the patient; an inner catheter for advancing into the uterus of the patient, concentric and slidable within the outer catheter, wherein the inner catheter is in fluid communication through a catheter connection port with the at least one pump, wherein a proximal end of the inner catheter is connected to the catheter connection port to place the inner catheter in fluid communication with the at least one pump, wherein the inner catheter comprises: at least one lumen to transport saline delivered from the at least one pump; at least one electrode positioned within the at least one lumen; at least two positioning elements separated along a length of the inner catheter, wherein a distal positioning element is advanced until the distal end of the distal positioning element contacts fundus of the uterus, and a proximal positioning element is advanced for positioning proximate an internal os of the patient and for creating a partial seal or contact with the internal os; and at least one openings proximate to the distal positioning element of the inner catheter; a controller having at least one processor in data communication with the at least one pump, wherein, upon activating, the controller is configured to: control the delivery of saline into the at least one lumen in the coaxial catheter; control the delivery of an electrical current to the at least one electrode positioned within the at least one lumen of the inner catheter; and control vapor generated from the saline.

Optionally, the inner catheter is used to measure a length of the uterine cavity of the patient. Optionally, the measured length is used to determine an amount of the vapor to be used for ablating.

Optionally, the partial seal is a temperature dependent seal and breaks once the temperature inside the sealed portion of the uterus exceeds 90° C.

Optionally, the partial seal is a pressure dependent seal and breaks once the temperature inside the sealed portion of the uterus exceeds 101° C. and the pressure exceeds 0.5 psi. Optionally, the partial seal is a pressure dependent seal and breaks once the temperature inside the sealed portion of the uterus exceeds 102° C. and the pressure exceeds 1.0 psi. Optionally, the partial seal is a pressure dependent seal and breaks once the temperature inside the sealed portion of the uterus exceeds 103° C. and the pressure exceeds 1.5 psi.

Optionally, the controller controls the vapor to an amount to keep endometrial pressure below at least one of 50 mm Hg and 10% above atmospheric pressure. Optionally, the controller controls the vapor to an amount to keep endometrial pressure below at least one of 30 mm Hg and 10% above atmospheric pressure. Optionally, the controller controls the vapor to an amount to keep endometrial pressure below at least one of 15 mm Hg and 10% above atmospheric pressure.

Optionally, at least one of the inner and the outer catheter comprise a venting element to allow venting of the uterus. Optionally, the venting element comprises grooves.

Optionally, the proximal positioning element comprises at least one opening to allow venting of the uterus.

Optionally, the inner catheter includes a pressure sensor to allow maintaining a pressure of vapor within the uterus to less than 50 mm Hg. Optionally, the inner catheter includes a pressure sensor to allow maintaining a pressure of vapor within the uterus to less than 30 mm Hg. Optionally, the inner catheter includes a pressure sensor to allow maintaining a pressure of vapor within the uterus to less than 15 mm Hg.

Optionally, each positioning element comprises an uncovered wire mesh.

The present specification also discloses a method of ablating a prostatic tissue of a patient, comprising: providing an ablation system comprising: at least one pump; a catheter in fluid communication with the at least one pump, wherein a proximal end of the catheter is connected to the catheter connection port to place the catheter in fluid communication with the at least one pump, wherein the catheter comprises: at least one lumen configured to transport saline delivered from the at least one pump; at least one positioning element on a distal end of the at least one lumen; at least one electrode positioned within the at least one lumen; an outer sheath covering the at least one lumen; a plurality of openings on the outer sheath proximate to a distal end of the catheter; and a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to control the delivery of saline into the at least one lumen in the catheter, wherein the electrode is configured to receive an electrical current and convert said saline into vapor for ablation; inserting the distal end of the catheter into a urethra of said patient; extending the distal end of the catheter into a bladder of said patient; retracting the outer sheath to expose the at least one lumen and the positioning element; expanding the positioning element; extending said thermally conductive elements through said plurality of openings and into said prostatic tissue; and programming said controller to control a delivery of said vapor such that greater than 0% and less than 75% of a circumference of a prostatic tissue or proximate tissue is ablated.

Optionally, the thermally conductive elements comprise needles.

Optionally, said prostatic tissue or proximate tissue is a prostatic urethra.

Optionally, said prostatic tissue or proximate tissue is an ejaculatory duct.

Optionally, said prostatic tissue or proximate tissue is a rectal wall.

Optionally, expanding the positioning element comprises positioning the positioning element proximate the bladder neck.

Optionally, expanding the positioning element comprises positioning the positioning element within the prostatic urethra.

The present specification also discloses a method of ablating an endometrial tissue of a patient, comprising: providing an ablation system comprising: at least one pump; a coaxial catheter for inserting into a vagina of a patient towards the cervix, the coaxial catheter comprising: an outer catheter for advancing to the internal os of the cervix of the patient; an inner catheter for advancing into the uterus of the patient, concentric and slidable within the outer catheter, wherein the inner catheter is in fluid communication through a catheter connection port with the at least one pump, wherein a proximal end of the inner catheter is connected to the catheter connection port to place the inner catheter in fluid communication with the at least one pump, wherein the inner catheter comprises: at least one lumen to transport saline delivered from the at least one pump; at least one electrode positioned within the at least one lumen; at least two positioning elements separated along a length of the inner catheter, wherein a distal positioning element is advanced until the distal end of the distal positioning element contacts a fundus of the uterus, and a proximal positioning element is advanced for positioning proximate an internal os of the patient and for creating a partial seal with the internal os; and a plurality of openings positioned on said inner catheter and between said distal positioning element and said proximal positioning element for the delivery of vapor; a controller having at least one processor in data communication with the at least one pump, wherein, upon activating, the controller is configured to control the delivery of saline into the at least one lumen in the coaxial catheter, and control vapor generated from the saline; inserting the distal end of the catheter until the distal end of the distal positioning element contacts fundus of the uterus and a proximal positioning element is advanced for positioning proximate an internal os of the patient; expanding the distal positioning element; expanding the proximal positioning element for creating a partial seal within the internal os; and programming said controller to control a delivery of said vapor for ablating the endometrial tissue.

Optionally, the distal positioning element and the proximal positioning element each have a funnel shape.

The present specification also discloses a method of ablating a median lobe of a prostate in a patient with median lobe hyperplasia, the method comprising: passing a catheter with at least one needle into the patient's spongy urethra and through a prostatic urethra such that a distal end of the catheter is positioned within the patient's bladder; extending the at least one needle from the distal end of the catheter and passing the needle through a bladder or bladder neck wall and into the median lobe; delivering ablative agent through the at least one needle and into the median lobe to ablate prostatic tissue; and using a controller to control a flow of ablative agent to maintain a pressure in the bladder and median lobe below 5 atm.

Optionally, the catheter further comprises at least one positioning element and the method further comprises, prior to extending the at least one needle, deploying the at least one positioning element to position the catheter in the bladder and stabilize the at least one needle.

The present specification also discloses a method of ablating a median lobe of a prostate in a patient with median lobe hyperplasia, the method comprising: providing an ablation system comprising: at least one pump; a catheter in fluid communication with the at least one pump, wherein a proximal end of the catheter is connected to the catheter connection port to place the catheter in fluid communication with the at least one pump, wherein the catheter comprises: at least one lumen configured to transport saline delivered from the at least one pump; at least one electrode positioned within the at least one lumen; an outer sheath covering the at least one lumen; a plurality of openings on the outer sheath proximate to a distal end of the catheter; and a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to control the delivery of saline into the at least one lumen in the catheter, wherein the electrode is configured to receive an electrical current and convert said saline into vapor for ablation; inserting the catheter into the patient's spongy urethra and through a prostatic urethra such that a distal end of the catheter is positioned within the patient's bladder; extending the plurality of thermally conductive elements from the distal end of the catheter, through a bladder wall and into the median lobe; delivering ablative agent through the plurality of thermally conductive elements and into the median lobe to ablate prostatic tissue; and programming the controller to control to control a flow of ablative agent to maintain a pressure in the bladder and median lobe below 5 atm.

Optionally, the catheter further comprises at least one positioning element and the method further comprises, prior to extending the extending the plurality of thermally conductive elements, deploying the at least one positioning element to position the catheter in the bladder and stabilize the plurality of thermally conductive elements.

The present specification also discloses a method for ablating at least one of a target area within or proximate a urinary bladder of a patient, the method comprising: providing an ablation system comprising: at least one pump; a catheter in fluid communication with the at least one pump, wherein a proximal end of the catheter is connected to the catheter connection port to place the catheter in fluid communication with the at least one pump, wherein the catheter comprises: at least one lumen configured to transport saline delivered from the at least one pump; at least one electrode positioned within the at least one lumen; a plurality of openings proximate to a distal end of the catheter; and a plurality of thermally conductive elements extendable and retractable through the plurality of openings, wherein the plurality of thermally conductive elements are hollow and wherein each of the plurality of thermally conductive elements includes a port to allow delivery of vapor; and a controller having at least one processor in data communication with the at least one pump, wherein, upon activating, the controller is configured to control the delivery of saline into the at least one lumen in the catheter, wherein the electrode is configured to receive an electrical current and convert said saline into vapor for ablation; draining fluid within the urinary bladder from vicinity of the target area; inserting the catheter into a ureter of the patient; extending the thermally conductive elements through the plurality of openings and into or proximate the target area; and programming the controller to control a delivery of the vapor such that the target area is ablated.

Optionally, the target area is at least one of a tissue, a tumor, or a nerve. Optionally, target area is a tissue inside the urinary bladder. Optionally, the target area is within an adventitial space beneath a trigone of the patient. Optionally, the target area is within one of a bladder neck, an internal urinary sphincter (IUS), and nerves supplying the IUS and bladder neck, of the patient.

Optionally, draining the fluid comprises draining urine from the urinary bladder.

Optionally, draining the fluid further comprises performing at least one of: removing urine from the urinary bladder; insufflating air into the urinary bladder; and positioning the patient so as to have the target area positioned away from a dependent part of the urinary bladder, allowing for urine to drain away from the urinary bladder.

Optionally, the thermally conductive elements comprise needles.

Optionally, the method further comprises applying a positioning element proximate the target area and enclosing at least a portion of the target area.

Optionally, the method further comprises maintaining a pressure within the urinary bladder at below 5 atm.

The present specification also discloses a method for ablating at least one of a target area within or proximate a urinary bladder of a patient, the method comprising: providing an ablation system comprising: at least one pump; a coaxial catheter for inserting into a ureter the patient, the coaxial catheter comprising: an outer catheter for advancing to the ureter of the patient; an inner catheter for advancing into the ureter of the patient, concentric and slidable within the outer catheter, wherein the inner catheter is in fluid communication through a catheter connection port with the at least one pump, wherein a proximal end of the inner catheter is connected to the catheter connection port to place the inner catheter in fluid communication with the at least one pump, wherein the inner catheter comprises: at least one lumen to transport saline delivered from the at least one pump; at least one electrode positioned within the at least one lumen; at least one positioning element along a length of the inner catheter, wherein the at least one positioning element is advanced until the distal end of the positioning element encloses the target area; and at least one opening proximate to the positioning element of the inner catheter; a controller having at least one processor in data communication with the at least one pump, wherein, upon activating, the controller is configured to: control the delivery of saline into the at least one lumen in the coaxial catheter; control the delivery of an electrical current to the at least one electrode positioned within the at least one lumen of the inner catheter; and control vapor generated from the saline; draining fluid within the urinary bladder from vicinity of the target area; inserting the coaxial catheter into a ureter of the patient; applying the positioning element proximate the target area enclosing at least a portion of the target area; and programming the controller to control a delivery of the vapor such that the target area is ablated.

Optionally, the target area is at least one of a tissue, a tumor, or a nerve. Optionally, the target area is a tissue inside the urinary bladder. Optionally, draining the fluid comprises draining urine from the urinary bladder.

Optionally, draining the fluid further comprises performing at least one of: removing urine from the urinary bladder; insufflating air into the urinary bladder; positioning the patient so as to have the target area positioned away from a dependent part of the urinary bladder, allowing for urine to drain away from the urinary bladder.

Optionally, the method further comprises maintaining a pressure within the urinary bladder at below 5 atm.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings, wherein:

FIG. 1A illustrates an ablation system, in accordance with embodiments of the present specification;

FIG. 1B is a transverse cross-section view of a flexible heating chamber, in accordance with an embodiment of the present specification;

FIG. 1C illustrates transverse and longitudinal cross-section views of first and second arrays of electrodes of a flexible heating chamber, in accordance with an embodiment of the present specification;

FIG. 1D is a transverse cross-section view of the heating chamber of FIG. 1B, including assembled first and second arrays of electrodes, in accordance with an embodiment of the present specification;

FIG. 1E is a longitudinal cross-section view of the heating chamber of FIG. 1B, including assembled first and second arrays of electrodes, in accordance with an embodiment of the present specification;

FIG. 1F is a first longitudinal view of two heating chambers of FIG. 1B arranged in series in a catheter tip, in accordance with an embodiment of the present specification;

FIG. 1G is a second longitudinal view of two heating chambers of FIG. 1B arranged in series in a catheter tip, in accordance with an embodiment of the present specification;

FIG. 1H illustrates a multiple lumen balloon catheter incorporating one heating chamber of FIG. 1B, in accordance with an embodiment of the present specification;

FIG. 1I illustrates a multiple lumen balloon catheter incorporating two heating chambers of FIG. 1B, in accordance with an embodiment of the present specification;

FIG. 1J illustrates a catheter with proximal and distal positioning elements and an electrode heating chamber, in accordance with embodiments of the present specification;

FIG. 1K illustrates an ablation system for the ablation of prostatic tissue, in accordance with embodiments of the present specification;

FIG. 1L illustrates a catheter for use in the ablation of prostatic tissue, in accordance with embodiments of the present specification;

FIG. 1M illustrates a system for use in the ablation of prostatic tissue, in accordance with another embodiment of the present specification;

FIG. 1N illustrates an ablation system for the ablation of endometrial tissue, in accordance with embodiments of the present specification;

FIG. 1O illustrates a catheter for use in the ablation of endometrial tissue, in accordance with embodiments of the present specification;

FIG. 1P illustrates a system for use in the ablation of endometrial tissue, in accordance with another embodiment of the present specification;

FIG. 1Q illustrates a controller for use with an ablation system, in accordance with an embodiment of the present specification;

FIG. 1R illustrates a system for use in the ablation of prostatic tissue, in accordance with another embodiment of the present specification;

FIG. 1S illustrates a needle attachment component of a system for use in the ablation of prostatic tissue, in accordance with some embodiments of the present specification;

FIG. 1T illustrates a needle chamber of a system for use in the ablation of prostatic tissue, in accordance with some embodiments of the present specification;

FIG. 1U illustrates another needle chamber of a system for use in the ablation of tissue, in accordance with some embodiments of the present specification;

FIG. 2A illustrates a single lumen double balloon catheter comprising an in-line heating element, in accordance with an embodiment of the present specification;

FIG. 2B illustrates a coaxial lumen double balloon catheter comprising an in-line heating element, in accordance with an embodiment of the present specification;

FIG. 3A illustrates a typical anatomy of a prostatic region for descriptive purposes;

FIG. 3B illustrates an exemplary transparent view of prostate anatomy, highlighting a peripheral zone, in addition to other zones in the periphery of the prostate;

FIG. 3C illustrates an oblique top-down transparent view of a prostate, showing the various zones and prostatic urethra;

FIG. 4A is an illustration of a water cooled catheter, in accordance with another embodiment of the present specification;

FIG. 4B is a cross-section view of a tip section of the water cooled catheter of FIG. 4A;

FIG. 4C illustrates embodiments of a distal end of a catheter for use with the system of FIG. 1M;

FIG. 4D illustrates other embodiments of a distal end of a catheter for use with the system of FIG. 1M;

FIG. 4E illustrates an embodiment of a slit flap used to cover openings of FIGS. 4C and 4D, in accordance with some embodiments of the present specification;

FIG. 4F illustrates an embodiment of a positioning element to be positioned at a distal end of an ablation catheter, to position the ablation catheter in the prostatic urethra, in accordance with the present specification;

FIG. 4G illustrates a distal end of an ablation catheter advanced through a prostatic urethra, in accordance with an exemplary embodiment of the present specification;

FIG. 4H illustrates a distal end of an ablation catheter advanced into a bladder, in accordance with an exemplary embodiment of the present specification;

FIG. 4I illustrates a distal end of an ablation catheter advanced further into a bladder, in accordance with an exemplary embodiment of the present specification;

FIG. 4J illustrates a positioning element expanded at a distal end of an ablation catheter and being retracted to position the positioning element proximate a bladder neck or urethra, in accordance with an exemplary embodiment of the present specification;

FIG. 4K illustrates at least one needle extended from a distal end of an ablation catheter and into prostatic tissue, in accordance with an exemplary embodiment of the present specification;

FIG. 4L illustrates an ablative agent being delivered through one or more needles and into prostatic tissue, in accordance with an exemplary embodiment of the present specification;

FIG. 4M illustrates an ablation catheter advanced into a prostatic urethra and having a positioning element at a position proximal to needles positioned at the distal end of the catheter, in accordance with an alternative embodiment of the present specification;

FIG. 4N illustrates needles at a distal end of an ablation catheter deployed into prostatic tissue, in accordance with the alternative embodiment of the present specification;

FIG. 4O is a flow chart illustrating the steps involved in using an ablation catheter to ablate a prostate of a patient, in accordance with embodiments of the present specification;

FIG. 5A illustrates prostate ablation being performed on an enlarged prostrate in a male urinary system by using the device, in accordance with an embodiment of the present specification;

FIG. 5B is an illustration of transurethral prostate ablation being performed on an enlarged prostrate in a male urinary system using an ablation device, in accordance with one embodiment of the present specification;

FIG. 5C is an illustration of transurethral prostate ablation being performed on an enlarged prostrate in a male urinary system using an ablation device, in accordance with another embodiment of the present specification;

FIG. 5D is a flow chart listing the steps involved in a transurethral enlarged prostate ablation process using an ablation catheter, in accordance with one embodiment of the present specification;

FIG. 5E is an illustration of transrectal prostate ablation being performed on an enlarged prostrate in a male urinary system using an ablation device, in accordance with one embodiment of the present specification;

FIG. 5F is an illustration of transrectal prostate ablation being performed on an enlarged prostrate in a male urinary system using a coaxial ablation device having a positioning element, in accordance with another embodiment of the present specification;

FIG. 5G is a close-up illustration of the distal end of the catheter and needle tip of the ablation device;

FIG. 5H is a flow chart listing the steps involved in a transrectal enlarged prostate ablation process using an ablation catheter, in accordance with one embodiment of the present specification;

FIG. 6A is an illustration of an ablation catheter, in accordance with an embodiment of the present specification;

FIG. 6B is a cross-section view of a tip of the ablation catheter of FIG. 6A;

FIG. 6C is an illustration of transurethral prostate ablation being performed using the ablation catheter of FIG. 6A, in accordance with an embodiment;

FIG. 6D is a flow chart listing the steps involved in a transurethral enlarged prostate ablation process, in accordance with an embodiment;

FIG. 7A is an illustration of an ablation catheter, in accordance with another embodiment of the present specification;

FIG. 7B is a cross-section view of a tip of the ablation catheter of FIG. 7A;

FIG. 7C is an illustration of transurethral prostate ablation being performed using the ablation catheter of FIG. 7A, in accordance with an embodiment;

FIG. 7D is a flow chart listing the steps involved in a transurethral enlarged prostate ablation process, in accordance with an embodiment.

FIG. 8A is an illustration of one embodiment of a positioning element of an ablation catheter, depicting a plurality of thermally conducting elements attached thereto;

FIG. 8B is an illustration of one embodiment of a positioning element of an ablation catheter, depicting a plurality of hollow thermally conducting elements attached thereto;

FIG. 9 is a flowchart illustrating one embodiment of a method of ablation of a tissue using a needle catheter device;

FIG. 10 is a flowchart illustrating a method of ablation of a submucosal tissue using a needle catheter device, according to one embodiment of the present specification;

FIG. 11A is an exemplary illustration of shape changing needles, according to one embodiment of the present specification;

FIG. 11B illustrates different embodiments of needles, in accordance with the present specification;

FIG. 11C illustrates an exemplary process of delivery of an ablative agent from hollow openings at the edges of a pair of needles, such as double needles of FIG. 11B, in accordance with some embodiments of the present specification;

FIG. 11D illustrates exemplary depths of needles of different curvatures, in accordance with some embodiments of the present specification;

FIG. 11E illustrates exemplary depths of needles, relative to needles of FIG. 11D, in accordance with some embodiments of the present specification;

FIG. 11F illustrates exemplary lengths of needles of FIG. 11E, extending in a straight line from the port to the farthest distance reached by the body of the needles, in accordance with some embodiments of the present specification;

FIG. 11G illustrates different views of a single needle assembly extending from a port, in accordance with some embodiments of the present specification;

FIG. 11H illustrates one or more holes at the sharp edge of the needle in another horizontal view of the needle, in accordance with some embodiments of the present specification;

FIG. 11I illustrates different views of a double needle assembly extending from a port, in accordance with some embodiments of the present specification;

FIG. 11J illustrates different views of another double needle assembly extending from a port, in accordance with some embodiments of the present specification;

FIG. 11K illustrates an insulation on a single needle configuration and a double needle configuration, in accordance with some embodiments of the present specification;

FIG. 11L illustrates a single needle configuration with insulation, positioned inside a prostatic tissue in accordance with some embodiments of the present specification;

FIG. 11M illustrates a single needle configuration with insulation, positioned inside a uterine fibroid in accordance with some embodiments of the present specification;

FIG. 11N illustrates a double needle configuration where the two needles are inserted into separate prostate lobes, in accordance with some embodiments of the present specification;

FIG. 11O illustrates an exemplary embodiment of a steerable catheter shaft in accordance with some embodiments of the present specification;

FIG. 11P illustrates a needle with an open tip, in accordance with some embodiments of the present specification;

FIG. 11Q illustrates an alternative embodiment of a needle with an occluded tip and comprising holes or openings along an uninsulated length of the needle, in accordance with the present specification;

FIG. 12 is an illustration of transurethral prostate ablation being performed using an ablation device, in accordance with one embodiment of the present specification;

FIG. 13A is an illustration of one embodiment of a positioning element of an ablation catheter with needles attached to the catheter body;

FIG. 13B is an illustration of another embodiment of positioning elements for an ablation catheter;

FIG. 13C illustrates a cross section of the distal tip of a catheter, in accordance with an embodiment of the present specification;

FIG. 14 illustrates one embodiment of a handle mechanism that may be used for deployment and retrieval of ablation needles at variable depths of insertion;

FIG. 15A is a flowchart illustrating a method of ablation of prostatic tissue in accordance with one embodiment of the present specification;

FIG. 15B is a flowchart illustrating a method of ablation of prostatic tissue in accordance with another embodiment of the present specification;

FIG. 15C illustrates a compressed catheter with an expandable element being advanced into a prostatic urethra, in accordance with an embodiment of the present specification;

FIG. 15D illustrates an expanded expandable element of a catheter, pressing on urethral walls which presses on a prostate, and ablative agent being delivered through from inside the expandable member and into prostatic tissue, in accordance with an embodiment of the present specification;

FIG. 15E illustrates a widened prostatic urethra, after removing an expandable catheter, in accordance with an embodiment of the present specification;

FIG. 15F illustrates an expanded expandable element of a catheter and an exemplary use of one or more needles to allow delivery of an ablative agent, such as steam or vapor through a hollow exit at the edge of the needle, in accordance with some embodiments of the present specification;

FIG. 15G illustrates an ablation catheter used to ablate prostatic tissue of a patient with median lobe hyperplasia via a trans-cystic approach, in accordance with one embodiment of the present specification;

FIG. 15H illustrates an ablation catheter used to ablate prostatic tissue of a patient with median lobe hyperplasia via a trans-cystic approach, in accordance with another embodiment of the present specification;

FIG. 15I is a flowchart listing the steps in one method of using an ablation catheter to ablate prostatic tissue of a patient with median lobe hyperplasia via a trans-cystic approach, in accordance with one embodiment of the present specification;

FIG. 16A is an International Prostate Symptom Score (IPSS) Questionnaire;

FIG. 16B is a Benign Prostatic Hypertrophy Impact Index Questionnaire (BPHIIQ);

FIG. 17A illustrates a typical anatomy of a uterus and uterine tubes of a human female;

FIG. 17B illustrates the location of the uterus and surrounding anatomical structures within a female body;

FIG. 18A illustrates an exemplary ablation catheter arrangement for ablating the uterus, in accordance with some embodiments of the present specification;

FIG. 18B illustrates an exemplary embodiment of grooves configured in an inner catheter of FIG. 18A, in accordance with some embodiments of the present specification;

FIG. 18C is a flowchart of a method of using the catheter of FIG. 18A for ablation of endometrial tissue, in accordance with embodiments of the present specification;

FIG. 18D illustrates a catheter for endometrial ablation, in accordance with other embodiments of the present specification;

FIG. 18E illustrates a catheter having an expanded distal positioning element advanced through a cervical canal and into a uterus, in accordance with embodiments of the present specification;

FIG. 18F illustrates catheter having an expanded distal positioning element and an expanded proximal positioning element, further advanced into a uterus, in accordance with embodiments of the present specification;

FIG. 18G illustrates vapor delivered into a uterus through a plurality of ports on a catheter body and positioned between proximal and distal positioning elements, in accordance with embodiments of the present specification;

FIG. 18H is a flow chart illustrating the steps involved in using an ablation catheter to ablate an endometrium of a patient, in accordance with embodiments of the present specification;

FIG. 18I illustrates a side view, cross-section side view, and distal end front-on view of an endometrial ablation catheter, in accordance with some embodiments of the present specification;

FIG. 18J illustrates a perspective side view of the catheter of FIG. 18I with the stent extending over the inner catheter, and extending out from the outer catheter, in accordance with some embodiments of the present specification;

FIG. 18K illustrates a cross-section side view, a perspective side view, and a distal end front-on view of a braided stent, in accordance with some embodiments of the present specification;

FIG. 18L illustrates a side perspective view of a distal end of an inner catheter, in accordance with some embodiments of the present specification;

FIG. 18M illustrates a side front perspective view of a distal end of an inner catheter, in accordance with some embodiments of the present specification;

FIG. 18N illustrates a top perspective view of a distal end of an inner catheter, in accordance with some embodiments of the present specification;

FIG. 18O illustrates different views of a double-positioning element catheter with an atraumatic olive tip end, in accordance with another embodiment of the present specification;

FIG. 18P illustrates distal ends of ablation catheters having distal positioning elements and a plurality of ports along a length of the catheter shaft, in accordance with some embodiments of the present specification;

FIG. 18Q illustrates distal ends of ablation catheters having distal olive tips, positioning elements, and a plurality of ports along a length of the catheter shaft, in accordance with some embodiments of the present specification;

FIG. 18R illustrates a side view a distal end of an ablation catheter having a distal olive tip, two positioning elements, and a plurality of ports along a length of the catheter shaft, in accordance with some embodiments of the present specification;

FIG. 18S illustrates a rear perspective view of the catheter of FIG. 18R;

FIG. 18T illustrates a distal end of an ablation catheter with a half-circle openings at the distal end and a distal positioning element, in accordance with some embodiments of the present specification;

FIG. 18U illustrates a distal end of an ablation catheter having a spherical shaped distal positioning element and a cover extending over the entirety or a portion of the positioning element, in accordance with an exemplary embodiment of the present specification;

FIG. 18V illustrates a distal end of an ablation catheter having a spherical shaped distal positioning element, in accordance with another exemplary embodiment of the present specification;

FIG. 18W illustrates a distal end of an ablation catheter having a conical shaped distal positioning element, in accordance with yet another exemplary embodiment of the present specification;

FIG. 18X illustrates an atraumatic soft tip of a catheter shaft that is used for insertion into a cervix, in accordance with some embodiments of the present specification;

FIG. 19A illustrates a configuration of a disc for use with the catheter arrangement of FIG. 18A, in accordance with one embodiment of the present specification;

FIG. 19B illustrates a configuration of a disc for use with the catheter arrangement of FIG. 18A, in accordance with another embodiment of the present specification;

FIG. 19C illustrates multiple configurations of discs for use with the catheter arrangement of FIG. 18A, in accordance with yet other embodiments of the present specification;

FIG. 19D illustrates an assembly of catheter with a handle, and a cervical collar, in accordance with some embodiments of the present specification;

FIG. 19E illustrates a position of the cervical collar as it sits at an external os, outside the uterus and cervix, before deployment of the catheter, in accordance with some embodiments of the present specification;

FIG. 19F illustrates an exemplary position of hands to hold the catheter for deploying the proximal positioning element, in accordance with some embodiments of the present specification;

FIG. 19G illustrates expanding of the proximal positioning element while the user pushes the handle of the catheter to extend the inner catheter within the uterus, in accordance with some embodiments of the present specification;

FIG. 19H illustrates deploying of the distal positioning element, which may be uncoated or selectively coated with silicone, in accordance with some embodiments of the present specification;

FIG. 19I illustrates turn of a dial to further retract first positioning element to partially seal cervical os, so as to isolate the uterus, in accordance with some embodiments of the present specification;

FIG. 19J illustrates a distal end of an ablation catheter having two positioning elements and a plurality of ports along a length of the catheter shaft, in accordance with some embodiments of the present specification;

FIG. 19K illustrates a distal end of an ablation catheter having two positioning elements a distal olive tip, and a plurality of ports along a length of the catheter shaft, in accordance with some embodiments of the present specification;

FIG. 19L illustrates a connector for connecting a distal positioning element to a distal end of an ablation catheter, in accordance with some embodiments of the present specification;

FIG. 19M illustrates another connector for connecting a distal positioning element to a distal end of an ablation catheter, in accordance with other embodiments of the present specification;

FIG. 19N illustrates a connector for connecting a proximal positioning element to a distal end of an ablation catheter, in accordance with some embodiments of the present specification;

FIG. 19O illustrates another connector for connecting a proximal positioning element to a distal end of an ablation catheter, in accordance with other embodiments of the present specification;

FIG. 19P illustrates a shaft of an ablation catheter depicting a plurality of ports, in accordance with some embodiments of the present specification;

FIG. 20A illustrates endometrial ablation being performed in a female uterus by using an ablation device, in accordance with an embodiment of the present specification;

FIG. 20B is an illustration of a coaxial catheter used in endometrial tissue ablation, in accordance with one embodiment of the present specification;

FIG. 20C is a flow chart listing the steps involved in an endometrial tissue ablation process using a coaxial ablation catheter, in accordance with one embodiment of the present specification;

FIG. 20D is an illustration of a bifurcating coaxial catheter used in endometrial tissue ablation, in accordance with one embodiment of the present specification;

FIG. 20E is a flowchart listing the steps of a method of using the ablation catheter of FIG. 20D to ablate endometrial tissue, in accordance with one embodiment of the present specification;

FIG. 20F is an illustration of a bifurcating coaxial catheter with expandable elements used in endometrial tissue ablation, in accordance with one embodiment of the present specification;

FIG. 20G is an illustration of the catheter of FIG. 20F inserted into a patient's uterine cavity for endometrial tissue ablation;

FIG. 20H is a flowchart listing the steps of a method of using the ablation catheter of FIG. 20F to ablate endometrial tissue, in accordance with one embodiment of the present specification;

FIG. 20I is an illustration of a bifurcating coaxial catheter used in endometrial tissue ablation, in accordance with another embodiment of the present specification;

FIG. 20J is an illustration of a bifurcating coaxial catheter used in endometrial tissue ablation, in accordance with yet another embodiment of the present specification;

FIG. 20K is an illustration of a water cooled catheter used in endometrial tissue ablation, in accordance with one embodiment of the present specification;

FIG. 20L is an illustration of a water cooled catheter used in endometrial tissue ablation and positioned in a uterus of a patient, in accordance with another embodiment of the present specification;

FIG. 20M is an illustration of a water cooled catheter used in cervical ablation, in accordance with one embodiment of the present specification;

FIG. 20N is an illustration of the catheter of FIG. 20M positioned in a cervix of a patient;

FIG. 20O is a flowchart listing the steps involved in cervical ablation performed using the catheter of FIG. 20M;

FIG. 21A is a flowchart illustrating a method of ablation of endometrial tissue in accordance with one embodiment of the present specification;

FIG. 21B is a flowchart illustrating a method of ablating a uterine fibroid in accordance with one embodiment of the present specification;

FIG. 21C illustrates exemplary configurations for a distal end of a catheter for endometrial ablation, in accordance with some embodiments of the present specification;

FIG. 21D illustrates a distal end of a catheter for endometrial ablation with a positioning element attached thereto, in accordance with some embodiments of the present specification;

FIG. 21E illustrates a deployed configuration and a compressed configuration of a distal end of a catheter for endometrial ablation, in accordance with some embodiments of the present specification;

FIG. 21F illustrates an embodiment of a catheter for endometrial ablation with a positioning element in its deployed configuration, in accordance with some embodiments of the present specification;

FIG. 21G illustrates different three-dimensional views of the positioning element of FIG. 21F in its deployed configuration, and a connector, in accordance with some embodiments of the present specification;

FIG. 21H illustrates different views of another embodiment of a positioning element in its deployed configuration, in accordance with some embodiments of the present specification;

FIG. 21I illustrates a coaxial, telescopic movement of an inner catheter within an outer sheath when a positioning element is deployed to the fully expanded configuration and compressed, in accordance with some embodiments of the present specification;

FIG. 21J illustrates a system for use in the ablation of endometrial tissue, in accordance with some embodiments of the present specification;

FIG. 21K is a flow chart illustrating the steps involved in using an ablation catheter to ablate an endometrium of a patient, in accordance with embodiments of the present specification;

FIG. 22A illustrates different stages of cancer of a bladder, as known in the medical field;

FIG. 22B illustrates a system for use in the ablation of bladder tissue, in accordance with an embodiment of the present specification;

FIG. 23 illustrates an exemplary catheter for insertion into a urinary bladder for ablating bladder tissue, in accordance with some embodiments of the present specification;

FIG. 24A illustrates a front end view of a positioning element, in accordance with some embodiments of the present specification;

FIG. 24B illustrates a side view of a distal end of an ablation catheter and positioning element of FIG. 24A;

FIG. 24C illustrates a front side perspective view of the distal end of an ablation catheter and positioning element of FIG. 24B;

FIG. 25A illustrates a close-up view of a connection between a positioning element and a distal end of an ablation catheter, in accordance with some embodiments of the present specification;

FIG. 25B illustrates a side view of the positioning element attached to a distal end of an ablation catheter of FIG. 25A;

FIG. 25C illustrates different types of configurations of positioning elements which may be used in accordance with the various ablation catheters of the embodiments of the present specification;

FIG. 26A illustrates positioning of a needle ablation catheter for delivering vapor to selectively ablate nerve-rich layers of deep detrusor and adventitial space beneath trigone, in accordance with embodiments of the present specification;

FIG. 26B illustrates positioning of a needle ablation device for delivering vapor to selectively ablate the bladder neck, internal urinary sphincter (IUS), and nerves supplying the IUS and bladder neck, in accordance with embodiments of the present specification;

FIG. 27A illustrates different views of a coaxial needle that may be used for ablation for treatment of OAB, in accordance with some embodiments of the present specification;

FIG. 27B illustrates the distal ends of coaxial needles comprising inner and outer tubes with lumens, in accordance with some embodiments of the present specification;

FIG. 28 is a flow chart illustrating an exemplary process of ablating the urinary bladder and/or its peripheral areas, in accordance with some embodiments of the present specification;

FIG. 29 illustrates a system for use in the ablation and imaging of prostatic tissue, in accordance with an embodiment of the present specification;

FIG. 30 illustrates a system for use in the ablation and imaging of endometrial tissue, in accordance with an embodiment of the present specification;

FIG. 31 illustrates a system for use in the ablation and imaging of bladder tissue, in accordance with an embodiment of the present specification;

FIG. 32 illustrates various components of an optical/viewing system for direct visualization of ablation in accordance with the embodiments of the present specification;

FIG. 33 illustrates components of a distal end of an ablation system that may be used in treatment of benign prostatic hyperplasia (BPH), and abnormal uterine bleeding (AUB), for use in accordance with the embodiments of the present specification;

FIG. 34 illustrates an image of a distal end of an ablation catheter viewed on a display device, in accordance with some embodiments of the present specification;

FIG. 35A depicts a cross-sectional view of an embodiment of a combination catheter comprising lumens for an optical/electrical catheter alongside a lumen for an ablation catheter, in accordance with some embodiments of the present specification;

FIG. 35B depicts a cross-sectional view of another embodiment of a combination catheter comprising lumens for an optical/electrical catheter alongside a lumen for an ablation catheter, in accordance with some embodiments of the present specification;

FIG. 35C depicts a cross-sectional view of yet another embodiment of a combination catheter comprising a lumen for an optical/electrical catheter alongside a lumen for an ablation catheter, in accordance with some embodiments of the present specification;

FIG. 36A illustrates an embodiment of a handle mechanism that may be used for deployment and retrieval of ablation needles at variable depths of insertion;

FIG. 36B illustrates another embodiment of a handle mechanism that may be used for deployment and retrieval of ablation needles at variable depths of insertion;

FIG. 36C illustrates another embodiment of a handle mechanism that may be used for deployment and retrieval of ablation needles at variable depths of insertion;

FIG. 36D illustrates another embodiment of a handle mechanism that may be used for deployment and retrieval of ablation needles at variable depths of insertion;

FIG. 36E illustrates another embodiment of a handle mechanism that may be used for deployment and retrieval of ablation needles at variable depths of insertion;

FIG. 36F illustrates another embodiment of a handle mechanism that may be used for deployment and retrieval of ablation needles at variable depths of insertion;

FIG. 36G illustrates another embodiment of a handle mechanism that may be used for deployment and retrieval of ablation needles at variable depths of insertion;

FIG. 36H illustrates another embodiment of a handle mechanism that may be used for deployment and retrieval of ablation needles at variable depths of insertion;

FIG. 36I illustrates another embodiment of a handle mechanism that may be used for deployment and retrieval of ablation needles at variable depths of insertion;

FIG. 36J illustrates another embodiment of a handle mechanism that may be used for deployment and retrieval of ablation needles at variable depths of insertion;

FIG. 37A illustrates another embodiment of a handle mechanism that may be used for deployment and retrieval of ablation needles at variable depths of insertion;

FIG. 37B illustrates another embodiment of a handle mechanism that may be used for deployment and retrieval of ablation needles at variable depths of insertion;

FIG. 37C illustrates another embodiment of a handle mechanism that may be used for deployment and retrieval of ablation needles at variable depths of insertion;

FIG. 37D illustrates another embodiment of a handle mechanism that may be used for deployment and retrieval of ablation needles at variable depths of insertion;

FIG. 37E illustrates another embodiment of a handle mechanism that may be used for deployment and retrieval of ablation needles at variable depths of insertion;

FIG. 37F illustrates another embodiment of a handle mechanism that may be used for deployment and retrieval of ablation needles at variable depths of insertion;

FIG. 38 illustrates another embodiment of a handle mechanism that may be used for deployment and retrieval of ablation needles at variable depths of insertion;

FIG. 39A illustrates another embodiment of a handle mechanism that may be used for deployment and retrieval of ablation needles at variable depths of insertion;

FIG. 39B illustrates another embodiment of a handle mechanism that may be used for deployment and retrieval of ablation needles at variable depths of insertion;

FIG. 39C illustrates another embodiment of a handle mechanism that may be used for deployment and retrieval of ablation needles at variable depths of insertion;

FIG. 39D illustrates another embodiment of a handle mechanism that may be used for deployment and retrieval of ablation needles at variable depths of insertion;

FIG. 40A illustrates another embodiment of a handle mechanism that may be used for deployment and retrieval of ablation needles at variable depths of insertion;

FIG. 40B illustrates another embodiment of a handle mechanism that may be used for deployment and retrieval of ablation needles at variable depths of insertion;

FIG. 41 illustrates another embodiment of a handle mechanism that may be used for deployment and retrieval of ablation needles at variable depths of insertion;

FIG. 42 illustrates another embodiment of a handle mechanism that may be used for deployment and retrieval of ablation needles at variable depths of insertion;

FIG. 43 illustrates yet another embodiment of a handle mechanism that may be used for deployment and retrieval of ablation needles at variable depths of insertion;

FIG. 44A illustrates an embodiment of a handle to be used with the endometrial ablation systems of the present specification;

FIG. 44B illustrates another embodiment of a handle to be used with the endometrial ablation systems of the present specification;

FIG. 44C illustrates another embodiment of a handle to be used with the endometrial ablation systems of the present specification;

FIG. 44D illustrates another embodiment of a handle to be used with the endometrial ablation systems of the present specification;

FIG. 44E illustrates another embodiment of a handle to be used with the endometrial ablation systems of the present specification;

FIG. 44F illustrates another embodiment of a handle to be used with the endometrial ablation systems of the present specification;

FIG. 44G illustrates another embodiment of a handle to be used with the endometrial ablation systems of the present specification;

FIG. 44H illustrates another embodiment of a handle to be used with the endometrial ablation systems of the present specification;

FIG. 44I illustrates another embodiment of a handle to be used with the endometrial ablation systems of the present specification;

FIG. 44J illustrates rendered views of the embodiment of the handle of FIG. 44I to be used with the endometrial ablation systems of the present specification;

FIG. 44K illustrates another embodiment of a handle to be used with the endometrial ablation systems of the present specification;

FIG. 44L illustrates another embodiment of a handle to be used with the endometrial ablation systems of the present specification;

FIG. 44M illustrates rendered views of the embodiment of the handle of FIG. 44L to be used with the endometrial ablation systems of the present specification;

FIG. 44N illustrates another embodiment of a handle to be used with the endometrial ablation systems of the present specification;

FIG. 44O illustrates another embodiment of a handle to be used with the endometrial ablation systems of the present specification;

FIG. 44P illustrates another embodiment of a handle to be used with the endometrial ablation systems of the present specification;

FIG. 44Q illustrates another embodiment of a handle to be used with the endometrial ablation systems of the present specification;

FIG. 44R illustrates rendered views of the embodiment of the handle of FIG. 44Q to be used with the endometrial ablation systems of the present specification;

FIG. 45 illustrates an ablation system using at least one microwave antenna to convert a liquid to a vapor in a heating chamber, in accordance with embodiments of the present specification;

FIG. 46 illustrates a multiple lumen balloon catheter incorporating one heating chamber of FIG. 45, in accordance with an embodiment of the present specification;

FIG. 47 illustrates a multiple lumen balloon catheter incorporating two heating chambers of FIG. 45, in accordance with an embodiment of the present specification;

FIG. 48 illustrates a catheter with proximal and distal positioning elements and a microwave heating chamber, in accordance with embodiments of the present specification;

FIG. 49 illustrates an ablation system with a microwave heating chamber for the ablation of prostatic tissue, in accordance with embodiments of the present specification;

FIG. 50 illustrates a catheter with a microwave heating chamber for use in the ablation of prostatic tissue, in accordance with embodiments of the present specification;

FIG. 51 illustrates a system with a microwave chamber for use in the ablation of prostatic tissue, in accordance with another embodiment of the present specification;

FIG. 52 illustrates a system with a microwave chamber for use in the ablation of prostatic tissue, in accordance with another embodiment of the present specification;

FIG. 53 illustrates an ablation system with a microwave heating chamber for the ablation of endometrial tissue, in accordance with embodiments of the present specification;

FIG. 54 illustrates a catheter with a microwave heating chamber for use in the ablation of endometrial tissue, in accordance with embodiments of the present specification;

FIG. 55 illustrates a system with a microwave heating chamber for use in the ablation of endometrial tissue, in accordance with another embodiment of the present specification; and

FIG. 56 illustrates a system with a microwave heating chamber for use in the ablation of bladder tissue, in accordance with an embodiment of the present specification.

### DETAILED DESCRIPTION

In various embodiments, the ablation devices and catheters described in the present specification are used in conjunction with any one or more of the heating systems described in U.S. patent application Ser. No. 14/594,444, entitled “Method and Apparatus for Tissue Ablation”, filed on Jan. 12, 2015 and issued as U.S. Pat. No. 9,561,068 on Feb. 7, 2017, which is herein incorporated by reference in its entirety. U.S. patent application Ser. No. 15/600,670, entitled “Ablation Catheter with Integrated Cooling” and filed on May 19, 2017; Ser. No. 15/144,768, entitled “Induction-Based Micro-Volume Heating System” filed on May 2, 2016, and issued as U.S. Pat. No. 10,064,697 on Sep. 4, 2018; Ser. No. 14/158,687, entitled “Method and Apparatus for Tissue Ablation”, filed on Jan. 17, 2014, and issued as U.S. Pat. No. 9,561,067 on Feb. 7, 2017; Ser. No. 13/486,980, entitled “Method and Apparatus for Tissue Ablation”, filed on Jun. 1, 2012, and issued as U.S. Pat. No. 9,561,066 on Feb. 7, 2017; and, Ser. No. 12/573,939, entitled “Method and Apparatus for Tissue Ablation” and filed on Oct. 6, 2009, are all herein incorporated by reference in their entirety.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims.

Unless otherwise specified, “a,” “an,” “the,” “one or more,” and “at least one” are used interchangeably and mean one or more than one.

The term “controller” refers to an integrated hardware and software system defined by a plurality of processing elements, such as integrated circuits, application specific integrated circuits, and/or field programmable gate arrays, in data communication with memory elements, such as random access memory or read only memory where one or more processing elements are configured to execute programmatic instructions stored in one or more memory elements.

The term “vapor generation system” refers to any or all of the heater or induction-based approaches to generating steam from water described in this application.

Embodiments of the present specification are useful in the treatment of genitourinary structures, where the term “genitourinary” includes all genital and urinary structures, including, but not limited to, the prostate, uterus, and urinary bladder, and any conditions associated therewith, including, but not limited to, benign prostatic hyperplasia (BPH), prostate cancer, uterine fibroids, abnormal uterine bleeding (AUB), overactive bladder (OAB), strictures, and tumors.

Any and all of the needles and needle configurations disclosed in the specification with regards to a particular embodiment, such as including but not limited to, single needles, double needles, multiple needles and insulated needles, are not exclusive to that embodiment and may be used with any other of the embodiments disclosed in the specification in any of the organ systems for any condition related to the organ system such as and not limited to ablation of prostate, uterus, and bladder.

For purposes of the present specification, ‘completely ablating’ is defined as ablating more than 55% of a surface area or a volume around an anatomical structure.

All of the methods and systems for treating the prostate, uterus, and bladder may include optics or visualization as described in the specification to assist with direct visualization during ablation procedures.

All ablation catheters disclosed in the specification, in some embodiments, include insulation at the location of the electrode(s) to prevent ablation of tissue proximate the location of the electrode within the catheter.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The devices and methods of the present specification can be used to cause controlled focal or circumferential ablation of targeted tissue to varying depth in a manner in which complete healing with re-epithelialization can occur. Additionally, the vapor could be used to treat/ablate benign and malignant tissue growths resulting in destruction, liquefaction and absorption of the ablated tissue. The dose and manner of treatment can be adjusted based on the type of tissue and the depth of ablation needed. The ablation devices can be used for the prostate and endometrial ablation and for the treatment of any mucosal, submucosal or circumferential lesion, such as inflammatory lesions, tumors, polyps and vascular lesions. The ablation devices can also be used for the urinary bladder ablation, and for treating an over-active bladder (OAB). The ablation device can also be used for the treatment of focal or circumferential mucosal or submucosal lesions of the genitourinary tract. The ablation device can be placed endoscopically, radiologically, surgically or under direct visualization. In various embodiments, wireless endoscopes or single fiber endoscopes can be incorporated as a part of the device. In another embodiment, magnetic or stereotactic navigation can be used to navigate the catheter to the desired location. Radio-opaque or sonolucent material can be incorporated into the body of the catheter for radiological localization. Ferromagnetic materials can be incorporated into the catheter to help with magnetic navigation.

Ablative agents such as steam, heated gas or cryogens, such as, but not limited to, liquid nitrogen are inexpensive and readily available and are directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. The microprocessor may use temperature, pressure or other sensing data to control the flow of the ablative agent. In addition, one or more suction ports are provided to suction the ablation agent from the vicinity of the targeted tissue. The targeted segment can be treated by a continuous infusion of the ablative agent or via cycles of infusion and removal of the ablative agent as determined and controlled by the microprocessor.

It should be appreciated that the devices and embodiments described herein are implemented in concert with a controller that comprises a microprocessor executing control instructions. The controller can be in the form of any computing device, including desktop, laptop, and mobile device, and can communicate control signals to the ablation devices in wired or wireless form.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

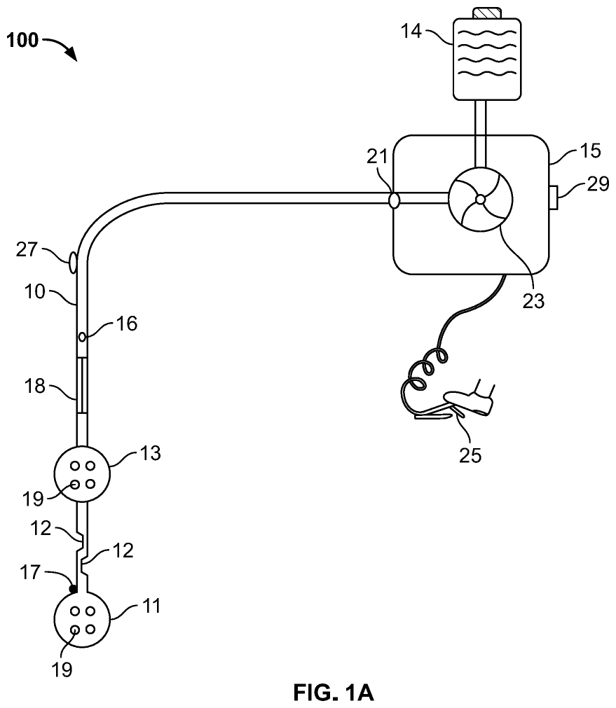

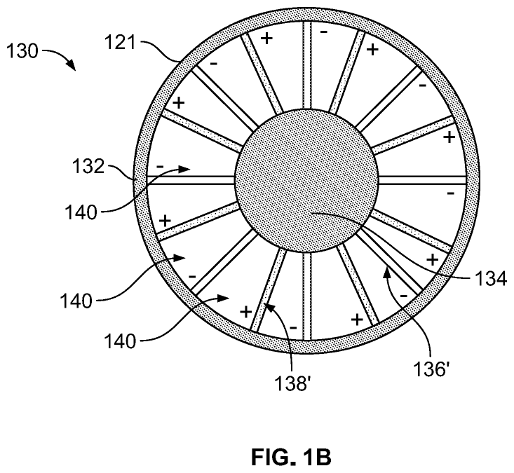

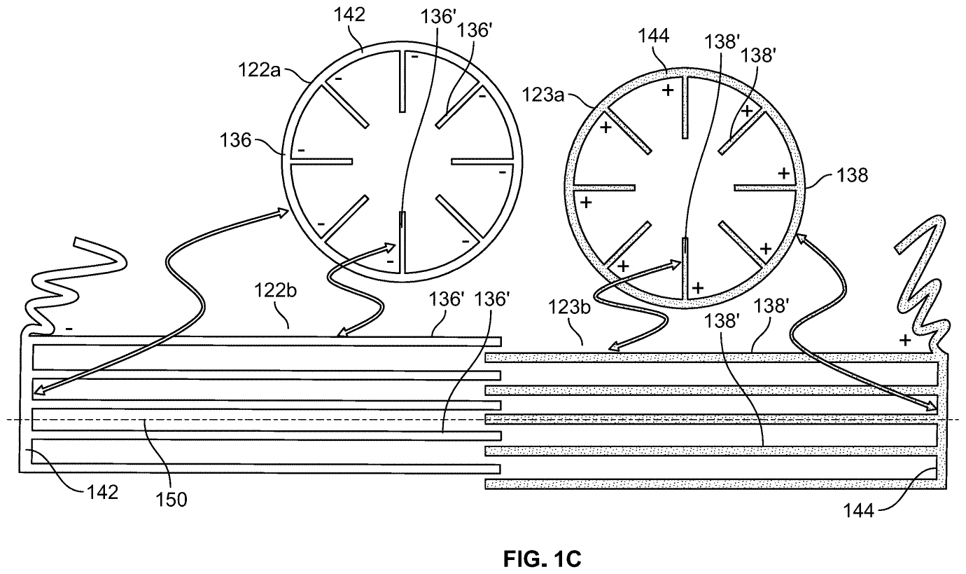

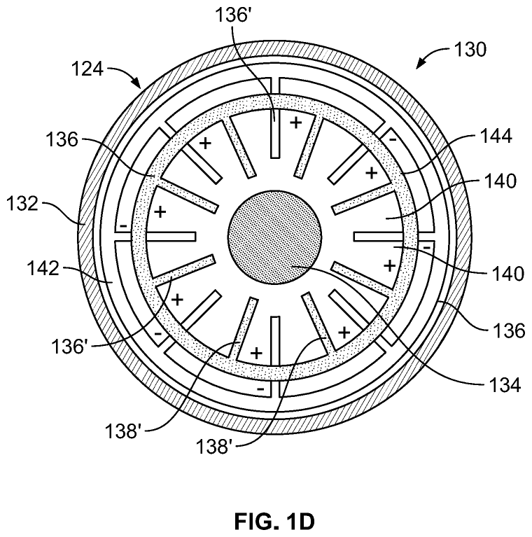

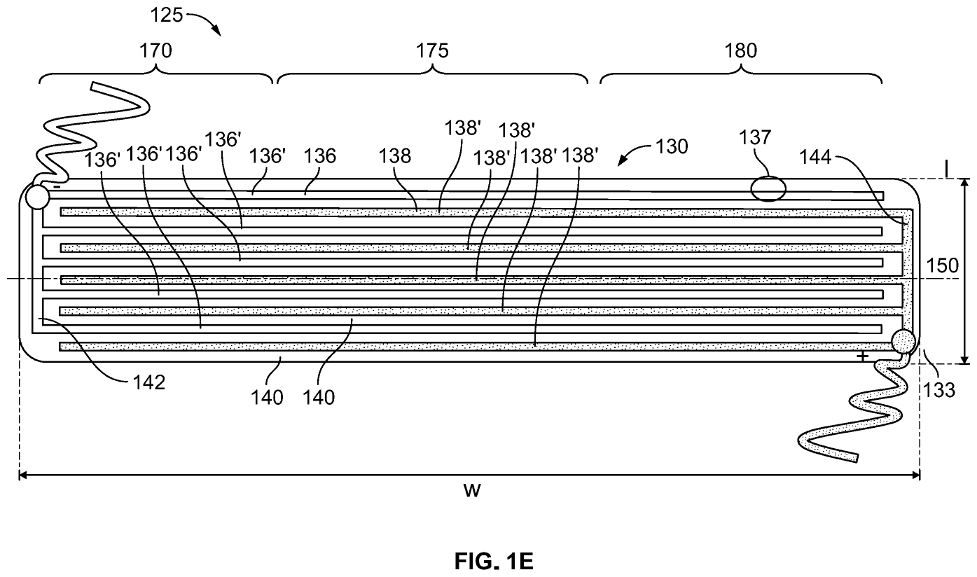

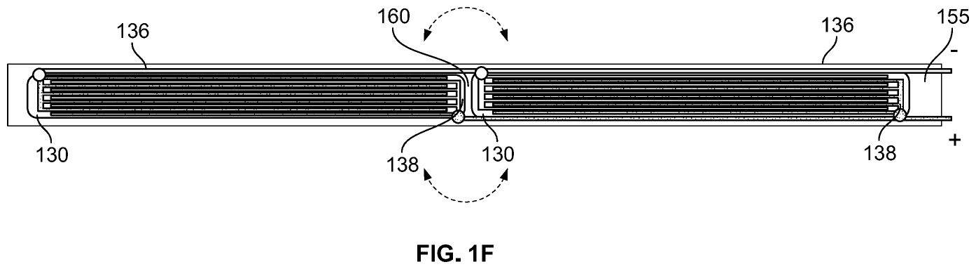

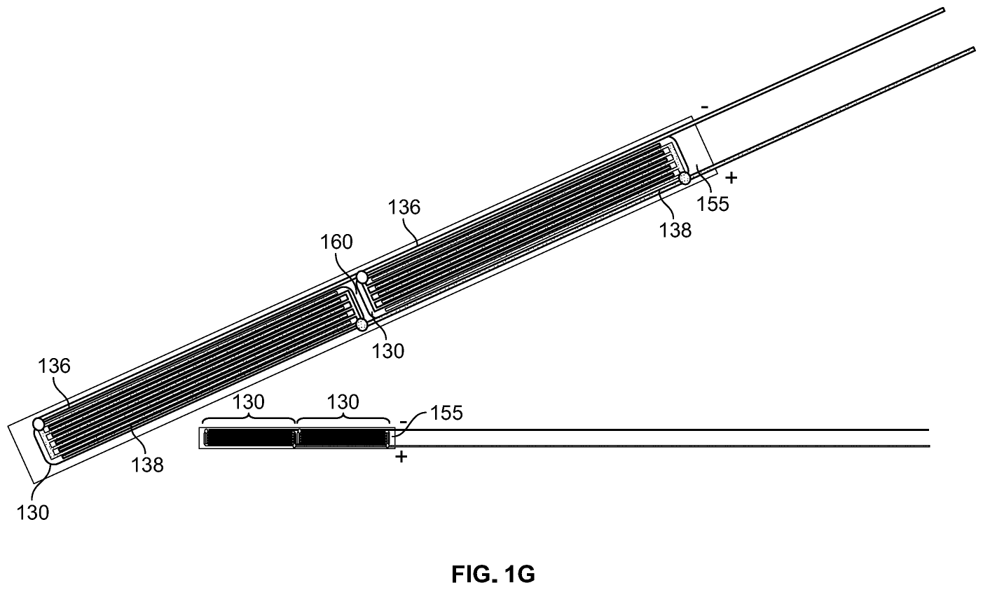

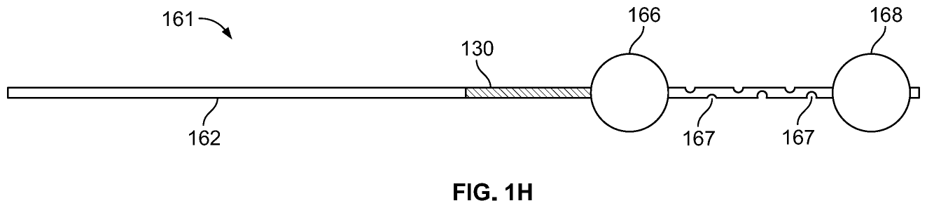

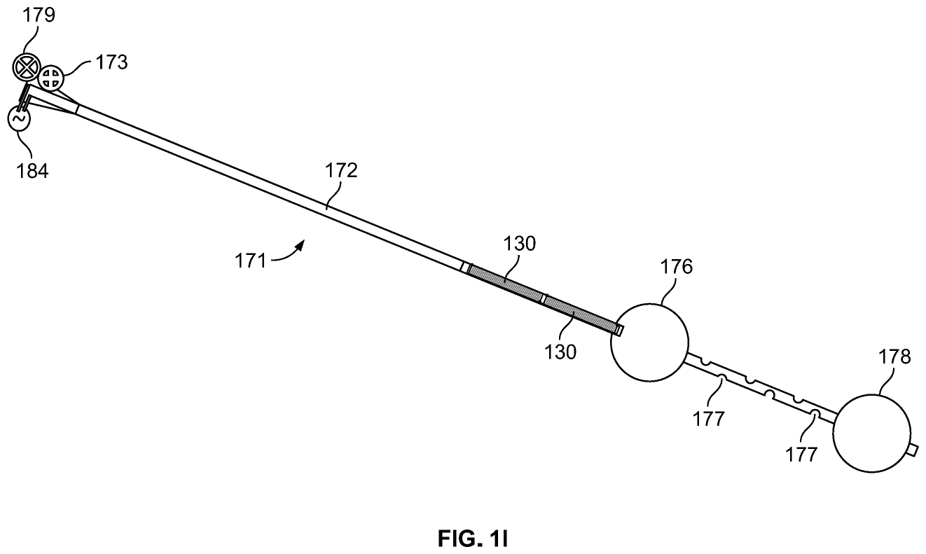

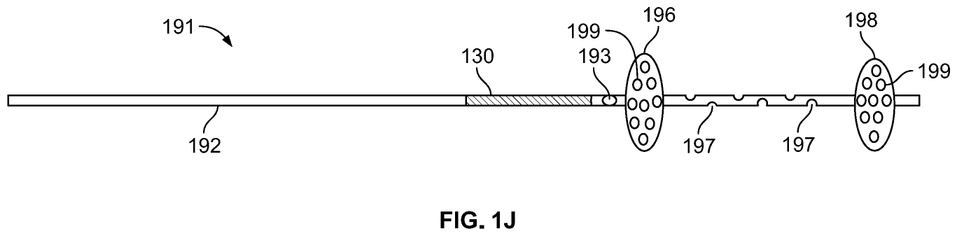

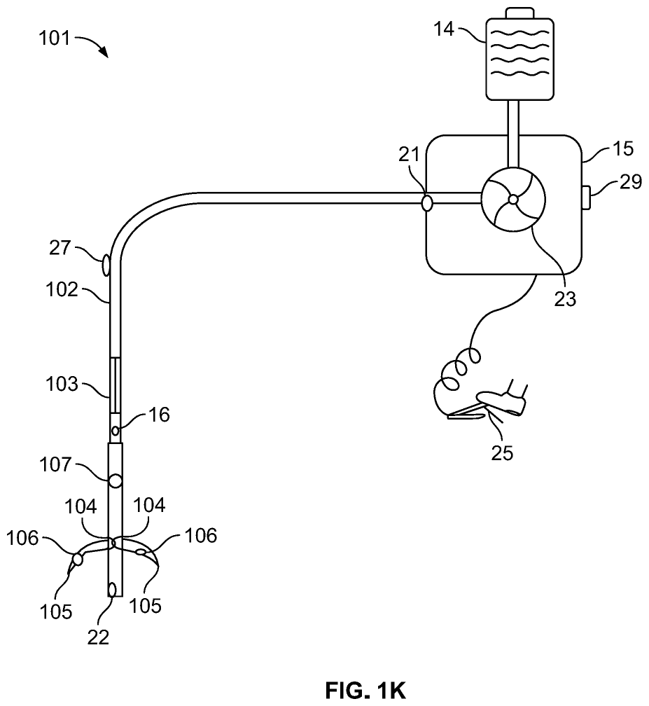

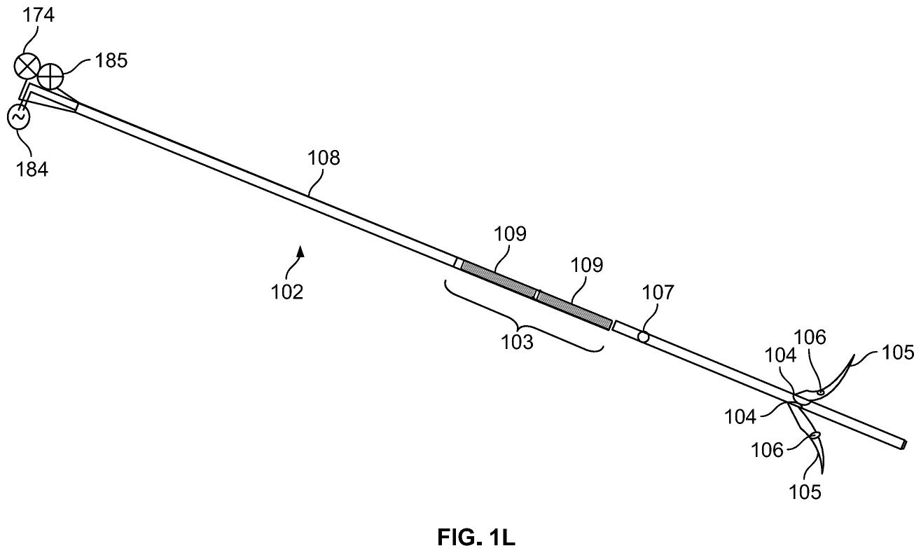

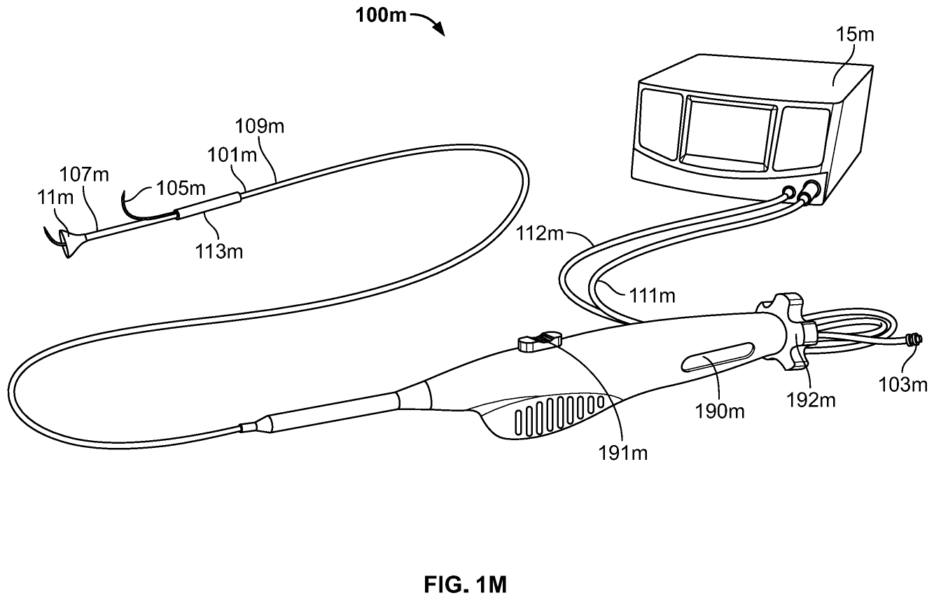

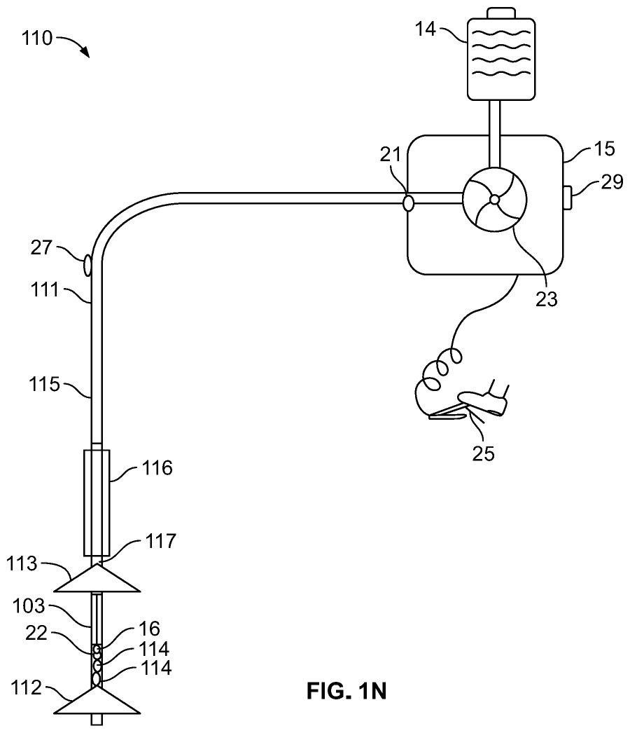

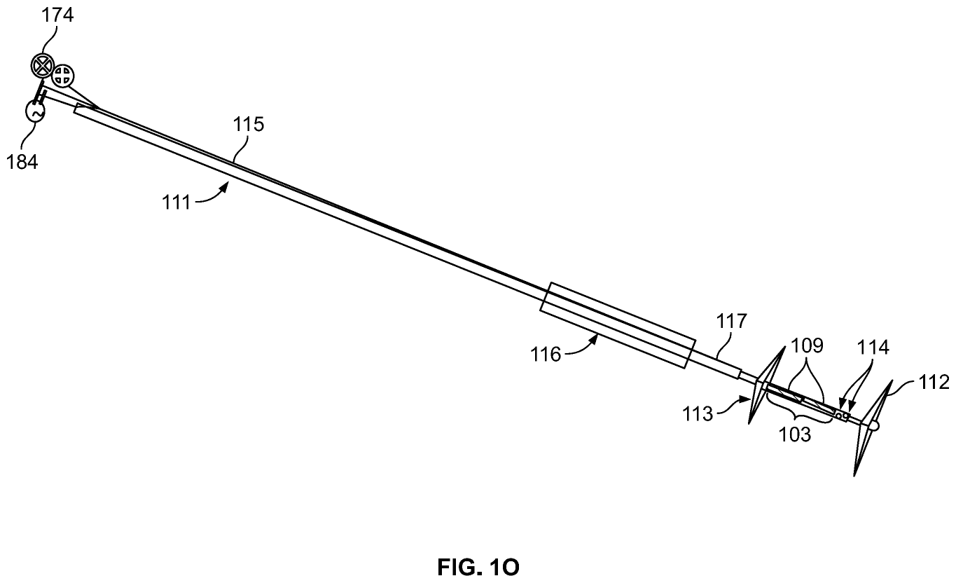

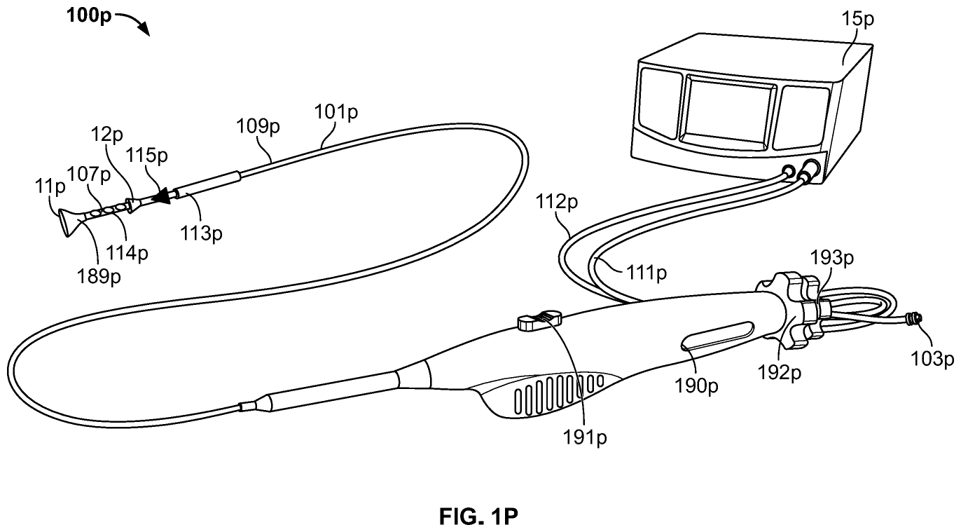

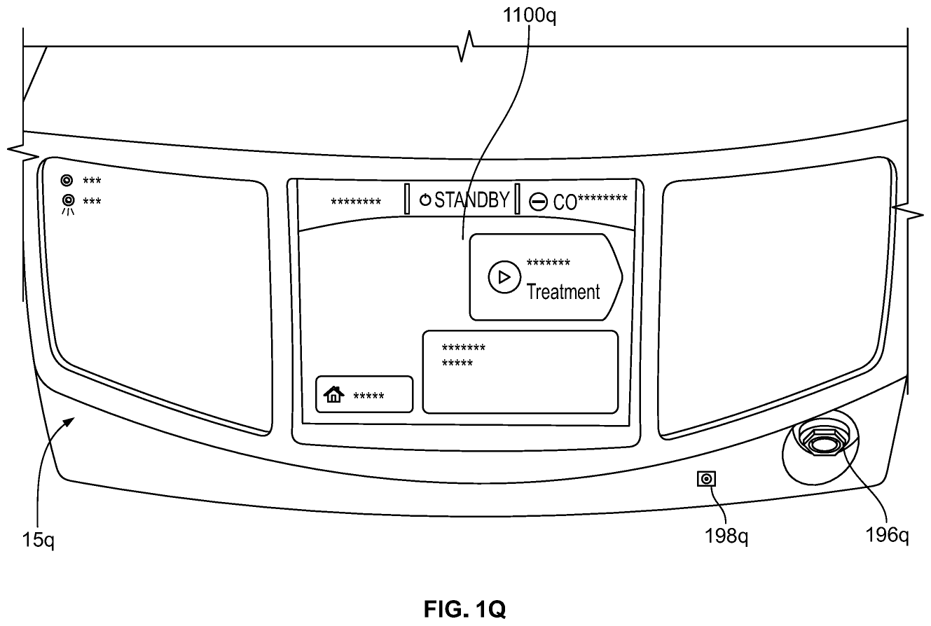

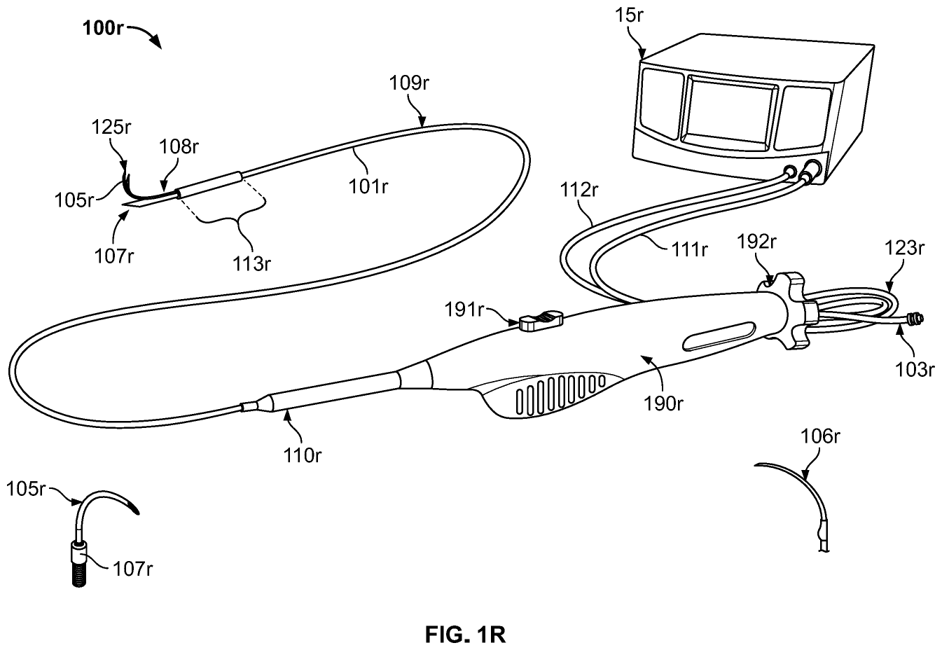

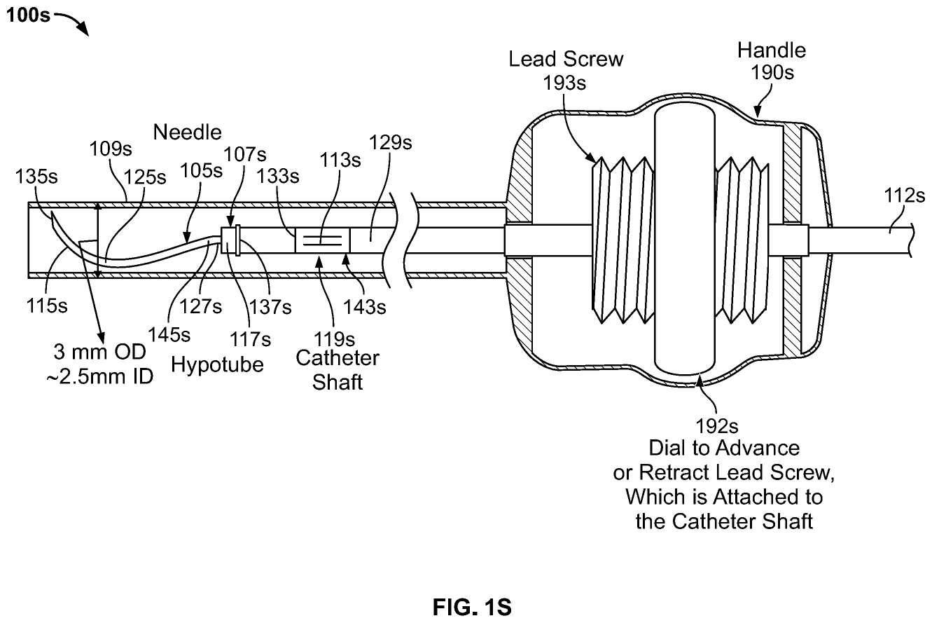

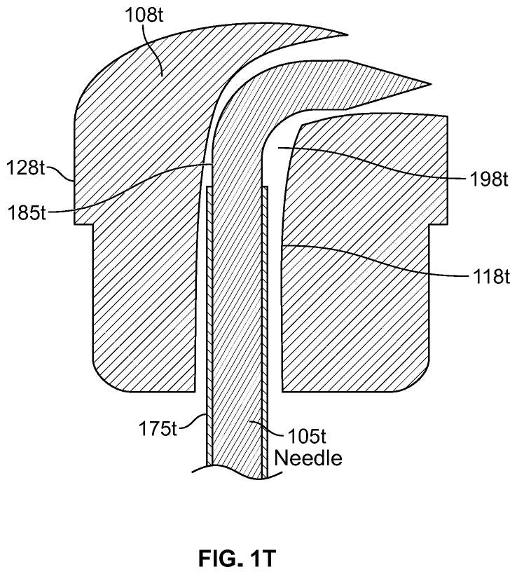

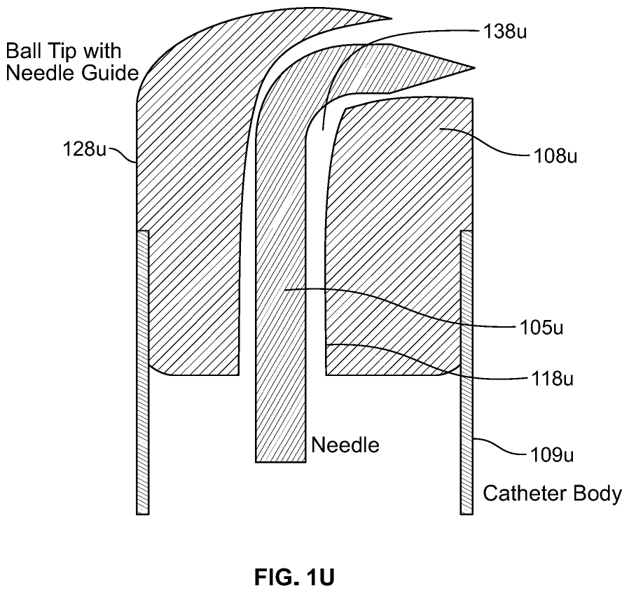

FIG. 1A illustrates an ablation system 100, in accordance with embodiments of the present specification. The ablation system comprises a catheter 10 having at least one first distal attachment or positioning element 11 and an internal heating chamber 18, disposed within a lumen of the catheter 10 and configured to heat a fluid provided to the catheter 10 to change said fluid to a vapor for ablation therapy. The internal heating chamber 18 comprises an electrode or an array of electrodes that are separated from thermally conductive element by a segment of the catheter 10 which is electrically non-conductive. In some embodiments, the catheter 10 is made of or covered with an insulated material to prevent the escape of ablative energy from the catheter body. The catheter 10 comprises one or more infusion ports 12 for the infusion of ablative agent, such as steam. In some embodiments, the one or more infusion ports 12 comprises a single infusion port at the distal end of a needle. In some embodiments, the catheter includes a second positioning element 13 proximal to the infusion ports 12. In various embodiments, the first distal attachment or positioning element 11 and second positioning element 13 may be any one of a disc, hood, cap, or inflatable balloon. In some embodiments the distal attachment or positioning element has a wire mesh structure with or without a covering membrane. In some embodiments, the first distal attachment or positioning element 11 and second positioning element 13 include pores 19 for the escape of air or ablative agent. A fluid, such as saline, is stored in a reservoir, such as a saline pump 14, connected to the catheter 10. Delivery of the ablative agent is controlled by a controller 15 and treatment is controlled by a treating physician via the controller 15. The controller 15 includes at least one processor 23 in data communication with the saline pump 14 and a catheter connection port 21 in fluid communication with the saline pump 14. In some embodiments, at least one optional sensor 17 monitors changes in an ablation area to guide flow of ablative agent. In some embodiments, optional sensor 17 comprises at least one of a temperature sensor or pressure sensor. In some embodiments, the catheter 10 includes a filter 16 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 25 in data communication with the controller 15, a switch 27 on the catheter 10, or a switch 29 on the controller 15, for controlling vapor flow. In various embodiments, the switch 29 is positioned on the generator or the catheter handle.

In one embodiment, a user interface included with the controller 15 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

FIG. 1B is a transverse cross-section view 121 of a flexible heating chamber 130 configured to be incorporated at or into a distal portion or tip of a catheter, in accordance with an embodiment of the present specification. FIG. 1C illustrates a transverse cross-section view 122a and a longitudinal cross-section view 122b of a first array of electrodes 136 along with a transverse cross-section view 123a and a longitudinal cross-section view 123b of a second array of electrodes 138 of a flexible heating chamber for a catheter, in accordance with an embodiment of the present specification. FIGS. 1D and 1E are, respectively, transverse and longitudinal cross-section views 124, 125 of the heating chamber 130 including assembled first and second electrodes 136, 138.

Referring now to FIGS. 1B, 1C, 1D, and 1E simultaneously, the heating chamber 130 comprises an outer covering 132 and a coaxial inner core, channel, or lumen 134. A plurality of electrodes, configured as first and second arrays of electrodes 136, 138, is disposed between the outer covering 132 and the inner lumen 134. In some embodiments, the first and second array of electrodes 136, 138 respectively comprise metal rings 142, 144 from which a plurality of electrode fins or elements 136′, 138′ extend radially into the space between the outer covering 132 and insulative inner core/lumen 134 (see 122a, 123a). The electrode fins or elements 136′, 138′ also extend longitudinally along a longitudinal axis 150 of the heating chamber 130 (see 122b, 123b). In other words, each of the electrode fins 136′, 138′ have a first dimension along a radius of the heating chamber 130 and a second dimension along a longitudinal axis 150 of the heating chamber 130. The electrode fins or elements 136′, 138′ define a plurality of segmental spaces 140 there-between through which saline/water flows and is vaporized into steam. Electrical current is directed from the controller, into the catheter, through a lumen, and to the electrodes 136, 138 which causes the fins or elements 136′, 138′ to generate electrical charge which is then conducted through the saline in order to heat the saline and convert the saline to steam. The first and second dimensions enable the electrodes 136, 138 to have increased surface area for heating the saline/water flowing in the spaces 140. In accordance with an embodiment, the first electrodes 136 have a first polarity and the second electrodes 138 have a second polarity opposite said first polarity. In an embodiment, the first polarity is negative (cathode) while the second polarity is positive (anode).

In embodiments, the outer covering 132 and the inner core/lumen 134 are comprised of silicone, Teflon, ceramic or any other suitable thermoplastic/electrically insulative elastomer known to those of ordinary skill in the art. The inner core/lumen 134, outer covering 132, electrodes 136, 138 (including rings 142, 144 and fins or elements 136′, 138′) are all flexible to allow for bending of the distal portion or tip of the catheter to provide better positioning of the catheter during ablation procedures. In embodiments, the inner core/lumen 134 stabilizes the electrodes 136, 138 and maintains the separation or spacing 140 between the electrodes 136, 138 while the tip of the catheter flexes or bends during use preventing the electrodes from physically contacting one another and shorting out.

As shown in FIGS. 1D and 1E, when the heating chamber 130 is assembled, the electrode fins or elements 136′, 138′ interdigitate or interlock with each other (similar to fingers of two clasped hands) such that a cathode element is followed by an anode element which in turn is followed by a cathode element that is again followed by an anode element and so on, with a space 140 separating each cathode and anode element. In various embodiments, each space 140 has a distance from a cathode element to an anode element ranging from 0.01 mm to 2 mm. In some embodiments, the first array of electrodes 136 has a range of 1 to 50 electrode fins 136′, with a preferred number of 4 electrode fins 136′, while the second array of electrodes 138 has a range of 1 to 50 electrode fins 138′, with a preferred number of 4 electrode fins 138′. In various embodiments, the heating chamber 130 has a width w in a range of 1 to 5 mm and a length/in a range of 5 to 500 mm.

In accordance with an aspect of the present specification, multiple heating chambers 130 can be arranged in the catheter tip. FIGS. 1F and 1G are longitudinal cross-section views of a catheter tip 155 wherein two heating chambers 130 are arranged in series, in accordance with an embodiment of the present specification. Referring to FIGS. 1F and 1G, the two heating chambers 130 are arranged in series such that a space 160 between the two heating chambers 130 acts as a hinge to impart added flexibility to the catheter tip 155 to allow it to bend. The two heating chambers 130 respectively comprise interdigitated first and second arrays of electrodes 136, 138. Use of multiple, such as two or more, heating chambers 130 enables a further increase in the surface area of the electrodes 136, 138 while maintaining flexibility of the catheter tip 155.

Referring now to FIGS. 1B through 1G, for generating steam, fluid is delivered from a reservoir, such as a syringe, to the heating chamber 130 by a pump or any other pressurization means. In embodiments, the fluid is sterile saline or water that is delivered at a constant or variable fluid flow rate. An RF generator, connected to the heating chamber 130, provides power to the first and second arrays of electrodes 136, 138. As shown in FIG. 1E, during vapor generation, as the fluid flows through spaces 140 in the heating chamber 130 and power is applied to the electrodes 136, 138 causing the electrodes to charge which is conducted through the saline, resistively heating the saline and vaporizing the water in the saline. In other embodiments, conductive heating, convection heating, microwave heating, or inductive heating are used to convert the saline to vapor. The fluid is warmed in a first proximal region 170 of the heating chamber 130. When the fluid is heated to a sufficient temperature, such as 100 degrees Centigrade at atmospheric pressure, the fluid begins to transform into a vapor or steam in a second middle region 175. All of the fluid is transformed into vapor by the time it reaches a third distal region 180, after which it can exit a distal end 133 of the heating chamber 130 and exit the catheter tip 155. If the pressure in the heating chamber is greater than atmospheric pressure, higher temperatures will be required and if it is lower than atmospheric pressure, lower temperatures will generate vapor. When there is no saline flow through the chamber, the flow of the current through the chamber will be interrupted (dry electrode) and no heat will be generated. Measurement of the electrode impedance can be used to measure the flow of the saline and dry versus wet electrode.

In one embodiment, a sensor probe may be positioned at the distal end of the heating chambers within the catheter. During vapor generation, the sensor probe communicates a signal to the controller. The controller may use the signal to determine if the fluid has fully developed into vapor before exiting the distal end of the heating chamber. Sensing whether the saline has been fully converted into vapor may be particularly useful for many surgical applications, such as in the ablation of various tissues, where delivering high quality (low water content) steam results in more effective treatment. In some embodiments, the heating chamber includes at least one sensor 137. In various embodiments, said at least one sensor 137 comprises an impedance, temperature, pressure or flow sensor, with the pressure sensor being less preferred. In one embodiment, the electrical impedance of the electrode arrays 136, 138 can be sensed. In other embodiments, the temperature of the fluid, temperature of the electrode arrays, fluid flow rate, pressure, or similar parameters can be sensed.

FIG. 1H and FIG. 1I illustrate multiple lumen balloon catheters 161 and 171 respectively, in accordance with embodiments of the present specification. The catheters 161, 171 each include an elongate body 162, 172 with a proximal end and a distal end. The catheters 161, 171 include at least one positioning element proximate their distal ends. In various embodiments, the positioning element is a balloon. In some embodiments, the catheters include more than one positioning element.

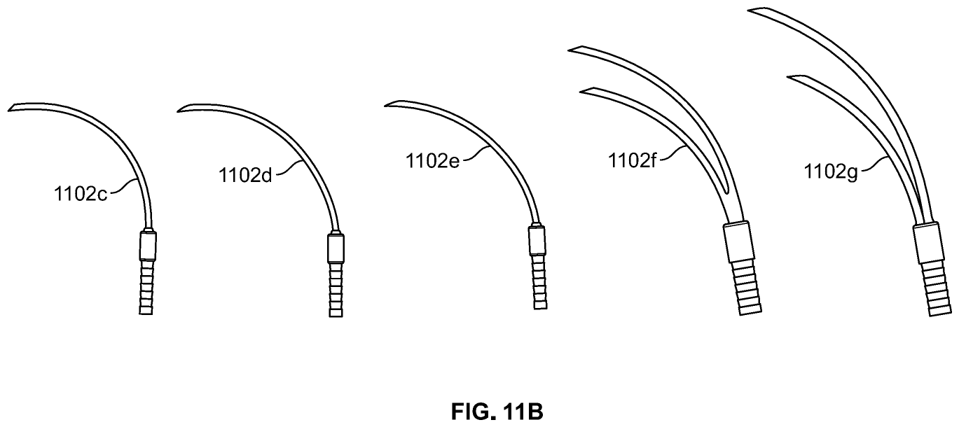

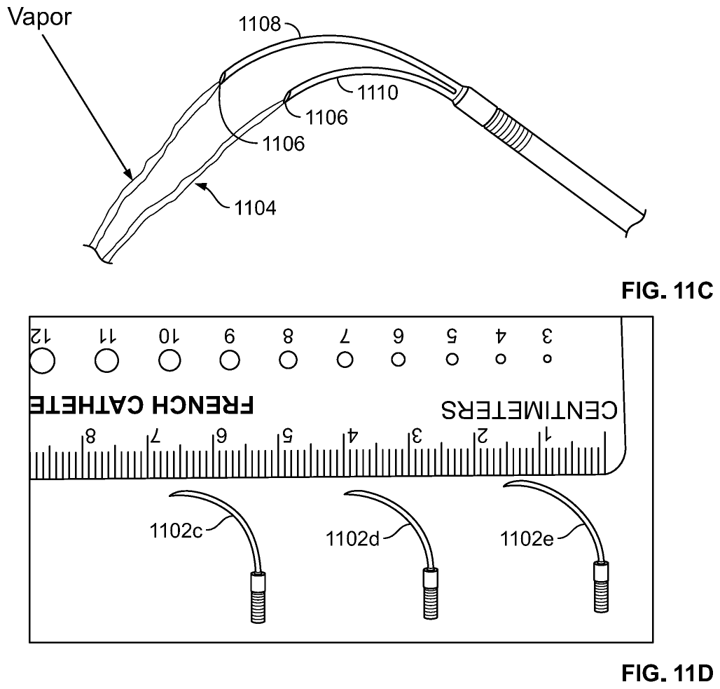

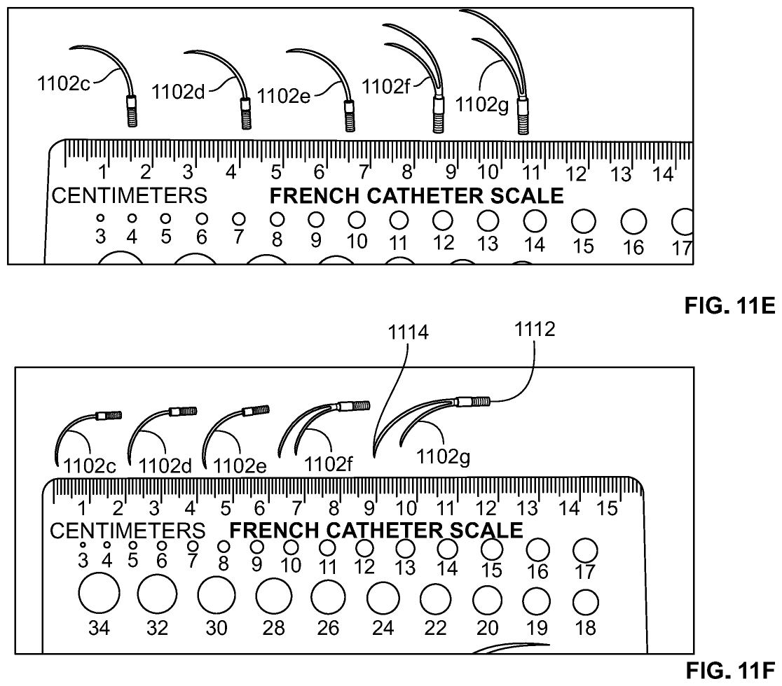

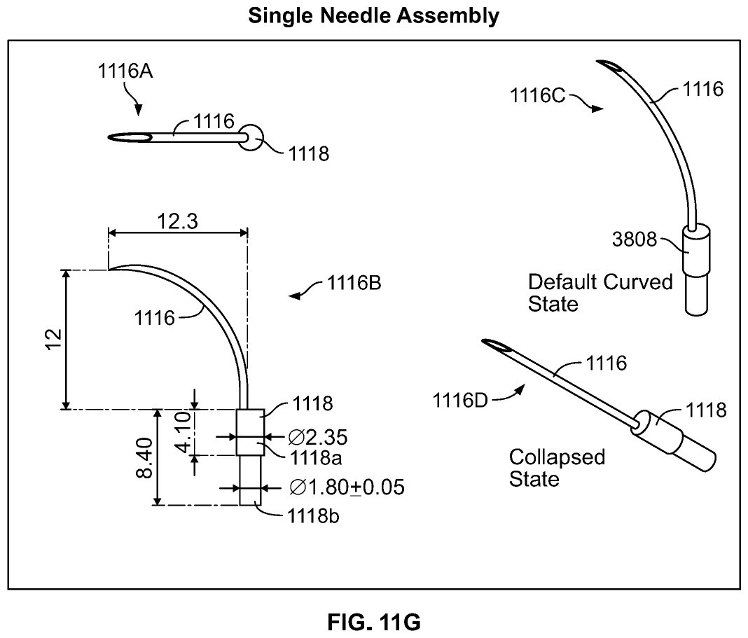

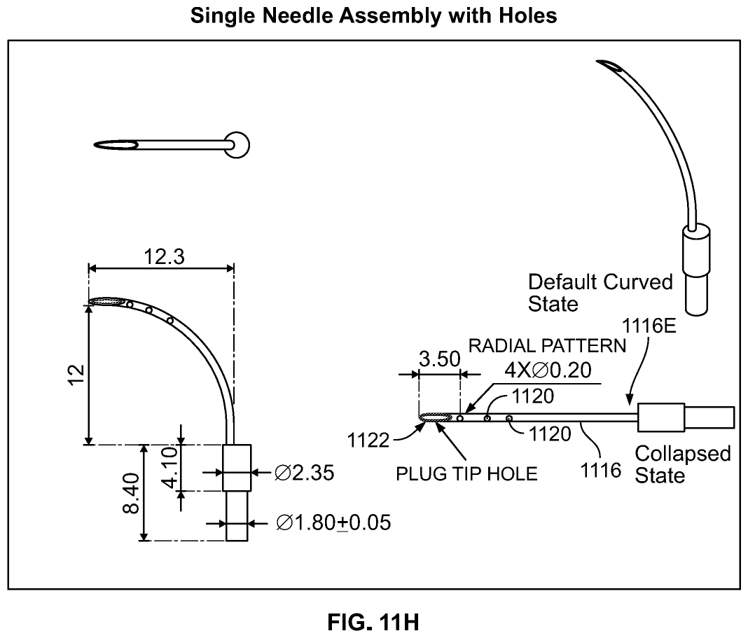

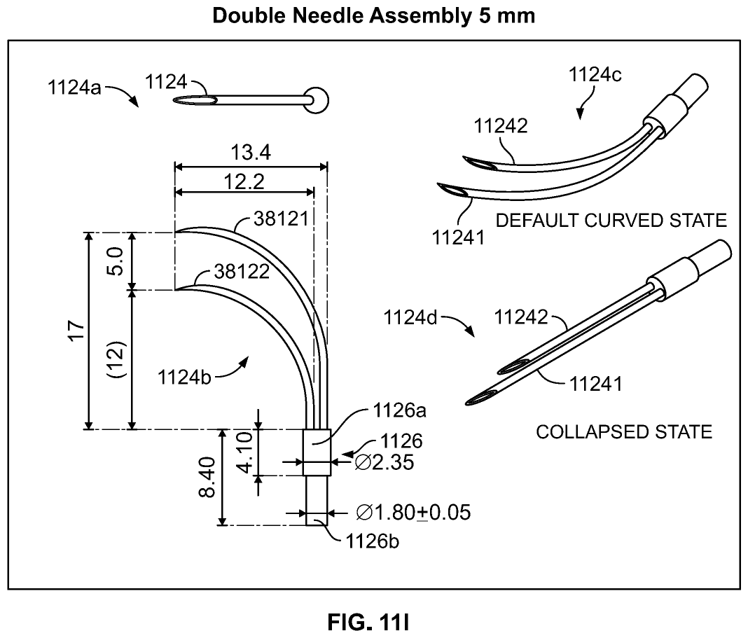

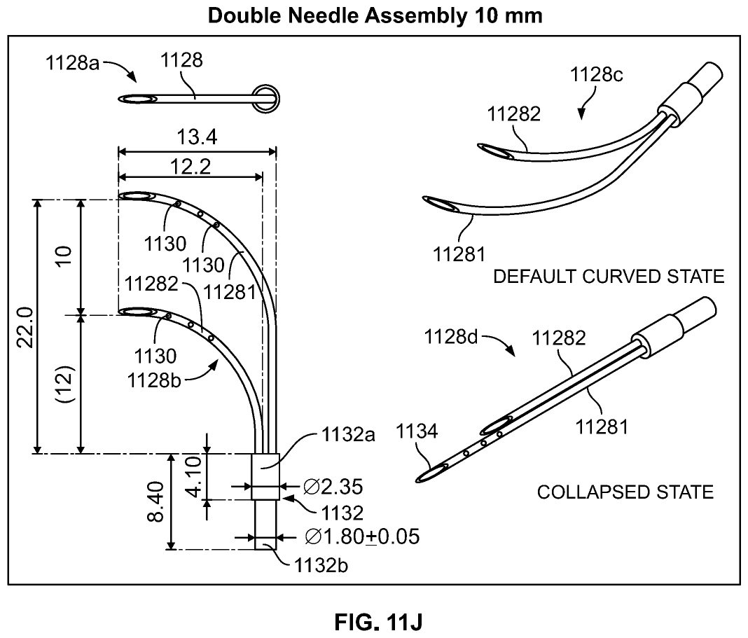

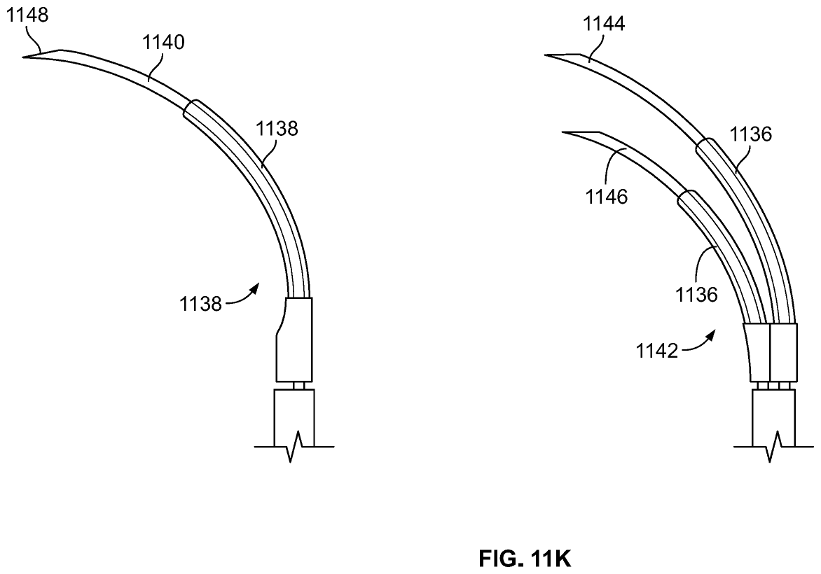

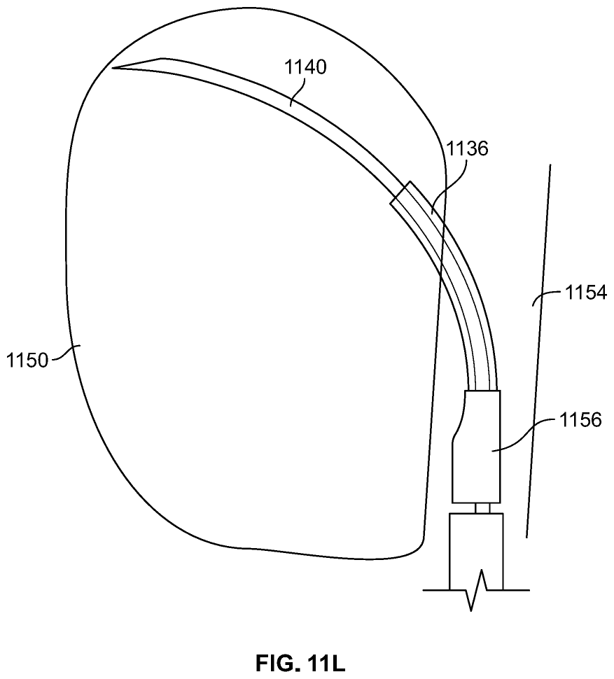

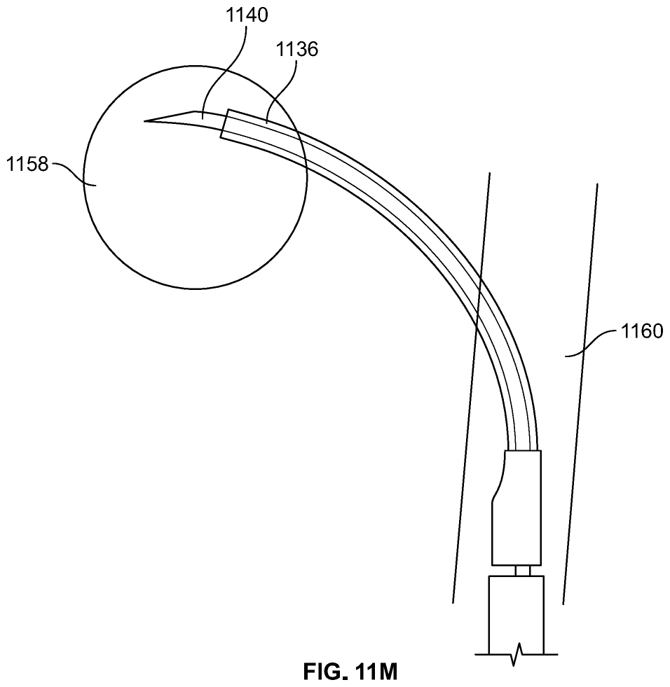

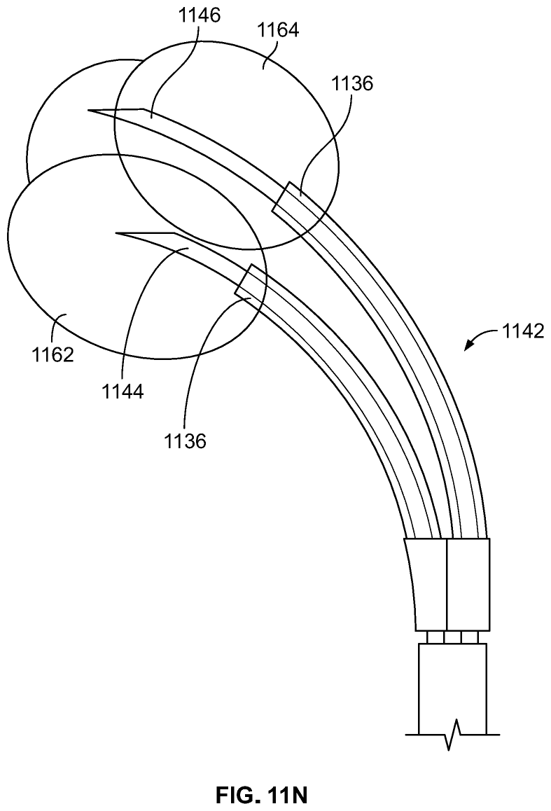

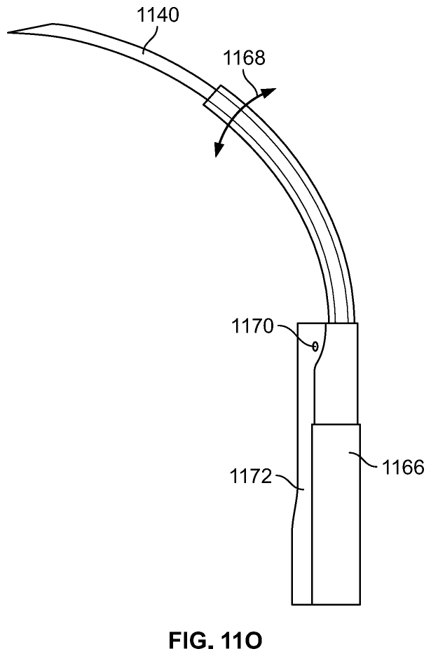

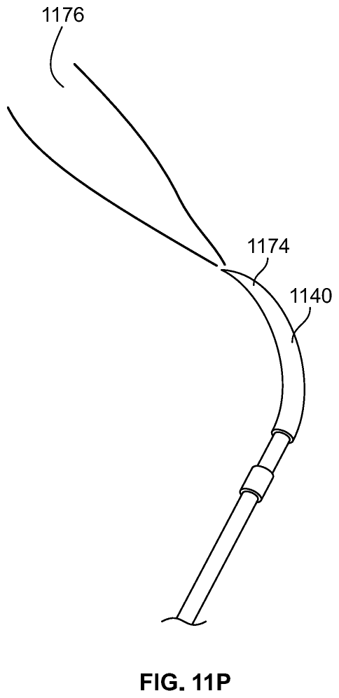

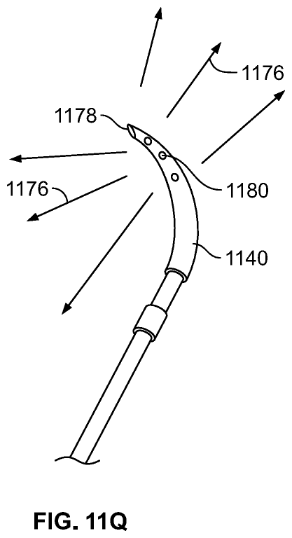

In the embodiments depicted in FIGS. 1H and 11, the catheters 161, 171 each include a proximal balloon 166, 176 and a distal balloon 168, 178 positioned proximate the distal end of the body 162, 172 with a plurality of infusion ports 167, 177 located on the body 162, 172 between the two balloons 166, 176, and 168, 178. The body 162, 172 also includes at least one heating chamber 130 proximate and just proximal to the proximal balloon 166, 176. The embodiment of FIG. 1H illustrates one heating chamber 130 included in the body 165 proximate and just proximal to the proximal balloon 166. In some embodiments, multiple heating chambers are arranged in series in the body of the catheter.

In the embodiment of FIG. 1I, two heating chambers 130 are arranged in the body 172 proximate and just proximal to the proximal balloon 176. Referring to FIG. 1I, for inflating the balloons 176, 178 and providing electrical current and liquid to the catheter 171, a fluid pump 179, an air pump 173 and an RF generator 184 are coupled to the proximal end of the body 172. The air pump 173 pumps air via a first port through a first lumen (extending along a length of the body 172) to inflate the balloons 176, 178 so that the catheter 171 is held in position for an ablation treatment. In another embodiment, the catheter 171 includes an additional air-port and an additional air lumen so that the balloons 176, 178 may be inflated individually. The fluid pump 179 pumps the fluid through a second lumen (extending along the length of the body 172) to the heating chambers 130. The RF generator 184 supplies electrical current to the electrodes 136, 138 (FIGS. 1G, 1H), causing the electrodes 136, 138 to generate heat and thereby converting the fluid flowing through the heating chambers 130 into vapor. The generated vapor flows through the second lumen and exits the ports 177. The flexible heating chambers 130 impart improved flexibility and maneuverability to the catheters 161, 171, allowing a physician to better position the catheters 161, 171 when performing ablation procedures, such as ablating Barrett's esophagus tissue in an esophagus of a patient. FIG. 1J illustrates a catheter 191 with proximal and distal positioning elements 196, 198 and an electrode heating chamber 130, in accordance with embodiments of the present specification. The catheter 191 includes an elongate body 192 with a proximal end and a distal end. The catheter 191 includes a proximal positioning element 196 and a distal positioning element 198 positioned proximate the distal end of the body 192 with a plurality of infusion ports 197 located on the body 192 between the two positioning elements 196, 198. The body 192 also includes at least one heating chamber 130 within a central lumen. In some embodiments, the proximal positioning element 196 and distal positioning element 198 comprises compressible discs which expand on deployment. In some embodiments, the proximal positioning element 196 and distal positioning element 198 are comprised of a shape memory metal and are transformable from a first, compressed configuration for delivery through a lumen of an endoscope and a second, expanded configuration for treatment. In embodiments, the discs include a plurality of pores 199 to allow for the escape of air at the start of an ablation procedure and for the escape of steam once the pressure and/or temperature within an enclosed treatment volume created between the two positioning elements 196, 198 reaches a predefined limit, as described above. In some embodiments, the catheter 191 includes a filter 193 with micro-pores which provides back pressure to the delivered steam, thereby pressurizing the steam. The predetermined size of micro-pores in the filter determine the backpressure and hence the temperature of the steam being generated.

It should be appreciated that the filter 193 may be any structure that permits the flow of vapor out of a port and restricts the flow of vapor back into, or upstream within, the catheter. Preferably, the filter is a thin porous metal or plastic structure, positioned in the catheter lumen and proximate one or more ports. Alternatively, a one-way valve may be used which permits vapor to flow out of a port but not back into the catheter. In one embodiment, this structure 193, which may be a filter, valve or porous structure, is positioned within 5 cm of a port, preferably in a range of 0.1 cm to 5 cm from a port, and more preferably within less than 1 cm from the port, which is defined as the actual opening through which vapor may flow out of the catheter and into the patient.

FIG. 1K illustrates an ablation system 101 suitable for use in ablating prostate tissue, in accordance with some embodiments of the present specification. The ablation system 101 comprises a catheter 102 having an internal heating chamber 103, disposed within a lumen of the catheter 102 and configured to heat a fluid provided to the catheter 102 to change said fluid to a vapor for ablation therapy. In one embodiment the fluid is electrically conductive saline and is converted into electrically non-conductive or poorly conductive vapor. In one embodiment, there is at least a 25% decrease in the conductivity, preferably a 50% decrease and more preferably a 90% decrease in the conductivity, of the fluid as determined by comparing the conductivity of the fluid, such as saline, prior to passing through the heating chamber to the conductivity of the ablative agent, such as steam, after passing through the heating chamber. It should further be appreciated that, for each of the embodiments disclosed in this specification, the term ablative agent preferably refers solely to the heated vapor, or steam, and the inherent heat energy stored therein, without any augmentation from any other energy source, including a radio frequency, electrical, ultrasonic, optical, or other energy modality.

In some embodiments, the catheter 102 is made of or covered with an insulated material to prevent the escape of ablative energy from the catheter body. A plurality of openings 104 are located proximate the distal end of the catheter 102 for enabling a plurality of associated thermally conductive elements, such as needles 105, to be extended (at an angle from the catheter 102, wherein the angle ranges between 30 to 90 degrees) and deployed or retracted through the plurality of openings 104. In accordance with an aspect, the plurality of retractable needles 105 are hollow and include at least one infusion port 106 to allow delivery of an ablative agent, such as steam or vapor, through the needles 105 when the needles 105 are extended and deployed through the plurality of openings 104 on the elongated body of the catheter 102. In some embodiments, the infusion ports are positioned along a length of the needles 105. In some embodiments, the infusion ports 106 are positioned at a distal tip of the needles 105. During use, cooling fluid such as water, air, or CO₂ is circulated through an optional port 107 to cool the catheter 102. Vapor for ablation and cooling fluid for cooling are supplied to the catheter 102 at its proximal end. A fluid, such as saline, is stored in a reservoir, such as a saline pump 14, connected to the catheter 102. Delivery of the ablative agent is controlled by a controller 15 and treatment is controlled by a treating physician via the controller 15. The controller 15 includes at least one processor 23 in data communication with the saline pump 14 and a catheter connection port 21 in fluid communication with the saline pump 14. In some embodiments, at least one optional sensor 22 monitors changes in an ablation area to guide flow of ablative agent. In some embodiments, the optional sensor comprises at least one of a temperature sensor or pressure sensor. In some embodiments, the catheter 102 includes a filter 16 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 25 in data communication with the controller 15, a switch 27 on the catheter 102, or a switch 29 on the controller 15, for controlling vapor flow. In some embodiment, the needles have attached mechanism to change their direction from being relatively parallel to the catheter to being at an angle between 30°-90° to the catheter. In one embodiment, the aforementioned mechanism is a pull wire. In some embodiments, the openings in the catheter are shaped to change the direction of the needle from being relatively parallel to the catheter to being at an angle between 30°-90° to the catheter.

In one embodiment, a user interface included with the microprocessor 15 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

FIG. 1L illustrates another view of a catheter 102 of FIG. 1K, in accordance with some embodiments of the present specification. The catheter 102 includes an elongate body 108 with a proximal end and a distal end. A plurality of openings 104 are located proximate the distal end of the catheter 102 for enabling a plurality of associated thermally conductive elements, such as needles 105, to be extended (at an angle from the catheter 102, wherein the angle ranges between 10 to 90 degrees) and deployed or retracted through the plurality of openings 104. In accordance with an aspect, the plurality of retractable needles 105 are hollow and include at least one infusion port 106 to allow delivery of an ablative agent, such as steam or vapor, through the needles 105 when the needles 105 are extended and deployed through the plurality of openings 104 on the elongated body of the catheter 102. In some embodiments, the infusion ports are positioned along a length of the needles 105. In some embodiments, the infusion ports 106 are positioned at a distal tip of the needles 105. Optionally, during use, cooling fluid, such as water, air, or CO₂ is circulated through an optional port 107 to cool the catheter 102. The body 108 includes at least one heating chamber 103 proximate and just proximal to the optional port 107 or openings 104. In embodiments, the heating chamber 103 comprises two electrodes 109 configured to receive RF current, heat, and convert supplied fluid, such as saline, to vapor or steam, for ablation.

Referring to FIG. 1L, for providing electrical current, fluid for ablation, and optional cooling fluid to the catheter 102, an RF generator 184, a first fluid pump 174, and a second fluid pump 185 are coupled to the proximal end of the body 108. The first fluid pump 174 pumps a first fluid, such as saline, through a first lumen (extending along the length of the body 108) to the heating chamber 103. The RF generator 184 supplies electrical current to the electrodes 109, causing the electrodes 109 to generate heat and thereby converting the fluid flowing through the heating chamber 103 into vapor. The generated vapor flows through the first lumen, openings 104, needles 105, and exits the infusion ports 106 to ablate prostatic tissue. Optionally, in some embodiments, a second fluid pump 185 pumps a second fluid, such as water, through a second lumen (extending along a length of the body 108) to optional port 107, where the second fluid exits the catheter 102 to circulate in and cool the area of ablation. The flexible heating chamber 103 imparts improved flexibility and maneuverability to the catheter 102, allowing a physician to better position the catheter 102 when performing ablation procedures, such as ablating prostate tissue of a patient.

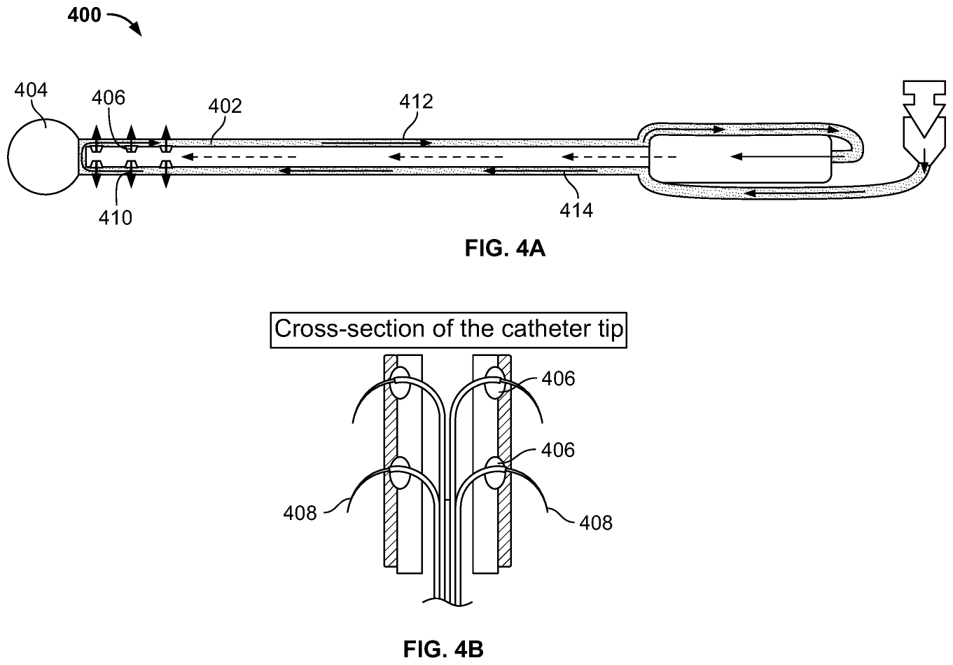

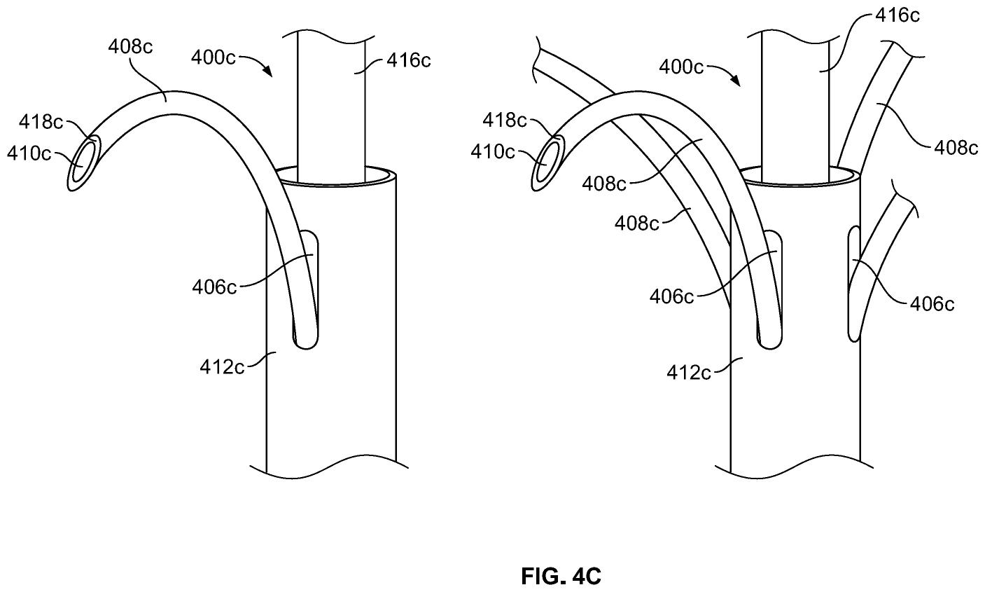

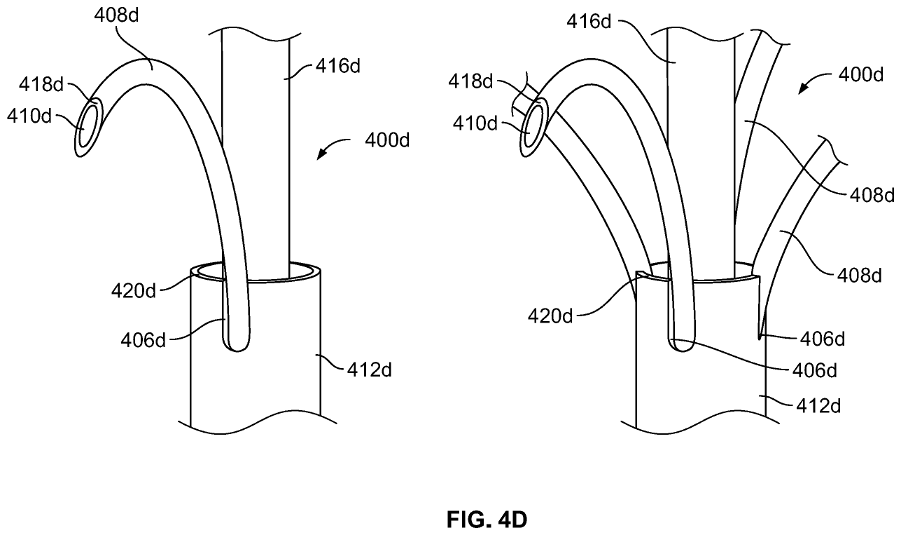

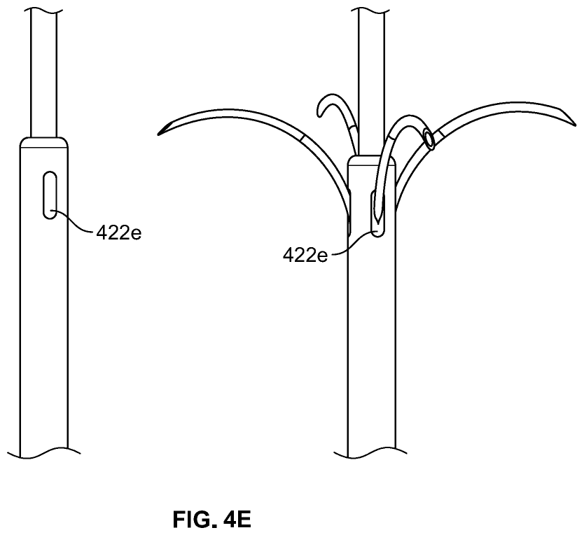

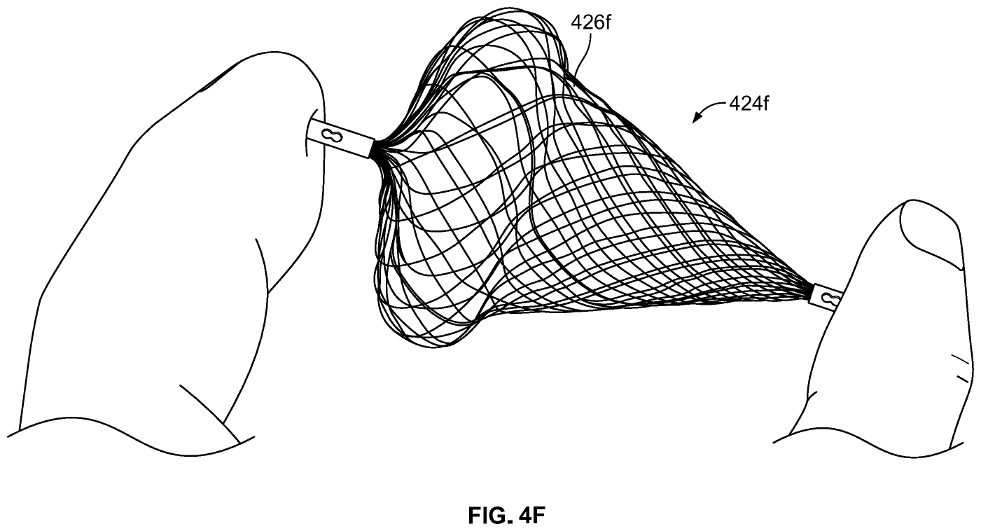

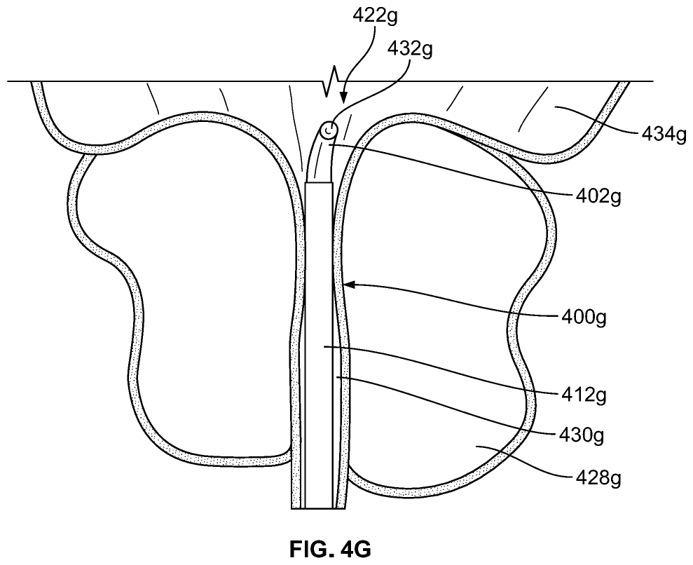

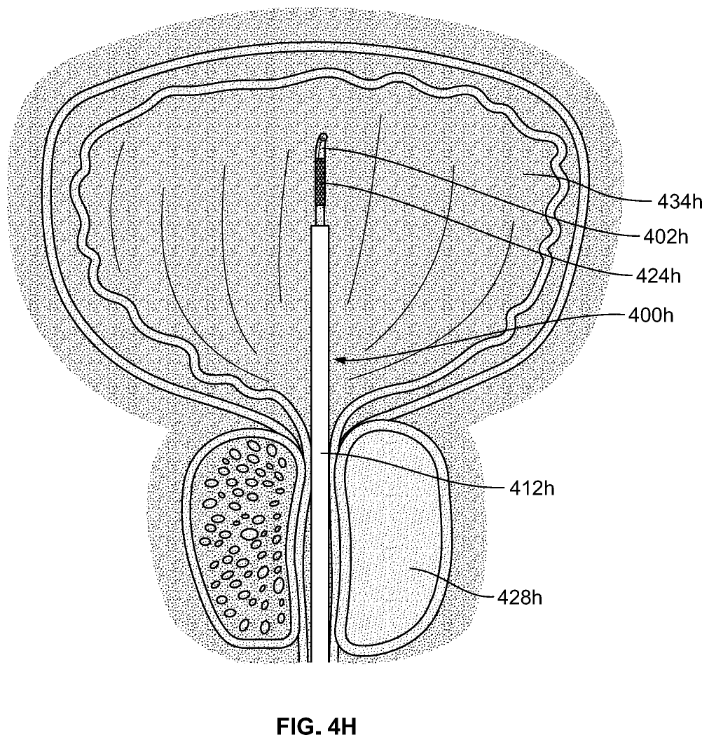

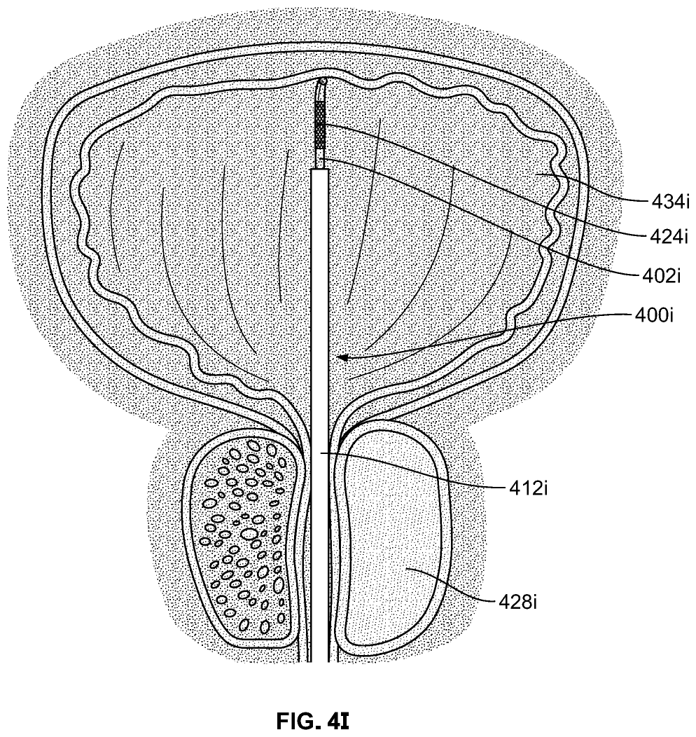

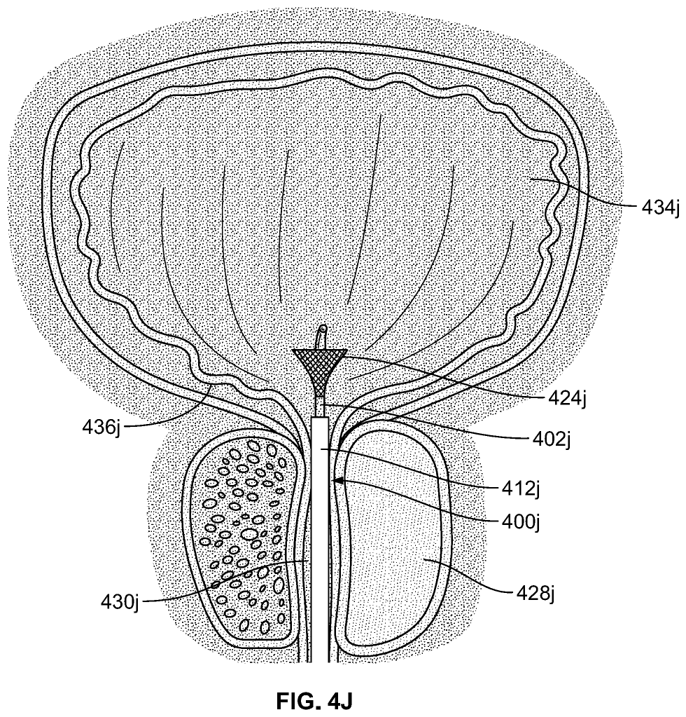

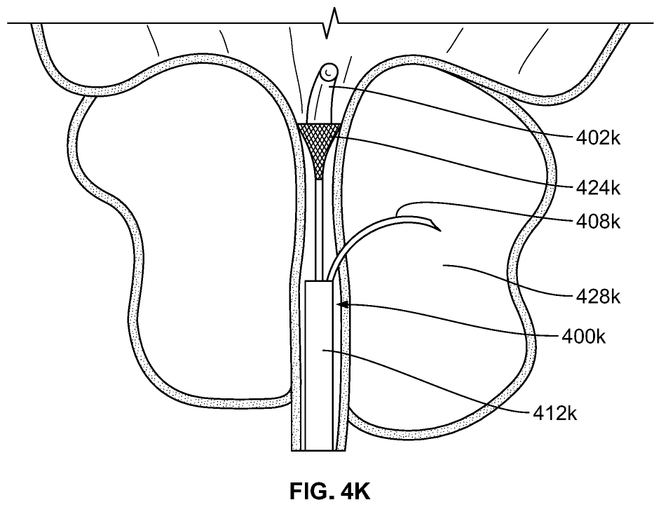

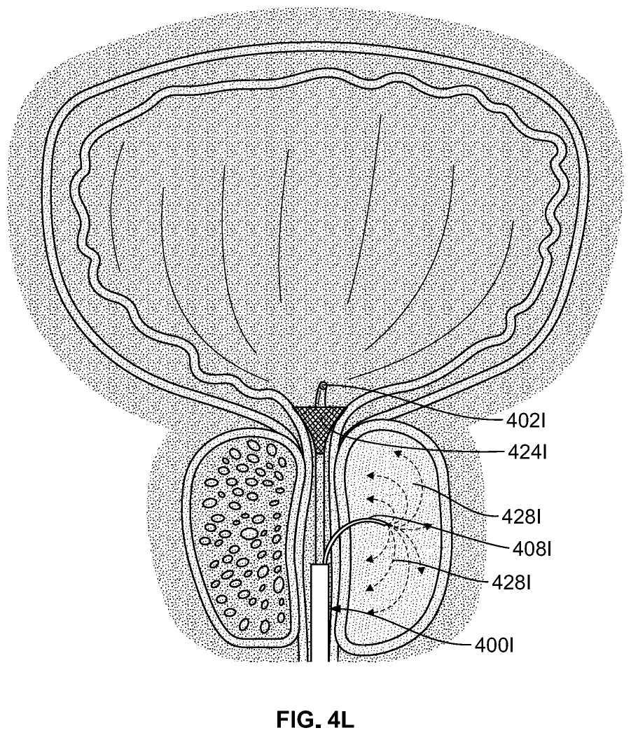

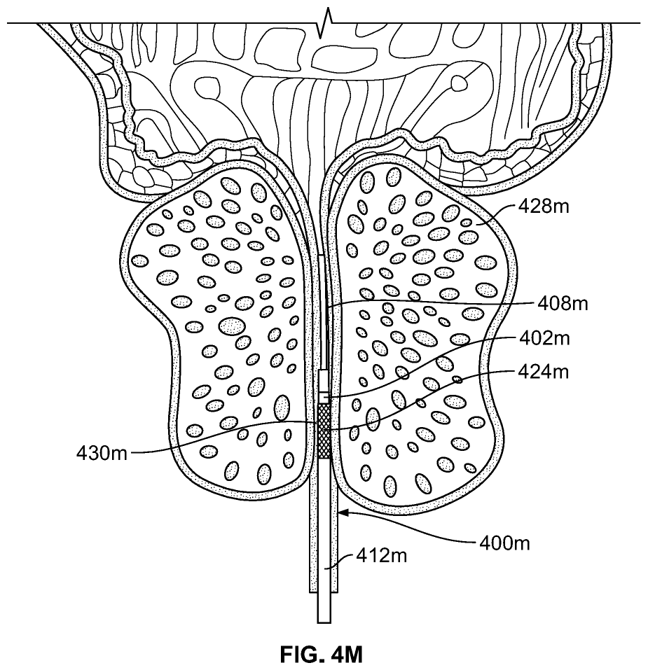

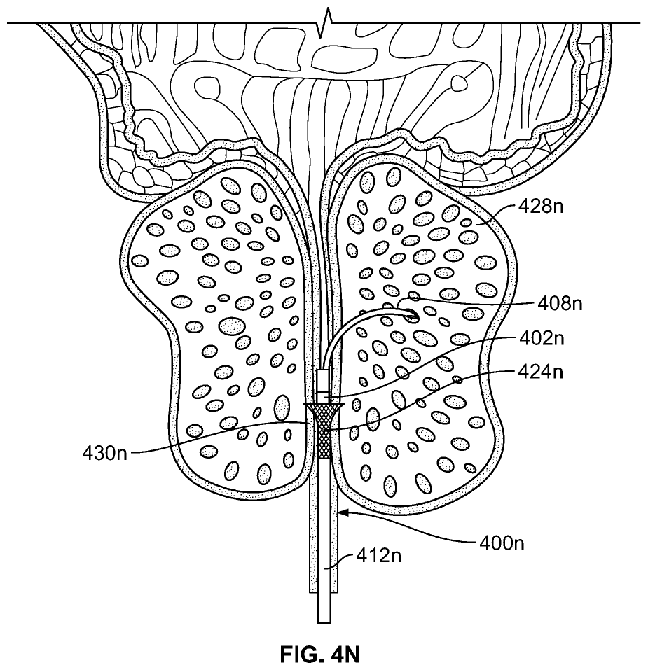

FIG. 1M illustrates a system 100m for use in the ablation of prostatic tissue, in accordance with another embodiment of the present specification. The system 100m comprises a catheter 101m which, in some embodiments, includes a handle 190m having actuators 191m, 192m for extending at least one needle 105m or a plurality of needles from a distal end of the catheter 101m and expanding a positioning element 11m at a distal end of the catheter 101m. In some embodiments, actuators 191m and 192m may be one of a knob or a slide or any other type of switch or button to enable extending of the at least one needle 105m or plurality of needles. Delivery of vapor via the catheter 101m is controlled by a controller 15m. In embodiments, the catheter 101m comprises an outer sheath 109m and an inner catheter 107m. The needle 105m extends from the inner catheter 107m at the distal end of the sheath 109m or, in some embodiments, through openings proximate the distal end of the sheath 109m. In embodiments, the positioning element 11m is expandable, positioned at the distal end of the inner catheter 107m, and may be compressed within the outer sheath 109m for delivery. In some embodiments, actuator 191m comprises a knob which is turned by a first extent, for example, by a quarter turn, to pull back the outer sheath 109m. As the outer sheath 109m retracts, the positioning element 11m is revealed. In embodiments, the positioning element 11m comprises a disc or cone configured as a bladder anchor. In embodiments, actuator/knob is turned by a second extend, for example, by a second quarter turn, to pull back the outer sheath further 109m to deploy the needle 105m. In some embodiments, the number of needles that is deployed is two or more than two. In some embodiments, referring to FIGS. 1M, 4C and 4E simultaneously, the needle or needles 105m, 3116a are deployed out of an internal lumen of the inner catheter 107m, 3111a through slots or openings 3115a in the outer sheath 109m, 3110a, which helps control the needle path and insulates the urethra from steam. In some embodiments, the openings are covered with slit covers 3119. In another embodiment, for example, as seen in FIG. 4D, the sleeves 3116b naturally fold outward as the outer sheath 3110b is pulled back.

Referring again to FIG. 1M, in some embodiments, the catheter 101m includes a port 103m for the delivery of fluid, for example cooling fluid, during ablation. In some embodiments, port 103m is also configured to provide for fluid collection, provide vacuum, and provide CO₂ for an integrity test. In some embodiments, the port 103m is positioned on the handle 190m. In some embodiments, at least one electrode 113m is positioned at a distal end of the catheter 101m proximal to the needles 105m. The electrode 113m is configured to receive electrical current, supplied by a connecting wire 111m extending from the controller 15m to the catheter 101m, to heat and convert a fluid, such as saline supplied via tubing 112m extending from the controller 15m to the catheter 101m. Heated fluid or saline is converted to vapor or steam to be delivered by needle 105m for ablation.

FIG. 1R illustrates a system 100r for use in the ablation of prostatic tissue, in accordance with another embodiment of the present specification. The system 100r comprises a catheter 101r which, in some embodiments, includes a handle 190r having actuators 191r, 192r for extending at least one needle 105r or a plurality of needles from a distal end of the catheter 101r. A drive mechanism configured within the handle 190r deploys and retracts the needle 105r in and out of a tip of the catheter shaft 101r. In some embodiments, actuators 191r and 192r may be one of a knob or a slide or any other type of switch or button to enable extending of the at least one needle 105r or plurality of needles. In some embodiments, actuator 191r is a button or a switch that allows a physician to activate treatment using system 100r from the handle 190r as well as a foot pedal (not shown). In some embodiments, a strain relief mechanism 110r is configured at a distal end of the handle 190r that connects the handle 190r to the catheter 101r. The strain relief mechanism 110r provides support to the catheter shaft 101r. Delivery of vapor via the catheter 101r is controlled by a controller 15r. A cable sub-assembly 123r including an electrical cable, in the handle 190r, connects the catheter 101r to the controller 15r. In embodiments, the catheter 101r comprises an outer sheath 109r and an inner catheter (not shown).

In various embodiments, the controller 15r (and 15, 15m, 15p, 15q, and 2252 of FIGS. 1A, 1K, & 1N, 1M, 1P, 1Q, and 22B respectively) of the systems of the present specification comprises a computing device having one or more processors or central processing units, one or more computer-readable storage media such as RAM, hard disk, or any other optical or magnetic media, a controller such as an input/output controller, at least one communication interface and a system memory. The system memory includes at least one random access memory (RAM) and at least one read-only memory (ROM). In embodiments, the memory includes a database for storing raw data, images, and data related to these images. The plurality of functional and operational elements is in communication with the central processing unit (CPU) to enable operation of the computing device. In various embodiments, the computing device may be a conventional standalone computer or alternatively, the functions of the computing device may be distributed across a network of multiple computer systems and architectures and/or a cloud computing system. In some embodiments, execution of a plurality of sequences of programmatic instructions or code, which are stored in one or more non-volatile memories, enable or cause the CPU of the computing device to perform various functions and processes as described in the present specification. In alternate embodiments, hard-wired circuitry may be used in place of, or in combination with, software instructions for implementation of the processes of systems and methods described in this application. Thus, the systems and methods described are not limited to any specific combination of hardware and software.

A needle tip assembly 125r is positioned within a needle chamber 108r, within the outer sheath 109r. The needle chamber 108r may be a metal or plastic sleeve that is configured to house the needle 105r during delivery to assist in needle deployment and retraction and is further described with reference to FIG. 1T. The needle tip assembly 125r, including the needle 105r, extends from the inner catheter when pushed out of its chamber 108r, at the distal end of the sheath 109r or, in some embodiments, through openings proximate the distal end of the sheath 109r. In embodiments, a positioning element is also provided at the distal end of the inner catheter. The positioning element may be expandable, and may be compressed within the outer sheath 109r for delivery. In some embodiments, actuator 192r comprises a knob which is turned by a first extent, for example, by a quarter turn, to pull back the outer sheath 109r. As the outer sheath 109r retracts, the positioning element is revealed. In embodiments, actuator/knob 192r is turned by a second extend, for example, by a second quarter turn, to pull back the outer sheath 109r further to deploy the needle 105r. In some embodiments, the number of needles that is deployed is two or more than two. In some embodiments, referring to FIGS. 1R, 4C and 4E simultaneously, the needle or needles 105r, 3116a are deployed out of an internal lumen of the inner catheter 3111a through slots or openings 3115a in the outer sheath 109r, 3110a, which helps control the needle path and insulates the urethra from steam. In some embodiments, the openings are covered with slit covers 3119. In another embodiment, for example, as seen in FIG. 4D, the sleeves 3116b naturally fold outward as the outer sheath 3110b is pulled back.

FIG. 1R illustrates an expanded view of a needle tip assembly 125r, which includes a needle 105r attached to a needle attachment component 107r which, in some embodiments, comprises a metal threaded fitting, and is described in further detail with reference to FIG. 1S. The needle attachment component, or threaded fitting, 107r connects the needle 105r to the catheter 101r. In embodiments, the needle attachment component 107r comprises a threaded surface fixedly attached to a tip of the catheter 101r and configured to have a needle 105r screwed thereto. In some embodiments, the needle 105r is a 22 to 25 G needle. In some embodiments, needle 105r has a gradient of coating for insulation or echogenicity. An insulation coating 106r may be ceramic, polymer, or any other material suitable for coating the needle 105r and providing insulation and/or echogenicity to the needle 105r. The coatings are provided at the base of the needle 105r to varying lengths to the needle tip.

Referring again to FIG. 1R, in some embodiments, the catheter 101r includes a tubing and connector sub-assembly (port) 103r for the delivery of fluid, for example cooling fluid, during ablation. In some embodiments, port 103r is also configured to provide for fluid collection, provide vacuum, and provide CO₂ for an integrity test. In some embodiments, the port 103r is positioned on the handle 190r. In some embodiments, one or more electrodes 113r is positioned at a distal end of the catheter 101r proximal to the one or more needles 105r. The one or more electrodes 113r is configured to receive electrical current, supplied by a connecting wire 111r extending from the controller 15r to the catheter 101r, to heat and convert a fluid, such as saline, supplied via tubing 112r extending from the controller 15r to the catheter 101r. Heated fluid or saline is converted to vapor or steam to be delivered by needle 105r for ablation.

FIG. 1S illustrates a needle attachment component 107s of a system 100s for use in the ablation of prostatic tissue, in accordance with some embodiments of the present specification. In a preferred embodiment, a needle attachment component 107s, having a lumen 117s defining an internal cavity, is fixedly attached to the end of the inner catheter 119s such that the lumen 129s of the inner catheter 119s is in fluid communication with the lumen 117s of the needle attachment component 107s. Preferably the needle attachment component 107s has, on its distal external surface 127s, a plurality of threads on to which a needle 105s may be screwed. Also preferably, the needle attachment component 107s is made of the same material as the needle 105s, preferably metal and more preferably stainless steel.

It is important for the proximal portion 137s of the needle attachment component 107s to be spaced in a very specific range from the one or more electrodes 113s. Too close and the electricity from the electrode(s) 113s may flow into the needle attachment component 107s, into the needle 105s, and to tissue of the patient. Too far and the vapor generated by the electrode(s) 113s may heat the length of the inner catheter 119s and outer catheter 109s between the electrode 113s and the needle attachment component 107s, exposing tissue that should not be ablated to excessive heat, possibly causing strictures, and also causing vapor to prematurely condense before passing through the needle 105s, therefore resulting in an insufficient amount of vapor reaching the tissue to be ablated. Therefore, in a preferred embodiment, the most distal electrode 133s of the plurality of electrodes 113s positioned within the catheter lumen 129s is separated by the most proximal part 137s of the needle attachment component 107s by a distance of at least 0.1 mm to a distance of no more than 60 mm. These distance ranges insure that a) no electricity will be communicated to the tissue along with, or independent of, the vapor, b) that sufficient amounts of vapor will be communicated to the tissue to be ablated, and c) that the distance separating the point of generation of vapor and the needle attachment component 107s is small, thereby insuring the associated catheter length is not excessively heated and that the tissue contacting the catheter length is not unduly ablated.

The needle 105s is defined by a metal casing 115s, a lumen passing therethrough 125s, a sharp, and preferably tapered, tip 135s, and a proximal base 145s configured to thread, or otherwise attach to, the needle attachment component 107s. The needle 105s is also curved in a first direction that extends away from the axial length of the catheter 109s. In one embodiment, the needle 105s has a bending capacity that changes depending on the direction of bending. For example, the needle 150s may be more easily bent in a plane parallel to the first direction as opposed to a plane perpendicular to the first direction. Optionally, the needle 105s may be more easily bent in a plane perpendicular to the first direction as opposed to a plane parallel to the first direction. Optionally, a housing 143s of the electrode 113s may also be more easily bent in one direction versus another direction. For example, the electrode housing 143s may also be more easily bent in a plane perpendicular to the first direction as opposed to a plane parallel to the first direction. Optionally, the electrode housing 143s may also be more easily bent in a plane parallel to the first direction as opposed to a plane perpendicular to the first direction. In embodiments, a length of tubing 112s at a proximal end of the catheter handle 190s provides saline to the catheter for conversion to vapor. In embodiments, a dial 192s on the handle 190s may be turned by a user to advance or retract a lead screw 193s attached to the inner catheter 119s to expose or retract the needle 105s from the outer catheter 109s. In some embodiments, the outer catheter 109s comprises a hypo-tube having an outer diameter of 3 mm and an inner diameter of approximately 2.5 mm. In some embodiments, the needle 105s is a 25 gauge needle.

FIG. 1T illustrates a needle chamber 108t of a system for use in the ablation of prostatic tissue, in accordance with some embodiments of the present specification. In embodiments, the catheter further comprises a retractable needle chamber 108t configured to be positioned over the needle 105t and needle attachment component (107s in FIG. 1S). The needle chamber 108t may be retracted using a control on the handle and, once retracted, will expose the needle 105t. To ensure the needle 105t maintains the right radius, degree or extent of curvature, in operation, preferably the needle 105t, pre-deployment and before being positioned in the needle chamber 108t, has a first radius, degree or extent of curvature. Before being positioned in a patient, the needle 105t, having the first radius, degree or extent of curvature, is encased within, and covered by, the needle chamber 108t, resulting in the needle 105t adopting a second radius, degree or extent of curvature. Finally, in use and when inside the patient, the needle chamber 108t may be retracted to expose the needle. Upon doing so, the needle 105t would adopt a third radius, degree or extent of curvature. In this embodiment, the first radius, degree or extent of curvature is greater than the third radius, degree or extent of curvature which is greater than the second radius, degree or extent of curvature. Stated differently, the first radius, degree or extent of curvature is the largest, the third radius, degree or extent of curvature is the smallest and the second radius, degree or extent of curvature is in between the two.

The needle chamber 108t is preferably cylindrical having an internal surface 118t with a higher degree of hardness or stiffness relative to its outside surface 128t. Preferably, the outside surface 128t is made of a polymer while the inside surface 118t comprises a metal. This permits the outside needle chamber surface 128t to be atraumatic, and decrease the possibility of injuring the patient, while the inside needle chamber surface 118t protects from inadvertent puncturing or damage by the needle 105t itself.

In another embodiment, the needle chamber 108t may be configured to receive the needle 105t such that it conforms to the curvature of the needle 105t. Accordingly, in one embodiment, the internal lumen 138t of the needle chamber 108t is curved, reflecting, at least to some degree, the curvature of the needle 105t.

Finally, insulation 175t is positioned along the length of the needle 105t and on the external surface 185t of the needle 105t. A sufficient amount of insulation 175t serves to protect tissue that should not be ablated and improves the dynamics of vapor distribution. Measured from the proximal end of the needle 105t, it is preferred to have the insulation extend at least 5% along the length of the needle 105t and no more than 90% along the length of the needle 105t, and more preferably at least 5% along the length of the needle 105t and no more than 75% along the length of the needle 105t.

FIG. 1U illustrates a needle chamber 108u of a system for use in the ablation of prostatic tissue, in accordance with some embodiments of the present specification. The needle chamber 108u is provided in the form of a ball tip that is attached at the distal end of a catheter body 109u. In embodiments, the catheter comprises a retractable needle chamber 108u configured to be positioned over the needle 105u and needle attachment component (107s in FIG. 1S). The needle chamber 108u may be retracted using a control on the handle and, once retracted, will expose the needle 105u. To ensure the needle 105u maintains the right radius, degree or extent of curvature, in operation, preferably the needle 105u, pre-deployment and before being positioned in the needle chamber 108u, has a first radius, degree or extent of curvature. Before being positioned in a patient, the needle 105u, having the first radius, degree or extent of curvature, is encased within, and covered by, the needle chamber 108u, resulting in the needle 105u adopting a second radius, degree or extent of curvature. Finally, in use and when inside the patient, the needle chamber 108u may be retracted to expose the needle. Upon doing so, the needle 105u would adopt a third radius, degree or extent of curvature. In this embodiment, the first radius, degree or extent of curvature is greater than the third radius, degree or extent of curvature which is greater than the second radius, degree or extent of curvature. Stated differently, the first radius, degree or extent of curvature is the largest, the third radius, degree or extent of curvature is the smallest and the second radius, degree or extent of curvature is in between the two.

The needle chamber 108u is preferably cylindrical having an internal surface 118u with a higher degree of hardness or stiffness relative to its outside surface 128u. Preferably, the outside surface 128u is made of a polymer while the inside surface 118u comprises a metal. This permits the outside needle chamber surface 128u to be atraumatic, and decrease the possibility of injuring the patient, while the inside needle chamber surface 118u protects from inadvertent puncturing or damage by the needle 105u itself.

In another embodiment, the needle chamber 108u may be configured to receive the needle 105u such that it conforms to the curvature of the needle 105u. Accordingly, in one embodiment, the internal lumen 138u of the needle chamber 108u is curved, reflecting, at least to some degree, the curvature of the needle 105u.

FIG. 1N illustrates an ablation system 110 suitable for use in ablating an endometrial tissue, in accordance with embodiments of the present specification. The ablation system 110 comprises a catheter 111 having a catheter body 115 comprising an outer catheter 116 with an inner catheter 117 concentrically positioned inside and extendable outside from a distal end of the outer catheter 116. The inner catheter 117 includes at least one first distal attachment or positioning element 112 and a second proximal attachment or positioning element 113. The inner catheter 117 is positioned within the outer catheter 116 during positioning of the catheter 111 within a cervix or uterus of a patient. The first and second positioning elements 112, 113, in first, compressed configurations, are constrained by, and positioned within, the outer catheter 116 during positioning of the catheter 111. Once the distal end of the outer catheter 111 has been positioned within a cervix of a patient, the inner catheter 117 is extended distally from the distal end of the outer catheter 116 and into a uterus of the patient. The first and second positioning elements 112, 113 expand and become deployed within the uterus. In embodiments, the first and second positioning elements 112, 113 comprise shape memory properties, allowing them to expand once deployed. In some embodiments, the first and second positioning elements 112, 113 are comprised of Nitinol. In some embodiments, once deployed, the first, distal positioning element 112 is configured to contact a uterine wall, positioning the inner catheter 117 within the uterus, and the second, proximal positioning element 113, once deployed, is configured to abut a distal portion of the cervix just within the uterus, blocking passage of ablative vapor back into the cervical os. An internal heating chamber 103 is disposed within a lumen of the inner catheter 117 and configured to heat a fluid provided to the catheter 111 to change said fluid to a vapor for ablation therapy. In some embodiments, the internal heating chamber is positioned just distal to the second positioning element 113. In some embodiments, the catheter 111 is made of or covered with an insulated material to prevent the escape of ablative energy from the catheter body. The inner catheter 117 comprises one or more infusion ports 114 for the infusion of an ablative agent, such as steam. In some embodiments, the one or more infusion ports 114 are positioned on the catheter 111 between the first and second positioning elements 112 and 113. In various embodiments, the first distal attachment or positioning element 112 and second positioning element 113 comprise discs. A fluid, such as saline, is stored in a reservoir, such as a saline pump 14, connected to the catheter 111. Delivery of the ablative agent is controlled by a controller 15 and treatment is controlled by a treating physician via the controller 15. The controller 15 includes at least one processor 23 in data communication with the saline pump 14 and a catheter connection port 21 in fluid communication with the saline pump 14. In some embodiments, at least one optional sensor 22 monitors changes in an ablation area to guide flow of ablative agent. In some embodiments, the optional sensor comprises at least one of a temperature sensor or pressure sensor. In some embodiments, the catheter 111 includes a filter 16 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 25 in data communication with the controller 15, a switch 27 on the catheter 111, or a switch 29 on the controller 15, for controlling vapor flow.

In one embodiment, a user interface included with the microprocessor 15 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

In another embodiment, the outer catheter 116 abuts a cervical canal mucosa without blocking the cervix and the outflow from the uterine cavity. A space between the outer catheter 116 and inner catheter 117 allows for venting via a channel for heated air, vapor or fluid to escape out of the uterine cavity without contacting and damaging the cervical canal.

The ablation device also includes safety mechanisms to prevent users from being burned while manipulating the catheter, including insulation, and optionally, cool air flush, cool water flush, and alarms/tones to indicate start and stop of treatment.

FIG. 1O illustrates another view of a catheter 111 of FIG. 1N, in accordance with some embodiments of the present specification. The catheter 111 includes an elongate body 115 with a proximal end and a distal end. At the distal end, the catheter body 115 includes an outer catheter 116 with an inner catheter 117 concentrically positioned inside and extendable outside from a distal end of the outer catheter 116. The inner catheter 117 includes a distal positioning element 112, proximate its distal end, and a proximal positioning element 113 proximal to the distal positioning element 112. In various embodiments, the positioning elements are discs. The outer catheter 116 is configured to receive the inner catheter 117 and constrain the positioning elements 112, 113 before positioning, as described above. A plurality of infusion ports 114 are located on the inner catheter 117 between the two positioning elements 112, 113. The inner catheter 117 also includes at least one heating chamber 103 just distal to the proximal disc 113. In some embodiments, the heating chamber 103 includes two electrodes 109 configured to receive RF current, heat, and convert supplied fluid, such as saline, to vapor, or steam, for ablation.

Referring to FIG. 1O, for providing electrical current and liquid to the catheter 111, a fluid pump 174 and an RF generator 184 are coupled to the proximal end of the body 115. The fluid pump 174 pumps the fluid, such as saline, through a first lumen (extending along the length of the body 115) to the heating chamber 103. The RF generator 184 supplies electrical current to the electrodes 109, causing the electrodes 109 to generate heat and thereby converting the fluid flowing through the heating chamber 103 into vapor. The generated vapor flows through the first lumen and exits the ports 114 to ablate endometrial tissue. The flexible heating chamber 103 imparts improved flexibility and maneuverability to the catheter 111, allowing a physician to better position the catheter 111 when performing ablation procedures, such as ablating endometrial tissue of a patient.

In various embodiments, the heating electrode 109 is proximal to the proximal positioning element 113, extends beyond the distal end of the proximal positioning element 113, or is completely distal to the distal end of the proximal positioning element 113 but does not substantially extend beyond a proximal end of the distal positioning element 112.

FIG. 1P illustrates a system 100p for use in the ablation of endometrial tissue, in accordance with another embodiment of the present specification. The ablation system 100p comprises a catheter 101p which, in some embodiments, includes a handle 190p having actuators 191p, 192p, 193p for pushing forward a distal bulbous tip 189p of the catheter 101p and for deploying a first distal positioning element 11p and a second proximal positioning element 12p at the distal end of the catheter 101p. In embodiments, the catheter 101p comprises an outer sheath 109p and an inner catheter 107p. In embodiments, the catheter 101p includes a cervical collar 115p configured to rest against an external os once the catheter 101p has been inserted into a uterus of a patient. In embodiments, the distal first positioning element 11p and proximal second positioning element 12p are expandable, positioned at the distal end of the inner catheter 107p, and may be compressed within the outer sheath 109p for delivery. In some embodiments, actuators 192p and 193p comprise knobs. In some embodiments, actuator/knob 192b is used to deploy the distal first positioning element 11p. For example, in embodiments, actuator/knob 192p is turned one quarter turn to deploy the distal first positioning element 11p. In some embodiments, actuator/knob 193b is used to deploy the proximal second positioning element 12p. For example, in embodiments, actuator/knob 193p is turned one quarter turn to deploy the proximal second positioning element 12p. In some embodiments, the handle 190p includes only one actuator/knob 192p which is turned a first quarter turn to deploy the first distal positioning element 11p and then a second quarter turn to deploy the second proximal positioning element 12p. In other embodiments, other combinations of actuators/knobs are used to deploy one or both of the first distal positioning element 11p and second proximal positioning element 12p. In some embodiments, the catheter 101p includes a port 103p for the delivery of fluid, for example cooling fluid, during ablation. In some embodiments, port 103p is also configured to provide for fluid collection, provide vacuum, and provide CO₂ for an integrity test. In some embodiments, the port 103p is positioned on the handle 190p. In some embodiments, at least one electrode 113p is positioned at a distal end of the catheter 101p proximal to the proximal second positioning element 12p. The electrode 113p is configured to receive electrical current, supplied by a connecting wire 111p extending from the controller 15p to the catheter 101p, to heat and convert a fluid, such as saline supplied via tubing 112p extending from the controller 15p to the catheter 101p. Heated fluid or saline is converted to vapor or steam to be delivered by ports 114p ablation. In some embodiments, the catheter 101p is made of or covered with an insulated material to prevent the escape of ablative energy from the catheter body. A plurality of small delivery ports 114p is positioned on the inner catheter 107p between the distal first positioning element 11p and the second proximal positioning element 12p. Ports 114p are used for the infusion of an ablative agent, such as steam. Delivery of the ablative agent is controlled by the controller 15p and treatment is controlled by a treating physician via the controller 15p.

FIG. 1Q illustrates a controller 15q for use with an ablation system, in accordance with an embodiment of the present specification. Controller 15q, similar to controllers 15m, 15r, and 15p, controls the delivery of the ablative agent to the ablation system. The controller 15q therefore provides a control interface to a physician for controlling the ablation treatment. An input port 196q on the controller 15q provides a port to connect the controller 15q to the catheter and provide electrical signal to the catheter. A fluid port 198q on the controller 15q provides a port for connecting a supply to fluid such as saline through a tubing to the catheter. In embodiments, a graphical user interface (GUI) 1100q on the controller 15q shows the settings for operating the ablation system, which may be in use and/or modified by the physician during use. In some embodiments, the GUI is a touchscreen allowing for control of the system by a user.

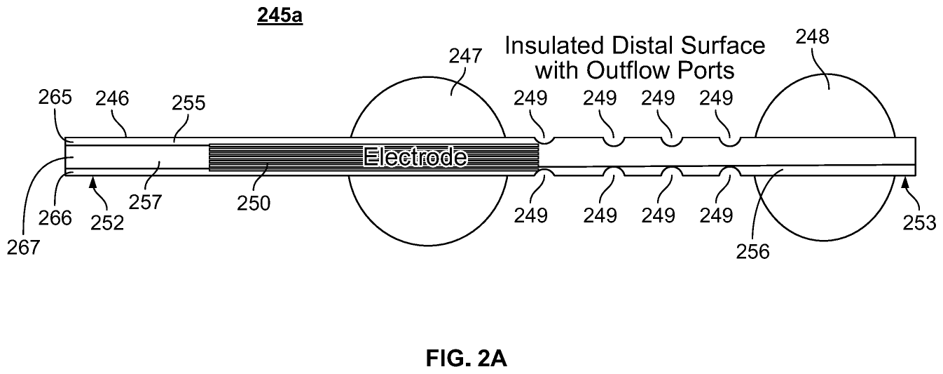

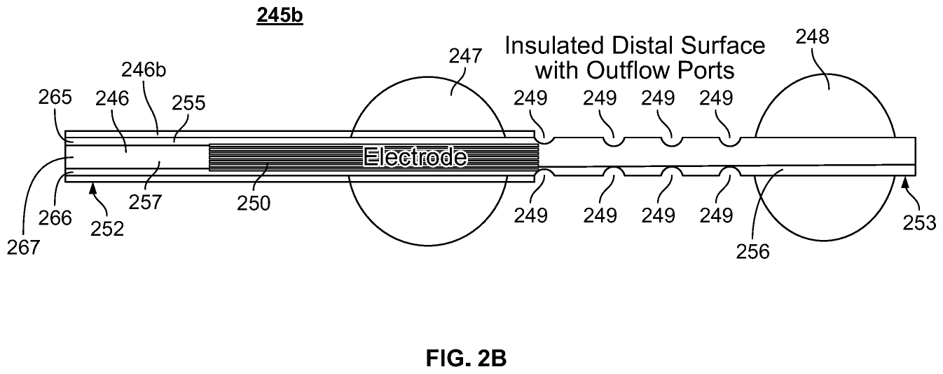

FIGS. 2A and 2B illustrate single and coaxial double balloon catheters 245a, 245b in accordance with embodiments of the present specification. The catheters 245a, 245b include an elongate body 246 with a proximal end 252 and a distal end 253 and a first lumen 255, a second lumen 256, and a third lumen 257 within. In an embodiment, the elongate body 246 is insulated. The catheters 245a, 245b include at least one positioning element 248 proximate their distal end 253. In various embodiments, the positioning element is an inflatable balloon. In some embodiments, the catheters include more than one positioning element. As shown in FIG. 2B, the coaxial catheter 245b includes an outer catheter 246b that accommodates the elongate body 246.

In the embodiments depicted in FIGS. 2A, 2B, the catheters 245a, 245b include a proximal first inflatable balloon 247 and a distal second inflatable balloon 248 positioned proximate the distal end of the body 246 with a plurality of infusion ports 249 located on the body 246 between the two balloons 247, 248. It should be appreciated that, while balloons are preferred, other positioning elements, as previously described, may be used.

The body 246 includes a first lumen 255 (extending along a portion of the entire length of the body 246) in fluid communication with a first input port 265 at the proximal end 252 of the catheter body 246 and with said proximal first balloon 247 to inflate or deflate the proximal first balloons 247, 248 by supplying or suctioning air through the first lumen 255. In an embodiment, use of a two-balloon catheter as shown in FIGS. 2A and 2B results in the creation of a seal and formation of a treatment area having a radius of 3 cm, a length of 9 cm, a surface area of 169.56 cm² and a treatment volume of 254.34 cm³. The body 246 includes a second lumen 256 (extending along the entire length of the body 246) in fluid communication with a second input port 266 at the proximal end 252 of the catheter body 246 and with said distal second balloon 248 to inflate or deflate the distal second balloon 248 by supplying or suctioning air through the second lumen 256. In another embodiment, the body includes only a first lumen for in fluid communication with the proximal end of the catheters and the first and second balloons for inflating and deflating said balloons. The body 246 also includes an in-line heating element 250 placed within a second third lumen 257 (extending along the length of the body 246) in fluid communication with a third input port 267 at the proximal end 252 of the catheter body 246 and with said infusion ports 249. In one embodiment, the heating element 250 is positioned within the third lumen 257, proximate and just proximal to the infusion ports 249. In an embodiment, the heating element 250 comprises a plurality of electrodes. In one embodiment, the electrodes of the heating element 250 are folded back and forth to increase a surface contact area of the electrodes with a liquid supplied to the third lumen 257. The second third lumen 257 serves to supply a liquid, such as water/saline, to the heating element 250.

In various embodiments, a distance of the heating element 250 from a nearest port 249 ranges from 1 mm to 50 cm depending upon a type of therapy procedure to be performed.

A fluid pump, an air pump and an RF generator are coupled to the proximate end of the body 246. The air pump propels air via said first and second inputs 265, 266 through the first and second lumens to inflate the balloons 247, 248 so that the catheters 245a, 245b are held in position for an ablation treatment. The fluid pump pumps a liquid, such as water/saline, via said third input 267 through the second third lumen 257 to the heating element 250. The RF generator supplies power and electrical current to the electrodes of the heating element 250, thereby causing the electrodes to heat and converting the liquid (flowing through around the heating element 250) into vapor. In other embodiments, the electrodes heat the fluid using resistive heating or ohmic heating. The generated vapor exits the ports 249 for ablative treatment of target tissue. In embodiments, the supply of liquid and electrical current, and therefore delivery of vapor, is controlled by a microprocessor.

### Prostate Ablation

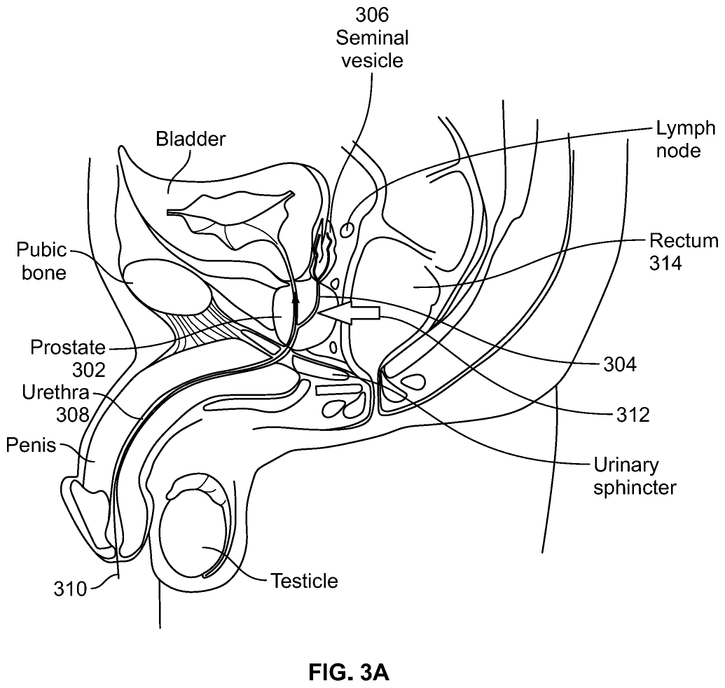

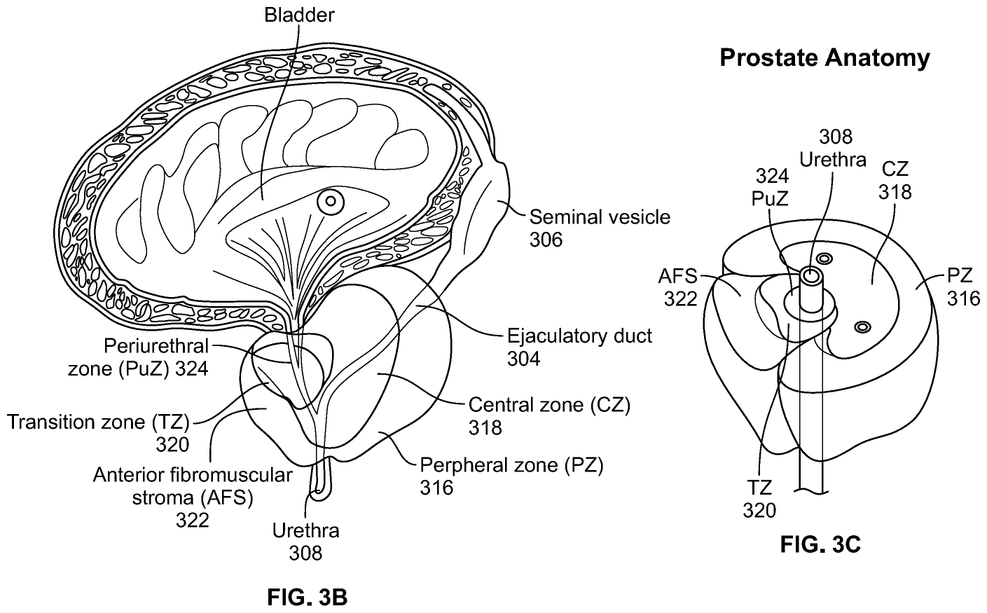

FIG. 3A illustrates a typical anatomy of a prostatic region for descriptive purposes. FIGS. 3B and 3C illustrate exemplary transparent views of prostate 302 anatomy, highlighting the peripheral zone (PZ) 316, in addition to other zones in the periphery of the prostate 302. Referring to the figures, embodiments of the present specification permit the ablation of prostate 302, by ablating PZ 316 prostatic tissue. In accordance with the various embodiments of the present specification, the embodiments enable ablating a prostate 302 tissue without completely ablating a central zone (CZ) 318 prostate tissue so as not to damage an ejaculatory duct 304, emerging from the duct of the seminal vesicle 306, which could cause a stricture of the ejaculatory duct 304. For purposes of the present specification, ‘completely ablating’ is defined as ablating more than 55% of a surface area or a volume around an anatomical structure.

Embodiments of the present specification enable ablating a prostate 302 tissue by ablating one of the numerous anatomical structures along various treatment pathways to treat the prostate 302. FIG. 3A illustrates a pathway 310 along the urethra as an exemplary pathway into the prostatic region for ablation also known as the transurethral approach. An alternative pathway 312 is illustrated through a wall between rectum 314 and prostate 302. In embodiments, prostate 302 tissue is ablated through urethra 308 or through a wall from rectum 314. In either case, the embodiments of the present specification ensure greater than 0% and less than 75% of the circumference of the periurethral zone 324, CZ 318, or any other zone, is ablated during the ablation of the prostate 302. In another embodiment, the prostate is accessed from the base of a bladder around a bladder neck without needing to go through a prostatic urethra, thereby avoiding the risk of ablating and stricturing the prostatic urethra. This approach is best reserved for ablation of benign or malignant obstruction caused by disease in the central zone of the prostate or median lobe hypertrophy.

In one embodiment, the ejaculatory duct 304 is the anatomical structure that is ablated. In another embodiment, the urethra 308 is ablated without completely ablating a circumference of the urethra 308 so as not to cause a stricture of the urethra 308. In other embodiments, the anatomical structures ablated may include the capsule of the prostate, including a rectal wall. In some embodiments, a portion of the prostate 302 or a portion of one or more of the CZ 318, the PZ 316, a transitional zone (TZ) 320, and an Anterior Fibromuscular Strauma (AFS) 322, are ablated. The different anatomical structures are ablated without ablating a contiguous circumference of periurethral zone (PuZ) 324 that surrounds the urethra 308. In some embodiments, greater than 0% and no more than 90% of the contiguous PuZ 324 circumference is ablated. In some embodiments, greater than 0% and less than 75% of the contiguous PuZ 324 circumference is ablated. In some embodiments, greater than 0% and less than 25% of the contiguous PuZ 324 circumference is ablated.

Therefore, in one embodiment, CZ 318 of a prostate 302 is ablated while ablating greater than 0% and less than 75% of a contiguous circumference of prostatic urethra 308. In another embodiment, CZ 318 of a prostate 302 is ablated while ablating greater than 0% and less than 75% of a contiguous circumference of ejaculatory duct 304. In one embodiment, TZ 320 of a prostate 302 is ablated while ablating greater than 0% and less than 75% of a contiguous circumference of prostatic urethra 308. In another embodiment, TZ 320 of a prostate 302 is ablated while ablating greater than 0% and less than 75% of a contiguous circumference of ejaculatory duct 304. In another embodiment, a median lobe of prostate 302 is ablated while ablating greater than 0% and less than 75% of a contiguous circumference of ejaculatory duct 304. In an embodiment a majority of the median lobe or CZ 318, ranging from more than 25% to more than 75%, is ablated without ablating a majority (>75%) of PuZ 324. In an embodiment a majority of TZ 320, ranging from more than 25% to more than 75%, is ablated without ablating a majority (>75%) of AFS 322. In some embodiments, preferably a range of 1% to 25%, and every increment therein, of a contiguous circumference of a prostatic urethra is ablated. In some embodiments, preferably a range of 1% to 25%, and every increment therein, of a contiguous circumference of an ejaculatory duct is ablated. In some embodiments, preferably a range of 1% to 25%, and every increment therein, of a thickness of the rectal wall is ablated. In the various embodiments, a mucosal layer of the rectal wall is not ablated.

FIG. 4A is an illustration of a water-cooled catheter 400 while FIG. 4B is a cross-section of the tip of the catheter 400, in accordance with another embodiment of the present specification. Referring now to FIGS. 4A and 4B, the catheter 400 comprises an elongate body 402 having a proximal end and a distal end. The distal end includes a positioning element 404, such as an inflatable balloon. A plurality of openings 406 are located proximate the distal end for enabling a plurality of associated thermally conductive elements 408, such as needles, to be extended (at an angle from the catheter 400, wherein the angle ranges between 10 to 150 degrees) and deployed or retracted through the plurality of openings 406. In accordance with an aspect, the plurality of retractable needles 408 are hollow and include at least one opening to allow delivery of an ablative agent, such as steam or vapor 410, through the needles 408 when the needles 408 are extended and deployed through the plurality of openings 406. This is illustrated in context of FIGS. 1L and 1M. A sheath 412 extends along the body 402 of the catheter 400, including the plurality of openings 406, to the distal end. The plurality of openings 406 extend from the body 402 and through the sheath 412 to enable the plurality of needles 408 to be extended beyond the sheath 412 when deployed. During use, cooling fluid such as water or air 414 is circulated through the sheath 412 to cool the catheter 400. Vapor 410 for ablation and cooling fluid 414 for cooling are supplied to the catheter 400 at its proximal end.

It should be noted that alternate embodiments may include two positioning elements or balloons—one at the distal end and the other proximate the openings 406 such that the openings 406 are located between the two balloons.

FIG. 4C illustrates embodiments of a distal end of a catheter 400c for use with the system 101m of FIG. 1M. In the embodiments shown in FIG. 4C, one or a plurality of openings 406c is located proximate the distal end of an outer sheath 412c for enabling one or a plurality of associated thermally conductive elements 408c, such as needles, to be extended from an inner catheter 416c (at an angle from the catheter 400c, wherein the angle ranges between 10 to 90 degrees) and deployed or retracted through the one or plurality of openings 406c. Each needle 408c includes a beveled sharp edge 418c for puncturing a prostatic tissue and an opening 410c for the delivery of ablative agent. In some embodiments, each needle 408c has a gradient of coating for insulation or echogenicity. The coating may be ceramic, polymer, or any other material suitable for coating the needles and providing insulation and/or echogenicity to the needles 408c. The coatings are provided at the base of the needle 408c to varying lengths to the tips. In some embodiments, each needle 408c includes a physical gradient in its shape, such as a taper, beveled tip, or any other structural gradient, to regulate and manage steam distribution. In some embodiments, the physical shape of the needle is configured for tissue cutting. The needle edge is configured for puncturing the tissue without causing shearing or damage to the tissue.

In the multi-needle embodiment illustrated in FIG. 4C, the openings 406c are circumferentially positioned at an equal distance from each other on the outer sheath 412c. In various embodiments, an opening 406c may be used to extend one or more needles 408c. In other embodiments, the openings 406c and needles 408c are offset, or circumferentially positioned at an unequal distance from each other on the outer sheath 412c. FIG. 4D illustrates other embodiments of a distal end of a catheter 400d for use with the system 101m of FIG. 1M. One or a plurality of openings 406d are circumferentially positioned around a sheath 412d at an equal distance from each other and at the distal edge 420d of sheath 412d. In some multi-needle embodiments, the plurality of openings 406d circumferentially positioned around sheath 412d, are offset, and not always at an equal distance, from each other at the distal edge 420d of sheath 412d. The distal end of catheter 400d can also have gradients of coating for insulation or echogenicity. The coatings may cover the needle surface in a range of 0 to 100% of the needle surface. In embodiments, coatings are concentrated at a proximal end of the needles 408d to provide insulation to the needles. In some embodiments, coatings are concentrated at a distal end of the needles 408d to impart the needles 408d with echogenicity. The coatings may be ceramic, polymer, or any other material that may provide the needles 408d with insulation and/or echogenicity. The coatings are provided at the base of the needle to varying lengths to the tips. In some embodiments, the needles have a physical gradient (shape, taper, or any other) to regulate and manage steam distribution. In some embodiments, the needle tips are shaped for cutting tissue. One or a plurality of associated thermally conductive elements 408d, such as needles, is configured to be extended from an inner catheter 416d (at an angle from the catheter 400d, wherein the angle ranges between 10 to 90 degrees) and deployed or retracted through the one or plurality of openings 406d. Each needle 408d includes a beveled sharp edge 418d for puncturing a prostatic tissue and an opening 410d for the delivery of ablative agent. Referring simultaneously to FIGS. 4C and 4D, in accordance with an aspect, each of the retractable needles 408c, 408d is hollow and includes at least one opening 410c, 410d to allow delivery of an ablative agent, such as steam or vapor, through the one or more needles 408c, 408d when the needles 408c, 408d are extended and deployed through the one or plurality of openings 406c, 406d. This is further illustrated in the context of FIGS. 1L and 1M. Outer sheath 412c, 412d extends along a body of the catheter 400c, 400d, including the plurality of openings 406c, 406d, to the distal end. The plurality of openings 406c, 406d extends from the body and through the sheath 412c, 412d to enable each of the plurality of needles 408c, 408d to be extended beyond the sheath 412c, 412d when deployed. In some embodiments, openings 406c, 406d are provided with a locking mechanism for locking the needles 408c, 408d in their deployed position so that the needles 408c, 408d are prevented from being compressed. In some embodiments, the locking mechanisms are operated independently to provide the user with the ability to customize positions of needles 408c, 408d for disease location, amount of ablation, and orientation of the needles. The locking mechanism is deployed in all the embodiments of the present specification, for treating various conditions including BPH and AUB. In all of these embodiments, the needles are electrically separated from the vapor generation chamber by a length of the catheter so as to electrically insulate the tissue from the RF electrical current being delivered to the vapor generation chamber.

In different embodiments, size and numbers of openings 406c, 406d may vary. Further, in various embodiments, openings 406c, 406d that provide exit ports for the steam can be all the same size along the length of the sheath 412c, 412d, and may have different patterns such as and not limited to: spiral, circular, or any other pattern. Further, openings 406c, 406d may have a gradient of dimensions to force steam distribution into certain regions of the anatomy. In an exemplary embodiment, the diameters of the openings 406c, 406d may vary by at least 10% (but not limited to) from top to bottom or from bottom to top. Additionally, the openings 406c, 406d may be of different shapes such as round, oval, or any other shape.

FIG. 4E illustrates an embodiment of a slit flap used to cover openings 406c, 406d of FIGS. 4C and 4D, in accordance with some embodiments of the present specification. In embodiments, the slit flap 422e is made from material such as, but not limited to, silicone or polyurethane (PU). A flap 422e is positioned over each opening 406c, 406d. Needles 408c, 408d may be extended (at an angle from the catheter 400c, 400d, wherein the angle ranges between 30 to 90 degrees) and deployed or retracted through the plurality of flaps 422e.

FIG. 4F illustrates an embodiment of a positioning element 424f to be positioned at a distal end of an ablation catheter, to position the ablation catheter in the prostatic urethra, in accordance with the present specification. In some embodiments, positioning elements having the same shape as element 424f are used in the uterus as well for endometrial ablation, as described in embodiments of the present specification. In embodiments, the positioning element comprises a plurality of wires 426f woven into a pattern, for example a spiral pattern. In embodiments, the wires 426f are composed a shape memory material to allow for compression of the positioning element 424f during delivery. In some embodiments, the shape memory material is Nitinol. In various embodiments, the positioning element 424f has a funnel, bell, spherical, oval, ovoid, or acorn shape and is substantially cylindrical when compressed. The positioning element 424f, when deployed, abuts and rests in the bladder or the bladder neck.

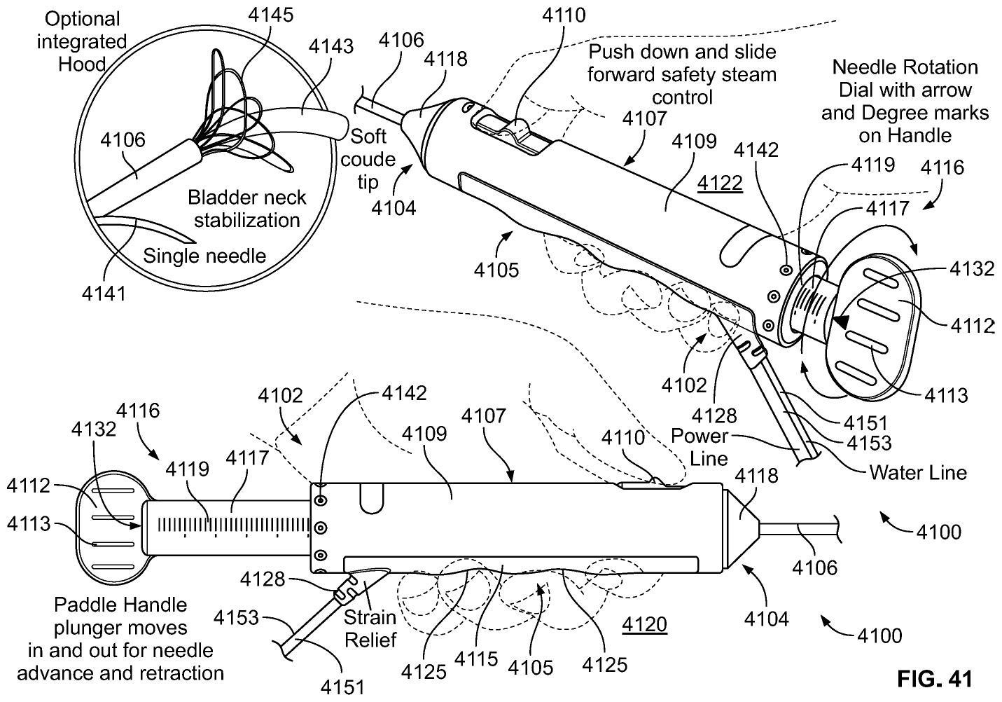

FIGS. 4G to 4L illustrate exemplary steps showing one embodiment of using a catheter 400, similar to the catheters of FIGS. 4C, 4D, and 4E, to ablate prostate tissue 428, in accordance with the present specification. An outer catheter or sheath 412 encompasses an inner catheter 402. FIG. 4G illustrates advancing a distal end of catheter 400g through a prostatic urethra 430g. In embodiments, the catheter 400g includes a coude tip 432g at its distal end 422g configured to push through and position against a patient's bladder 434g. In embodiments, the coude tip 432g is bent or elbow tipped. FIG. 4H illustrates advancing a distal end of catheter 400h into a bladder 434h, and FIG. 4I illustrates even further advancing a distal end of catheter 400i into the bladder 434i. As shown in FIGS. 4H and 41, outer sheath 412h/412i is retracted slightly to expose a distal end of inner catheter 402h/402i with a positioning element 424h/424i in a compressed configuration. Referring to FIG. 4J, positioning element 424j is expanded and catheter 400j is retracted to position positioning element 424j proximate a bladder neck 436j or the distal end of the prostatic urethra 430j. Referring to FIG. 4K, a needle 408k is extended from the catheter 400k and into the prostatic tissue 428k. In embodiments, needle 408k refers to at least one, and in some embodiments, more than one needle. In embodiments, the needle 408k is deployed and extended according to the embodiments illustrated in FIGS. 4A, 4C, and 4D. Referring to FIG. 4L, an ablative agent 4381 is delivered through the needle 408l into prostate tissue 428l.

In an alternative embodiment, referring to FIG. 4M, a catheter 400m has a positioning element 424m positioned on the catheter 400m proximal to needle 408m, which in turn is positioned at the distal end of the catheter 400m. In other embodiments, the catheter includes more than one needle. The catheter includes an outer sheath 412m and an inner catheter 402m. The positioning element 424m and needle 408m are positioned on the inner catheter 402m, with the needle 408m distal to the positioning element 424m. As shown in FIG. 4M, the catheter 400m is advanced into a prostatic urethra 430m with both the needle 408m and positioning element 424m in collapsed configurations. Referring to FIG. 4N, the positioning element 424n is expanded to hold the catheter 400n within the prostatic urethra 430n and the needle 408n is deployed into the prostatic tissue 428n for delivery of ablative agent. In various embodiments, the positioning element 424n has a funnel, bell, spherical, oval, ovoid, or acorn shape when deployed and is substantially cylindrical when compressed.

FIG. 4O is a flow chart illustrating the steps involved in using an ablation catheter to ablate a prostate of a patient, in accordance with embodiments of the present specification. At step 440, a coude tip of the catheter is used to push through a patient's prostatic urethra and position a distal end of the catheter against the patient's bladder. At step 442, an outer sheath of the catheter is retracted, using an actuator, to reveal a positioning element, or bladder anchor, and the positioning element is positioned, for example in the bladder neck, to position the catheter for ablation. At step 444, the outer sheath is retracted further to deploy a needle or plurality of needles from the catheter and into prostatic tissue. In some embodiments, the one or more needles are deployed out of an internal lumen of an inner catheter of the catheter and through slots in the outer sheath. In another embodiment, the sleeves naturally fold outward as the outer sheath is retracted. At step 446 vapor or steam is delivered through the one or more needles to ablate the prostatic tissue.

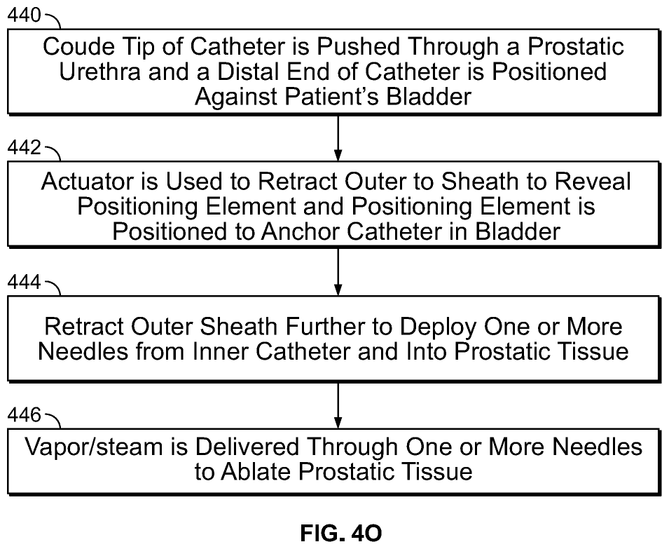

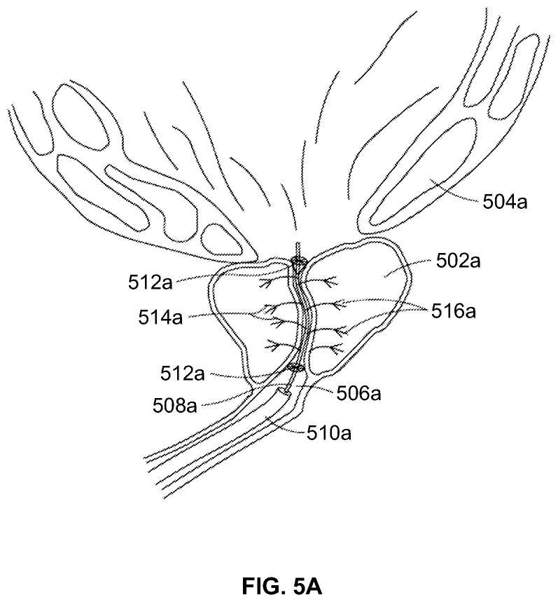

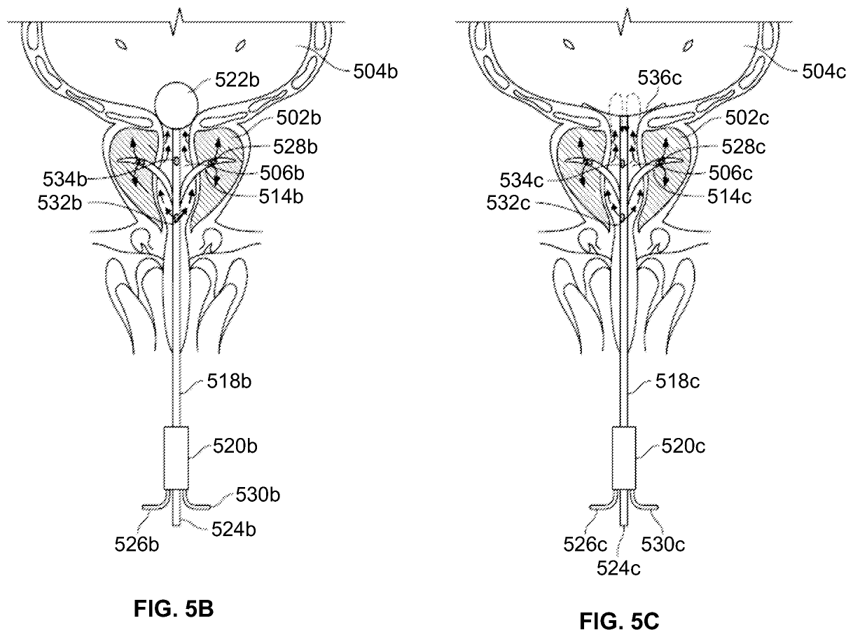

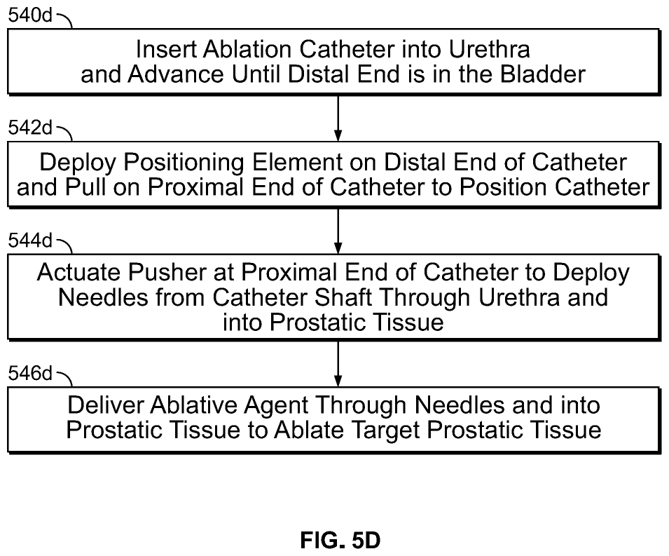

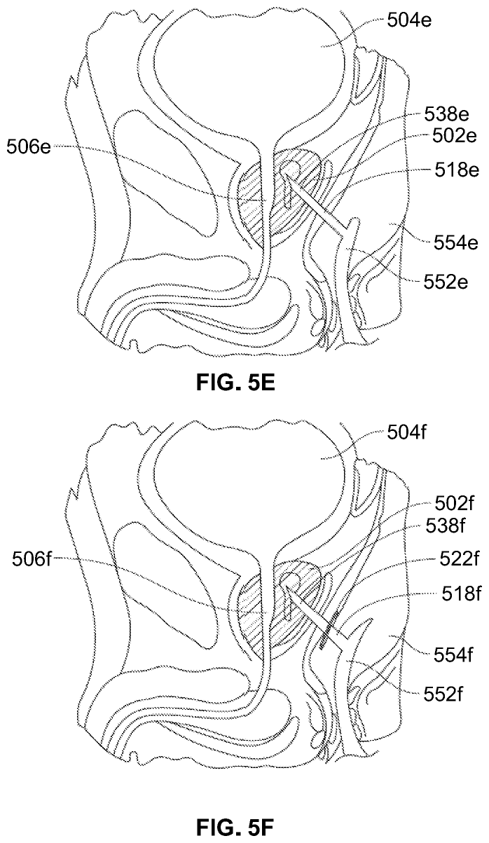

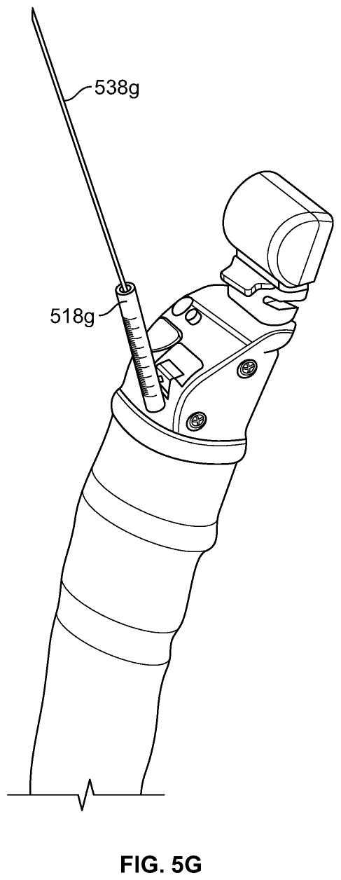

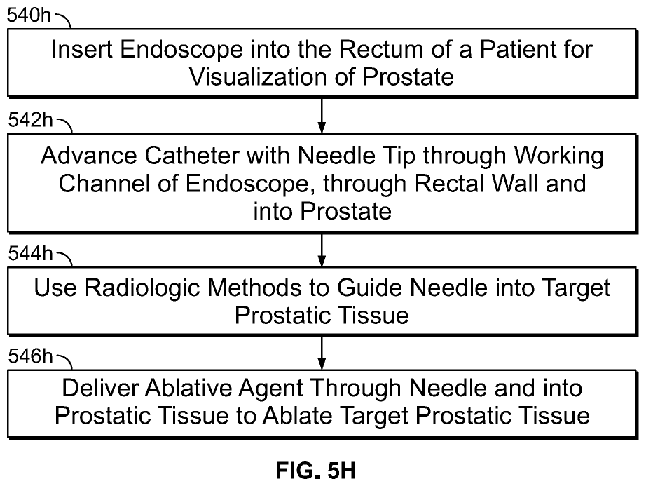

FIG. 5A illustrates prostate ablation being performed on an enlarged prostrate in a male urinary system by using a catheter (such as the catheter 400 of FIG. 4A—with two positioning elements), in accordance with an embodiment of the present specification. A cross-section of a male genitourinary tract having an enlarged prostate 502a, bladder 504a, and urethra 506a is illustrated. The urethra 506a is compressed by the enlarged prostate 502a. The ablation catheter 508a is passed through the cystoscope 510a positioned in the urethra 506a distal to the obstruction. The positioning elements 512a are deployed to center the catheter in the urethra 506a and one or more insulated needles 514a are passed to pierce the prostate 502a. The vapor ablative agent 516a is passed through the insulated needles 514a thus causing ablation of the diseased prostatic tissue resulting in shrinkage of the prostate. In one embodiment, only the proximal positioning element is used while in another embodiment, only the distal positioning element is used.

The size of the enlarged prostate could be calculated by using the differential between the extra-prostatic and intra-prostatic urethra. Normative values could be used as baseline. Additional ports for infusion of a cooling fluid into the urethra can be provided to prevent damage to the urethra while the ablative energy is being delivered to the prostrate for ablation, thus preventing complications such as stricture formation.

In one embodiment, the positioning attachment must be separated from the ablation region by a distance of greater than 0.1 mm, preferably 1 mm to 5 mm and no more than 2 cm. In another embodiment, the positioning attachment can be deployed in the bladder and pulled back into the urethral opening/neck of the bladder thus fixing the catheter. In one embodiment, the positioning device is between 0.1 mm and 10 cm in diameter.

FIG. 5B is an illustration of transurethral prostate ablation being performed on an enlarged prostrate 502b in a male urinary system using an ablation device (such as the catheter 400 of FIG. 4A—with one positioning element), in accordance with one embodiment of the present specification. Also depicted in FIG. 5B are the urinary bladder 504b and prostatic urethra 506b. An ablation catheter 518b with a handle 520b and a positioning element 522b is inserted into the urethra 506b and advanced into the bladder 504b. The positioning element 522b is inflated and pulled to the junction of the bladder with the urethra, thus positioning needles 514b at a predetermined distance from the junction. In some embodiments, the positioning element 522b is inflated to a first volume in the bladder 504b proximate the junction of the bladder 504b with the urethra 506b, to position needles 514b proximate to prostate 502b; and to a second volume, different from the first volume, to position needles 514b at a different position proximate to prostate 504b. Using a balloon as the positioning element 522b, provides counter-traction while the needles 514b are being deployed.

Using a pusher 524b, the needles 514b are then pushed out at an angle between 10 and 90 degrees from the catheter 518b through the urethra 506b into the prostate 504b. Vapor is administered through a port 526b that travels through the shaft of the catheter 518b and exits from openings 528b in the needles 514b into the prostatic tissue, thus ablating the prostatic tissue. In embodiments, the vapor is delivered for a predetermined time, to a predetermined pressure, and to deliver a predetermined amount of energy. In some embodiments, the vapor is delivered for a period that is less than five minutes, and preferably for a period within a range between 2 seconds to 120 seconds, and more preferably for a period of time of 60 to 90 seconds. In embodiments, the vapor is delivered at a pressure that is less than 5 atm and in some cases less than 1 atm, and preferably at a pressure no greater than 10% above atmospheric pressure. In embodiments, the vapor is delivered at an energy in a range of 10 cal to 10,000 cal.

In one embodiment, the needles 514b are insulated so as to prevent damage to a prostatic urethra 506b or a periurethral zone. Additionally, in embodiments, the needles are deployed to deliver vapor at a location that is preferentially away from the ejaculatory duct. In some embodiments, a shape of needles 514b is different during delivery of vapor compared to their shape prior to delivery of vapor.

Optional port 530b allows for insertion of cool fluid at a temperature <37 degree C. through opening 532b to cool the prostatic urethra 506b or the periurethral zone. Optional temperature sensors 534b can be installed to detect the temperature of the prostatic urethra and modulate the delivery of vapor.

FIG. 5C is an illustration of transurethral prostate ablation being performed on an enlarged prostrate 502c in a male urinary system using an ablation device, in accordance with another embodiment of the present specification. Also depicted in FIG. 5C are the urinary bladder 504c and prostatic urethra 506c. An ablation catheter 518c with a handle 520c and a positioning element 536c is inserted into the urethra 506c and advanced into the bladder 504c. The positioning element 536c is a compressible disc that is expanded in the bladder 504c and pulled to the junction of the bladder with the urethra, thus positioning needles 514c at a predetermined distance from the junction. In some embodiments, the positioning element 536c is expanded to a first size in the bladder 504c proximate the junction of the bladder 504c with the urethra 506c, to position needles 514c proximate to prostate 502c; and to a second size, different from the first size, to position needles 514c at a different position proximate to prostate 502c.

Using a pusher 524c, the needles 514c are then pushed out at an angle between 10 and 90 degree from the catheter 518c through the urethra 506c into the prostate 502c. Vapor is administered through a port 526c that travels through the shaft of the catheter 518c and exits through openings 528c in the needles 514c into the prostatic tissue, thus ablating the prostatic tissue. In embodiments, the vapor is delivered for a predetermined time, to a predetermined pressure, to deliver a predetermined amount of energy. In some embodiments, the vapor is delivered for a period of time that is less than five minutes, and preferably for a period of time within a range of 60 to 90 seconds. In other embodiments, the vapor is delivered for a period of time with a range between 2 seconds and 30 seconds. In another embodiment, the vapor is delivered for a period of time with a range between 30 seconds and 60 seconds. In embodiments, the vapor is delivered at a pressure that is less than 5 atm and in some cases less than 1 atm, and preferably at a pressure no greater than 10% above atmospheric pressure.

In one embodiment, the needles 514c are insulated so as to prevent damage to a prostatic urethra 506c or a periurethral zone. Additionally, in embodiments, the needles are deployed to deliver vapor at a location that is preferentially away from the ejaculatory duct. In some embodiments, a shape of needles 514c is different during delivery of vapor compared to their shape prior to delivery of vapor.

Optional port 530c allows for insertion of cool fluid at a temperature <37 degree C. through opening 532c to cool the prostatic urethra 506c or the periurethral zone. Optional temperature sensors 534c can be installed to detect the temperature of the prostatic urethra and modulate the delivery of vapor.

FIG. 5D is a flow chart listing the steps involved in a transurethral enlarged prostate ablation process using an ablation catheter, in accordance with one embodiment of the present specification. At step 540d, an ablation catheter (such as the catheter 400 of FIG. 4A) is inserted into the urethra and advanced until its distal end is in the bladder. A positioning element is then deployed on the distal end of the catheter, at step 542d, and the proximal end of the catheter is pulled so that the positioning element abuts the junction of the bladder with the urethra, thereby positioning the catheter shaft within the urethra. A pusher at the proximal end of the catheter is actuated to deploy needles from the catheter shaft through the urethra and into the prostatic tissue at step 544d. At step 546d, an ablative agent is delivered through the needles and into the prostate to ablate the target prostatic tissue.

FIG. 5E is an illustration of transrectal prostate ablation being performed on an enlarged prostrate in a male urinary system using an ablation device, in accordance with one embodiment of the present specification. Also depicted in FIG. 5E are the urinary bladder 504e and prostatic urethra 506e. The ablation device comprises a catheter 518e with a needle tip 538e. An endoscope 552e is inserted into the rectum 554e for the visualization of the enlarged prostate 502e. In various embodiments, the endoscope 552e is an echoendoscope or a transrectal ultrasound such that the endoscope can be visualized using radiographic techniques. The catheter 518e with needle tip 538e is passed through a working channel of the endoscope and the needle tip 538e is passed transrectally into the prostate 502e. A close-up illustration of the distal end of the catheter 518e (518g) and needle tip 538e (538g) is depicted in FIG. 5G. An ablative agent is then delivered through the needle tip 538e into the prostatic tissue for ablation. In embodiments, the prostatic tissue is ablated without ablating a full thickness of the rectal wall. In some embodiments, no more than 90% of a thickness of the rectal wall is ablated. In some embodiments, greater than 0% and less than 75% of a thickness of the rectal wall is ablated. In some embodiments, preferably a range of 1% to 25%, and every increment therein, of a thickness of the rectal wall is ablated. In some embodiments, a mucosal layer of the rectal wall is not ablated.

In one embodiment, the catheter 518e and needle tip 538e are composed of a thermally insulated material. In various embodiments, the needle tip 538e is an echotip or sonolucent tip that can be observed using radiologic techniques for accurate localization in the prostate tissue. In one embodiment, an optional catheter (not shown) can be placed in the urethra to insert fluid to cool the prostatic urethra 506e. In one embodiment, the inserted fluid has a temperature less than 37° C.

FIG. 5F is an illustration of transrectal prostate ablation being performed on an enlarged prostrate in a male urinary system using a coaxial ablation device having a positioning element, in accordance with another embodiment of the present specification. Also depicted in FIG. 5F are the urinary bladder 504f and prostatic urethra 506f. The ablation device comprises a coaxial catheter 518f having an internal catheter with a needle tip 538f and an external catheter with a positioning element 522f. An endoscope 552f is inserted into the rectum 554f for the visualization of the enlarged prostate 502f. In various embodiments, the endoscope 552f is an echoendoscope or a transrectal ultrasound such that the endoscope can be visualized using radiographic techniques. The coaxial catheter 518f with needle tip 538f and positioning element 522f is passed through a working channel of the endoscope such that the positioning element 522f comes to rest up against the rectal wall and the internal catheter is advanced transrectally, thereby positioning the needle tip 538f at a predetermined depth in the prostate 502f. A close-up illustration of the distal end of the catheter 518f (518g) and needle tip 538f (538g) is depicted in FIG. 5G. In one embodiment, the positioning element is a compressible disc that has a first, compressed pre-employment configuration and a second, expanded deployed configuration once it has passed beyond the distal end of the endoscope 552f. An ablative agent is then delivered through the needle tip 538f into the prostatic tissue for ablation. In embodiments, the prostatic tissue is ablated without ablating a full thickness of the rectal wall. In some embodiments, no more than 90% of a thickness of the rectal wall is ablated. In some embodiments, greater than 0% and less than 75% of a thickness of the rectal wall is ablated. In some embodiments, preferably a range of 1% to 25%, and every increment therein, of a thickness of the rectal wall is ablated. In some embodiments, a mucosal layer of the rectal wall is not ablated.

In one embodiment, the coaxial catheter 518f, needle tip 538f, and positioning element 522f are composed of a thermally insulated material. In various embodiments, the needle tip 538f is an echotip or sonolucent tip that can be observed using radiologic techniques for accurate localization in the prostate tissue. In one embodiment, an optional catheter (not shown) can be placed in the urethra to insert fluid to cool the prostatic urethra 506f In one embodiment, the inserted fluid has a temperature less than 37° C.

FIG. 5H is a flow chart listing the steps involved in a transrectal enlarged prostate ablation process using an ablation catheter, in accordance with one embodiment of the present specification. At step 540h, an endoscope is inserted into the rectum of a patient for visualization of the prostate. A catheter with a needle tip is then advanced, at step 542h, through a working channel of the endoscope and through the rectal wall and into the prostate. Radiologic methods are used to guide the needle into the target prostatic tissue at step 544h. At step 546h, an ablative agent is delivered through the needle and into the prostate to ablate the target prostatic tissue. In embodiments, the prostatic tissue is ablated without ablating a full thickness of the rectal wall. In some embodiments, no more than 90% of a thickness of the rectal wall is ablated. In some embodiments, greater than 0% and less than 75% of a thickness of the rectal wall is ablated. In some embodiments, preferably a range of 1% to 25%, and every increment therein, of a thickness of the rectal wall is ablated. In some embodiments, a mucosal layer of the rectal wall is not ablated.

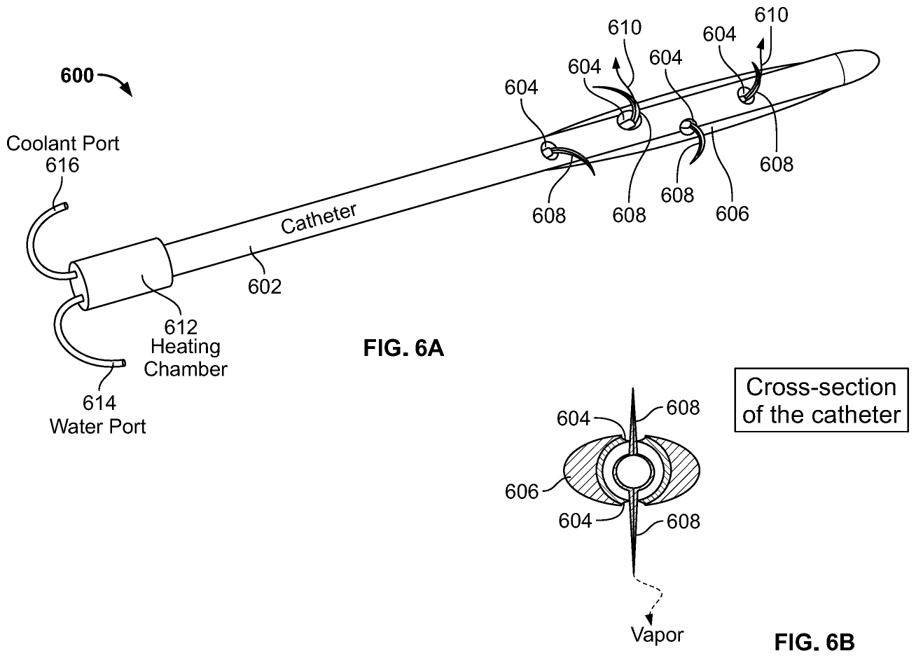

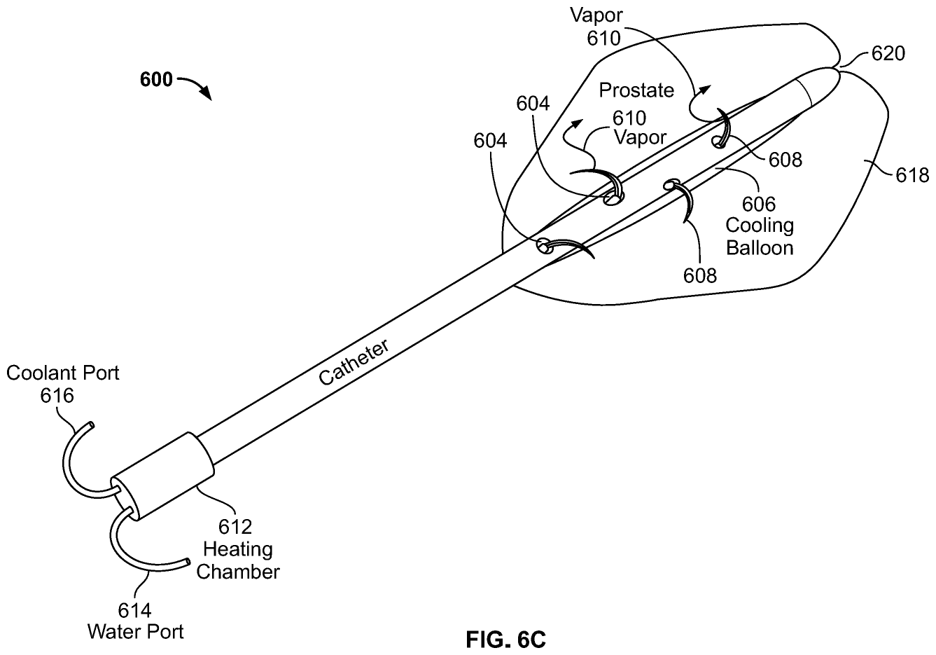

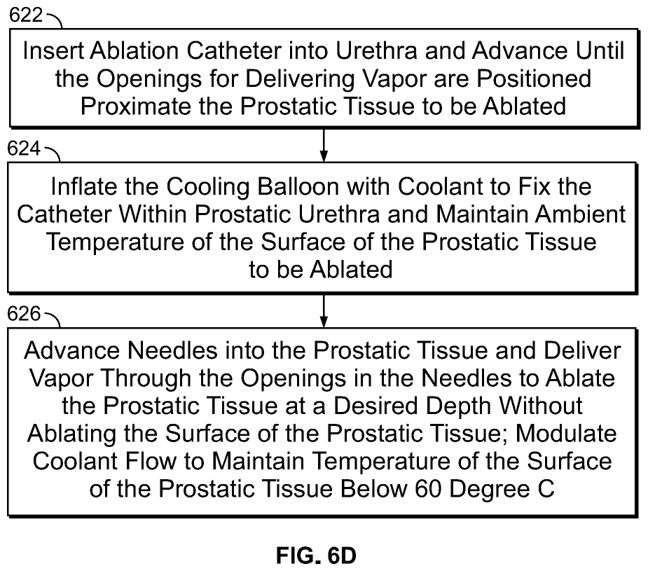

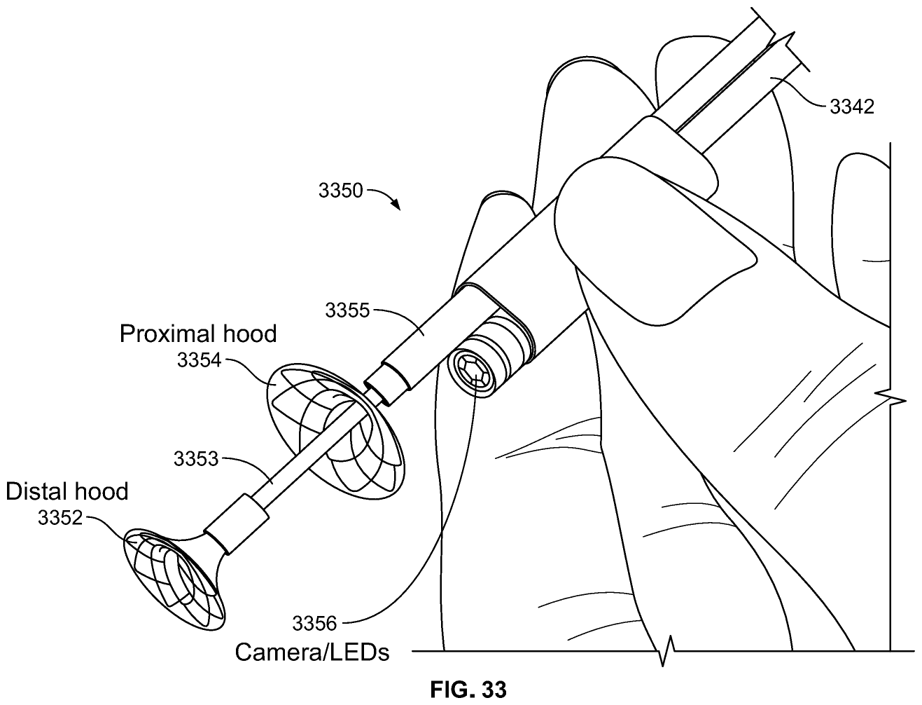

FIG. 6A illustrates an ablation catheter 600 while FIG. 6B is a cross-section of the tip of the catheter 600, in accordance with an embodiment of the present specification. Referring now to FIGS. 33A and 33B, the catheter 600 comprises an elongate body 602 having a proximal end and a distal end. A plurality of openings 604 and an inflatable balloon 606 are located proximate the distal end. The plurality of openings 604 enable a plurality of associated thermally conductive elements 608, such as needles, to be extended (at an angle from the catheter 600, wherein the angle ranges between 30 to 90 degrees) or retracted through the plurality of openings 604. In accordance with an aspect, the plurality of retractable needles 608 are hollow and include at least one opening to allow delivery of an ablative agent, such as steam or vapor 610, through the needles 608 when the needles are extended and deployed through the plurality of openings 604. The plurality of openings 604 extend from the body 602 and through the balloon 606 to enable the plurality of needles 608 to be extended beyond the balloon 606 when deployed.

A heating chamber 612 is located at the proximal end of the catheter 600. The heating chamber 612 comprises a metal coil wound about a ferromagnetic core. The chamber 612 is filled with water via a water inlet port 614 at a proximal end of the chamber 612. Alternating current is provided to the coil creating a magnetic field that induces electric current flow in the ferromagnetic core thereby heating the chamber 612 and causing the water within to vaporize. The resulting steam or vapor 610 exits the needles 608 to ablate target tissue. The balloon 606 is inflated by filling it with a coolant that is supplied to the balloon 606 through a coolant port 616 at the proximal end of the chamber 612. During use, the balloon 606 is inflated with the coolant while vapor or steam 610, generated in the chamber 612, is delivered through the plurality of needles 608. Since the needles 608 pierce into the target tissue during use, the steam or vapor 610 delivered through the pierced needles 608 cause ablation of tissue located deep within the target tissue. The coolant filled inflated balloon 606 contacts the surface of the non-target tissue and maintains the ambient temperature on the surface of the non-target tissue to a desired level, such as below 60 degrees C. in some embodiments. This enables the vapor 610 to ablate deeper target tissue without circumferentially ablating the non-target tissue at the surface. In some embodiments, the heating chamber 612 is at the distal end of the catheter proximal to the most proximal needle 608 and the plurality of openings 604, and is configured to use RF energy to generate vapor using resistive or ohmic heating of saline. In all embodiments, the plurality of the needles is electrically isolated from the heating chamber 612 by a segment of the catheter 602 so as to prevent the RF electrical current from the electrode passing into the tissues and into the human body. In various embodiments a conductive fluid such as saline is heated to a non-conductive ablative agent such as steam so as to minimize the chances of RF electrical current from passing from the heating chamber into the prostatic tissue and patient's body. It is desirable to isolate the patient from the RF electrical current so as not to interfere with any implanted electromedical devices.

FIG. 6C is an illustration of prostate ablation being performed on an enlarged prostrate in a male urinary system using the ablation catheter 600 of FIG. 6A, in accordance with an embodiment of the present specification. Also depicted in FIG. 6C are the prostate 618 and prostatic urethra 620. Referring now to FIGS. 6A and 6C, the ablation catheter 600 with the heating chamber 612 and the inflatable cooling balloon 606 is inserted into the patient's urethra and advanced into the prostatic urethra 620 so as to position the plurality of openings 604 proximate the tissue to be ablated. The cooling balloon 606 is inflated by filling it with coolant supplied from the coolant port 616, so that the inflated cool balloon 606 abuts the surface of the prostatic urethra proximate to the prostatic tissue to be ablated. Using a pusher, the needles 608 are then pushed out at an angle (ranging between 10 and 90 degrees, in various embodiments) from the catheter 600 into the prostate 618. Water (through the water inlet port 614) is administered into the chamber 612 where it is converted into steam or vapor 610. The steam or vapor 610 travels through the body 602 of the catheter and exits from openings in the needles 608 into the prostatic tissue, thus ablating the prostatic tissue. In one embodiment, the needles 608 are insulated. The coolant filled inflated balloon 606 maintains the ambient temperature on the surface of the prostatic urethra tissue to a desired level, such as below 60 degree C. in some embodiments. This enables the vapor 610 to ablate deeper prostatic tissue without circumferentially ablating the prostatic urethra tissue at the surface. Optional temperature sensors can be installed to detect the temperature of the prostatic urethra and modulate the delivery of vapor. In some embodiments, the heating chamber 612 is at the distal end of the catheter proximal to the most proximal needle 608 and the plurality of openings 604, and is configured to use RF energy to generate vapor using resistive or ohmic heating of saline. In embodiments, the needles are separated from the RF electrode by an insulative segment of the catheter to minimize or prevent passage of RF current into the patient's tissue and prevent electrical interference with electromedical implants.

FIG. 6D is a flow chart listing the steps involved in a transurethral enlarged prostate ablation process using the ablation catheter 600 of FIG. 6A, in accordance with one embodiment of the present specification. Referring now to FIGS. 6A and 6D, at step 622, the ablation catheter 600 is inserted into the urethra and advanced until the plurality of openings 604 are positioned proximate the prostatic tissue to be ablated within the prostatic urethra. At step 624, the cooling balloon 606 is inflated, with coolant supplied from the coolant port 616, to fix the catheter 600 within the prostatic urethra and maintain ambient temperature on the surface of the tissue to be ablated. Using a pusher, at step 626, the needles 608 are then pushed out at an angle (between 30 and 90 degrees, in various embodiments) from the catheter 600 through the prostatic urethra and into the prostate up to a desirable depth. Vapor is delivered, from openings in the needles 608, into the prostatic tissue at the desirable depth, thus ablating the prostatic tissue, without ablating the surface of the prostatic urethra. An optional temperature sensor is utilized to monitor the temperature of the surface of the prostatic urethra and control or modulate the flow of the coolant to maintain the temperature of the surface of the prostatic urethra below, say, 60 degrees C.

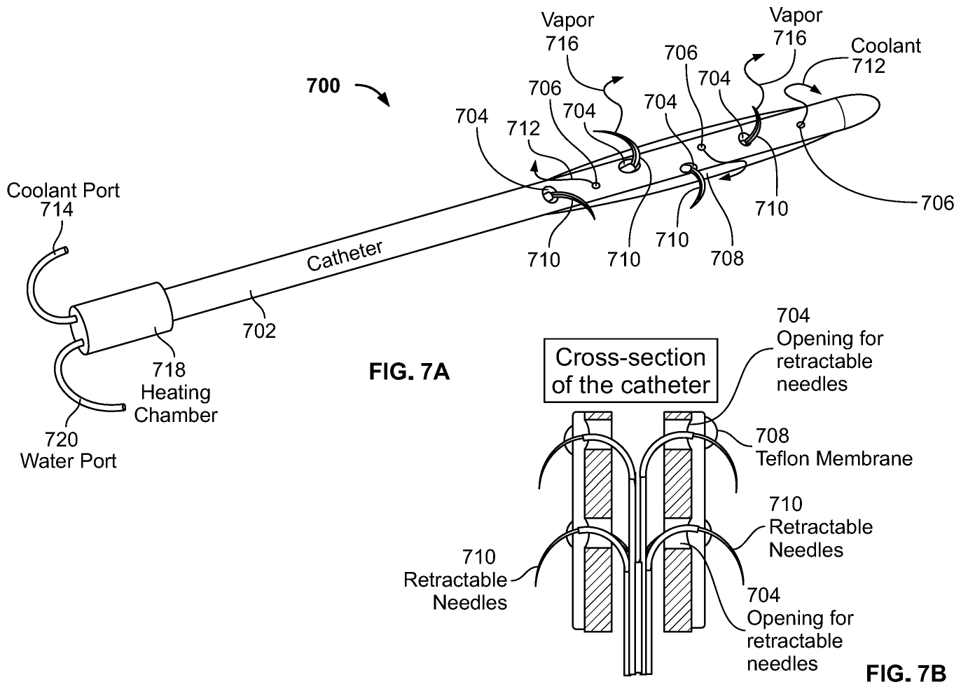

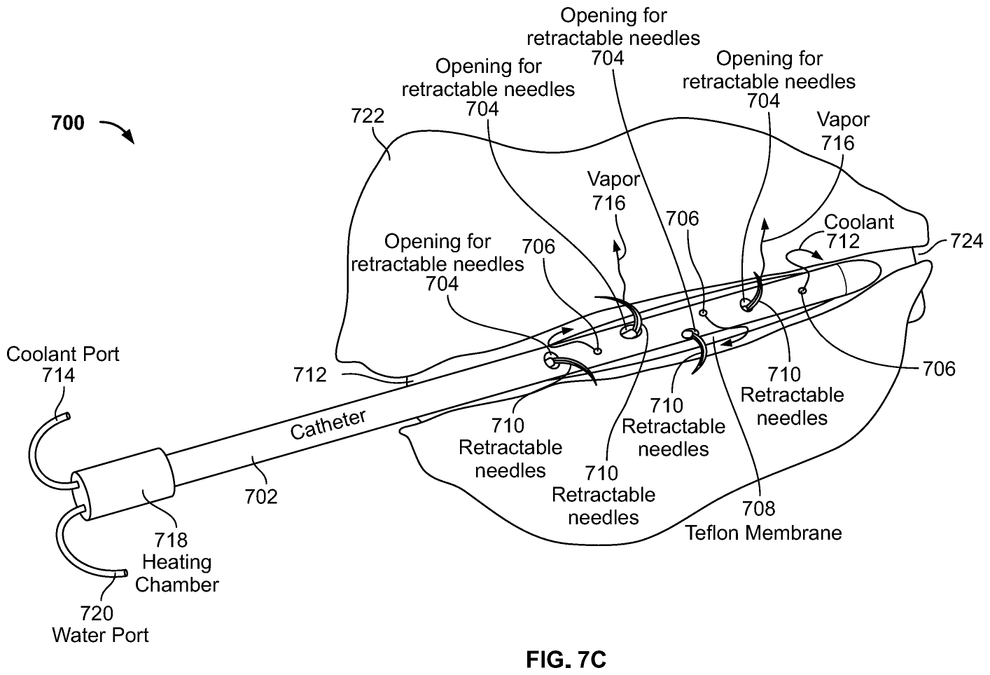

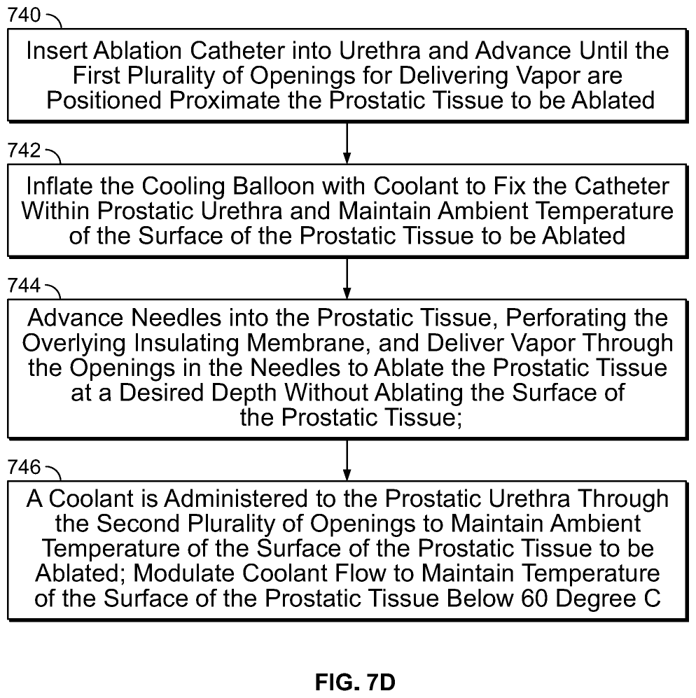

FIG. 7A illustrates an ablation catheter 700 while FIG. 7B is a cross-section of the tip of the catheter 700, in accordance with an embodiment of the present specification. Referring now to FIGS. 7A and 7B, the catheter 700 comprises an elongate body 702 having a proximal end and a distal end. A first plurality of openings 704, a second plurality of openings 706, and a silicone or Teflon membrane 708, covering the first and second pluralities of openings, are located proximate the distal end. The first plurality of openings 704 enables a plurality of associated thermally conductive elements 710, such as needles, to be extended (at an angle from the catheter 700, wherein the angle ranges between 30 to 90 degrees) or retracted through the plurality of openings 704. The second plurality of openings 706 enables a coolant 712, supplied via coolant port 714 at the proximal end of the catheter 700, to be delivered to the ablation zone. In accordance with an aspect, the plurality of retractable needles 710 are hollow and include at least one opening to allow delivery of an ablative agent, such as steam or vapor 716, through the needles 710 when the needles are extended and deployed through the first plurality of openings 704. The plurality of openings 704 extend from the body 702 and through the balloon 708 to enable the plurality of needles 710 to be extended beyond the membrane 708 when deployed. The needles 710 pierce through the membrane 708, when deployed, such that the membrane 708 insulates the needles 710 as these are being deployed and pierced into a target tissue.

A heating chamber 718 is located at the proximal end of the catheter 700. The heating chamber 718 comprises a metal coil wound about a ferromagnetic core. The chamber 718 is filled with water via a water inlet port 720 at a proximal end of the chamber 718. Alternating current is provided to the coil creating a magnetic field that induces electric current flow in the ferromagnetic core thereby heating the chamber 718 and causing the water within to vaporize. The resulting steam or vapor 716 exits the needles 710 to ablate target tissue. The coolant port 714, at the proximal end of the chamber 718, supplies coolant 712 for delivery through the second plurality of openings 706 into the prostatic urethra. During use, the coolant 712 is delivered to the ablation zone through coolant openings 706, while vapor or steam 716, generated in the chamber 718, is delivered through the plurality of needles 710. In some embodiments, the heating chamber 718 is located in the catheter body proximate opening 704 and is configured to use RF resistive heating for generating steam or vapor.

Since the needles 710 pierce into the target tissue during use, the steam or vapor 716 delivered through the pierced needles 710 cause ablation of tissue located deep within the target tissue. The coolant 712 directly contacts the surface of the non-target urethral tissue and maintains the ambient temperature on the surface of the non-target tissue to a desired level, such as below 60 degrees C. in some embodiments, preventing or diminishing clinically significant or circumferential thermal injury to the non-target tissue. This enables the vapor 716 to ablate deeper prostatic tissue without circumferentially ablating the urethral tissue at the surface. Also, the membrane 708 insulates the piercing needles 710 and prevents the coolant 712 from significantly cooling the needles 710. In some embodiments, the heating chamber 718 is at the distal end of the catheter proximal to the most proximal needle 710 and plurality of openings 704, and is configured to use RF energy to generate vapor using resistive or ohmic heating of saline. The catheter is optimized to minimize any leakage of RF electrical current into the tissue. In no situation is leakage sufficient to create a clinically significant ablation lesion.

FIG. 7C is an illustration of prostate ablation being performed on an enlarged prostrate in a male urinary system using the ablation catheter 700 of FIG. 7A, in accordance with an embodiment of the present specification. Also depicted in FIG. 7C are the prostate 722 and prostatic urethra 724. Referring now to FIGS. 7A and 7C, the ablation catheter 700 with the heating chamber 718 and the inflatable cooling balloon 708 is inserted into the patient's urethra and advanced into the prostatic urethra 724 so as to position the first plurality of openings 704 and second plurality of openings 706 proximate the prostatic tissue to be ablated. The coolant 712 is delivered, through the second plurality of openings 706, to the prostatic urethra 724. Using a pusher, the needles 710 are then pushed out at an angle (ranging between 30 and 90 degrees, in various embodiments) from the catheter 700 into the prostate 722. The pushed out needles 710 also perforate or traverse the insulating membrane 708 covering the openings 704.

Water or saline (through the water inlet port 720) is administered into the chamber 718 where it is converted into steam or vapor 716. The steam or vapor 716 travels through the body 702 of the catheter and exits from openings in the needles 710 into the prostatic tissue, thus ablating the prostatic tissue. The needles 710 are insulated by the membrane 708 while the needles 710 perforate the membrane 708. The coolant filled inflated balloon 708, as well as the coolant 712 delivered to the prostatic urethra 724, via the second plurality of openings 706, maintain the ambient temperature on the surface of the prostatic tissue to a desired level, such as below 60 degree C. in some embodiments and, preferably, below 40 degree C. in other embodiments. This enables the vapor 716 to ablate deeper prostatic tissue without ablating the prostatic urethra tissue at the surface in clinically significant or circumferential fashion. Optional temperature sensors can be installed to detect the temperature of the prostatic urethra and modulate the delivery of vapor 716 and/or coolant 712.

FIG. 7D is a flow chart listing the steps involved in a transurethral enlarged prostate ablation process using the ablation catheter 700 of FIG. 7A, in accordance with one embodiment of the present specification. Referring now to FIGS. 7A and 7D, at step 740, the ablation catheter 700 is inserted into the urethra and advanced until the first plurality of openings 704 is positioned proximate the prostatic tissue to be ablated within the prostatic urethra. At step 742, the cooling balloon 708 is inflated, with coolant supplied from the coolant port 714, to fix the catheter 700 within the prostatic urethra and maintain ambient temperature on the surface of the prostatic tissue to be ablated. Using a pusher, at step 744, the needles 710 are then pushed out at an angle (between 10 and 90 degrees, in various embodiments) from the catheter 700 to pierce through the insulating membrane 708, through the prostatic urethra and into the prostate up to a desirable depth. Vapor 716 is delivered, from openings in the needles 710, into the prostatic tissue at the desirable depth, thus ablating the prostatic tissue, without ablating the surface of the prostatic tissue. At step 746, coolant 712 is administered into the prostatic urethra, via the second plurality of openings 706, to maintain ambient temperature on the surface of the prostatic tissue to be ablated. The membrane 708 insulates the piercing needles 710 from the coolant 712 administered into the prostatic urethra. An optional temperature sensor is utilized to monitor the temperature of the surface of the prostatic tissue and control or modulate the flow of the coolant to maintain the temperature of the surface of the prostatic tissue below a specific temperature, which, in some embodiments, is 60 degree C.

Referring back to FIGS. 6A and 7A, in accordance with some embodiments, a pump, such as a syringe pump or a peristaltic pump, is used to control the flow of water to the heating chamber 612, 718.

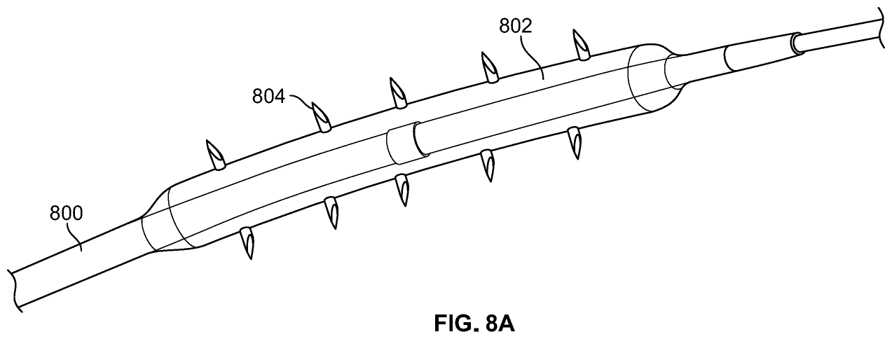

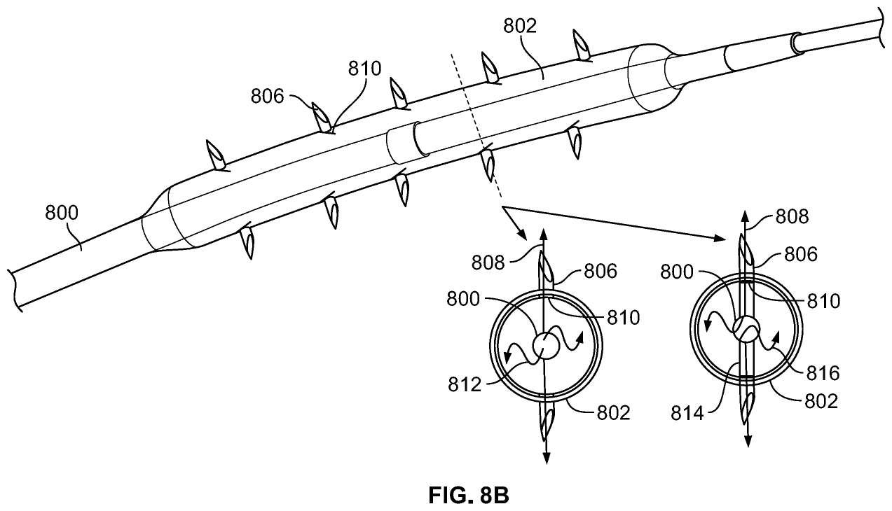

In various embodiments, the catheters of the present specification further include at least one thermally conducting element attached to the positioning element. The at least one thermally conducting element is configured to physically contact, and, in some embodiments, penetrate, a target tissue and enhance the delivery of thermal energy into the target tissue for ablation. FIG. 8A is an illustration of one embodiment of a positioning element 802 of an ablation catheter 800, depicting a plurality of thermally conducting elements 804 attached thereto. In various embodiments, the positioning element 802 is an inflatable balloon. The positioning element, or balloon 802, is inflated to a first volume to bring the thermally conducting elements 804 into contact with a target tissue. An ablative agent is then delivered to the target tissue through the catheter 800 and out via at least one delivery port at the distal end of the catheter 800. Thermal energy from the ablative agent is transferred from the lumen of the catheter 800 into the air in the balloon 802, further expanding the volume of the balloon 802 and pushing the thermally conducting elements 804 further into the target tissue. Thermal energy from the air in the balloon 802 is transferred to the thermally conducting elements 804 and is released into the target tissue for ablation. In various embodiments, the thermally conducting elements 804 comprise solid or hollow metal spikes or needles. In various embodiments, the balloon 802 is composed of a thermally insulating material so that ablative thermal energy is predominantly transferred from the thermally conducting elements 804 into the target tissue.

FIG. 8B is an illustration of one embodiment of a positioning element 802 of an ablation catheter 800, depicting a plurality of hollow thermally conducting elements 806 attached thereto. In one embodiment, each hollow thermally conducting element 806 includes a valve 806 at the inlet from a lumen of the positioning element 802 to a lumen of the hollow thermally conducting element 806. In various embodiments, the positioning element 802 is an inflatable balloon. The positioning element, or balloon 802, is inflated to a first volume to bring the thermally conducting elements 804 into contact with a target tissue. An ablative agent is then delivered to the target tissue through the catheter 800 and out via at least one delivery port at the distal end of the catheter 800. Thermal energy from the ablative agent is transferred from the lumen of the catheter 800 into the air in the balloon 802, further expanding the volume of the balloon 802 and pushing the thermally conducting elements 806 further into the target tissue. Thermal energy from the air in the balloon 802 is transferred to the thermally conducting elements 806 and is released into the target tissue for ablation. In various embodiments, the thermally conducting elements 806 comprise hollow metal spikes or needles. The thermally conducting elements 806 include at least one opening at their distal ends which are in fluid communication with a lumen of the thermally conducting elements 806, which, in turn, is in fluid communication with the interior of the balloon 802. As seen in the cross section of the catheter 800, vapor follows a first pathway 808 to pass from the interior of the balloon 802, through the thermally conducting elements 806, and out to the target tissue. In one embodiment, each thermally conducting element 806 includes a valve 810 positioned at its junction with the balloon 802 to control the flow of vapor into each hollow thermally conducting element 806. In one embodiment, the vapor also follows a second pathway 812 into the interior of the balloon 802 to transmit thermal energy and assist in balloon expansion 802. In another embodiment, flexible tubes 814 connect the lumen of each thermally conducting element 806 with a lumen of the catheter 800, bypassing the interior of the balloon 802. In one embodiment, the tubes 814 are composed of silicone. In this embodiment, the vapor can only travel via the first pathway 808 and air 816 is used to expand the balloon 802. In various embodiments, the balloon 802 is composed of a thermally insulating material so that ablative thermal energy is predominantly transferred from the thermally conducting elements 806 into the target tissue. In various embodiments, the thermally conducting elements 806 possess shape memory properties such that they change shape from being generally parallel to the catheter 800 at a temperature below a patient's body temperature to being generally perpendicular to the catheter 800 at temperatures above the patient's body temperature.

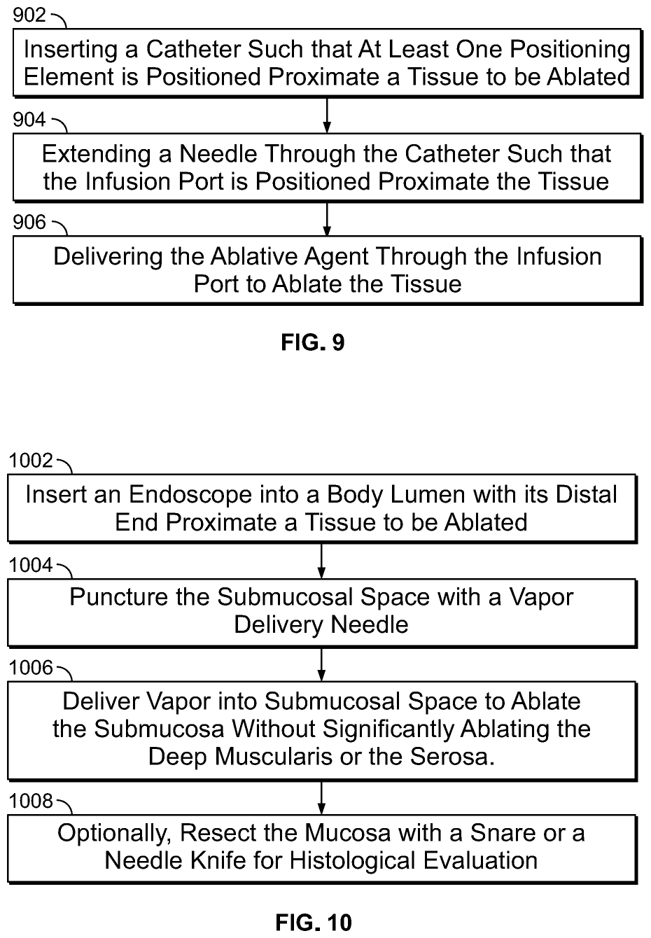

FIG. 9 is a flowchart illustrating one embodiment of a method of ablation of a tissue using a needle catheter device as described above. The device includes a thermally insulated catheter having a hallow shaft and a retractable needle through which an ablative agent can travel, at least one infusion port on the needle for delivery of the ablative agent, at least one positioning element on a distal end of the catheter, and a controller comprising a microprocessor for controlling the delivery of ablative agent. Referring to FIG. 9, in the first step 902, a catheter is inserted such that a positioning element is positioned proximate to the tissue to be ablated. The next step 904 involves extending the needle through the catheter such that the infusion port is positioned proximate to the tissue. Finally in step 906, an ablative agent is delivered through the infusion port to ablate the tissue. In another embodiment, the device does not include a positioning element and the method does not include a step of positioning the positioning element proximate the tissue to be ablated.

In one embodiment, the needle catheter device described in FIGS. 8A and 8B is also used for vapor ablation of submucosal tissue.

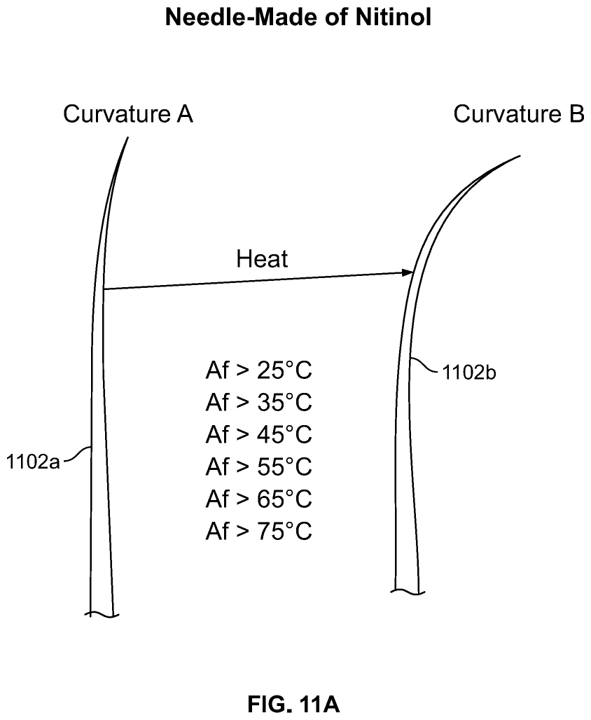

FIG. 10 is a flowchart illustrating a method of ablation of a submucosal tissue using a needle catheter device similar to those as described above. Referring to FIG. 10, in the first step 1002, an endoscope is inserted into a body lumen with its distal end proximate a tissue to be ablated. Next, in step 1004, the submucosal space is punctured using a vapor delivery needle, which is passed by means of a catheter through a working channel of the endoscope. Next, in step 1006, vapor is delivered into a submucosal space, predominantly ablating the submucosa and/or mucosa without irreversibly or significantly ablating the deep muscularis or the serosa. In one embodiment, the mucosa can be optionally resected with a snare or a needle knife for histological evaluation in step 1008. In some embodiments, the submucosa is pre-treated to create a submucosal lift with either a saline injection, glucose solution, glycerol, sodium hyaluronate (SH), colloids, hydroxypropyl methylcellulose, fibrinogen solution, autologous blood, or other alternatives or injection of other agents known in the art, such as Eleview™.

In another embodiment, the present specification discloses shape changing needles for ablation of prostatic tissue. FIG. 11A is an exemplary illustration of shape changing needles. Referring to FIG. 11A, needle 1102a is made up of a flexible material, such as nitinol, and has a curvature in the range of −30 to 120°. In some embodiments, the needle tip curves from 0 to 180°. In one embodiment, when heat is applied to the needle 1102a, its curvature increases, as shown by 1102b. In one embodiment, for an increase in temperature in the range of 25 degrees C. to 75 degrees C., the increase in the curvature of the needle ranges from −30 to 120°. In accordance with an aspect, the needle 1102a is hollow and includes at least one opening to allow delivery of an ablative agent, such as steam or vapor through the needle. In some embodiments, tension wires fixed to the needle can be pulled to change the shape of the needle or stabilize the needle to assist in puncture. In some embodiments, pulling on these tension wires can assist with making the puncture or help drive the needle deep into the prostatic tissue.

FIG. 11B illustrates different embodiments of needles, in accordance with the present specification. Referring to FIG. 11B, needles 1102c, 1102d, and 1102e, are single needles of different curvatures. Needles 1102f and 1102g are double needles of different sizes. In some embodiments, the needles 1102c, 1102d, 1102e, 1102f and 1102g are covered in an outer insulation layer, described subsequently in FIGS. 11K to 11Q. Needles 1102f and 1102g illustrate exemplary embodiments of two needles that are extended from a single port. In some embodiments, needles of FIG. 11B are made from 22 gauge stainless steel. FIG. 11C illustrates an exemplary process of delivery of an ablative agent 1104 from hollow openings 1106 at the edges of a pair of needles 1108, 1110 of a double needle, such as double needles 1102f or 1102g of FIG. 11B, in accordance with some embodiments of the present specification.

FIG. 11D illustrates exemplary depths or penetrating depths of needles 1102c, 1102d, and 1102e of different curvatures, in accordance with some embodiments of the present specification. The depth increases with the increase in the curvature. In some embodiments, the needles 1102c, 1102d, and 1102e have a curvature that varies between 0 and 150 degrees, with a diameter from 15 to 30 Gauge, and a length of each needle 1102c, 1102d, and 1102e ranging from 0.2 to 5 centimeters (cm). FIG. 11E illustrates exemplary depths or penetrating depths of needles 1102f and 1102g, relative to needles 1102c, 1102d, and 1102e of FIG. 11D, in accordance with some embodiments of the present specification. FIG. 11F illustrates exemplary lengths of needles 1102c, 1102d, 1102e, 1102f, and 1102g of FIG. 11E, extending in a straight line from a proximal port 1112 to the farthest distal point 1114 reached by the body of the needles, in accordance with some embodiments of the present specification.

FIG. 11G illustrates different views of a single needle assembly 1116 extending from a port 1118, in accordance with some embodiments of the present specification. In embodiments, the port 1118 includes two cylindrical portions, a first portion 1118a and a second portion 1118b, where the second portion 1118b is connected to an inner catheter (such as inner catheter 107m of FIG. 1M), while the first portion 1118a is attached to the second portion 1118b and a distal edge of first portion 1118a provides for an exit of one or more needles, such as needle 1116. Additionally, FIG. 11G illustrates a top view 1116A, a side view 1116B, and a front perspective view 1116C of the needle 1116 in its default curved state. A side perspective view 1116D of the needle 1116 in a linear, collapsed state is also illustrated. In an embodiment, a length of the needle 1116 extending in a straight line from the a distal edge of first portion 1118a to the farthest point of the needle 1116 is approximately 12 mm, and a depth from a sharp edge of the needle 1116 to the port, measured in a straight line, is approximately 12.3 mm. In some embodiments, the first portion 1118a of the port 1118 has a length of approximately 4.10 mm and a diameter of approximately 2.35 mm. In some embodiments, the second portion 1118b of the port 1118 has a length of approximately 4.30 mm and a diameter in a range of approximately 1.75 to 1.85 mm. FIG. 11H illustrates one or more holes 1120 at the sharp edge of the needle 1116 in another horizontal view of the needle 1116, in accordance with some embodiments of the present specification. In some embodiments, each of the holes 1120, used to deploy ablative vapor, extends for a length of about 3.50 mm at one side of tip of the hollow cylindrical needle 1116. The holes are positioned on a side along the length of the needle 1116, while a distal tip of the needle 1116 is occluded. In some embodiments, the distal tip may be occluded with a plug 1122 made from biocompatible material, such as for example stainless steel. In some embodiments, the distal tip is occluded and the vapor comes exits from the sides of the distal tip.

FIG. 11I illustrates different views of a double needle assembly 1124 extending from a port 1126, in accordance with some embodiments of the present specification. FIG. 11J illustrates different views of another double needle assembly 1128 extending from a port 1132, in accordance with some embodiments of the present specification. Referring simultaneously to FIGS. 111 and 11J, the port 1126, 1132 may include two cylindrical portions, a first portion 1126a, 1132a and a second portion 1126b, 1132b, where the second portion 1126b, 1132b is connected to an inner catheter (such as inner catheter 107m of FIG. 1M), while the first portion 1126a, 1132a is attached to the second portion 1126b, 1132b and a distal edge of first portion 1126a, 1132a provides for an exit of a double needle assembly 1124, 1128. The double needle assembly includes a first needle 11241, 11281 and a second needle 11242, 11282. FIGS. 11I and 11J illustrate a top view 1124a, 1128a, a side view 1124b, 1128b, and a top side perspective view 1124c, 1128c of the needles 1124, 1128 in their default curved states. A side perspective view 1124d, 1128d of the needles 1124, 1128 in a linear, collapsed state is also illustrated. Referring to FIG. 11I, a length of the needle 11241 extending in a straight line from a distal edge of port 1126 to the farthest point of the needle 11241 is approximately 17 mm, and a depth from a sharp edge of the needle 11241 to the port 1126 measured in a straight line, is approximately 13.4 mm. A length of the needle 11242 extending in a straight line from a distal edge of port 1126 to a farthest point of the needle 11242 is approximately 12 mm, and a length from a sharp edge of the needle 11242 to the port 1126 measured in a straight line, is approximately 12.2 mm. In embodiments, the port 1126 is configured similarly to port 3808. The distance between the sharp edges of needles 11241 and 11242 is approximately 5 mm. Referring to FIG. 11J, a length of the needle 11281 extending in a straight line from a distal edge of port 1132 to the farthest point of the needle 11281 is approximately 22 mm, and a length from a sharp edge of the needle 11281 to the port 1132 measured in a straight line, is approximately 13.4 mm. A length of the needle 11282 extending in a straight line from a distal edge of port 1132 to the farthest point of the needle 11282 is approximately 12 mm, and a length from a sharp edge of the needle to the port 1132 measured in a straight line, is approximately 12.2 mm. In embodiments the port 1132 is configured similarly to port 3808. The distance between the sharp edges of needles 11281 and 11282 is approximately 10 mm. In some embodiments, one or both of needles 11281 and 11282 has one or more openings or holes 1130 on the sides, along their length, while distal tip of the one or both needles 11281 and 11282 that has the holes 1130 is occluded with a plug 1134. The holes 1130 provide an exit for ablation vapors.

FIG. 11K illustrates an insulation 1136 on a single needle configuration 1138 comprising a needle 1140, and a double needle configuration 1142 comprising needles 1144 and 1146. Each of needle 1140, 1144, and 1146 may have one or more openings, such as an opening 1148 at the tip of the needle 1140, to enable an exit for vapor during ablation. The insulation 1136 insulates a portion of the needles' 1140, 1144, and 1146 outer length. The insulation 1136 can be added, in some embodiments, as a shrink tube or as spray on. In different embodiments, insulation 1136 extends along any portion of a length of needles 1140, 1144, and 1146, from their distal tip to their base, but do not cover any openings at the distal tip or along the length of the needles. An ablation area may be modified by changing distribution of insulation 1136 on the needles. This is illustrated with reference to FIGS. 11L, 11M, and 11N.

FIG. 11L illustrates a single needle configuration 1140 with insulation 1136 positioned inside a prostatic tissue 1150 in accordance with some embodiments of the present specification. The insulation 1136 covers the portion of the needle 1140 that extends from a catheter 1156 to the length of the needle 1140 before a tip of the needle, so that a portion of the insulation 1136 extends into the prostatic tissue from a urethra 1152, therefore protecting the urethra 1152. FIG. 11M illustrates a single needle configuration 1140 with insulation 1136 positioned inside a uterine fibroid 1158 in accordance with some embodiments of the present specification. The needle 1140 extends from a uterus 1160 into the fibroid 1158. The insulation 1136 covers a greater extent of the needle 1140, relative to the extent shown in FIG. 11L, so that the insulation 1136 extends into the fibroid 1158 along with a small portion of the tip of the needle 1140 and delivers ablation vapor only to the fibroid 1158, while protecting parts of the anatomy outside the fibroid. FIG. 11N illustrates a double needle configuration 1142 where the two needles 1144 and 1146 are inserted into separate prostate lobes 1162 and 1164, in accordance with some embodiments of the present specification. The insulation 1136 covering both needles 1144 and 1146 extends into the lobes 1162 and 1164 along with the non-insulated distal tips of the needles.

FIG. 11O illustrates an exemplary embodiment of a steerable catheter shaft 1166 in accordance with some embodiments of the present specification. Catheter shaft 1166 is configured to be flexible so that it may be steered by a user to direct a needle 1140 in a required direction. Referring to the figure, an arrow 1168 indicates the ability to steer the needle in different directions, using the catheter shaft 1166. In embodiments, a viewing device 1170 is configured at the tip of the catheter shaft 1166 at the base of the needle 1140 to help the user articulate direct visualization of the needle 1140. In embodiments, the viewing device 1170 includes a camera, lens, LEDs, or any other equipment to facilitate direct visualization of the needle's 1140 position and movement within the anatomy of a patient, thereby aiding the physician in steering the needle 1140. In embodiments, a channel 1172 in the catheter shaft 1166 provides for containing optical and electrical wires that connect the viewing device 1170 to a controller, such as controller 15q, for power and for displaying the captured images on a screen or split-screen for both viewing the ablation area and controlling the ablation delivery. In some other embodiments, the viewing device 1170 interfaces with a peripheral computing and/or imaging device, for example, an iPhone, to display the images captured by its camera. In embodiments, controls of the viewing device 1170 are provided in a handle of the catheter shaft 1166. In one embodiment, the needle is steered using a plurality of tension wires attached to the needle and pulling on those tension wires allow to manipulate the position or direction of the needle tip.

FIG. 11P illustrates a needle 1140 with an open tip 1174, in accordance with some embodiments of the present specification. The figure also shows steam 1176 that sprays out from the opening at the distal tip 1174. In practice, needle 1140 is first flushed with water to get any air out, prior to spraying ablation vapor or steam 1176. FIG. 11Q illustrates an alternative embodiment of a needle 1140 with a plug 1178 at its distal tip to occlude the tip and comprising holes or openings 1180 along an uninsulated length of the needle 1140, close to the tip, to provide a sprinkler-style spray of steam 1176, in accordance with the present specification.

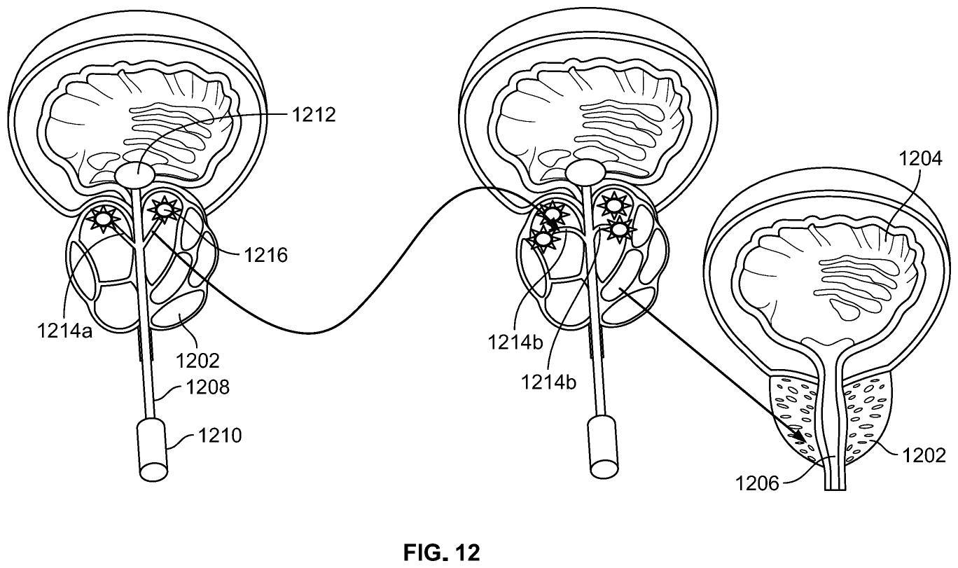

FIG. 12 is an illustration of transurethral prostate ablation being performed on an enlarged prostrate 1202 in a male urinary system using an ablation device, which makes use of shape changing needles, in accordance with one embodiment of the present specification. Also depicted in FIG. 12 are the urinary bladder 1204 and prostatic urethra 1206. An ablation catheter 1208 with a handle 1210 and a positioning element 1212 is inserted into the urethra 1206 and advanced into the bladder 1204. In one embodiment, the positioning element 1212 is inflated and pulled to the junction of the bladder with the urethra, thus positioning needles 1214a at a predetermined distance from the junction. Using a pusher (not shown) coupled to the handle 1210, the needles 1214a are then pushed out at an angle between 10 and 90 degrees from the catheter 1208 through the urethra 1206 into the prostate 1202. Vapor is administered through a port (not shown) that travels through the shaft of the catheter 1208 and exits from openings 1216 in the needles 1214a into the prostatic tissue, thus ablating the prostatic tissue. According to an embodiment, vapor delivery heats the needles and the needles change shape from substantially straight 1214a to curved in 1214b, while vapor is being delivered. On cessation of vapor delivery, the needles revert back to their original straight shape, which allows for easy retraction into the catheter. The mechanical shape change of needles allows for more effective distribution of the ablative energy within the prostatic tissue. In embodiments, the vapor is generated in the handle 1210 or the body 1208 of the catheter using inductive heating or resistive heating.

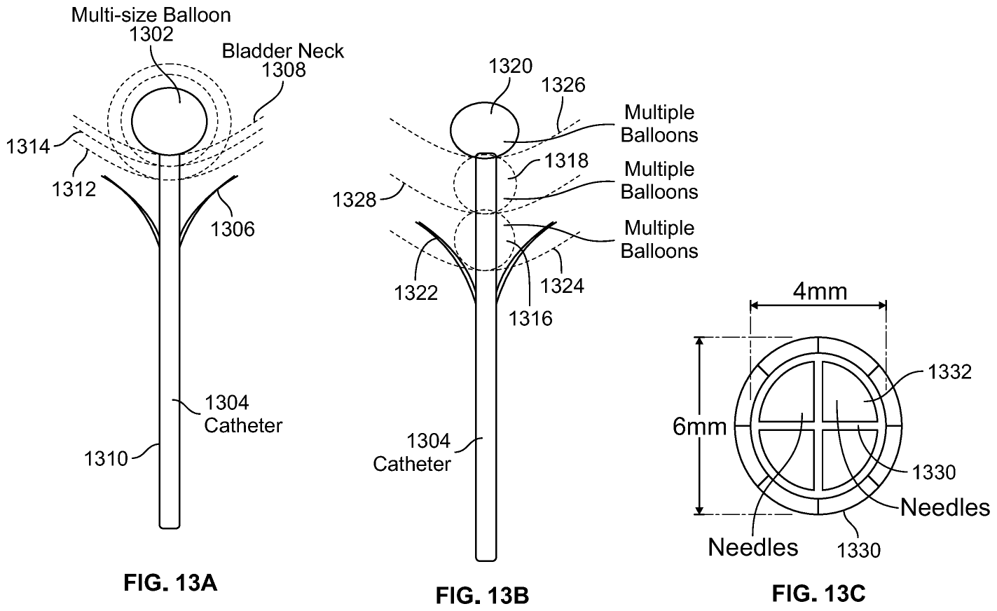

FIG. 13A is an illustration of one embodiment of a positioning element 1302 of an ablation catheter 1304 with needles 1306 attached to the catheter body. In various embodiments, the positioning element 1302 is an inflatable balloon. The positioning element, or balloon 1302, is inflated to a first volume, thus positioning needles 1306 at a predetermined distance from the bladder neck 1308 and bringing them into contact with the target tissue. In one embodiment, an ablative agent, such as steam or vapor, is delivered to the target tissue through the catheter 1304. Travelling through the shaft 1310 of the catheter, the vapor exits from openings (not shown) in the needles 1306 into the prostatic tissue, thus ablating the prostatic tissue. In one embodiment, the balloon 1302 is capable of being expanded to different sizes. This feature is used, in one embodiment, to progressively or sequentially inflate the balloon 1302 to different sizes, thereby positioning the needles at various fixed distances 1312, 1314 from the bladder neck 1308, allowing for treatment of discrete regions of the prostate tissue. In one embodiment, the predetermined distance at which the balloon may be used to place the needles ranges from 1 mm to 50 mm from the bladder neck. In one embodiment, the positioning element 1302 can be moved relative to the needle 1306, adjusting the range of the needles from 1 mm to 50 mm from the positioning element 1306. In another embodiment, the positioning element 1302 can engage with a length of the needle 1306, applying mechanical force helping the needle pierce the target tissue.

In another embodiment shown in FIG. 13B, a plurality of inflatable balloons 1316, 1318, 1320 are employed as positioning elements. These balloons may be used to position the needles 1322 at various fixed distances 1324, 1326 from the bladder neck 1328, allowing for treatment of discrete regions of the prostate tissue. It may be noted that any one of the plurality of balloons may be inflated, depending on the region of tissue to be ablated. The balloons may also be ablated in a sequential manner, to allow comprehensive coverage of target tissue. In one embodiment, the number of balloons ranges from one to five.

FIG. 13C illustrates a cross section of the distal tip of a catheter 1330, in accordance with an embodiment of the present specification. In one embodiment, for ablation of prostatic tissue, an inner diameter (ID) of the catheter employed is about 4 mm, and an outer diameter (OD) is about 6 mm. A plurality of thermally conductive elements 1332, such as needles, extend at an angle from the catheter 1330, wherein the angle ranges between 30 to 90 degrees. In one embodiment, the needles may be retracted into the catheter after ablation.

In one embodiment, the balloon is inflated prior to ablation. In another embodiment, the ablative agent, such as steam or vapor also transmits thermal energy and assists in balloon expansion. That is, thermal energy from the ablative agent is transferred from the lumen of the catheter into the air in the balloon, further expanding the volume of the balloon and pushing the needles further into the target tissue. In yet another embodiment, the balloon is inflated by filling it with a coolant that is supplied to the balloon through a coolant port at the proximal end of catheter. During use, the balloon is inflated with the coolant while vapor or steam is delivered through the plurality of needles. Since the needles pierce into the target tissue during use, the steam or vapor delivered through the pierced needles cause ablation of tissue located deep within the target tissue. The coolant filled inflated balloon contacts the surface of the target tissue and maintains the ambient temperature on the surface of the target tissue to a desired level, such as below 60 degrees C. in some embodiments. This enables the vapor to ablate deeper tissue without ablating the tissue at the surface.

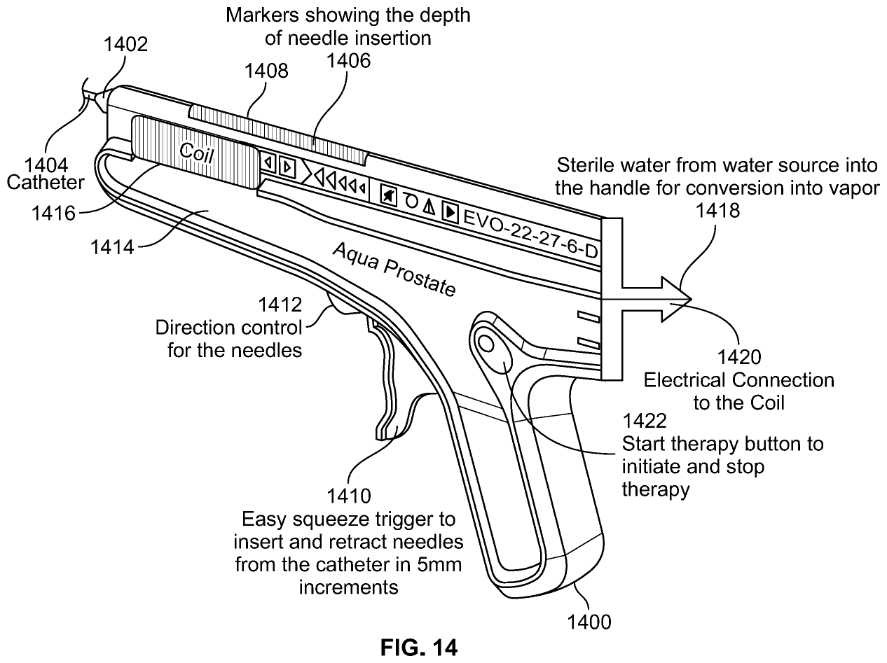

FIG. 14 illustrates one embodiment of a handle mechanism 1400 that may be used for deployment and retrieval of needles at variable depths of insertion, when ablating prostatic tissue. Referring to FIG. 14, in one embodiment, the handle 1400 is shaped like a handheld gun or pistol, which allows it to be conveniently operated by a physician for the treatment of prostatic tissue. The tip 1402 of the handle is equipped with a slot, into which an ablation catheter 1404 may be inserted for passing into the urethra of the patient. Ablation needles are coupled to the catheter, as explained in the embodiments above, and are used to deliver steam vapor to target tissue. On the top of the handle 1400, markers 1406 are placed, which indicate the depth of insertion of the needles. The markers may be placed by printing, etching, painting, engraving, or by using any other means known in the art suitable for the purpose. In one embodiment, the ablation needles may be inserted or retracted in increments of a fixed distance—such as 5 mm, and therefore markers are placed correspondingly to reflect the increments. A button 1408 is provided on the markers, which advances or retracts by a mark, each time the catheter and the needles are advanced or retracted by the preset distance. In one embodiment, a trigger 1410 is provided on the handle mechanism, which may be pressed to advance the needles for the preset increment of distance. In one embodiment, once the needles are advanced to the maximum distance by repeatedly pressing the trigger—as indicated by the button 1408 on the markers, further pressing of the trigger results in retraction of the needles, one increment of distance at a time. It may be noted that as explained in the embodiments above, the catheter is also equipped with a positioning element, such as a balloon, which does not allow the catheter and the needles to be advanced beyond a fixed distance in the urethra.

In one embodiment, a knob or a button 1412 is provided which may be turned or pressed to control the direction of movement of the catheter and the needles. That is, the knob 1412 may be used to determine whether the catheter and needles are moved forward (advanced) or backward (retracted), each time the trigger 1410 is pressed.

In one embodiment, the handle mechanism 1400 also comprises a heating chamber 1414, which is used to generate steam or vapor for supplying to the catheter 1404. The heating chamber 1414 comprises a metal coil 14165 wound about a ferromagnetic core. The chamber is filled with water via a water inlet port 1418 located at a proximal end of the handle mechanism 1400. In one embodiment, sterile water is supplied from a water source into the handle for conversion into vapor. The handle is also equipped with an electrical connection 1420 to supply the coil 1416 with electrical current from a current generator. Alternating current is provided to the coil 1416, thereby creating a magnetic field that induces electric current flow in the ferromagnetic core. This causes heating in the chamber 1414 and causes the water within to vaporize. The resulting steam or vapor, generated in the chamber 1414, is delivered through the needles placed at the appropriate location to ablate target tissue.

In an embodiment, a start/stop button 1422 is also provided on the handle 1400 to initiate or stop ablation therapy as required.

The same functionality can be achieved by other handle form-factors known in the art and also described in this application.

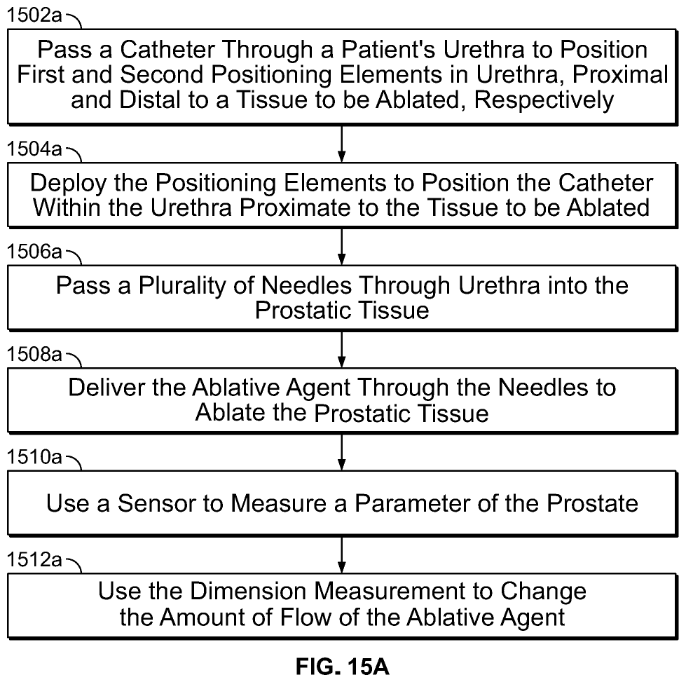

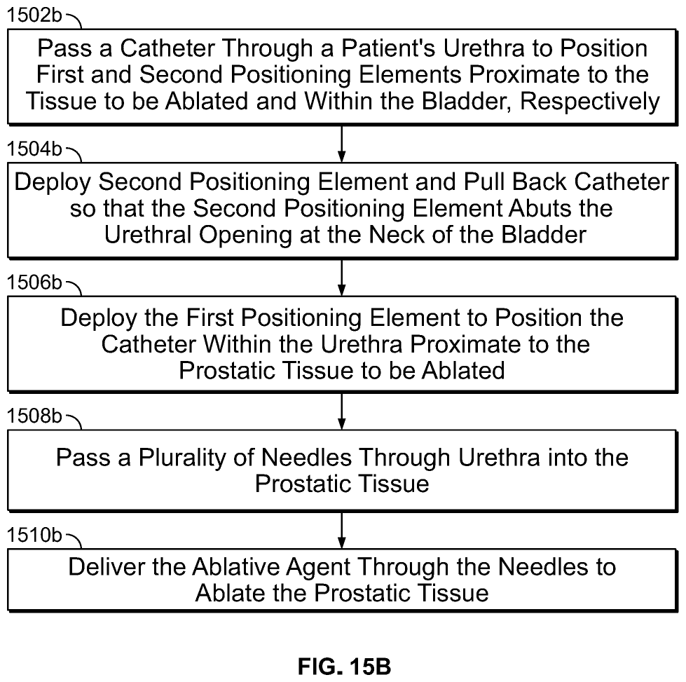

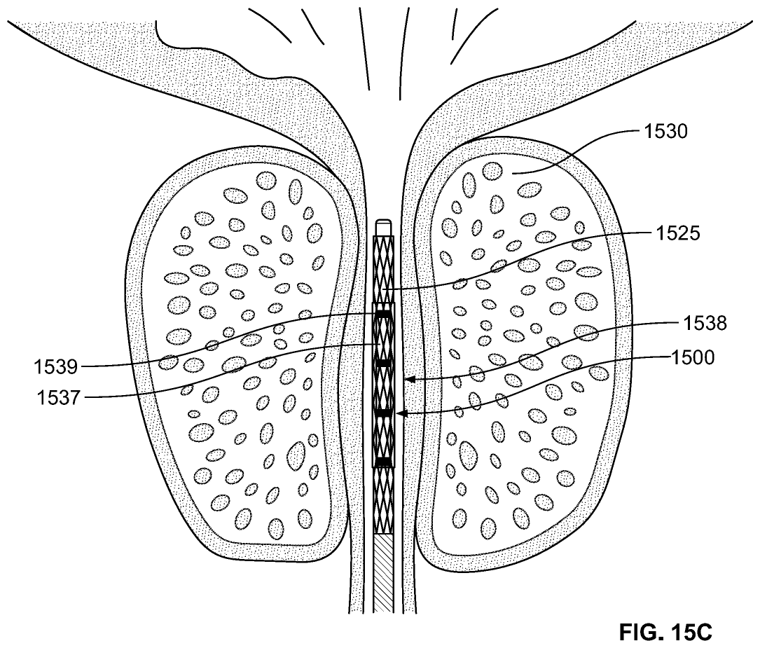

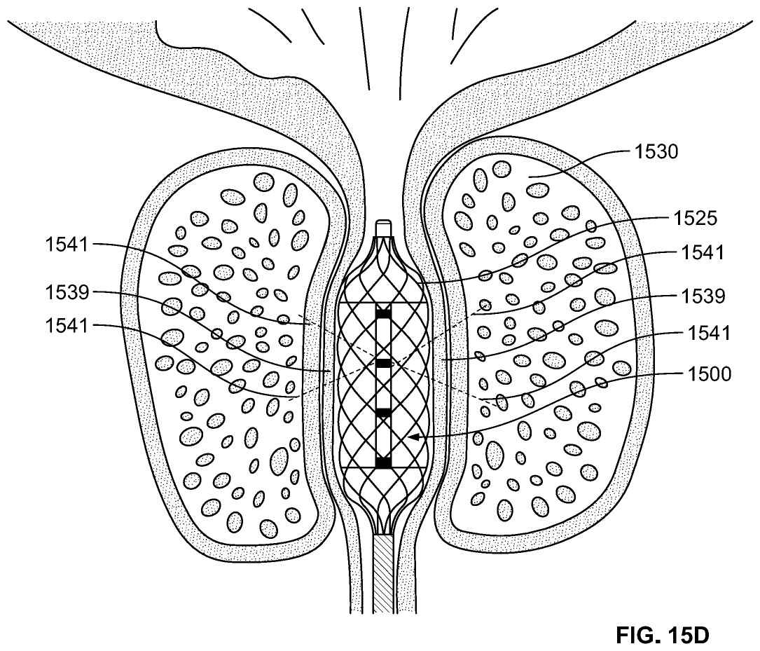

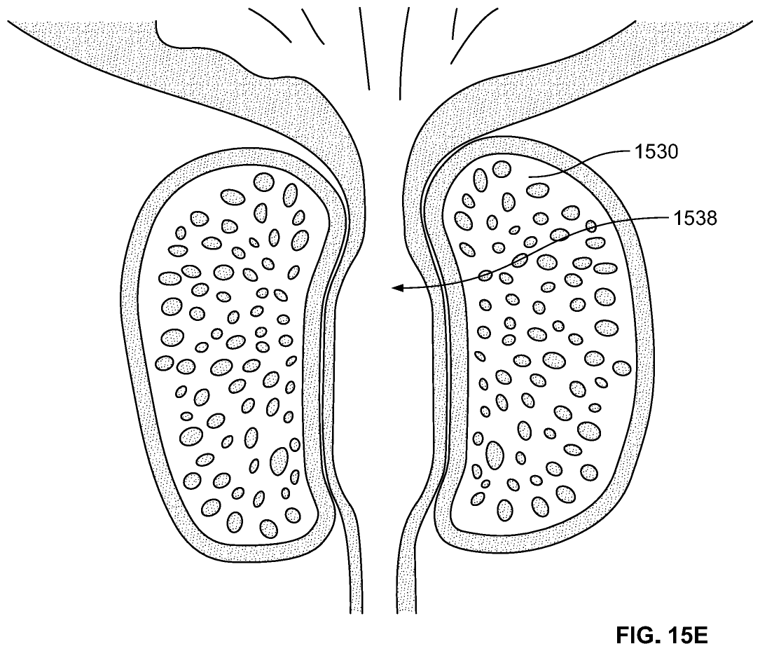

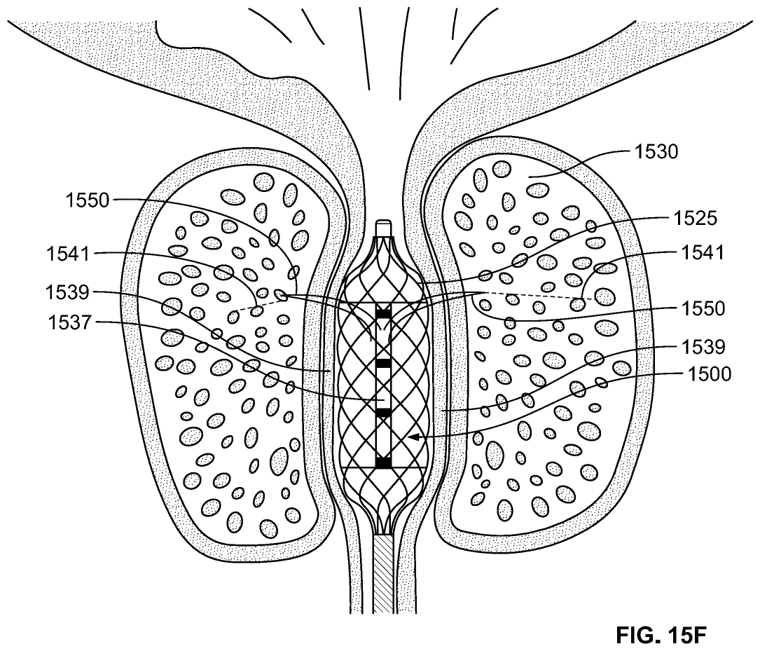

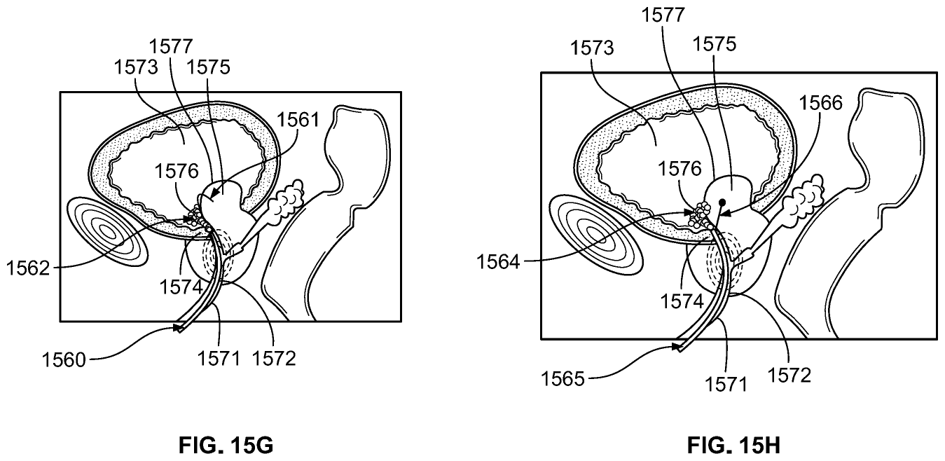

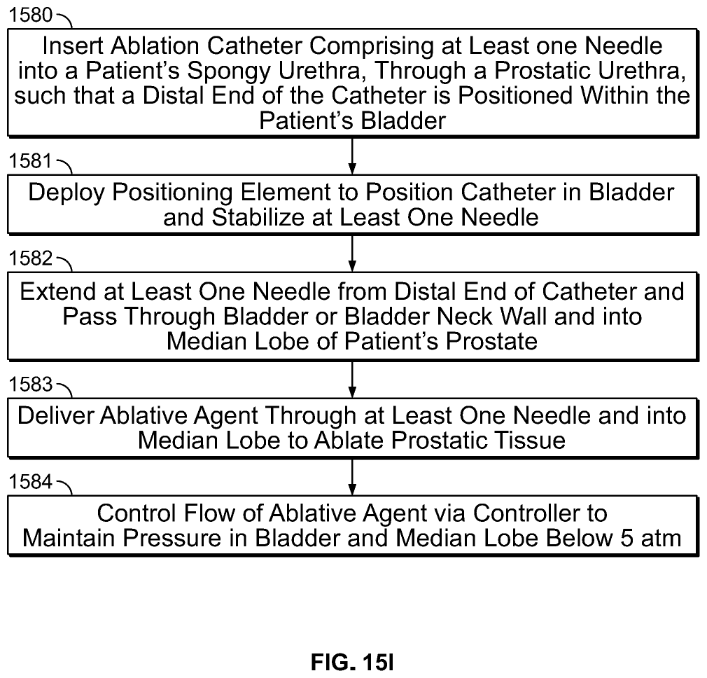

FIG. 15A is a flowchart illustrating a method of ablation of prostatic tissue in accordance with one embodiment of the present specification. Referring to FIG. 15A, the first step 1502a includes passing a catheter of an ablation device into a patient's urethra, wherein the catheter includes a hollow shaft through which an ablative agent can travel, at least one first positioning element, at least one second positioning element positioned distal to said at least one first positioning element, at least one input port for receiving an ablative agent, and a plurality of needles positioned on said catheter between said first and second positioning elements and configured to deliver ablative agent to a prostatic tissue. In an embodiment, the ablation device includes a controller comprising a microprocessor for controlling the delivery of the ablative agent. The catheter is passed through the urethra such that the first positioning element is positioned proximal to the prostatic tissue to be ablated and the second positioning element is positioned either in or distal to the prostatic tissue to be ablated. Next, in step 1504a, the positioning elements are deployed such that they contact the urethra and the catheter is positioned within the urethra, proximate the prostatic tissue to be ablated. In the next step 1506a, the plurality of needles is passed through the urethra into the prostatic tissue to be ablated. Finally, in step 1508a, an ablative agent is delivered through the needles to ablate the prostatic tissue. Optionally, a sensor is used to measure a parameter of the prostate in step 1510a and the measurement is used to increase or decrease the flow of ablative agent being delivered in step 1512a. Optionally, in an embodiment, a cystoscope is first inserted in the patient's urethra and the catheter is inserted through the cystoscope. In some embodiments, or more of the positioning elements is inflated with an insulative or a cooling fluid to insulate or cool the bladder neck or the prostatic urethra.

FIG. 15B is a flowchart illustrating a method of ablation of prostatic tissue in accordance with another embodiment of the present specification. Referring to FIG. 15B, the first step 1502b includes passing a catheter into a patient's urethra, wherein the catheter includes a hollow shaft through which an ablative agent can travel, at least one first positioning element, at least one second positioning element positioned distal to said at least one first positioning element, at least one input port for receiving an ablative agent, and a plurality of needles positioned on said catheter between said first and second positioning elements and configured to deliver ablative agent to a prostatic tissue. In an embodiment, the ablation device includes a controller comprising a microprocessor for controlling the delivery of the ablative agent. The catheter is passed through the urethra such that the first positioning element is positioned proximate the prostatic tissue to be ablated and the second positioning element is positioned within the bladder of the patient. Next, in step 1504b, the second positioning element is deployed and the catheter is pulled back such that the second positioning element abuts a urethral opening at the neck of the bladder. The first positioning element is deployed such that the catheter is positioned within the urethra proximate to the prostatic tissue to be ablated in 1506b. In the next step 1508b, the plurality of needles is passed through the urethra into the prostatic tissue to be ablated. Finally, in step 1510b, an ablative agent is delivered through the needles to ablate the prostatic tissue. Optionally, in an embodiment, a cystoscope is first inserted in the patient's urethra and the catheter is inserted through the cystoscope. In various embodiments, the order of deployment of the positioning elements can be reversed. In other embodiments, only one of the two positioning elements may be deployed to deliver the therapy.

FIGS. 15C to 15E illustrate an embodiment of using an expandable catheter 1500 to expand/widen a constricted prostatic urethra 1538, in accordance with some embodiments of the present specification. The prostatic urethra 1538 has been constricted by an enlarged prostate 1530. Referring to FIG. 15C, compressed catheter 1500 with an expandable element 1525 is advanced into a prostatic urethra 1538. In embodiments, the expandable element 1525 comprises an expandable balloon or a self-expanding balloon. In embodiments, the expandable element 1525 is covered, for example, by a semi-permeable sheath. In other embodiments, the expandable element 1525 is uncovered. In embodiments, the expandable catheter 1500 includes a center post 1537. The center post includes one or more rows 1533 each comprising a plurality of openings for the delivery of ablative agent. In embodiments, each plurality of openings has a pattern of openings which may vary in shape, diameter, and quantity of openings to regulate ablative agent distribution. Referring to FIG. 15D, the expandable element 1525 on catheter 1500 is expanded and presses on the urethral walls 1539 which presses on the prostate 1530. Ablative agent 1541, such as steam, is then delivered into the prostatic tissue from the plurality of openings. Referring to FIG. 15E, catheter 1500 is removed from urethra 1538, leaving a widened prostatic urethra 1538. FIG. 15F illustrates an expanded expandable element 1525 of a catheter 1500 and an exemplary use of one or more needles 1550 to allow delivery of an ablative agent 1541, such as steam or vapor, through a hollow exit at the edge of the needle 1550. The needles extend from the center post 1537 of the catheter 1500, through the urethral wall 1539 and into the prostate 1530 to deliver ablative agent 1541 to the prostatic tissue. While the illustration of FIG. 15F describes the placing of the element 1525 into the prostate, the same arrangement can be used for both BPH and urethral strictures. In embodiments, needles 1550 are one or the needles illustrated and described in context of FIGS. 11A to 11J. In some embodiments, the expandable element 1525 is a wire mesh stent which can be removed at a later date. In another embodiment, the expandable element 1525 is made of a bioresorbable material and resorbs after a predetermined time. In some embodiments, the expandable element 1525 has a constraining and/or removing mechanism attached to it for removal at a later date. In some embodiments, the constraining and or removing mechanism is a PTFE, ePTFE or silk suture. In some embodiments, the expandable element comprises extracellular matrix to help proper healing of the prostatic urethra post-ablation.

Median lobe hyperplasia is a benign condition in which the median lobe of the prostate become enlarged and presses into the base of the bladder, causing a ball valve type obstruction at the bladder neck. For ablation therapy, it is desirable to access the median lobe, especially the most affected part of the median lobe, trans-cystically rather than transurethrally. Accessing the median lobe of the prostate through the bladder, rather than through the urethra, has the advantage of not causing ablation damage to the urethra with subsequent urethral stricture. FIG. 15G illustrates an ablation catheter 1560 used to ablate prostatic tissue of a patient with median lobe hyperplasia via a trans-cystic approach, in accordance with one embodiment of the present specification. FIG. 15H illustrates an ablation catheter used to ablate prostatic tissue of a patient with median lobe hyperplasia via a trans-cystic approach, in accordance with another embodiment of the present specification. In the embodiment of FIG. 15G, the catheter 1560 includes at least one curved vapor delivery needle 1561 extending at its distal end. In the embodiment of FIG. 15H, the catheter 1565 includes at least one straight vapor delivery needle 1566 extending at its distal end. The one or more needles, and their composition and method of deployment, may be similar to the other needle embodiments discussed in the embodiments of the present specification. Referring to FIGS. 15G and 15H simultaneously, the catheter 1560, 1565 is depicted inserted into and through the patient's spongy or penile urethra 1571, through a prostatic urethra 1572, and into the patient's bladder 1573. In embodiments, the distal end of the catheter 1560, 1565 is advanced to be positioned just beyond a bladder neck 1574 and within the bladder 1573, just into the bladder 1573 past the internal urethral meatus 1576 (opening of the bladder into the prostatic urethra). At least one needle 1561, 1566 is extended from the distal end of the catheter 1560, 1565 into the bladder 1573 cavity, through the bladder wall 1577, into the medial lobe 1575. Ablative agent, in the form of vapor or steam, is delivered through the at least one needle 1561, 1566 to ablate the tissue of the median lobe 1575. In some embodiments, the catheter 1560, 1565 optionally includes at least one positioning element 1562, 1564 configured to position the catheter inside the bladder 1573 and to stabilize the needle 1561, 1566 to assist in the needle 1561, 1566 penetrating the median lobe 1575. In various embodiments, the positioning element 1562, 1564 comprises a shape memory material configurable between a first, collapsed configuration for delivery and a second, expanded configuration for positioning. In various embodiments, the positioning element 1562, 1564, in the second, expanded configuration, has a disc, cone, hood, ovoid, oval, square, rectangular, or flower shape. In various embodiments, tension wires attached to the needle may be used to manipulate the needle and assist in puncturing into the prostate.

FIG. 15I is a flowchart listing the steps in one method of using an ablation catheter to ablate prostatic tissue of a patient with median lobe hyperplasia via a trans-cystic approach, in accordance with one embodiment of the present specification. At step 1580, an ablation catheter comprising at least one needle is passed into a patient's spongy urethra and through a prostatic urethra such that a distal end of the catheter is positioned within the patient's bladder. Optionally, the ablation catheter further comprises at least one positioning element configured to position the catheter in the bladder and to stabilize the at least one needle for penetrating the median lobe. Optionally, at step 1581, the positioning element is deployed to position the catheter and stabilize the at least one needle. At step 1582, the at least one needle is extended from the distal end of the catheter and is passed through the bladder or bladder neck wall and into the median lobe of the prostate. At step 1583, an ablative agent is delivered through the at least one needle into the median lobe to ablate prostatic tissue. In embodiments, the ablation catheter is a part of an ablation system comprising a controller and a means for generating the ablative agent. At step 1584, the controller controls the delivery of ablative agent to maintain a pressure in the bladder and median lobe below 5 atm.

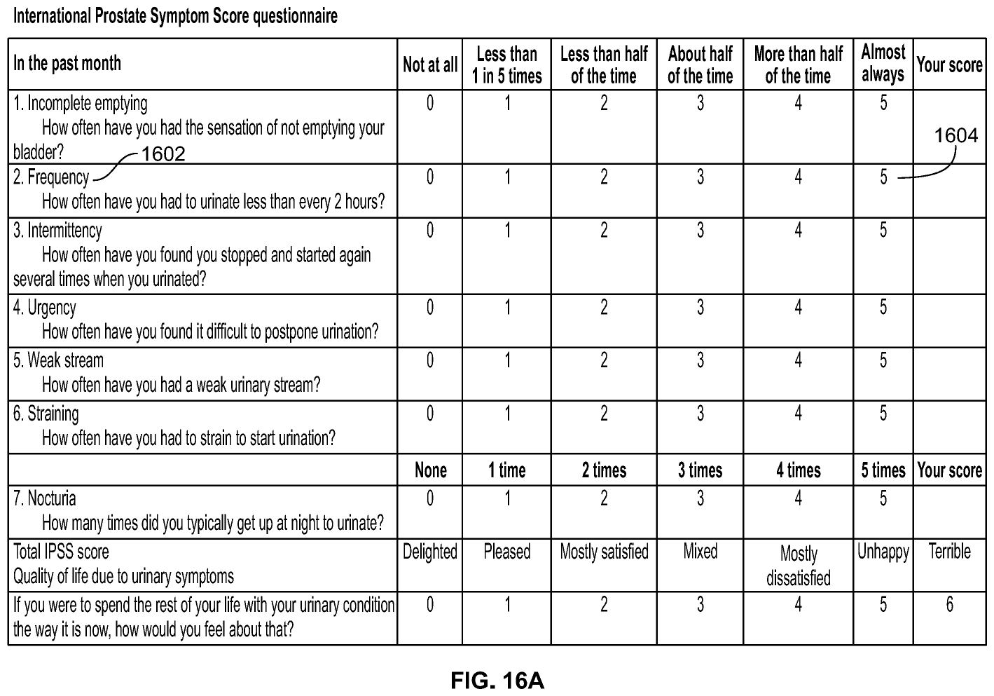

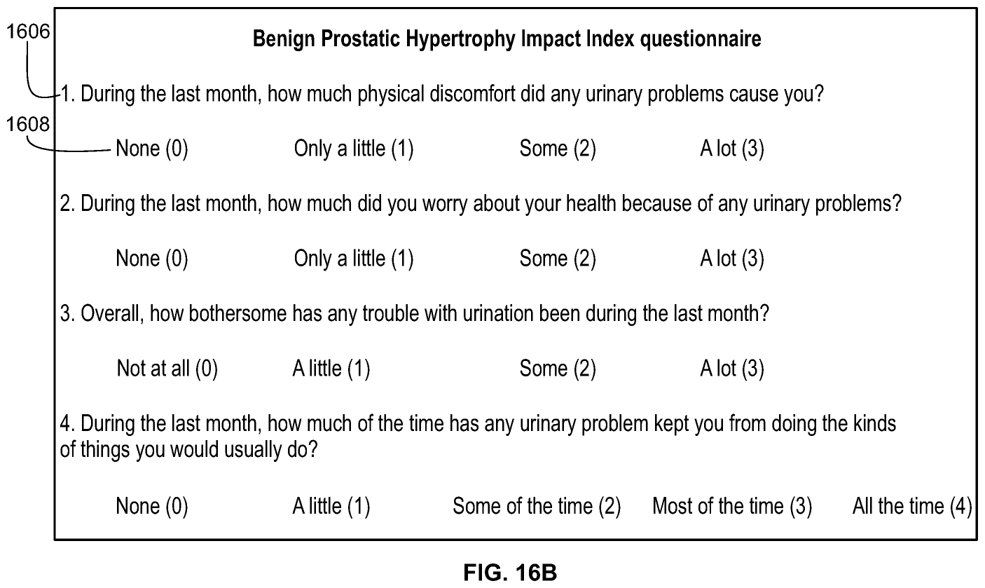

In various embodiments, ablation therapy provided by the vapor ablation systems of the present specification is delivered to achieve the following therapeutic endpoints for prostate ablation: maintain a tissue temperature at 100° C. or less; improve patient urine flow by at least 5% relative to pre-treatment urine flow at six-month follow-up from the treatment; decrease prostate volume by at least 5% relative to pre-treatment prostate volume at follow-up after six months from treatment; decrease in post-void residual by greater than 5% at the six-month follow-up; decrease in incidence of acute urinary retention by 5% at 12-month follow-up; decrease in prostate-specific antigen by 5% at six-month follow-up; improvement in the American Urological Association symptom index by more than 5% at the six-month follow-up; ablate the prostate tissue without circumferentially ablating a urethral tissue; improve International Prostate Symptom Score (IPSS) by at least 5% relative to a pre-treatment IPSS score, wherein the IPSS questionnaire, depicted in FIG. 16A, comprises a series of questions 1602 regarding a patient's urinary habits with numerical scores 1604 for each question; improve Benign Prostatic Hypertrophy Impact Index Questionnaire (BPHIIQ) score by at least 10% relative to a pre-treatment BPHIIQ score, wherein the BPHIIQ, depicted in FIG. 16B, comprises a series of questions 1606 regarding a patient's urinary problems with numerical scores 1608 for each question; and patient reported satisfaction with the ablation procedure of greater than 25%.

### Endometrial Ablation

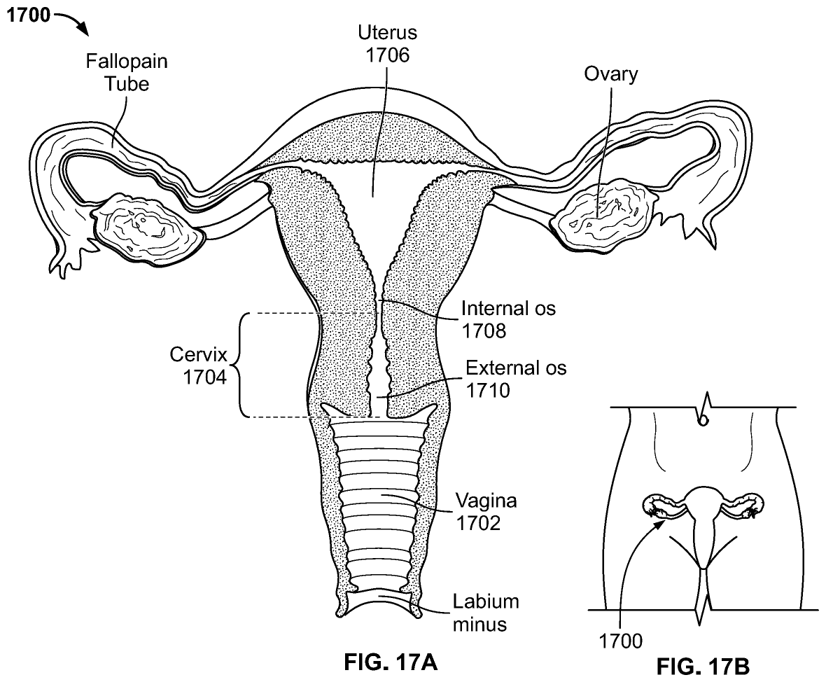

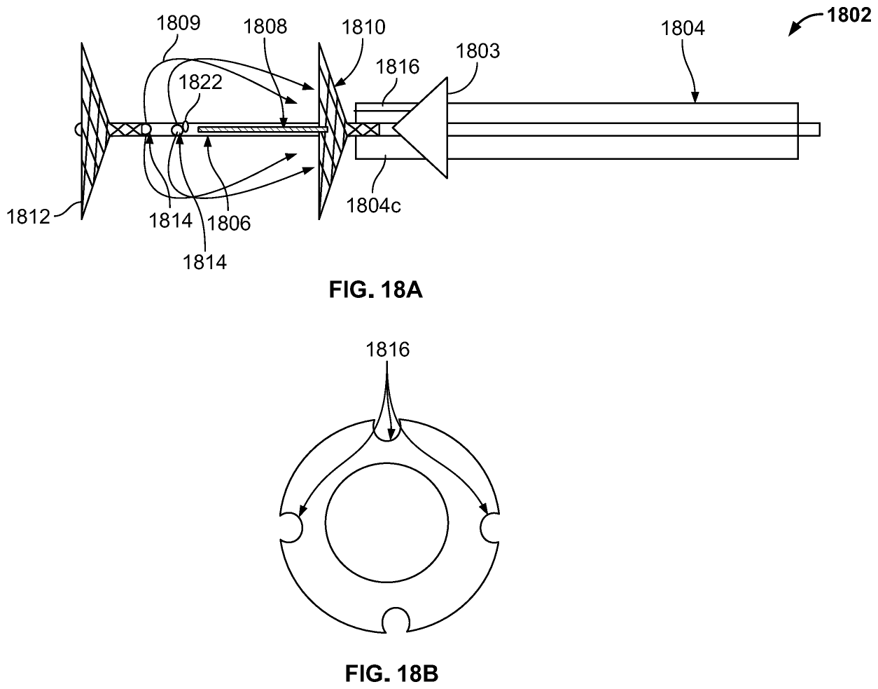

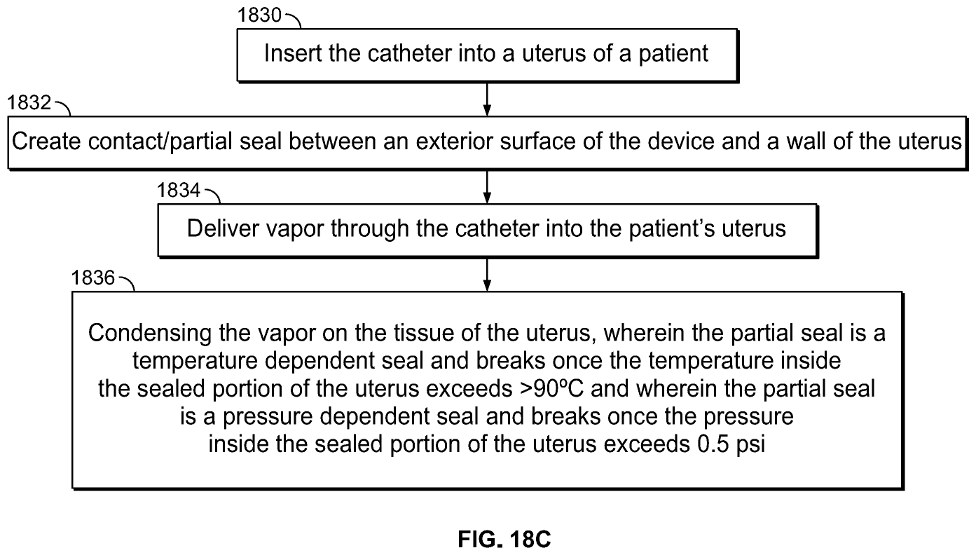

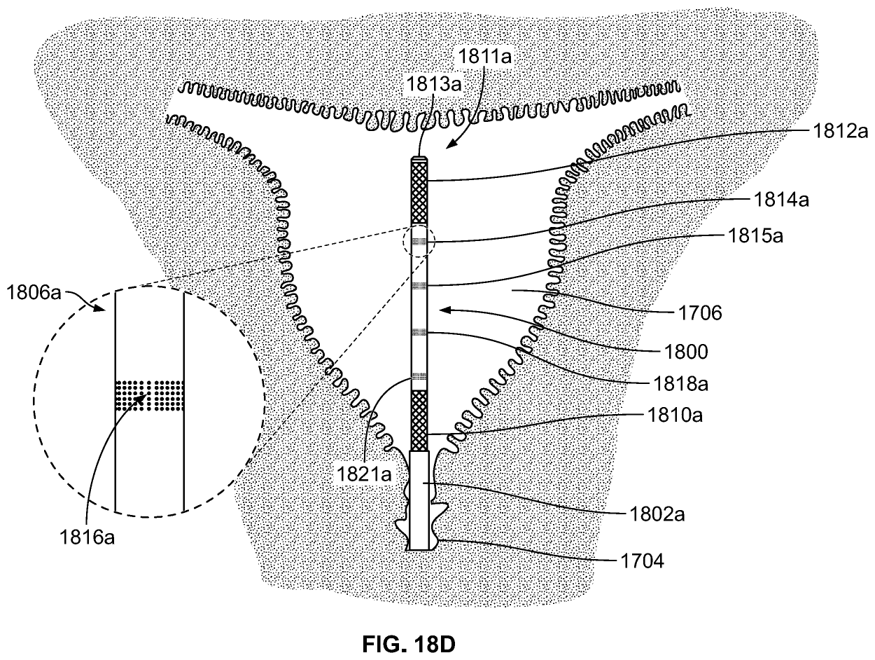

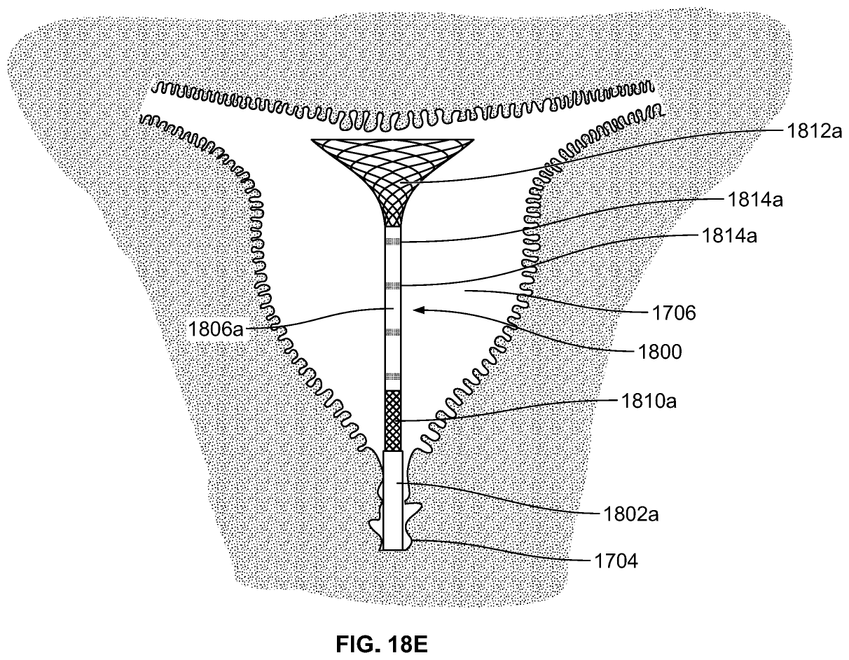

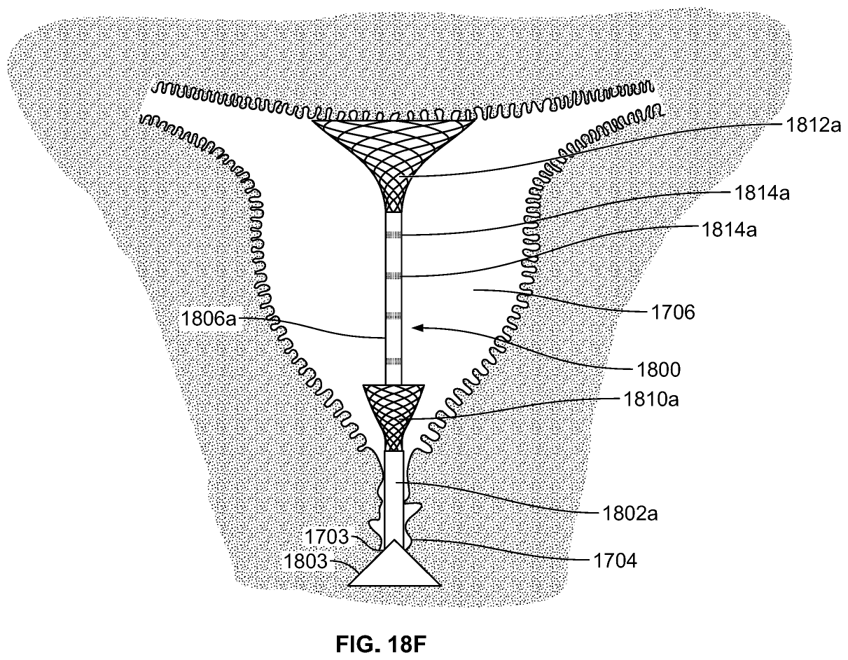

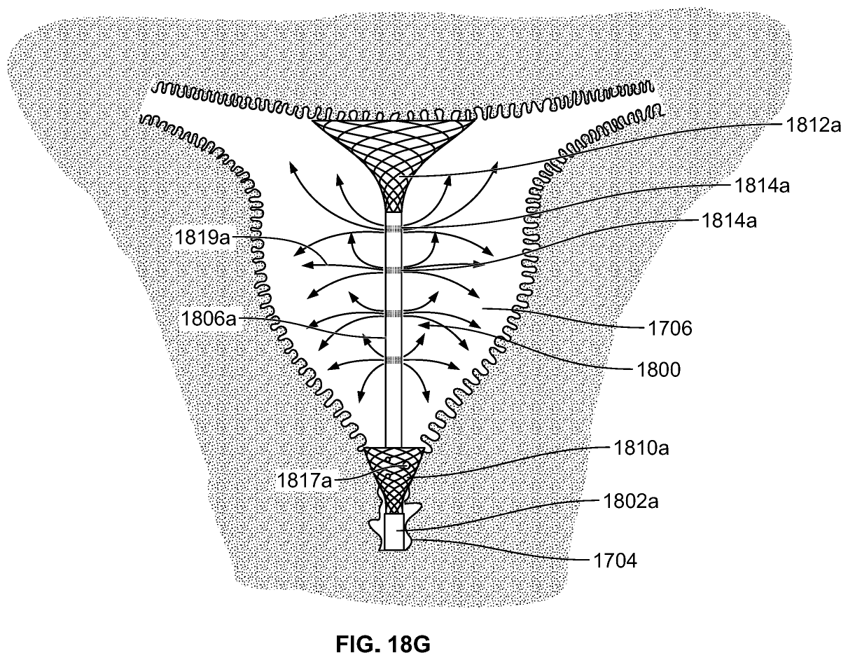

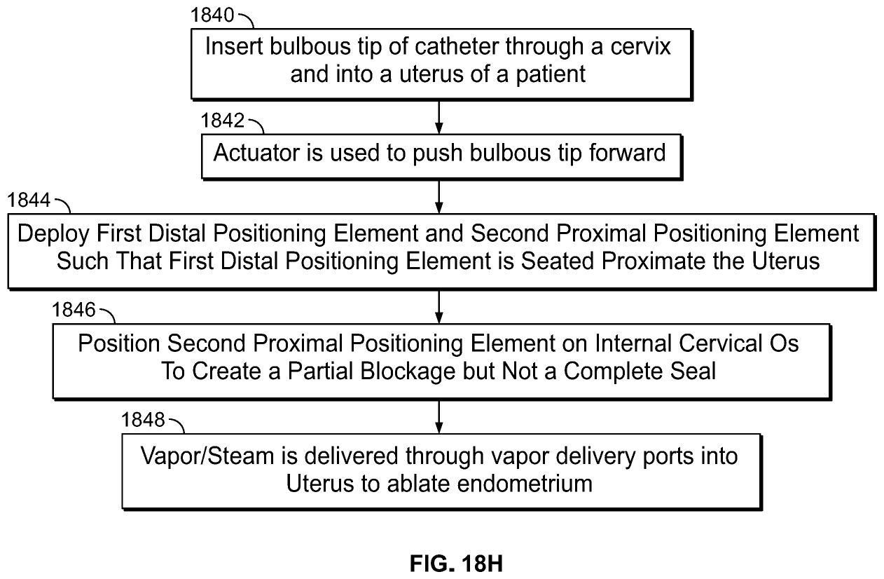

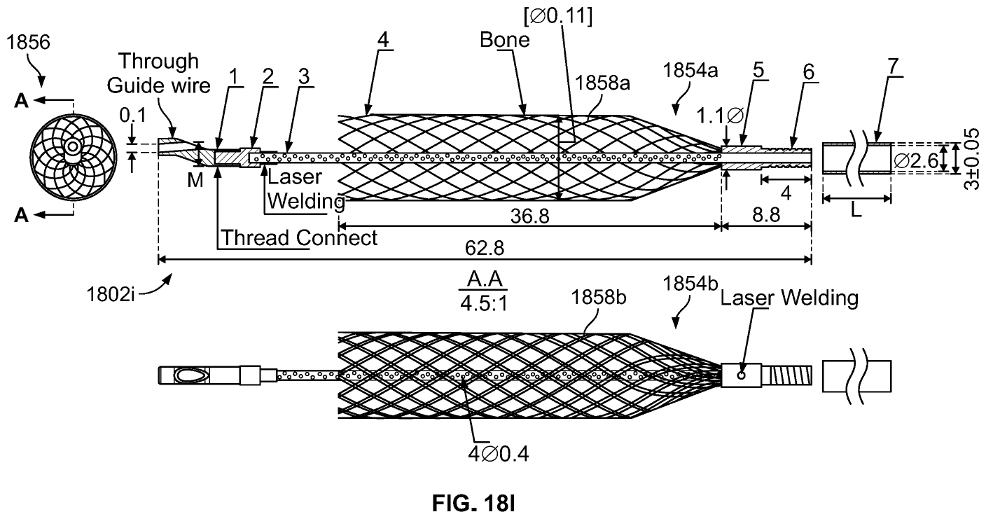

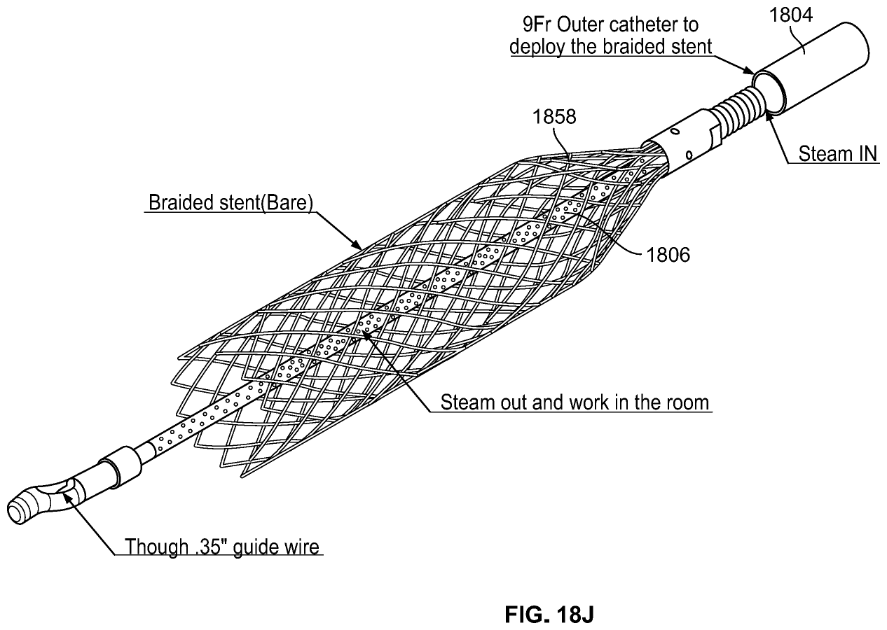

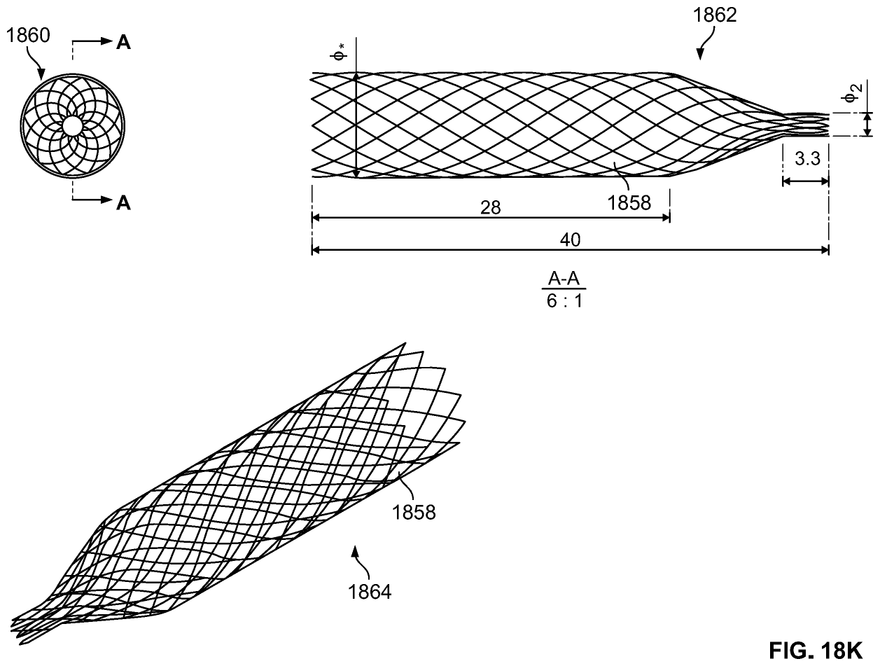

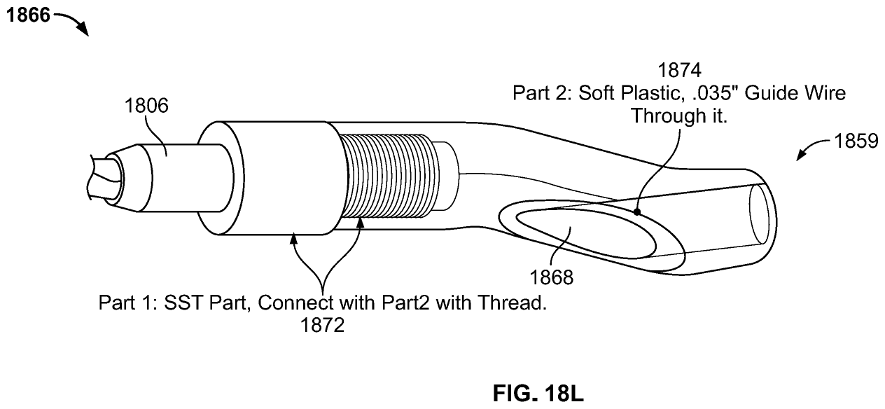

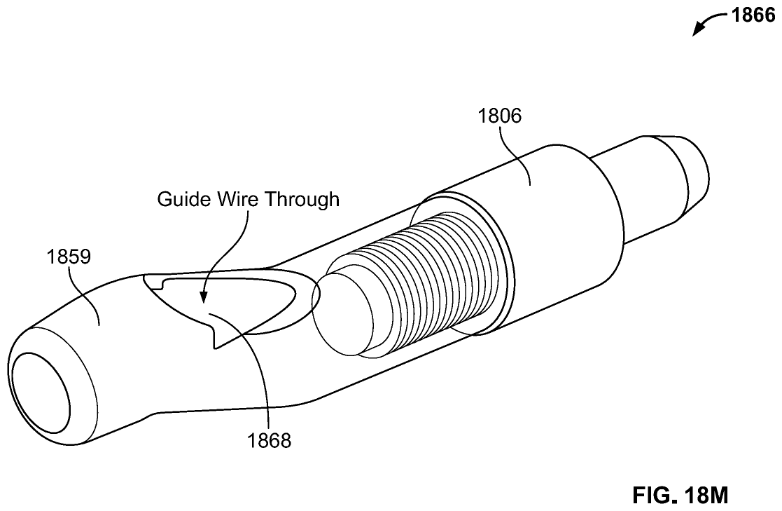

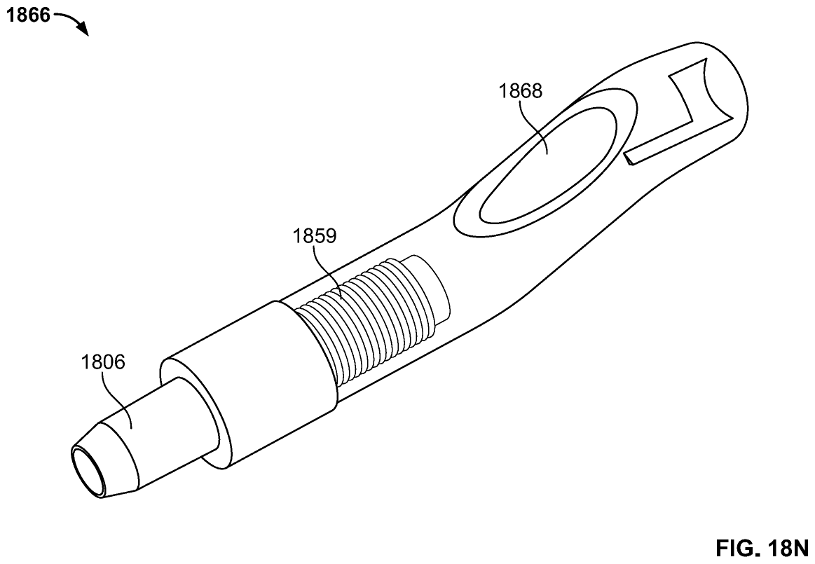

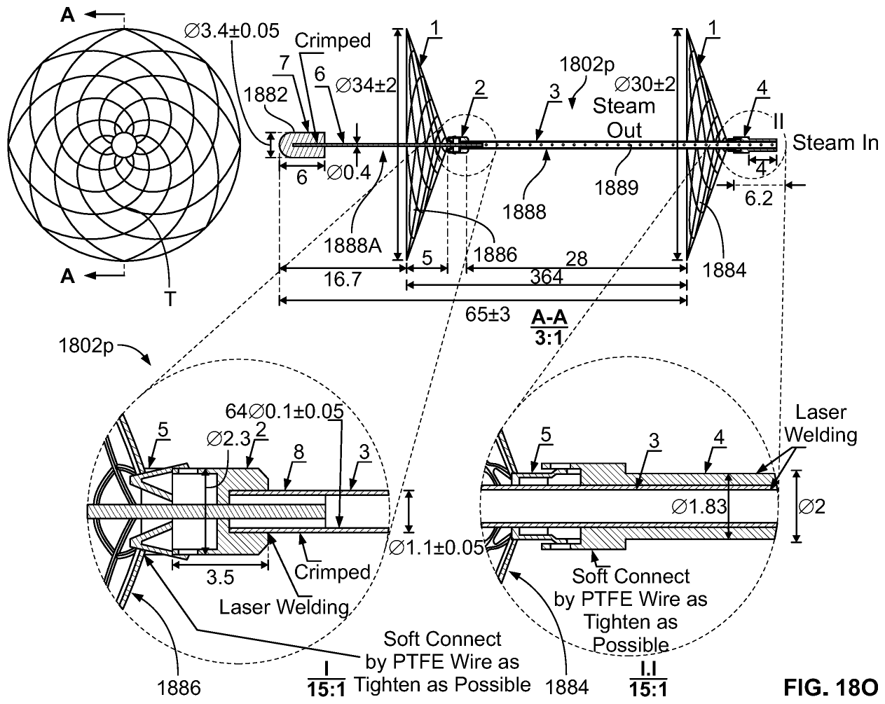

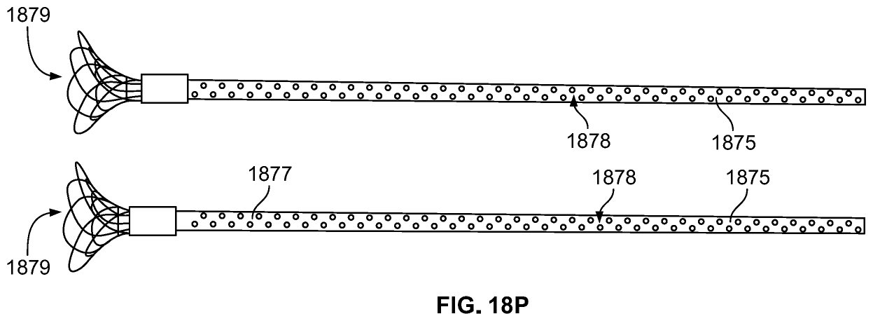

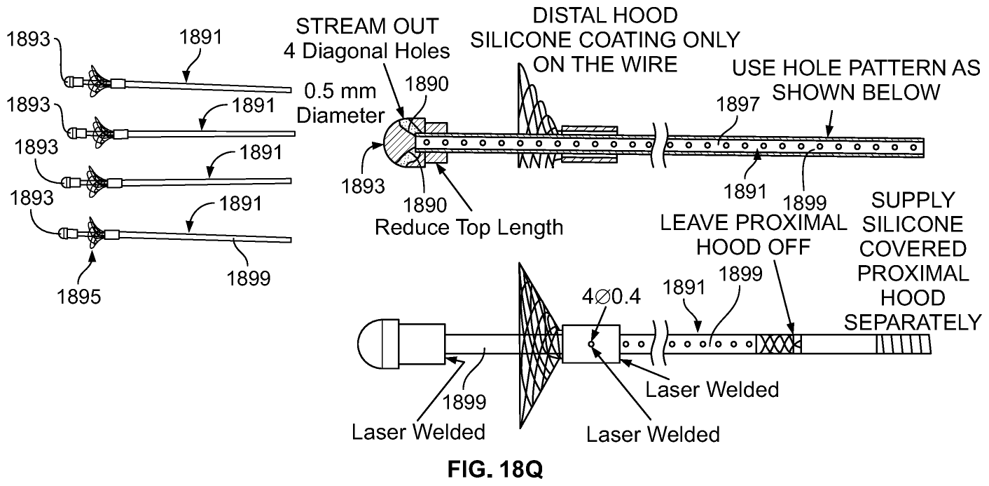

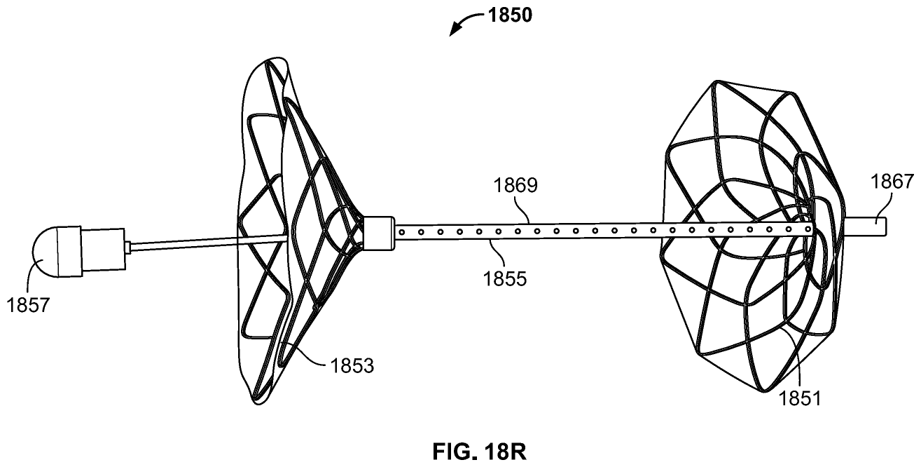

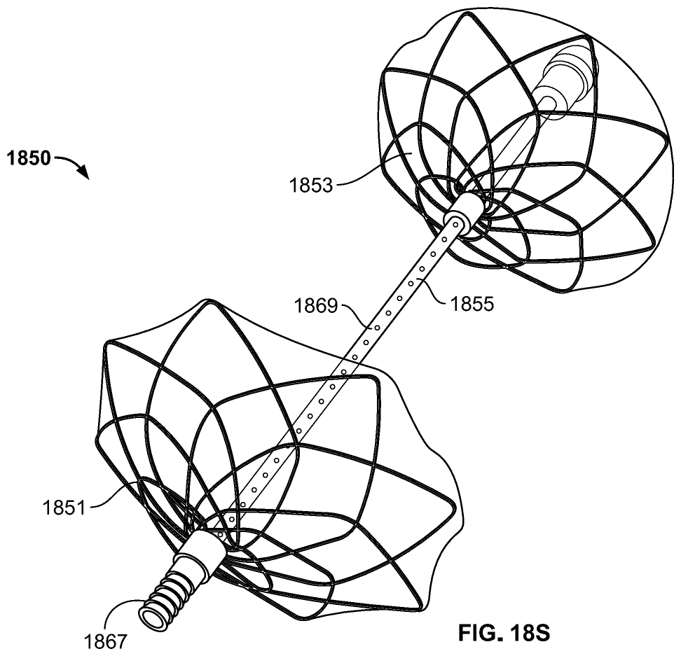

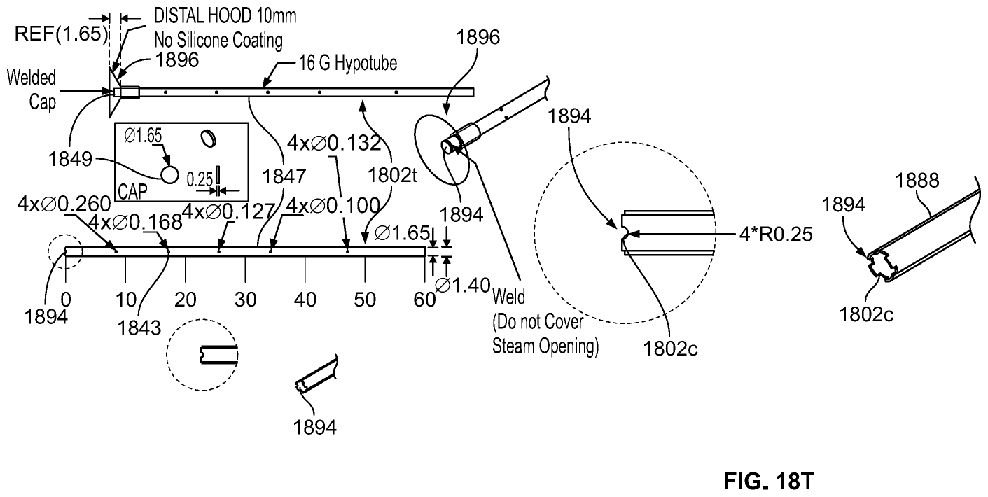

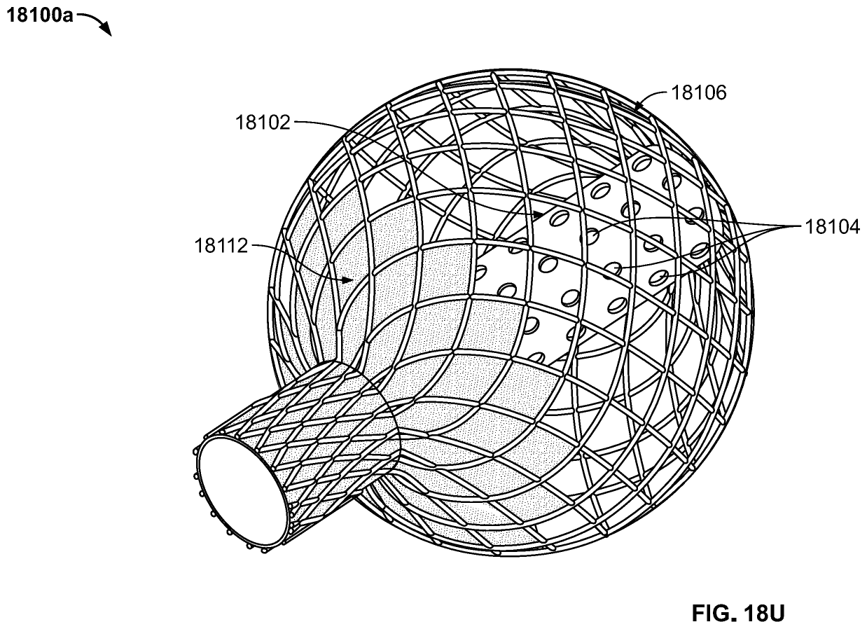

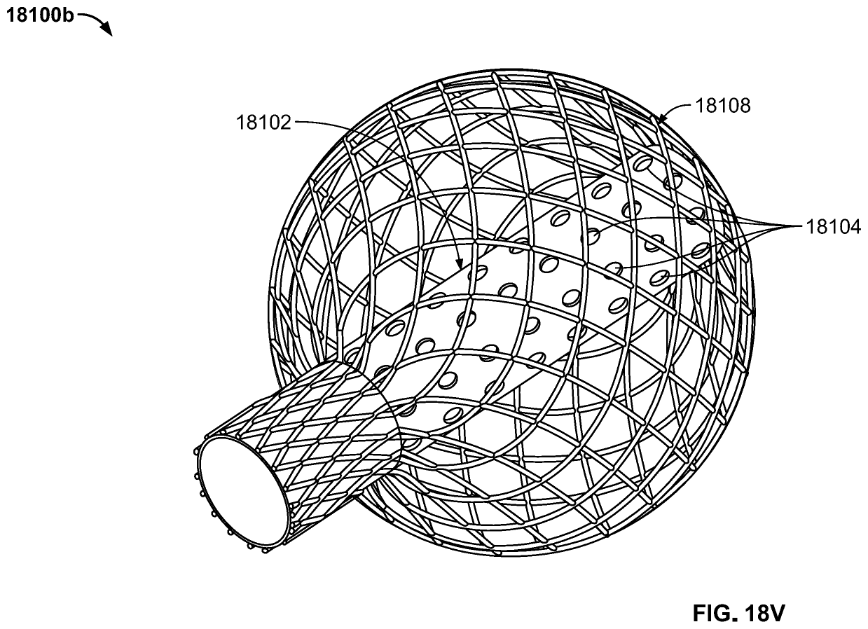

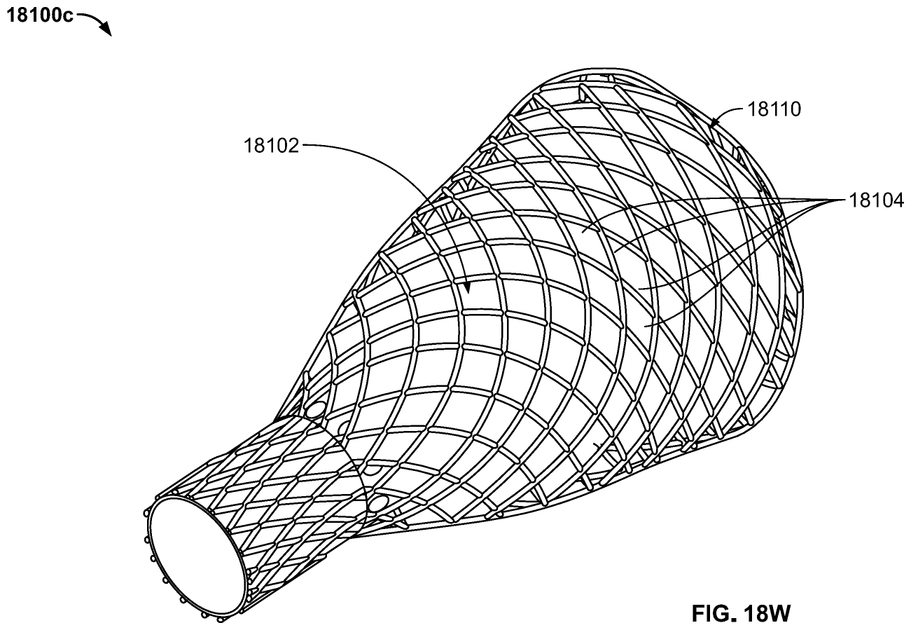

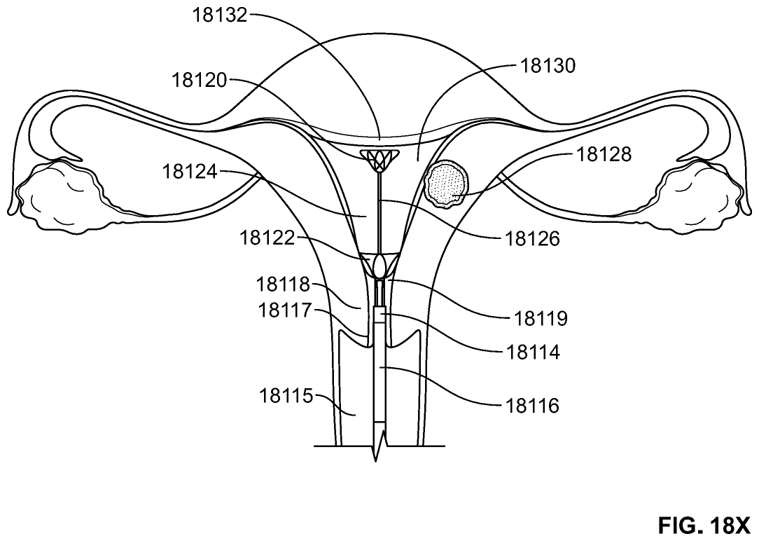

FIG. 17A illustrates a typical anatomy 1700 of the uterus 1706 and uterine tubes of a human female. FIG. 17B illustrates the location of the uterus and surrounding anatomical structures 1700 within a female body. FIG. 18A illustrates an exemplary ablation catheter 1802 arrangement for ablating the uterus 1706, in accordance with some embodiments of the present specification. Referring simultaneously to FIGS. 17A and 18A, in embodiments, a coaxial catheter 1802 is used to insert into vagina 1702 of a patient and advanced toward the cervix 1704. Catheter 1802 comprises an outer catheter 1804 and an inner catheter 1806. Inner catheter 1806 is concentric with and has a smaller radius than outer catheter 1804. An electrode 1808 for heating the catheter tip is located between the two positioning elements 1810, 1812. In some embodiments, the electrode 1808 is proximal to the proximal positioning element 1810. In some embodiments, the positioning elements are discs—a proximal disc 1810 and a distal disc 1812. For purposes of the present specification, discs 1810 and 1812 may also be referred to as hoods 1810 and 1812. In some embodiments, the distal hood 1812 has a smaller diameter than the proximal hood 1810. In some embodiments, the distal hood 1812 is approximately 5 mm smaller than the proximal hood 1810. In embodiments, the hoods 1810 and 1812 are made from wires with different wire stiffness. The distal hood 1812 is configured to contact fundus of the uterus 1706, and acts like a scaffolding to push to halves of uterus away from each other. The proximal hood 1810 is configured to occlude an internal cervical os 1708.

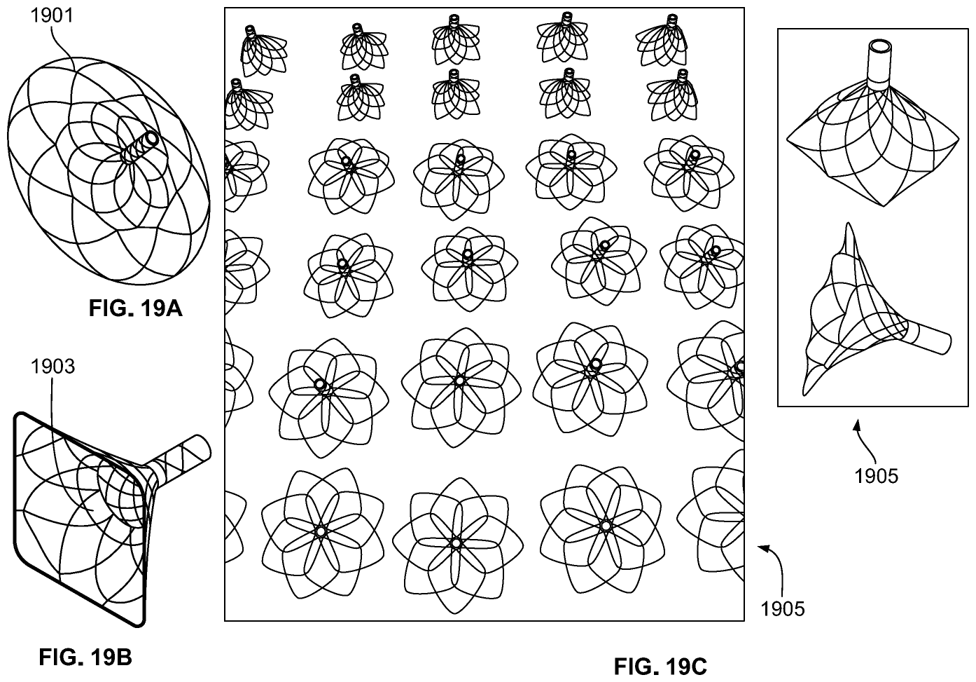

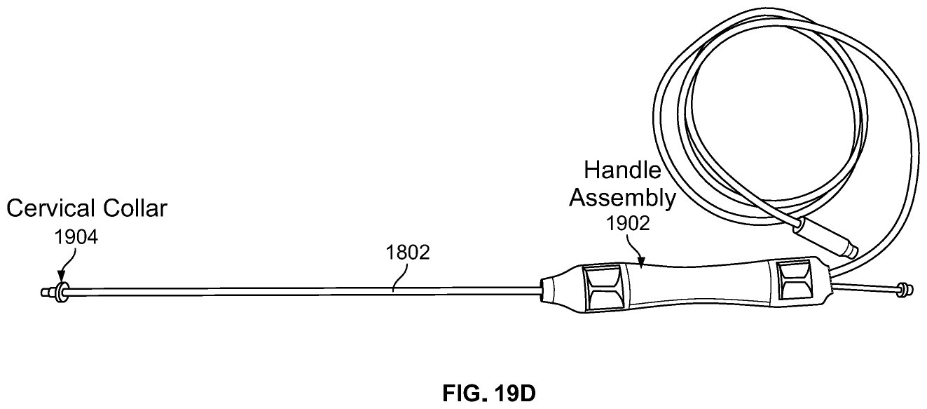

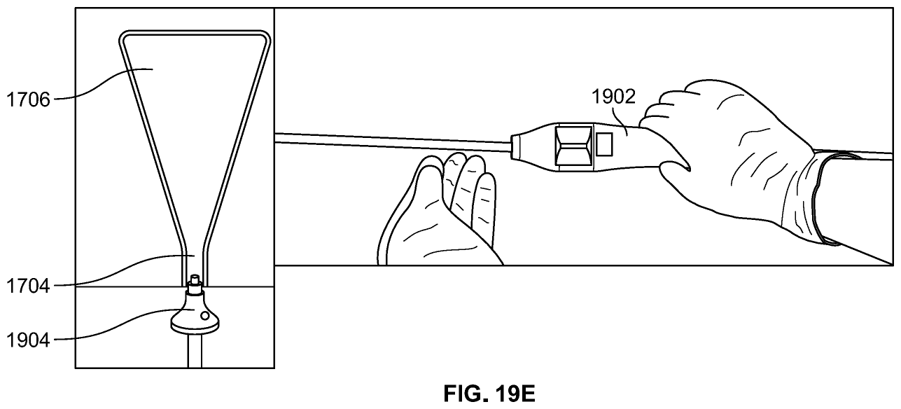

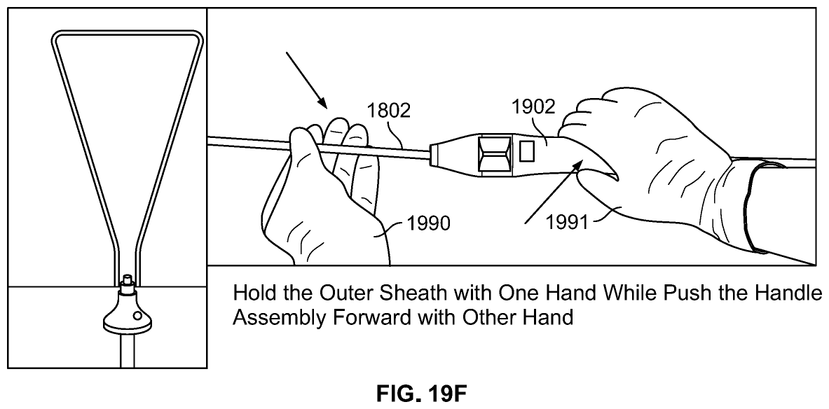

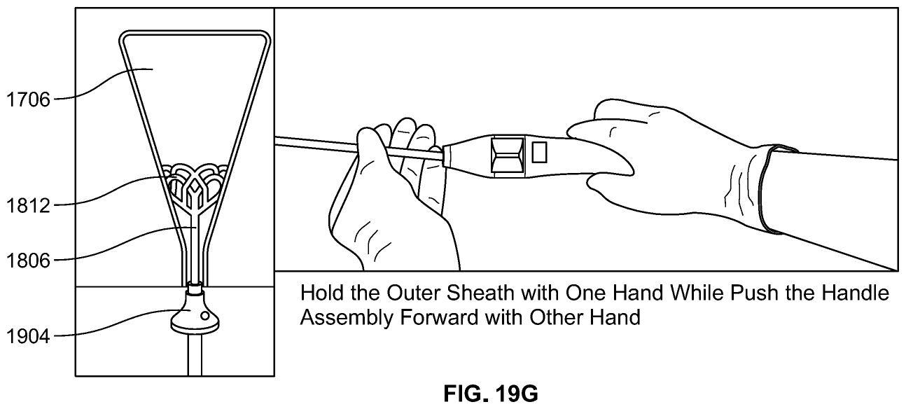

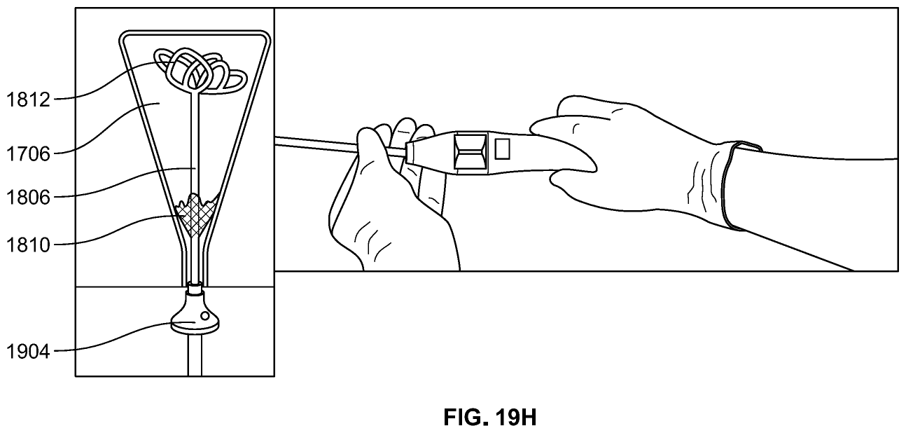

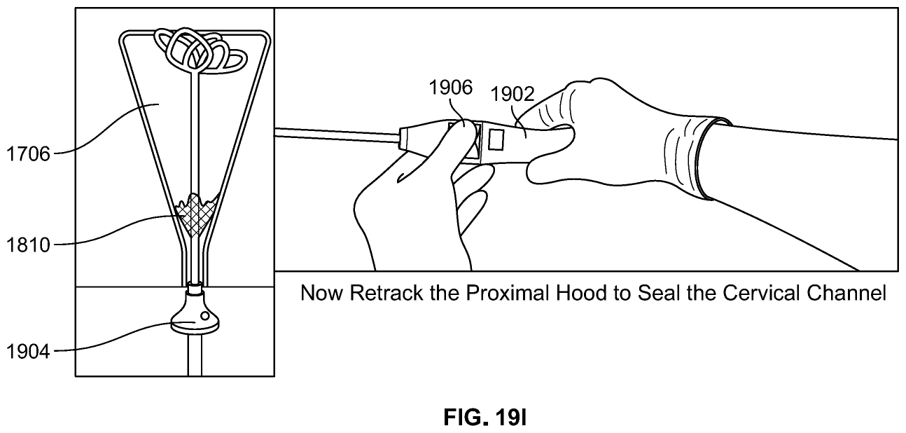

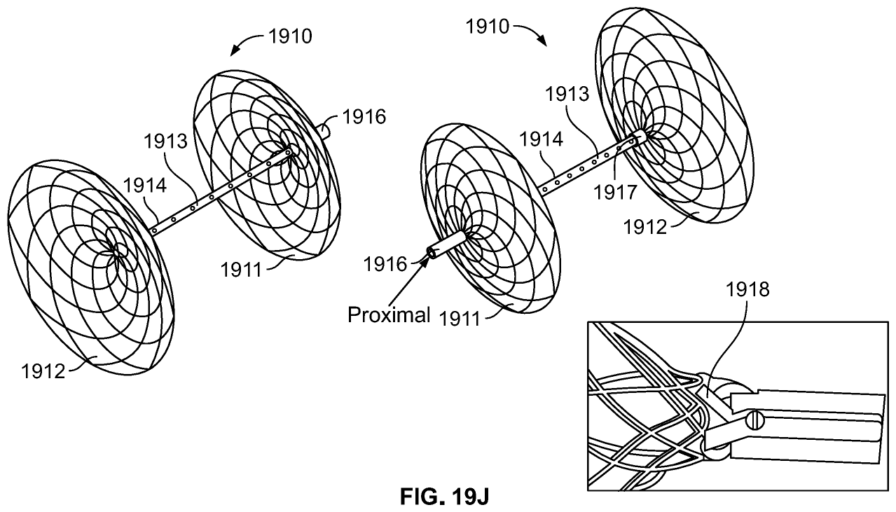

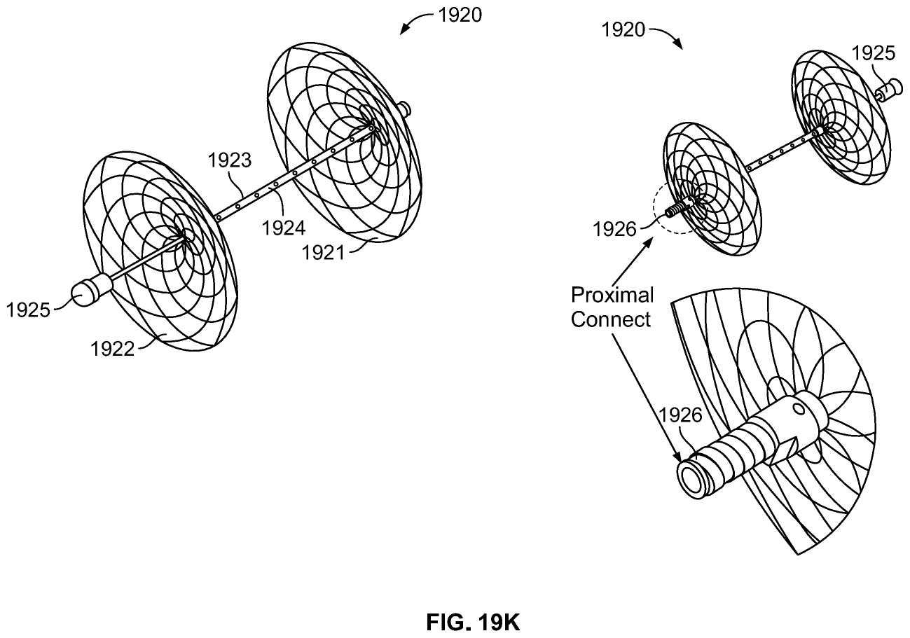

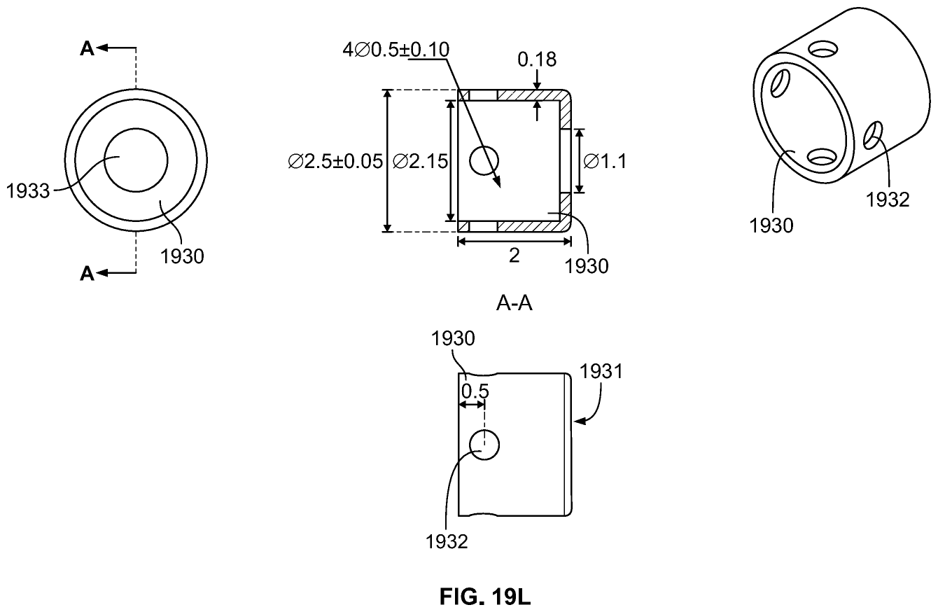

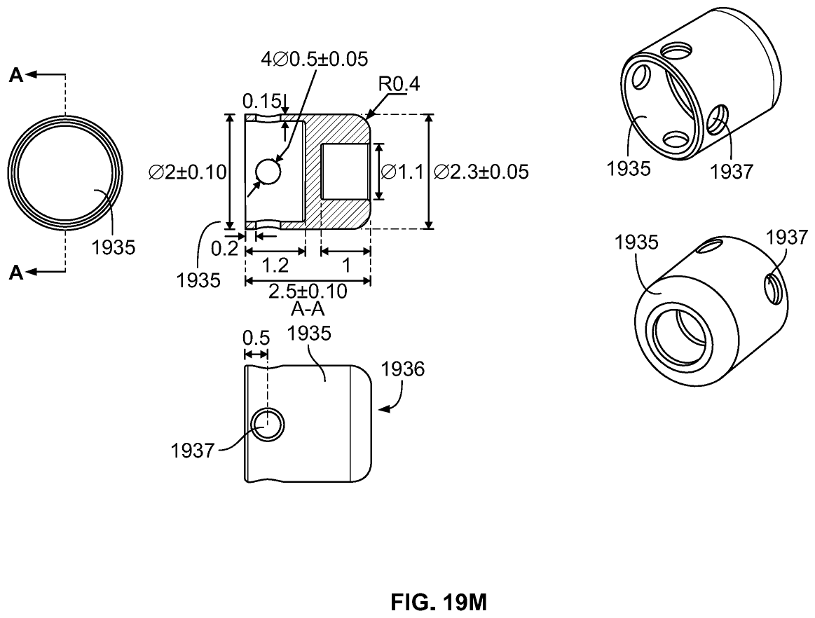

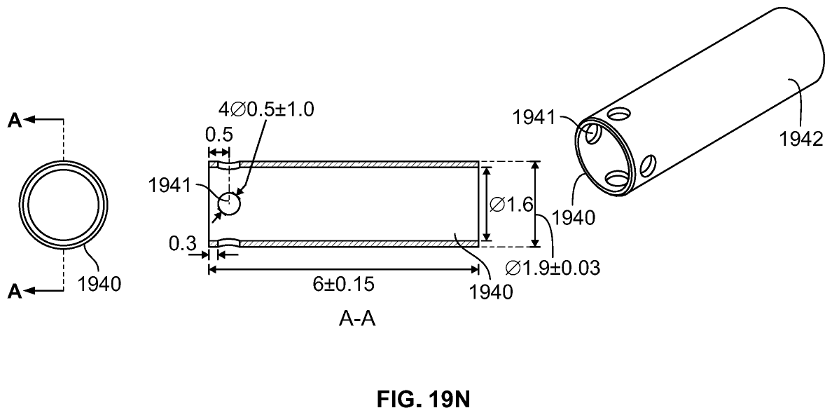

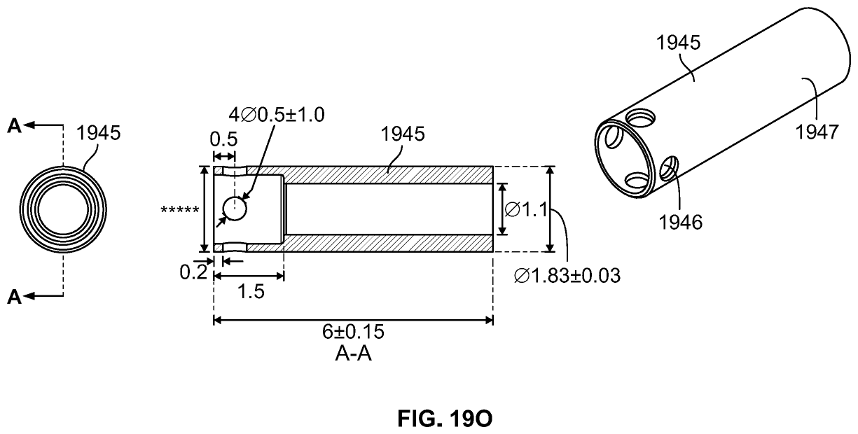

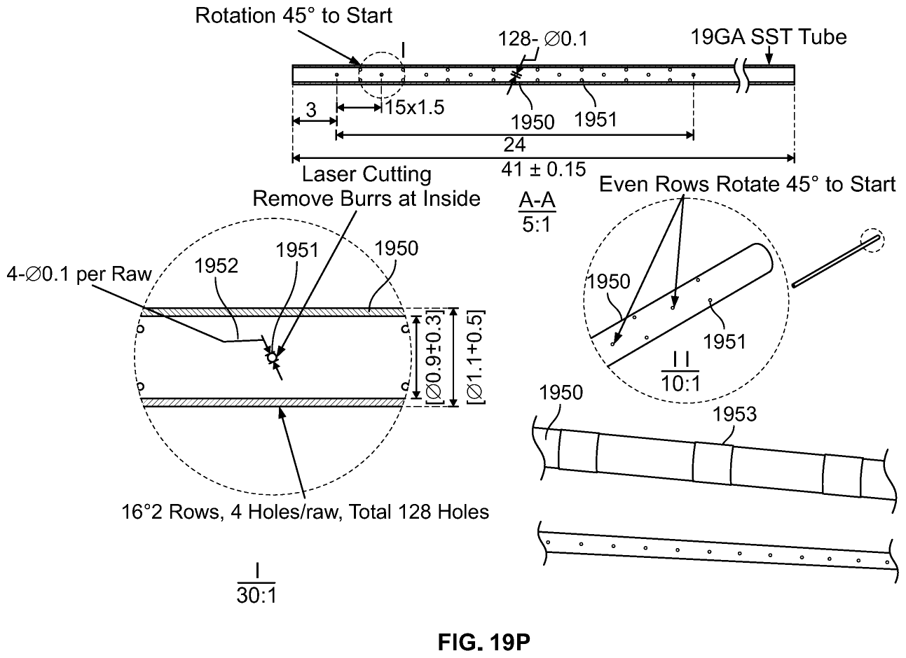

FIGS. 19A, 19B, and 19C illustrate different types of configurations 1901, 1903, 1905 of distal and proximal discs 1812, 1810, which may be used in accordance with the embodiments of the present specification. The discs may differ in stiffness and size and may be chosen by a physician based on the indication for treatment. In some embodiments, the discs are conical in shape with diameters varying from 5 mm to 50 mm. In some embodiments, the positioning elements are ovoid cones with a first proximal diameter of the cone less than a second distal diameter of the cone to approximate the shape or dimensions of a uterine cavity. In various embodiments, a first positioning element may have a different shape or size from a second positioning element. One or more positioning elements maybe used to accomplish the therapeutic purpose.

In some embodiments, the discs 1812, 1810 are formed with wire made from one or a combination of polymers and metal, such as including and not limited to Polyether ether ketone (PEEK) and Nickel Titanium (NiTi). In some embodiments, the wire is covered with elastomers such as PU and/or silicone in a variety of patterns. The various cells in the discs 1812, 1810 may be covered or uncovered based on hood functionality such as whether it is to be used for sealing, or for venting, or for any other purpose. In embodiments where the positioning elements 1812, 1810, are made from Nitinol wire meshes, the wires have a diameter in a range of 0.16 to 0.18 mm. In some embodiments, for the distal positioning element 1812, the wire mesh is coated with silicone but not the areas between wires in the mesh, therefore allowing steam to escape/vent from these spaces between the wires. In some embodiments, for proximal positioning elements 1810, wires and space between wires are covered with silicone.

In embodiments, the inner catheter 1806 is movable into and out of the outer catheter 1804 such that the outer catheter 1804 covers the inner catheter 1806 and restrains the positioning elements 1810, 1812 before insertion into a patient's uterus. The positioning elements 1810, 1812 are composed of a shape memory material such that they expand into a deployed configuration, as shown in FIG. 18A, once the inner catheter 1806 is extended beyond the distal end of the outer catheter 1804.

In embodiments, catheter 1802, with the inner catheter 1806 disposed within the outer catheter 1804 and the positioning elements 1810, 1812 in a first, restrained configuration, is inserted into the vagina 1702 so that the distal end of the outer catheter 1804 is positioned proximate the internal os 1708. The internal catheter is then advanced into the uterus 1706. The catheter 1802 is advanced until distal disc 1812 is within uterus 1706 and proximal disc 1810 occludes the uterus 1706 by positioning it proximate the internal os 1708. In embodiments, the catheter 1802 includes a cervical collar 1803 attached to the outer 1804 catheter. The cervical collar 1803 rests against an external os when the catheter 1802 is deployed in a patient's uterus and assists in maintaining the catheter 1802 in a correct position. A distal portion 1804c of the outer catheter 1804, which extends from the cervical collar 1803 to a point proximal to the proximal positioning element 1810 is positioned within the cervix or the cervical canal when the catheter 1802 is deployed. In embodiments, the distal 1812 and proximal 1810 positioning elements move independently, or expand and lock together. In embodiments, the inserted length of the internal catheter 1806 is used to measure the uterine depth and determine the amount of the vapor ablative to be used in order to keep the pressure inside the uterus 1706 below a predefined threshold. Vapor ports 1814 are positioned on the inner catheter 1806 between the distal disc 1812 and proximal disc 1810 to output vapor for ablation. The plurality of vapor ports is positioned in a circumferential pattern around a length of the catheter. Vapor ports may vary in size, shape or port density (number of ports/length of a catheter) to optimize vapor delivery into the uterine cavity. The vapor 1809 heats the endometrium proximate the distal disc 1812 and then travels in a direction toward the proximal disc 1810, while pushing the endometrial air out. In another embodiment, the vapor delivery ports are configured to heat the entire endometrial cavity simultaneously and uniformly. In embodiments, at least one of the inner catheter 1806 or outer catheter 1804 catheter include venting elements or grooves 1816 that allow venting of the uterus 1706, to allow escape of the endometrial air and preventing over-pressurization of the endometrial cavity. In some embodiments, the grooves may be present around a percentage of a total circumference of the inner catheter 1806 or outer catheter 1804. In some embodiments, the grooves are present around a total circumference of the inner catheter 1806 or the outer catheter 1804, and more preferably around 1 to 90% of a total circumference of the inner catheter 1806 or the outer catheter 1804. FIG. 18B illustrates an exemplary embodiment of grooves 1816 configured in the inner catheter 1806 walls, in accordance with some embodiments of the present specification. In some embodiments, openings in the proximal disc 1810 allow venting of the uterus 1706. In embodiments, the proximal disc 1810 is covered in an elastomer, such as PU or silicone, in a pattern having various cells or openings in the disc 1810 which are uncovered and allow for venting during ablation. In other embodiments, where a seal is desired, there are no uncovered cells or openings in the disc 1810 to allow for venting. In embodiments, a pressure sensor 1822 is used with catheter 1802 to check and subsequently maintain a pressure within the uterus 1706 below 50 mm Hg, preferably below 30 mm Hg, and more preferably below 15 mm Hg. In embodiments, the pressure is also maintained at no more than 10% above atmospheric pressure. As a result of the low pressure level that is maintained within the uterus, embodiments of the present specification are able to forego an integrity check, which is otherwise time consuming and involves risks, and is required in implementation of the prior art. In one embodiment, the endometrial cavity pressure is measured by a generator by measuring the back pressure on the saline being pushed through the inner catheter to the electrode and can be modulated by modulating the saline flow to maintain the endometrial cavity pressure at less than 5 atm. In some embodiments, the endometrial cavity pressure is maintained at less than 0.5 atm.

FIG. 18C is a flowchart of one method of using the catheter of FIG. 18A to ablate endometrial tissue, in accordance with some embodiments of the present specification. At step 1830, the catheter is inserted into a uterus of a patient. At step 1832, contact, or a partial seal is created between an exterior surface of the device and a wall of the uterus. Vapor is then delivered through the catheter into the patient's uterus at step 1834. At step 1836, the vapor condenses on the tissue of the uterus, wherein the partial seal is a temperature dependent seal and breaks once the temperature inside the sealed portion of the uterus exceeds >90° C. and wherein the partial seal is a pressure dependent seal and breaks once the pressure inside the sealed portion of the uterus exceeds 1.5 psi, preferably 1.0 psi, and more preferably 0.5 psi. In another embodiment, the partial seal breaks once the pressure inside the sealed portion of the uterus exceeds 2 psi or 10 mm Hg. In another embodiment, the partial seal breaks when the pressure exceeds 6 psi or 30 mm Hg. In some embodiments, the partial seal is a pressure dependent seal and breaks once the temperature inside the sealed portion of the uterus exceeds 101° C. and the pressure exceeds 0.5 psi. In some embodiments, the partial seal is a pressure dependent seal and breaks once the temperature inside the sealed portion of the uterus exceeds 102° C. and the pressure exceeds 1.0 psi. In some embodiments, the partial seal is a pressure dependent seal and breaks once the temperature inside the sealed portion of the uterus exceeds 103° C. and the pressure exceeds 1.5 psi.

FIGS. 18D to 18G illustrate an embodiment of an endometrial ablation catheter 1800 of the system of FIG. 1P, in accordance with the present specification. Referring to FIG. 18D, catheter 1800 has an outer catheter or sheath 1802a and an inner catheter 1806a. In some embodiments, an outer diameter of inner catheter 1806a is approximately 3.5 mm. In some embodiments, the distal end 1811a of the catheter 1800 has a bulbous tip 1813a to allow for atraumatic insertion into a patient's vagina, through the patient's cervical canal 1704, and into the uterus 1706, without the need of pre-dilation of the cervix. A plurality of rows 1814a, 1815a, 1818a, 1821a each having a plurality of vapor delivery ports 1816a, is positioned between a distal positioning element 1812a and a proximal positioning element 1810a. In different embodiments, the numbers of ports 1816a vary from 1 to 10,000. In some embodiments, the numbers of ports 1816a are within a range of 64 to 96 ports. In embodiments, size of the hole in each port 1816a is within a range of 0.01 to 1 mm. In an embodiment, size of the hole is 0.1 mm. In various embodiments, the vapor delivery ports 1816a are sized differently in the different rows 1814a, 1815a, 1818a, 1821a, creating a steam gradient along the catheter and within the organ volume. For example, in some embodiments, larger delivery ports are positioned at the distal row 1814a to maximize steam in a larger volume of cavity and smaller delivery ports are positioned at proximal row 1821a for a smaller volume of cavity. In embodiments, row 1815a includes ports smaller than those of row 1814a while row 1818a includes ports smaller than those of 1815a but larger than those of 1821a. In other embodiments, the ports in distal rows 1814a, 1815a, or distal half of the catheter 1800, have a total surface greater than a total surface area of the ports in the proximal rows 1818a, 1821a, or proximal half, of the catheter 1800. In another embodiment, port size remains consistent and the port density in the various rows or regions of the catheter varies.

Referring to FIG. 18E, catheter 1800 is advanced through a cervical canal 1704 and into a uterus 1706 such that the inner catheter 1806a is positioned within uterus 1706 and the outer sheath 1802a is positioned within cervical canal 1704. The distal positioning element 1812a is expanded. In embodiments, the positioning element 1812a may vary in size, shape, diameter, geometry, or any other structural feature, so as to regulate steam distribution in a desired manner. Referring to FIG. 18F, distal positioning element 1812a, having, in one embodiment, a funnel shape, is expanded and catheter 1800 is further advanced into the uterus 1706 so that the distal end of the outer catheter 1804a is positioned proximate the internal os 1708. Proximal positioning element 1810a, having, in one embodiment, a funnel shape with or without venting, is also expanded. Additionally, an external cervical stabilizing element, or cervical collar 1803, is positioned at an external cervical os 1703. Referring to FIG. 18G, vapor 1819a is delivered through the plurality of ports 1816a within rows 1814a. In some embodiments, areas on a surface of the proximal positioning element 1810a provide for venting of vapor or steam. In some embodiments, the proximal positioning element 1810a comprises a plurality of openings 1817a to allow for venting. In various embodiments, the proximal positioning element 1810a is covered by a gas permeable membrane or porous membrane to allow for venting. In some embodiments, for the distal positioning element 1812, the wire mesh is coated with silicone but not the areas between wires in the mesh, therefore allowing steam to escape/vent from these spaces between the wires. In some embodiments, for proximal positioning elements 1810, wires and space between wires are covered with silicone.

In some embodiments, the proximal positioning element may be attached to a middle catheter and allow for venting between the middle catheter and the inner catheter. In another embodiment, the proximal positioning element may be attached to the outer catheter and allow for venting between the outer catheter and the inner catheter.

FIG. 18H is a flow chart illustrating the steps involved in using an ablation catheter to ablate an endometrium of a patient, in accordance with embodiments of the present specification. In various embodiments, the catheter is similar to those described with reference to FIGS. 18D-18G. In some embodiments, the method does not require pre-dilation of the cervix before ablation. At step 1840, a physician inserts a bulbous tip (such as bulbous tip 1813a of catheter 1800 in FIG. 18D) into and through a patient's cervix and advances the catheter into the patient's uterus. The bulbous tip helps to guide the device through the cervix and allows for atraumatic insertion. In some embodiments, the bulbous tip includes an olive-shaped attachment 1882, described in context of FIG. 18O, for atraumatic insertion. In some embodiments, at step 1842, an actuator (such as actuator 191p on handle 190p in FIG. 1P) is used to push forward the bulbous tip. For example, referring to FIG. 1P, on a dorsal side of handle, an actuator 191p in the form of a slide, is moved forward to activate/push forward the bulbous tip. In some embodiments, the bulbous tip is advanced into the patient's uterus until its tip reaches the fundus. The device is then withdrawn by a small distance, such as, for example, 5 mm. Once the catheter is advanced into the uterus, at step 1844, the first and second positioning elements are deployed and the distal positioning element is seated proximate the uterus. In some embodiments, the positioning elements are deployed using actuators as described with reference to FIG. 1P. In some embodiments, once the device is withdrawn, the catheter sheath is moved backwards until the distal positioning element is fully deployed. The device is then advanced again until the distal positioning element touches the fundus. At step 1846, the second proximal positioning element is positioned on the internal cervical os to create a partial blockage but not a complete seal. As described with reference to FIG. 18G, areas on surface of the disc will provide for venting of pressurized air or steam. In one embodiment, the venting occurs through a neck of the positioning element. In another embodiment, the venting occurs between an inner and middle or an inner and outer catheter. At step 1848, vapor or steam is delivered through the plurality of vapor delivery ports on the catheter into the uterus to ablate the endometrium. In embodiments, the steam is delivered for a predetermined duration. The optimal time for steam ablation may be a function of uterine length. Conventional pre-procedure measurements of uterine cavity size may be used to calibrate the necessary quantity of water vapor. Subsequently, the proximal positioning element is unlocked and slid forward by a small distance, such as, for example, 5 mm. The catheter sheath is held still and the two positioning elements are withdrawn inside the sheath. Once the two positioning elements are inside the sheath, the catheter device is withdrawn from the patient.

FIGS. 19D to 19I illustrate an endometrial ablation catheter at different stages of an exemplary method of deployment of the catheter 1802, in accordance with some embodiments of the present specification. FIG. 19D illustrates an assembly of catheter 1802 with a handle 1902, and a cervical collar 1904, in accordance with some embodiments of the present specification. FIG. 19E illustrates a position of the cervical collar 1904 as it sits at an external os, outside the uterus 1706 and cervix 1704, before deployment of the catheter 1802. In the figure, the uterus 1706, cervix 1704, and cervical collar 1904 are shown on the left while specific hand movements on the handle 1902 are shown on the right to demonstrate deployment of the catheter 1802. FIG. 19F illustrates an exemplary position of hands 1990, 1991 to hold the catheter 1802 and handle 1902 for deploying the proximal positioning element 1810, in accordance with some embodiments of the present specification. A user holds the outer sheath of catheter 1802 with one hand 1990 while pushing the handle 1902 forward with the other hand 1991. FIG. 19G illustrates expanding of the distal positioning element 1812 while the user pushes the handle 1902 of the catheter 1802 to extend the inner catheter 1806 within the uterus 1706. FIG. 19H illustrates fully deploying the distal positioning element 1812, which may be uncoated or selectively coated with silicone, and deploying of the proximal positioning element 1810, in accordance with some embodiments of the present specification. So far, in the process of deploying the catheter 1802, nothing is seated yet. The user may decide to stop here or adjust the position of the catheter 1802 till a distance is achieved that is just short of the length of the uterus 1706 to prevent perforation. In some embodiments, the user may decide to push the catheter 1802 until its distal positioning element 1812 abuts a fundus of the uterus, indicating resistance at the fundus. The user may, in some embodiments, turn a dial, provided on the handle 1902, clockwise to retract the proximal positioning element 1810 and extend further the distal positioning element 1812. In some embodiments, when the proximal positioning element 1810 is expanded, it moves in a direction toward the cervical collar 1904 while the cervical collar 1904 moves in an opposite direction, toward the proximal positioning element 1810 (similar to a Chinese finger puzzle) FIG. 19I illustrates turn of a dial 1906 to further retract first positioning element 1810 to partially seal a cervical os, so as to isolate the uterus 1706. In some embodiments, the partial seal is not perfect (escape vents are provided in openings or holes of the proximal positioning element or venting elements/grooves are provided in one or both of the inner catheter or outer catheter/sheath) to allow for release of vapor out of the uterus, maintaining a low pressure. In some embodiments, the user ablates the uterus by delivering steam through the catheter 1802 for about a 40 second cycle. In embodiments, proximal positioning element 1810 has selective coating and it provides for a drain to collect water produced as the vapor condenses during and after ablation.

FIGS. 18I to 18N illustrate exemplary embodiments of distal end of an endometrial ablation catheter having a single positioning element, in accordance with the present specification. FIG. 18I illustrates a cross-section side view 1854a, side view 1854b, and distal end front-on view 1856, of the endometrial ablation catheter 1802i, in accordance with some embodiments of the present specification. The catheter 1802i is shown with a braided stent 1858. The stent 1858 functions as a positioning element described with reference to the endometrial ablation catheters of the present specification. In embodiments, the braided stent 1858 is made from Nitinol wire mesh, or any other shape memory material such that the stent 1858 expands into a deployed configuration, as shown in FIG. 18I. In embodiments, the stent 1858 is made from a single wire mesh 1858a. In some embodiments, the stent 1858 is made from a double wire mesh 1858b. FIG. 18J illustrates a perspective side view of the catheter of FIG. 18I with the stent 1858 extending over the inner catheter 1806, and extending out from the outer catheter 1804. Steam from within the inner catheter 1806 is deployed while the braided stent 1858 is in an expanded state and deployed within the uterus. The catheter includes an atraumatic distal tip 1859 with guide wire lumen, as described with reference to FIGS. 18L through 18N. The guidewire lumen could be large enough to accommodate a uterine sound. FIG. 18K illustrates a cross section view 1862, a perspective side view 1864, and a distal end front-on view 1860 of the braided stent 1858, in accordance with some embodiments of the present specification. The proximal conical end of the positioning element is either partially or completely covered by an insulating membrane made of silicone or PTFE.

FIG. 18L illustrates a side perspective view of an atraumatic tip 1859 for attaching to a distal end 1866 of an inner catheter 1806 of an endometrial ablation catheter, in accordance with some embodiments of the present specification. FIG. 18M illustrates a side front perspective view of the atraumatic tip 1859 attached to the distal end 1866 of an inner catheter of an endometrial ablation catheter, in accordance with some embodiments of the present specification. FIG. 18N illustrates a top perspective view of the atraumatic tip 1859 attached to the distal end 1866 of an inner catheter of an endometrial ablation catheter, in accordance with some embodiments of the present specification. Referring simultaneously to FIGS. 18L, 18M, and 18N, the atraumatic tip 1859 includes an opening 1868 for a passage of a guide wire. In embodiments, the opening 1868 is configured to receive a 0.035 inch guide wire. The atraumatic tip 1859 is connected to the inner catheter 1806 at its distal end 1866. In some embodiments, the atraumatic tip 1859 is connected to the distal end 1866 of the inner catheter 1806 via a threaded screw 1872. The atraumatic tip 1859 is made from a soft plastic material and includes grooves to receive and lock with the threaded screw 1872 to connect with the inner catheter 1806.

FIG. 18O illustrates different views of a double-positioning element ablation catheter 1802p with an atraumatic olive tip end 1882, in accordance with another embodiment of the present specification. The olive tip end 1882 ensures that the uterus is not punctured and provides for an atraumatic insertion of the catheter 1802p. In some embodiments, olive tip end attachment 1882 may include a hollow channel within its body, the channel opening at the distal edge of attachment 1882, to enable delivery of steam through the channel. In some embodiments, one or more holes in tip of the attachment 1882 enable delivery of steam. All the holes may be of similar or varying diameters. Two positioning elements—a proximal positioning element 1884 and a distal positioning element 1886, are provided with catheter 1802p. Positioning elements 1884 and 1886 are in the form of hoods, where distal hood 1886 may have a diameter ranging from 25 to 34 mm+/−2 mm, and the proximal hood 1884 may have a diameter ranging from 25 to 30 mm+/−2 mm. A distance between the two hoods 1884 and 1886 may be in a range of 28 to 36.4 mm. Each hood 1884/1886 may have a depth of approximately 5 mm along the length of the catheter 1802p. In embodiments, each hood 1884/1886 is attached to the shaft 1888 using a soft connect mechanism with a PTFE wire. A distance between distal end of the distal hood 1886 and a distal tip of the olive tip end 1882 may be approximately 16.7 mm. The shaft portion 1888a extending between the distal hood 1886 and the olive tip attachment 1882 can also include one or more holes for distributing steam during ablation. In some embodiments, holes may also be present before the distal hood 1886, between the distal hood 1886 and the proximal hood 1884, for disseminating steam. Length of the olive tip end 1882 may extend for approximately 6 mm. A diameter of the distal tip of the olive tip end 1882 may be in a range of 3.4+/−0.05 mm. Steam enters a catheter 1802p shaft 1888, and exits through openings 1889 along the shaft 1882 during ablation. The shaft 1888 between the two hoods 1884 and 1886 may have a diameter of approximately 1.1+/−0.05 mm. In embodiments, there are additional openings in the olive tip end 1882 and catheter shaft 1888a distal to the distal hood 1886. The shaft 1888a extending from the distal end of the distal hood 1886 to the olive tip end 1882 may be made from Nitinol and has a diameter of approximately 0.4 mm.

FIG. 18P illustrates distal ends of ablation catheters 1878 having distal positioning elements 1879 and a plurality of ports 1877 along a length of the catheter shaft 1875, in accordance with some embodiments of the present specification. FIG. 18Q illustrates distal ends of ablation catheters 1891 having distal olive tips 1893, positioning elements 1895, and a plurality of ports 1897 along a length of the catheter shaft 1899, in accordance with some embodiments of the present specification. The olive tip 1893 is rounded and bulbous and configured to by atraumatic to body tissues. A cross-sectional view of the olive tip end 1893 shows diagonal openings or holes 1890 inside the tip end 1893. In embodiments, the olive tip end 1893 has four identical and symmetrically configured openings within its distal spherical tip. Each opening 1890 is connected to and extends outwards from the hollow catheter shaft 1899 extending beyond the distal hood 1886. The openings 1890 provide an exit for steam out distal to the positioning element 1895 during ablation. FIG. 18R illustrates a side view a distal end of an ablation catheter 1850 having a distal olive tip 1857, a distal positioning element 1853, a proximal positioning element 1851, and a plurality of ports 1855 along a length of the catheter shaft 1869, in accordance with some embodiments of the present specification. FIG. 18S illustrates a rear perspective view of the catheter 1850 of FIG. 18R. The ablation catheter 1850 includes a connector 1867 at its proximal end for connecting to a proximal catheter portion.

FIG. 18T illustrates a distal end of an ablation catheter 1802t with half-circle openings 1802c at the distal end and a distal positioning element 1896, in accordance with some embodiments of the present specification. Although the figure illustrates half-circle opening 1802c, the openings could be of other shapes such as but not limited to, half-rectangular. In some embodiments, the positioning element 1896 is deformable, flattening out as it is pushed against the fundus of a uterus. The distal end 1894 of catheter 1802t may be open, or covered, but in either case includes half-circle openings 1802c. In some embodiments, the circular distal end of the shaft 1888 is configured to include at least three equidistant half-circle openings 1802c. In some embodiments, the distal end 1894 is closed with a cap 1849. In some embodiments, the cap 1849 has a diameter of approximately 1.65 mm. In some embodiments, the cap 1849 is welded to the distal end 1894. The cap 1849 serves to close the open distal end of catheter 1802t, while the half circles openings 1802c still allow an opening for the steam to exit during ablation and reach the fundus of the uterus. In some embodiments, the ablation catheter 1802t does not include a cap 1849. In embodiments, the shaft 1847 of the catheter 1802t includes a plurality of ports 1843 for the delivery of vapor to other portions of the uterus.

FIG. 18U illustrates a distal end of an ablation catheter 18100a having a spherical shaped distal positioning element 18106 and a cover 18112 extending over the entirety or a portion of the positioning element 18106, in accordance with an exemplary embodiment of the present specification. FIG. 18V illustrates a distal end of an ablation catheter 18100b having a spherical shaped distal positioning element 18108, in accordance with another exemplary embodiment of the present specification. FIG. 18W illustrates a distal end of an ablation catheter 18100c having a conical shaped distal positioning element 18110, in accordance with yet another exemplary embodiment of the present specification. Embodiments of FIGS. 18U, 18V, and 18W, may be used in catheter devices for endometrial ablation as well as for ablation of urinary bladder as described in subsequent figures. Referring simultaneously to FIGS. 18U, 18V, and 18W, a distal tip 18102 of the catheter shaft extends into the positioning elements 18106, 18108, 18110. The distal tip 18102 is an extension of the catheter shaft and is configured to have a smooth rounded tip at its most distal end. In some alternative embodiments, the distal tip 18102 is soft and is configured to have half circles, similar to embodiment of FIG. 18T. A portion of the distal tip 18102 has at least one or a plurality of openings 18104 to provide an exit for steam during ablation. In some embodiments, the openings 18104 are circular, slotted, semi-circular, or of any other shape. In some embodiments, 1 to 1000 openings 18104 are distributed over a length of 3 to 7 cm across the length and surface of the distal tip 18102, where each opening has a length or a diameter in a range of 0.1 to 1 mm. In some embodiments, 64 to 96 openings are distributed over the distal tip 18102. In embodiments, the distal tip 18102 of the catheter is encompassed within the positioning element, such as a spherical element 18106 of FIG. 18U, a spherical element 18108 of FIG. 18V, or an inverted 3-dimensional (3D) conical shaped wire mesh 18110 of FIG. 18WX. In embodiments, positioning elements 18106, 18108, and 18110 are configured to compress or deform when they contact the fundus of the uterus or bladder. A tip of each positioning element 18106, 18108, and 18110 is free floating and the positioning elements 18106, 18108, and 18110 are attached to the respective catheter at a proximal neck of the distal tip 18102. Therefore, the positioning elements 18106, 18108, and 18110 act as a ‘bumper’ and are atraumatic to the fundus, while also allowing for the distribution of steam at the fundus. Each positioning element 18106, 18108, and 18110 is configured from a wire mesh so that there is sufficient space between the wires of the mesh for steam to exit. Referring to FIG. 18U, a cover 18112 is provided to partially cover the openings through the wire mesh on a proximal (bottom) side of the spherical positioning element 18106 to prevent steam from flowing in this direction. In some embodiments, cover 18112 is silicone. FIG. 18V illustrates an alternative embodiment of the spherical positioning element 18106, in the form of the spherical positioning element 18108 that does not include a cover 18112. FIG. 18W illustrates use of a conical positioning element 18110, which is similar to an upside down Erlenmeyer Flask and is configured to approximate a shape of a uterus.

FIG. 18X illustrates an atraumatic soft tip 18114 of a catheter shaft 18116 that is used for insertion into a cervix 18118, in accordance with some embodiments of the present specification. In some embodiments of the present specification, a catheter shaft 18116 is inserted through a patient's vagina canal 18115 and into and through a portion of the patient's cervix. During delivery, a distal hood 18120, inner catheter shaft 18126, and proximal hood 18122 are all disposed within catheter shaft 18120 such that soft tip 18114 comprises the distal end of the catheter. Soft tip 18114 is configured to be soft and atraumatic to the vaginal canal 18115, external cervical os 18117, and cervix 18118 during positioning. During deployment, inner catheter shaft 18126 is extended from catheter shaft 18116, through the cervix 18118 and into a uterus 18124, such that inner catheter shaft 18126 is positioned within the uterus 18124 proximate a fibroid/tumor/lesion 18128 that is required to be treated with ablation. Distal hood 18120 is deployed proximate a fundus 18132 of the uterus 18124 and proximal hood 18122 is deployed proximate an internal cervical os 18119 to firmly position the inner catheter shaft 18126 within the uterus. Openings in the inner catheter shaft 18126 are then used to deliver steam or vapor 18130 to ablate the target area. In some embodiments, a 40 second cycle of vapor ablation is delivered to the uterus. During the ablation, the distal hood 18120 may be pulled back slightly to ensure complete coverage of the target area, including the fundus 18132 of the uterus. The atraumatic soft tip 18114 ensures that the body tissues of the patient are protected during insertion of the catheter and pulling back of the distal hood 18120.

The various endometrial ablation (EA) devices in accordance with embodiments of the present specification provide numerous advantages over the existing method of endometrial ablation. Steam ablation by embodiments of the present specification effectively ablates endometrial tissue for a wider variety of uterine shapes than current EA procedures allow. The systems and methods of the present specification ablate endometrial tissue even in the presence of fibroids or polyps.

FIG. 19J illustrates a distal end of an ablation catheter 1910 having a proximal positioning element 1911, distal positioning element 1912, and a plurality of ports 1913 along a length of the catheter shaft 1914, in accordance with some embodiments of the present specification. In embodiments, the catheter 1910 includes a proximal connector 1916 for connecting the proximal positioning element 1911 and for connecting the catheter 1910 to a proximal catheter portion, and a distal connector 1917 for connecting the distal positioning element 1912. In some embodiments, the positioning elements 1911, 1912 have conical or circular shapes. In some embodiments, the positioning elements are connected via sutures or wires 1918.

FIG. 19K illustrates a distal end of an ablation catheter 1920 having a proximal positioning element 1921, a distal positioning element 1922, a distal olive tip 1925, and a plurality of ports 1923 along a length of the catheter shaft 1924, in accordance with some embodiments of the present specification. In some embodiments, the catheter 1920 includes a proximal connector 1926 having a screw thread for connecting to a proximal catheter portion.

FIG. 19L illustrates a connector 1930 for connecting a distal positioning element to a distal end of an ablation catheter, in accordance with some embodiments of the present specification. In embodiments, the connector 1930 has a flat distal end 1931, is configured to fit coaxially over a distal portion of an ablation catheter, and includes a plurality of openings 1932 for passage of suture or wire for securing a distal positioning element. In embodiments, the connector 1933 includes an opening at its distal end to allow vapor to escape and reach a fundus of a uterus.

FIG. 19M illustrates another connector 1935 for connecting a distal positioning element to a distal end of an ablation catheter, in accordance with other embodiments of the present specification. In embodiments, the connector 1935 has a rounded distal end 1936 configured to be atraumatic to body tissues, is configured to fit coaxially over a distal portion of an ablation catheter, and includes a plurality of openings 1937 for passage of suture or wire for securing a distal positioning element.

FIG. 19N illustrates a connector 1940 for connecting a proximal positioning element to a distal end of an ablation catheter, in accordance with some embodiments of the present specification. In embodiments, a distal end of the connector 1940 includes a plurality of openings 1941 for passage of suture or wire for securing a proximal positioning element and a proximal end 1942 of the connector is configured to connect to a proximal catheter portion.

FIG. 19O illustrates another connector 1945 for connecting a proximal positioning element to a distal end of an ablation catheter, in accordance with other embodiments of the present specification. In embodiments, a distal end of the connector 1945 includes a plurality of openings 1946 for passage of suture or wire for securing a proximal positioning element and a proximal end 1947 of the connector is configured to connect to a proximal catheter portion.

FIG. 19P illustrates a shaft 1950 of an ablation catheter depicting a plurality of ports 1951, in accordance with some embodiments of the present specification. The ports 1951 are configured to allow the release of vapor from the shaft lumen 1952 into a uterus. In some embodiments, the ports 1951 are arranged in rows 1953.

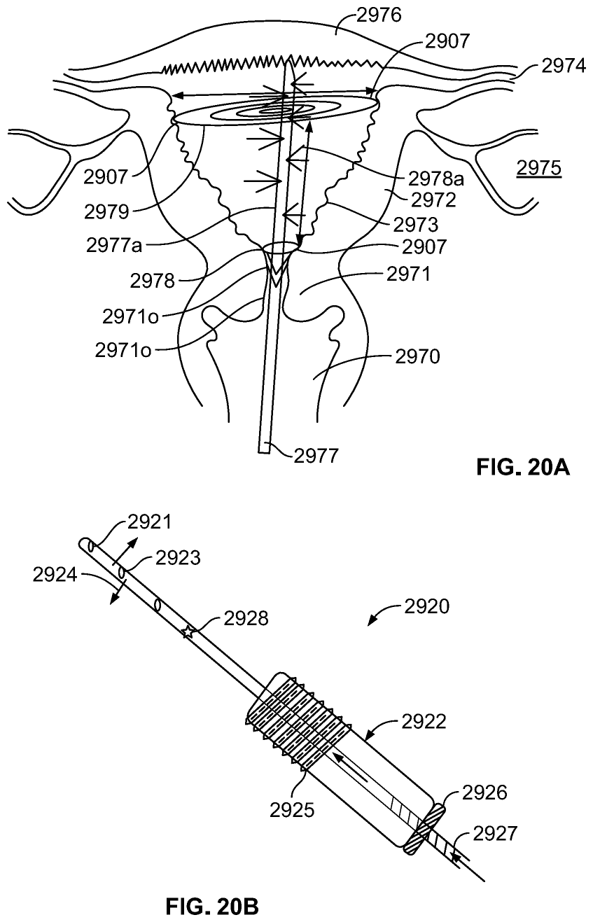

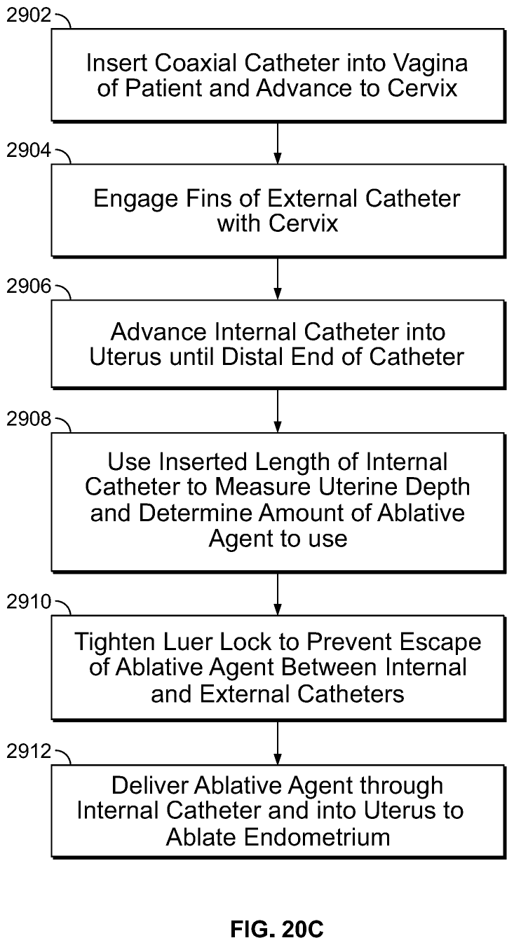

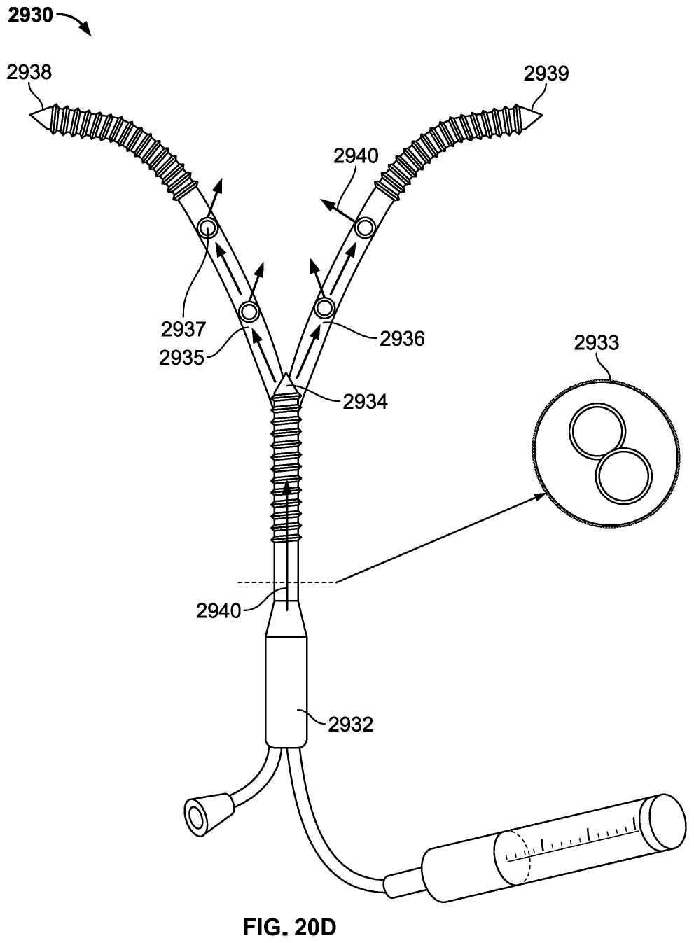

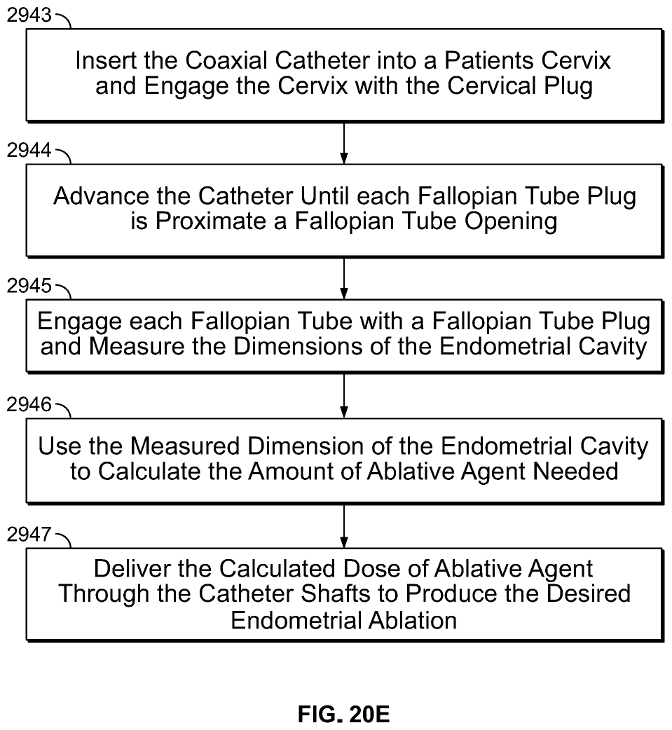

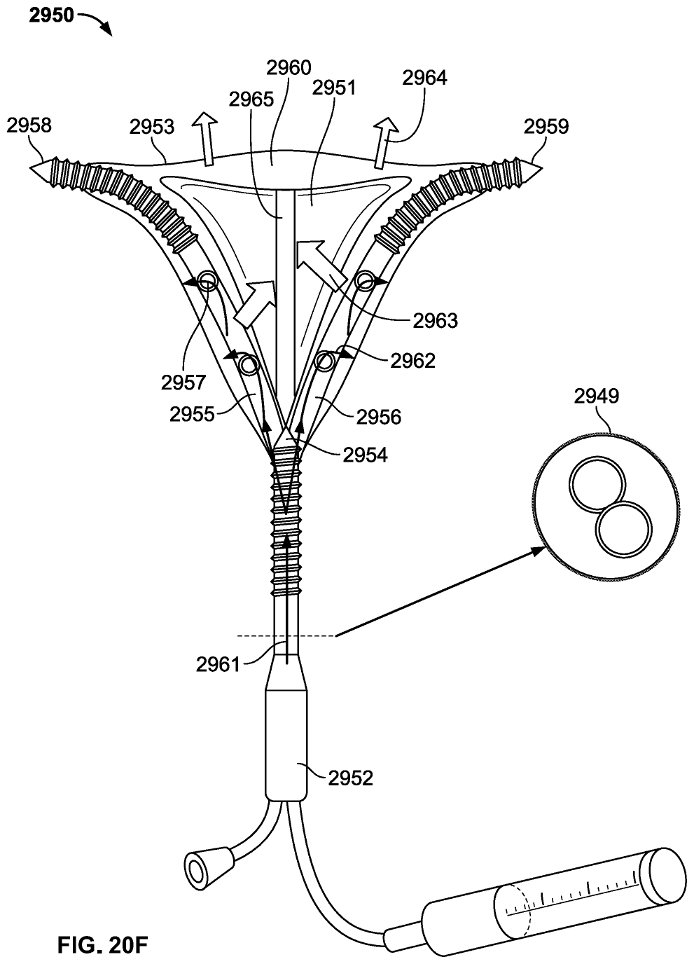

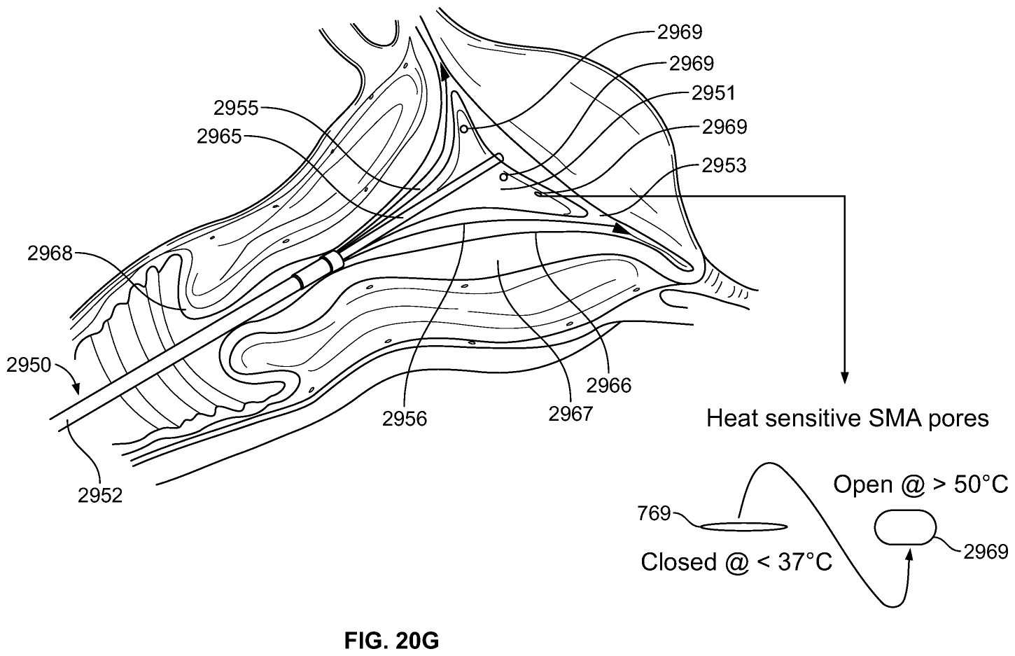

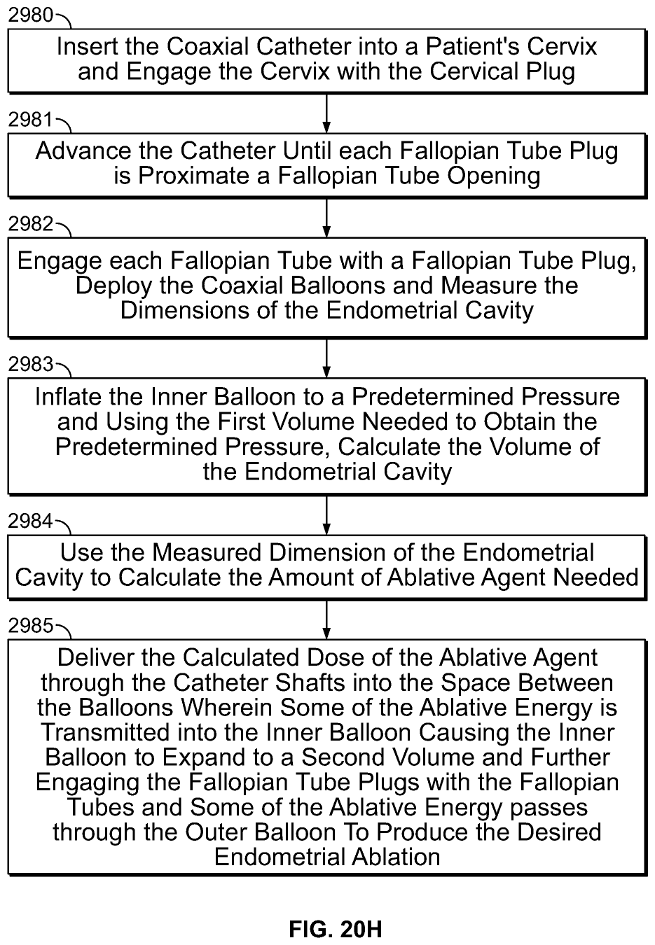

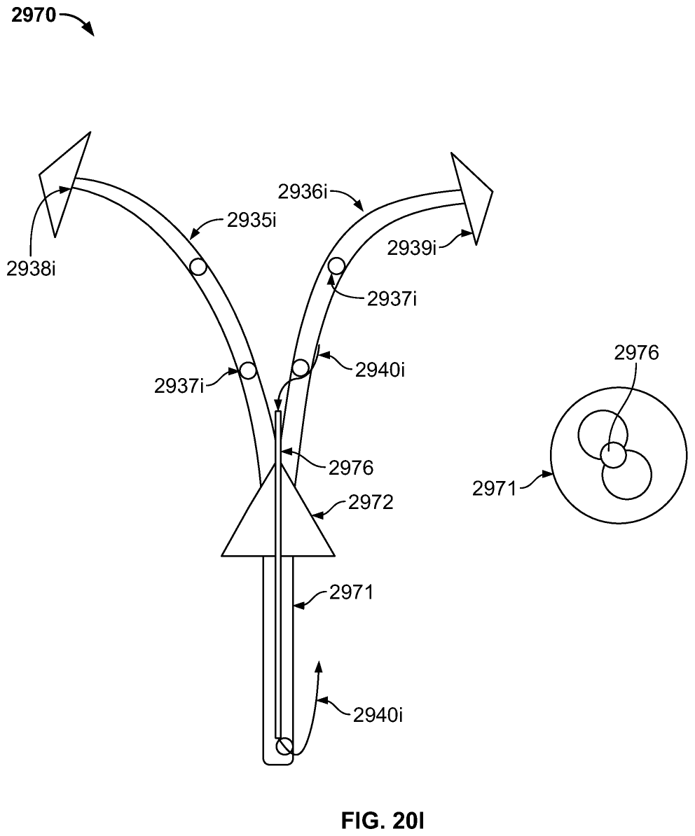

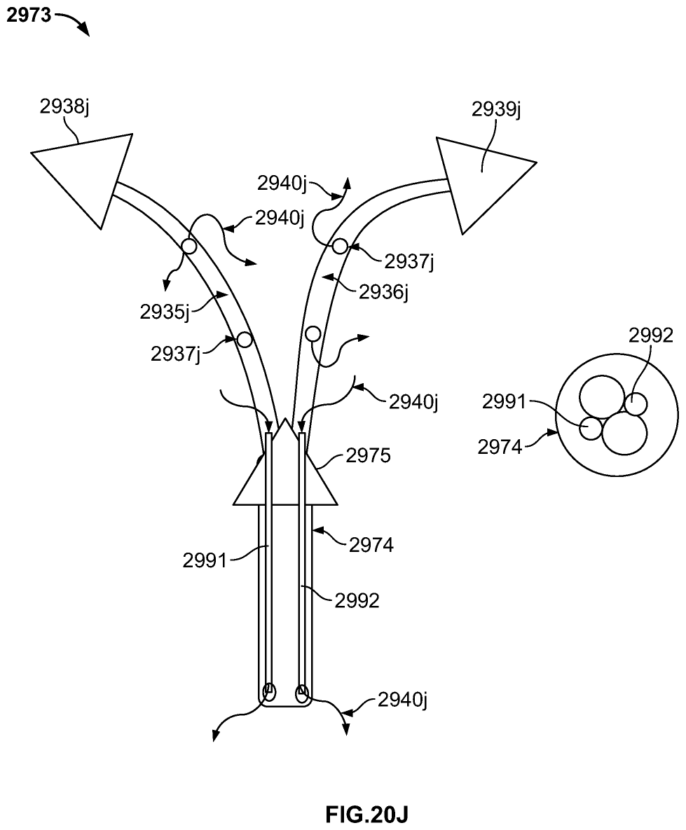

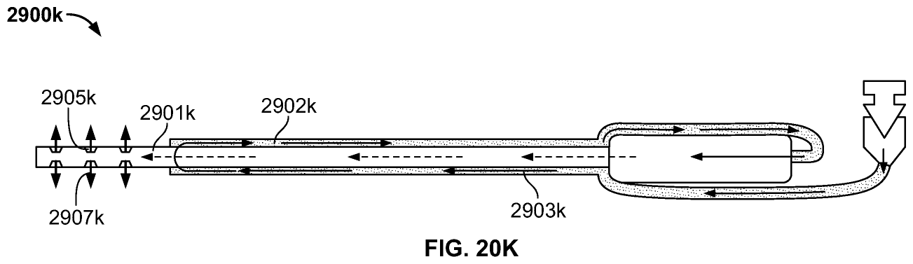

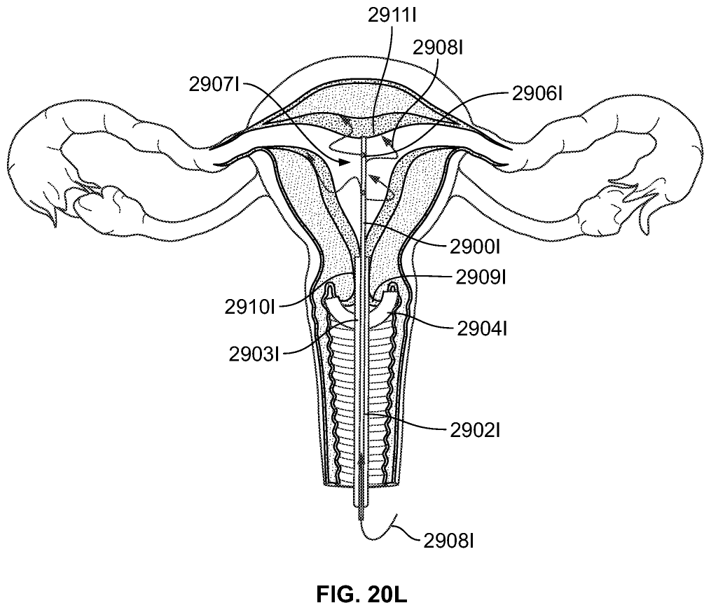

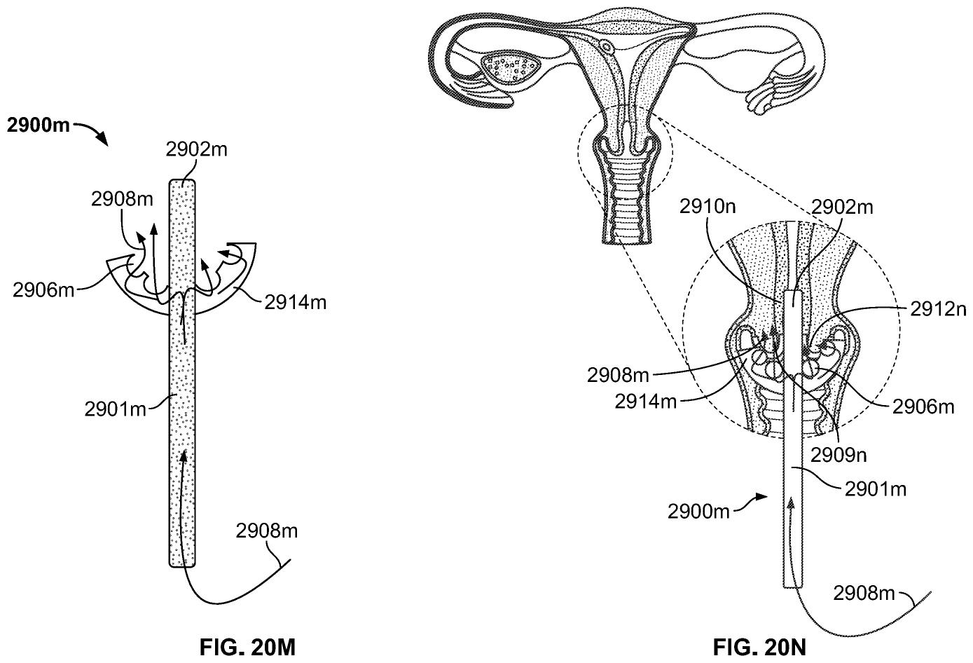

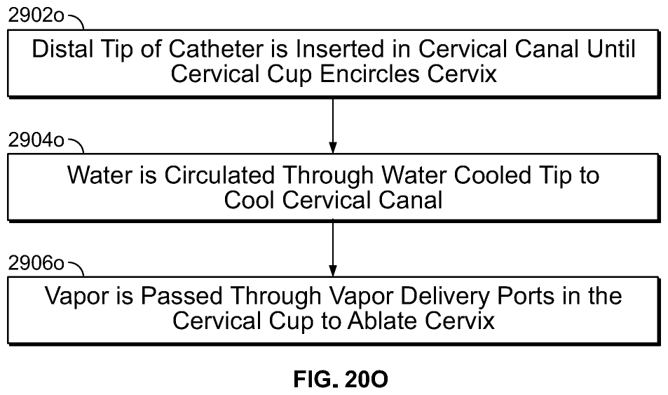

FIG. 20A illustrates endometrial ablation being performed in a female uterus by using the ablation device, in accordance with an embodiment of the present specification. A cross-section of the female genital tract comprising a vagina 2970, a cervix 2971, a uterus 2972, an endometrium 2973, fallopian tubes 2974, ovaries 2975 and the fundus of the uterus 2976 is illustrated. A catheter 2977 of the ablation device is inserted into the uterus 2972 through the cervix 2971 at the cervical os. In an embodiment, the catheter 2977 has two positioning elements, a conical positioning element 2978 and a disc shaped positioning element 2979. The positioning element 2978 is conical with an insulated membrane partially or completely covering the conical positioning element 2978. The conical element 2978 positions the catheter 2977 in the center of the cervix 2971 and the insulated membrane prevents the escape of thermal energy or ablative agent out the cervix 2971 through the os 2971o. The second disc shaped positioning element 2979 is deployed close to the fundus of the uterus 2976 positioning the catheter 2977 in the middle of the cavity. An ablative agent 2978a is passed through infusion ports 2977a for uniform delivery of the ablative agent 2977a into the uterine cavity. Predetermined length ‘l’ of the ablative segment of the catheter and diameter ‘d’ of the positioning element 2979 allows for estimation of the cavity size and is used to calculate the amount of thermal energy needed to ablate the endometrial lining. In one embodiment, the positioning elements 2978, 2979 also act to move the endometrial tissue away from the infusion ports 2977a on the catheter 2977 to allow for the delivery of ablative agent. Optional temperature sensors 2907 deployed close to the endometrial surface are used to control the delivery of the ablative agent 2978a. Optional topographic mapping using multiple infrared, electromagnetic, acoustic or radiofrequency energy emitters and sensors can be used to define cavity size and shape in patients with an irregular or deformed uterine cavity due to conditions such as fibroids. Additionally, data from diagnostic testing can be used to ascertain the uterine cavity size, shape, or other characteristics. In one embodiment, the distal positioning element 2979 is also conical and is either partially or completely covered with an insulating membrane. Various shapes of positioning elements described in this application can be used in various combinations to achieve the desired therapeutic goals.

In an embodiment, the ablative agent is vapor or steam which contracts on cooling. Steam/vapor turns to water which has a lower volume as compared to a cryogen that will expand or a hot fluid used in hydrothermal ablation whose volume stays constant upon contacting the tissue. With both cryogens and hot fluids, increasing energy delivery is associated with increasing volume of the ablative agent which, in turn, requires mechanisms for removing the agent, otherwise the medical provider will run into complications, such as perforation. However, steam, on cooling, turns into water which occupies significantly less volume; therefore, increasing energy delivery is not associated with an increase in volume of the residual ablative agent, thereby eliminating the need for continued removal. This further decreases the risk of leakage of the thermal energy via the fallopian tubes 2974 or the cervix 2971, thus reducing any risk of thermal injury to adjacent healthy tissue.

In one embodiment, the positioning attachment must be separated from the ablation region by a distance of greater than 0.1 mm, preferably 1 mm and more preferably 1 cm. In another embodiment, the positioning attachment can be in the ablated region as long as it does not cover a significant surface area. For endometrial ablation, 100% of the tissue does not need to be ablated to achieve the desired therapeutic effect. Hence, in some embodiments, the positioning element can contact and cover 5% or less of the endometrial surface area.

In one embodiment, the preferred distal positioning attachment is an uncovered wire mesh that is positioned proximate to the mid body region. In one embodiment, the preferred proximal positioning device is a covered wire mesh that is pulled into the cervix, centers the device, and occludes the cervix and or the internal os. FIGS. 19A, 19B, and 19C illustrates some of the various embodiments of the positioning devices. One or more such positioning devices may be helpful to compensate for the anatomical variations in the uterus. The distal positioning device is preferably oval, with a long axis between 0.1 mm and 10 cm (preferably 1 cm to 5 cm) and a short axis between 0.1 mm and 5 cm (preferably 0.5 cm to 1 cm). The proximal positioning device is preferably circular with a diameter between 0.1 mm and 10 cm, preferably 1 cm to 5 cm.

In another embodiment, the catheter is a coaxial catheter comprising an external catheter and an internal catheter wherein, upon insertion, the distal end of the external catheter engages and stops at the cervix while the internal extends into the uterus until its distal end contacts the fundus of the uterus. FIG. 18A illustrates an exemplary embodiment of the catheter configuration in accordance with the present specification. The length of the internal catheter that has passed into the uterus is then used to measure the depth of the uterine cavity and determines the amount of ablative agent to use. Ablative agent is then delivered to the uterine cavity via at least one port on the internal catheter. In one embodiment, during treatment, intracavitary pressure within the uterus is kept below 100 mm Hg, and preferably below 30 mm Hg (no more than 10% above atmospheric pressure). In one embodiment, the coaxial catheter further includes a pressure sensor to measure intracavitary pressure. In one embodiment, the coaxial catheter further includes a temperature sensor to measure intracavitary temperature. In one embodiment, the ablative agent is steam and the steam is released from the catheter at a pressure of less than 100 mm Hg, and preferably below 30 mm Hg. In one embodiment, the steam is delivered with a temperature between 90 and 100° C. In another embodiment the steam is delivered between the temperature of 100-110° C.

FIG. 20B is an illustration of a coaxial catheter 2920 used in endometrial tissue ablation, in accordance with one embodiment of the present specification. The coaxial catheter 2920 comprises an inner catheter 2921 and outer catheter 2922. In one embodiment, the inner catheter 2921 has one or more ports 2923 for the delivery of an ablative agent 2924. In one embodiment, the ablative agent is steam. In one embodiment, the outer catheter 2922 has multiple fins 2925 to engage the cervix to prevent the escape of vapor out of the uterus and into the vagina. In one embodiment, the fins are composed of silicone. The fins 2925 ensure that the cervix is not completely sealed. In embodiments, multiple holes are configured in the fins 2925, which direct the vapor escaping the uterus into a lumen of the outer catheter 2922. In one embodiment, the outer catheter 2922 includes a luer lock 2926 to prevent the escape of vapor between the inner catheter 2921 and outer catheter 2922. In one embodiment, the inner catheter 2921 includes measurement markings 2927 to measure the depth of insertion of the inner catheter 2921 beyond the tip of the outer catheter 2922. Optionally, in various embodiments, one or more sensors 2928 are incorporated into the inner catheter 2921 to measure intracavitary pressure or temperature.

FIG. 20C is a flow chart listing the steps involved in an endometrial tissue ablation process using a coaxial ablation catheter, in accordance with one embodiment of the present specification. At step 2902, the coaxial catheter is inserted into the patient's vagina and advanced to the cervix. Then, at step 2904, the coaxial catheter is advanced such that the fins of the outer catheter engage the cervix, effectively stopping advancement of the outer catheter at that point. The inner catheter is then advanced, at step 2906, until the distal end of the internal catheter contacts the fundus of the uterus. The depth of insertion is then measured using the measurement markers on the internal catheter at step 2908, thereby determining the amount of ablative agent to use in the procedure. At step 2910, the luer lock is tightened to prevent any escape of vapor between the two catheters. The vapor is then delivered, at step 2912, through the lumen of the inner catheter and into the uterus via the delivery ports on the internal catheter to ablate the endometrial tissue.

FIG. 20D is an illustration of a bifurcating coaxial catheter 2930 used in endometrial tissue ablation, in accordance with one embodiment of the present specification. The catheter 2930 includes a first elongate shaft 2932 having a proximal end, a distal end and a first lumen within. The first lumen splits in the distal end to create a coaxial shaft 2933. The distal end of the first shaft 2932 also includes a first positioning element, or cervical plug 2934, that occludes a patient's cervix. The catheter 2930 bifurcates as it extends distally from the cervical plug 2934 to form a second catheter shaft 2935 and a third catheter shaft 2936. The second and third catheter shafts 2935, 2936 each include a proximal end, a distal end, and a shaft body having one or more vapor delivery ports 2937. The second and third catheter shafts 2935, 2936 include second and third lumens respectively, for the delivery of ablative agent. The distal ends of the second and third catheter shafts 2935, 2936 include second and third positioning elements, or fallopian tube plugs 2938, 2939 respectively, designed to engage a patient's fallopian tubes during an ablation therapy procedure and prevent the escape of ablative energy. The fallopian tube plugs 2938, 2939 also serve to position the second and third shafts 2935, 2936 respectively, in an intramural portion or isthmus of a patient's fallopian tube. The second and third catheter shafts 2935, 2936 are independently coaxially extendable and the length of each shaft 2935, 2936 is used to determine the dimension of a patient's endometrial cavity. An ablative agent 2940 travels through the first catheter shaft 2932, through both second and third catheter shaft 2935, 2936, and out the vapor delivery ports 2937 and into the endometrial cavity to ablate endometrial tissue.

FIG. 20E is a flowchart listing the steps of a method of using the ablation catheter of FIG. 20D to ablate endometrial tissue, in accordance with one embodiment of the present specification. At step 2943, the coaxial catheter is inserted into a patient's cervix and the cervix is engaged with the cervical plug. The catheter is then advanced until each fallopian tube plug is proximate a fallopian tube opening at step 2944. Each fallopian tube is then engaged with a fallopian tube plug at step 2945 and the dimensions of the endometrial cavity are measured. The measurements are based on the length of each catheter shaft that has been advanced. At step 2946, the measured dimensions are used to calculate the amount of ablative agent needed to carry out the ablation. The calculated dose of ablative agent is then delivered through the catheter shafts and into the endometrial cavity to produce the desired endometrial ablation at step 2947.

FIG. 20F is an illustration of a bifurcating coaxial catheter 2950 with expandable elements 2951, 2953 used in endometrial tissue ablation, in accordance with one embodiment of the present specification. Similar to the catheter 2930 of FIG. 20D, the catheter 2950 depicted in FIG. 20F includes a first elongate coaxial shaft 2952 having a proximal end, a distal end and a first lumen within. The first lumen splits in the distal end to create a coaxial shaft 2949. The distal end of the first shaft 2952 also includes a first positioning element, or cervical plug 2954, that occludes a patient's cervix. The catheter 2950 bifurcates as it extends distally from the cervical plug 2954 to form a second catheter shaft 2955 and a third catheter shaft 2956. The second and third catheter shafts 2955, 2956 each include a proximal end, a distal end, and a catheter shaft body having one or more vapor delivery ports 2957. The second and third catheter shafts 2955, 2956 include second and third lumens respectively, for the delivery of ablative agent. The distal ends of the second and third catheter shafts 2955, 2956 include second and third positioning elements, or fallopian tube plugs 2958, 2959 respectively, designed to engage a patient's fallopian tubes during an ablation therapy procedure and prevent the escape of ablative energy. The fallopian tube plugs 2958, 2959 also serve to position the second and third shafts 2955, 2956 respectively, in an intramural portion or isthmus of a patient's fallopian tube. The second and third catheter shafts 2955, 2956 are independently coaxially extendable and the length of each catheter shaft 2955, 2956 is used to determine the dimension of a patient's endometrial cavity.

The catheter 2950 further includes a first expandable member or balloon 2951 and a second expandable member or balloon 2953 comprising a coaxial balloon structure. In one embodiment, the first balloon 2951 is a compliant balloon structure and the second balloon 2953 is a non-compliant balloon structure shaped to approximate the uterine cavity shape, size or volume. In another embodiment, the second balloon 2953 is partially compliant. In another embodiment, the compliance of the two balloons 2951, 2953 is substantially equivalent. The balloons 2951, 2953 are attached to the second and third catheter shafts 2955, 2956 along an inner surface of each shaft 2955, 2956. The first, inner balloon 2951 is positioned within the second, outer balloon 2953. The inner balloon 2951 is designed to be inflated with air and a first volume of the inner balloon 2951 is used to measure a dimension of a patient's endometrial cavity. An ablative agent 2961 is introduced into the catheter 2950 at its proximal end and travels through the first catheter shaft 2952 and into the second and third catheter shafts 2955, 2956. The second and third catheter shafts 2955, 2956 are designed to release ablative energy 2962 through delivery ports 2957 and into a space 2960 between the two balloons 2951, 2953. Some of the ablative energy 2963 is transferred to the air in the inner balloon 2951, expanding its volume from said first volume to a second volume, resulting in further expansion of said inner balloon 2951 to further occlude the patient's endometrial cavity for ideal vapor delivery. In one embodiment, the second volume is less than 25% greater than the first volume. The expansion also forces the fallopian tube plugs 2958, 2959 to further engage the openings of the fallopian tubes. A portion of the ablative agent or ablative energy 2964 diffuses out of the thermally permeable outer balloon 2953 and into the endometrial cavity, ablating the endometrial tissue. In various embodiments, the thermal heating of the air in the balloon occurs either through the walls of the inner balloon, through the length of the catheter, or through both. In one embodiment, the catheter 2950 includes an optional fourth catheter shaft 2965 extending from the first catheter shaft 2952 and between the second and third catheter shaft 2955, 2956 within the inner balloon 2951. Thermal energy from within the fourth catheter shaft 2965 is used to further expand the inner balloon 2951 and assist with ablation.

In one embodiment, the volume of the inner balloon 2951 is used to control the pressure exerted by the outer balloon 2953 on the wall of the uterus. The pressure in the inner balloon 2951 is monitored and air is added to or removed from the inner balloon 2951 to maintain a desirable therapeutic pressure in the outer balloon 2953.

FIG. 20G is an illustration of the catheter 2950 of FIG. 20F inserted into a patient's uterine cavity 2966 for endometrial tissue 2967 ablation, in accordance with one embodiment of the present specification. The catheter 2950 has been inserted with the first shaft 2952 extending through the patient's cervix 2968 such that the second shaft 2955 is positioned along a first side of the patient's uterine cavity 2966 and the third shaft 2956 is positioned along a second side opposite said first side. This positioning deploys the inner balloon 2951 and outer balloon 2953 between the second and third shafts 2955, 2956. In the pictured embodiment, the catheter 2950 includes an optional fourth shaft 2965 to further expand the inner balloon 2951 with thermal energy and assist with ablation of endometrial tissue 2967. In one embodiment, the inner balloon 2951 is optional and the outer balloon 2953 performs the function of both sizing and delivery of the ablative agent. In one embodiment, the outer balloon includes heat sensitive pores 2969 which are closed at room temperature and open at a temperature higher than the body temperature. In one embodiment, the pores are composed of a shape memory alloy (SMA). In one embodiment, the SMA is Nitinol. In one embodiment, the austenite finish (Af) temperature, or temperature at which the transformation from martensite to austenite finishes on heating (alloy undergoes a shape change to become an open pore 2969), of the SMA is greater than 37° C. In other embodiments, the Af temperature of the SMA is greater than 50° C. but less than 100° C.

FIG. 20H is a flowchart listing the steps of a method of using the ablation catheter of FIG. 20F to ablate endometrial tissue, in accordance with one embodiment of the present specification. At step 2980, the coaxial catheter is inserted into a patient's cervix and the cervix is engaged with the cervical plug. The catheter is then advanced until each fallopian tube plug is proximate a fallopian tube opening at step 2981. Each fallopian tube is then engaged with a fallopian tube plug at step 2982, which also deploys the coaxial balloons in the endometrial cavity, and the dimensions of the endometrial cavity are measured. The measurements are based on the length of each catheter shaft that has been advanced and a first volume needed to expand the inner balloon to a predetermined pressure. At step 2983, the inner balloon is inflated to said predetermined pressure and a first volume of the inner balloon at said pressure is used to calculate the volume of the endometrial cavity. The measured dimensions are then used at step 2984 to calculate the amount of ablative agent needed to carry out the ablation. The calculated dose of ablative agent is then delivered through the catheter shafts and into the space between the coaxial balloons at step 2985. Some of the ablative energy is transmitted into the inner balloon to expand the inner balloon to a second volume which further expands the endometrial cavity and, optionally, further pushes the fallopian tube plugs into the fallopian tube openings to prevent the escape of thermal energy. Another portion of the ablative energy passes through the thermally permeable outer balloon to produce the desired endometrial ablation.

In another embodiment, a vapor ablation device for ablation of endometrial tissue comprises a catheter designed to be inserted through a cervical os and into an endometrial cavity, wherein the catheter is connected to a vapor generator for generation of vapor and includes at least one port positioned in the endometrial cavity to deliver the vapor into the endometrial cavity. The vapor is delivered through the port and heats and expands the air in the endometrial cavity to maintain the endometrial cavity pressure below 200 mm Hg and ideally below 50 mm of Hg. In one embodiment, an optional pressure sensor measures the pressure and maintains the intracavitary pressure at the desired therapeutic level, wherein the endometrial cavity is optimally expanded to allow for uniform distribution of ablative energy without the risk of significant leakage of the ablative energy beyond the endometrial cavity and damage to the adjacent normal tissue.

FIG. 20I is an illustration of a bifurcating coaxial catheter 2970 used in endometrial tissue ablation, in accordance with another embodiment of the present specification. Forming a seal at the cervix is undesirable as it creates a closed cavity, resulting in a rise of pressure when vapor is delivered into the uterus. This increases the temperature of the intrauterine air, causing a thermal expansion and further rise of intracavitary pressure. This rise in pressure may force the vapor or hot air to escape out of the fallopian tubes, causing thermal injury to the abdominal viscera. This requires for continuous measurement of intracavitary pressure and active removal of the ablative agent to prevent leakage of thermal energy outside the cavity. Referring to FIG. 20I, the catheter 2970 includes a coaxial handle 2971, a first positioning element 2972, a first bifurcated catheter arm 2935i with a second positing element 2938i at its distal end, a second bifurcated catheter arm 2936i with a third positioning element 2939i at its distal end, and a plurality of infusion ports 2937i along each bifurcated catheter arm 2935i, 2936i. The catheter 2970 also includes a venting tube 2976 which extends through the coaxial handle 2971 and through the first positioning element 2972 such that the lumen of a patient's uterus is in fluid communication with the outside of the patient's body when the first positioning element 2972 is in place positioned against a cervix. This prevents formation of a tight seal when the catheter 2970 is inserted into the cervix. Since the cervix is normally in a closed position, insertion of any device will inadvertently result in formation of an undesirable seal. The venting tube allows for heated air or extra vapor 2940i to vent out as it expands with delivery of vapor and the intracavitary pressure rises. In some embodiments, the venting tube includes a valve for unidirectional flow of air.

FIG. 20J is an illustration of a bifurcating coaxial catheter 2973 used in endometrial tissue ablation, in accordance with yet another embodiment of the present specification. The catheter 2973 includes a coaxial handle 2974, a first positioning element 2975, a first bifurcated catheter arm 2935j with a second positing element 2938j at its distal end, a second bifurcated catheter arm 2936j with a third positioning element 2939j at its distal end, and a plurality of infusion ports 2937j along each bifurcated catheter arm 2935j, 2936j. The catheter 2973 also includes two venting tubes 2991, 2992 which extend through the coaxial handle 2974 and through the first positioning element 2975 such that the lumen of a patient's uterus is in fluid communication with the outside of the patient's body when the first positioning element 2975 is in place positioned against a cervix. This prevents formation of a tight or complete seal when the catheter 2973 is inserted into the cervix. The venting tubes 2991, 2992 allow for heated air or extra vapor 2940j to vent out as it expands with delivery of vapor and the intracavitary pressure rises. In some embodiments, the venting tubes 2991, 2992 include a valve for unidirectional flow of air.

FIG. 20K is an illustration of a water cooled catheter 2900k used in endometrial tissue ablation, in accordance with one embodiment of the present specification. The catheter 2900k comprises an elongate body 2901k having a proximal end and a distal end. The distal end includes a plurality of ports 2905k for the delivery of vapor 2907k for tissue ablation. A sheath 2902k extends along the body 2901k of the catheter 2900k to a point proximal to the ports 2905k. During use, water 2903k is circulated through the sheath 2902k to cool the catheter 2900k. Vapor 2907k for ablation and water 2903k for cooling are supplied to the catheter 2900k at its proximal end.

FIG. 20L is an illustration of a water cooled catheter 2900l used in endometrial tissue ablation and positioned in a uterus 2907l of a patient, in accordance with another embodiment of the present specification. The catheter 2900l comprises an elongate body 2901l, a proximal end, distal end, and a sheath 2902l covering a proximal portion of the body 2901l. Extending from, and in fluid communication with, the sheath 2902l is a cervical cup 2904l. The catheter 2900l further includes a plurality of ports 2906l at its distal end configured to deliver ablative vapor 2908l to the uterus 2907l. Vapor 2908l is supplied to the proximal end of the catheter 2900l. The ports 2906l are positioned on the catheter body 2901l distal to the sheath 2902l. The cervical cup 2904l is configured to cover the cervix 2909l and a distal end of the sheath 2902l extends into the cervical canal 2910l. Water 2903l is circulated through the sheath 2902l and cervical cup 2904l to cool the cervical canal 2910l and/or cervix 2909l while vapor 2908l is delivered through the vapor delivery ports 2906l to ablate the endometrial lining 2911l.

In various embodiments, ablation therapy provided by the vapor ablation systems of the present specification is delivered to achieve the following therapeutic endpoints for uterine ablation: maintain a tissue temperature at 100° C. or less; increase patient's hemoglobin by at least 5% or at least 1 gm % relative to pre-treatment hemoglobin; decrease menstrual blood flow by at least 5% as measured by menstrual pad weight relative to pre-treatment menstrual blood flow; ablation of endometrial tissue in a range of 10% to 99%; decrease in duration of menstrual flow by at least 5% relative to pre-treatment menstrual flow; decrease in amenorrhea rate by at least 10% relative to pre-treatment amenorrhea rate; and patient reported satisfaction with uterine ablation procedure of greater than 25%.

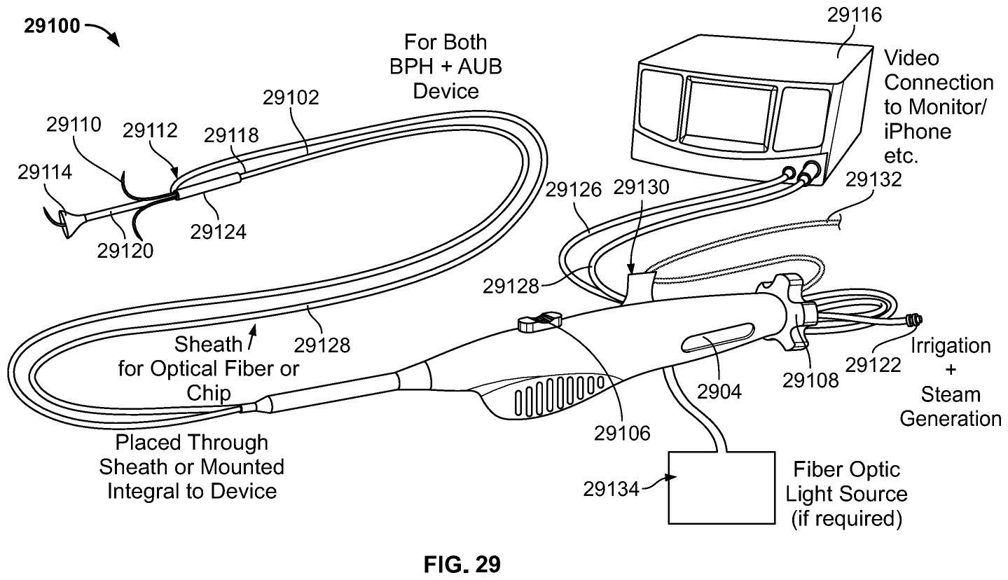

FIG. 20M is an illustration of a water cooled catheter 2900m used in cervical ablation, in accordance with one embodiment of the present specification, and FIG. 20N is an illustration of the catheter 2900m of FIG. 20M positioned in a cervix 2909n of a patient. Referring to FIGS. 29M and 29N simultaneously, the catheter 2900m comprises an elongate body 2901m, a proximal end, a distal end, and a water cooled tip 2902m at its distal end. A cervical cup 2914m is attached to the catheter body 2901m and includes a plurality of ports 2906m which are in fluid communication with the proximal end of the catheter 2900m. Vapor 2908m is provided at the proximal end of the catheter 2900m and is delivered to the cervix 2909n via ports 2906m. In an embodiment, the vapor 2908m ablates the transformation zone 2912n at the cervix 2909n. The water cooled tip 2902m of the catheter 2900m cools the cervical canal 2910n during ablation. In various embodiments, cooling methods known in the art can be used to cool the catheter tip.

FIG. 20O is a flowchart listing the steps involved in cervical ablation performed using the catheter of FIG. 29M. At step 2902o the distal tip of the catheter is inserted into the cervical canal until the cervical cup of the catheter encircles the cervix. Water is circulated through the water cooled tip to cool the cervical canal at step 2904o. At step 2906o vapor is passed through the vapor delivery ports in the cervical cup to ablate the cervix.

In various embodiments, ablation therapy provided by the vapor ablation systems of the present specification is delivered to achieve the following therapeutic endpoints for cervical ablation: maintain a tissue temperature at 100° C. or less; ablate a cervical mucosa without significant injury to the cervical canal; ablate at least 50% of a surface area of a targeted abnormal cervical mucosa such that, upon healing, said abnormal cervical mucosa is replaced by normal cervical mucosa; elimination of more than 25% of abnormal cervical mucosa as assessed by colposcopy; and ablate more than 25% of abnormal cervical mucosa and less than 25% of a total length of a cervical canal.

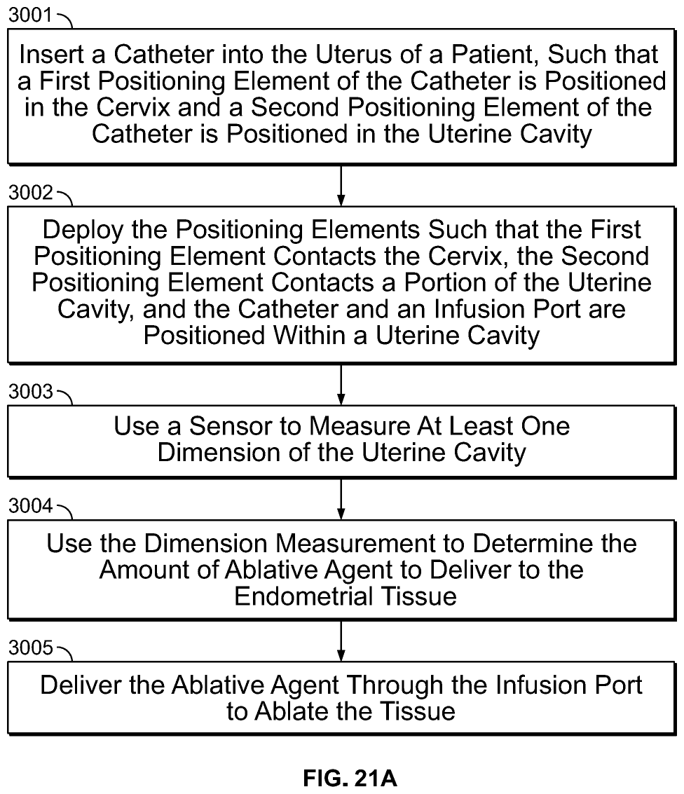

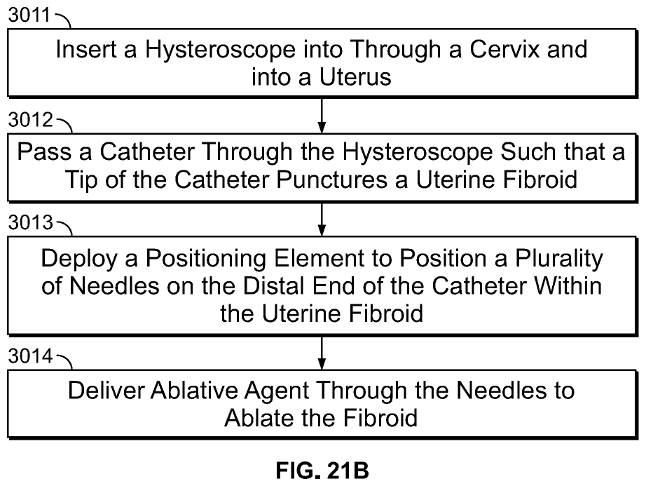

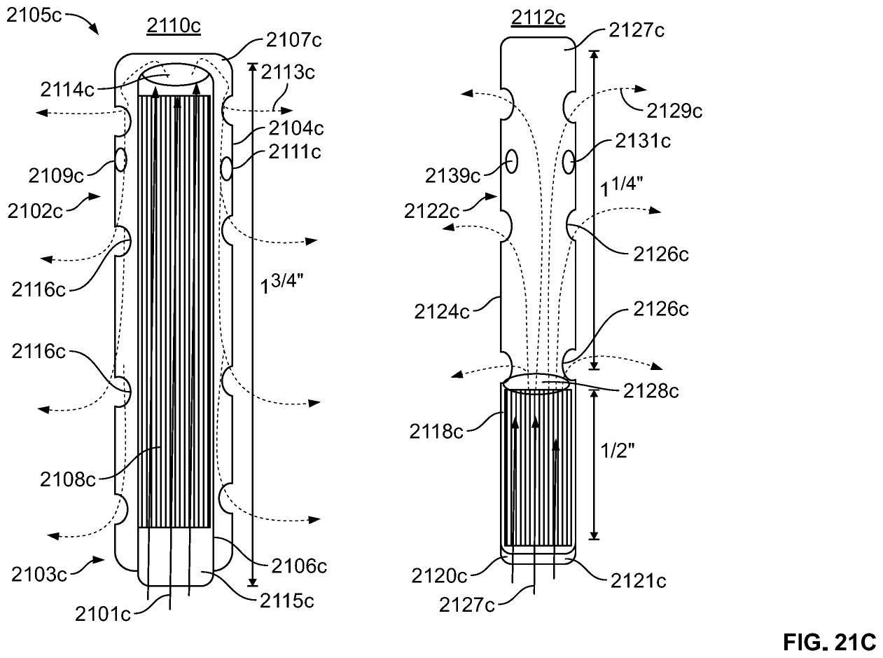

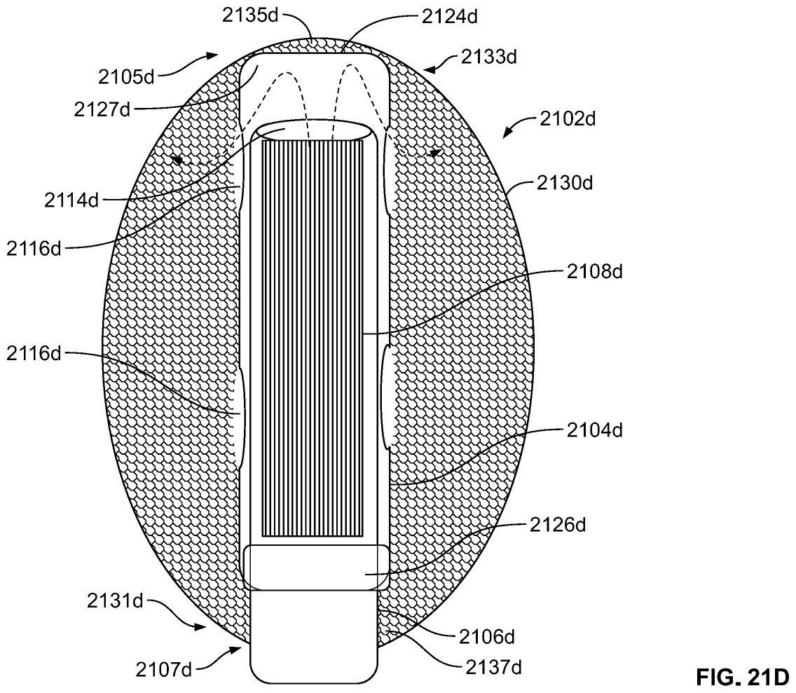

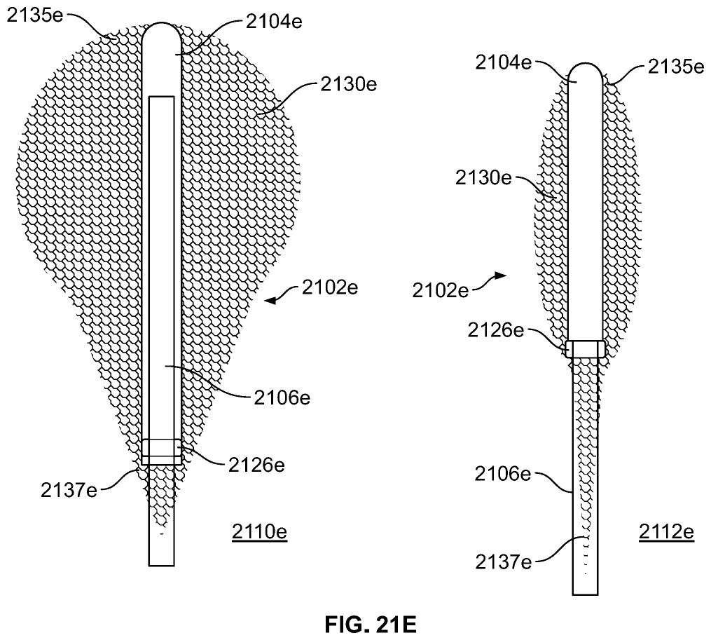

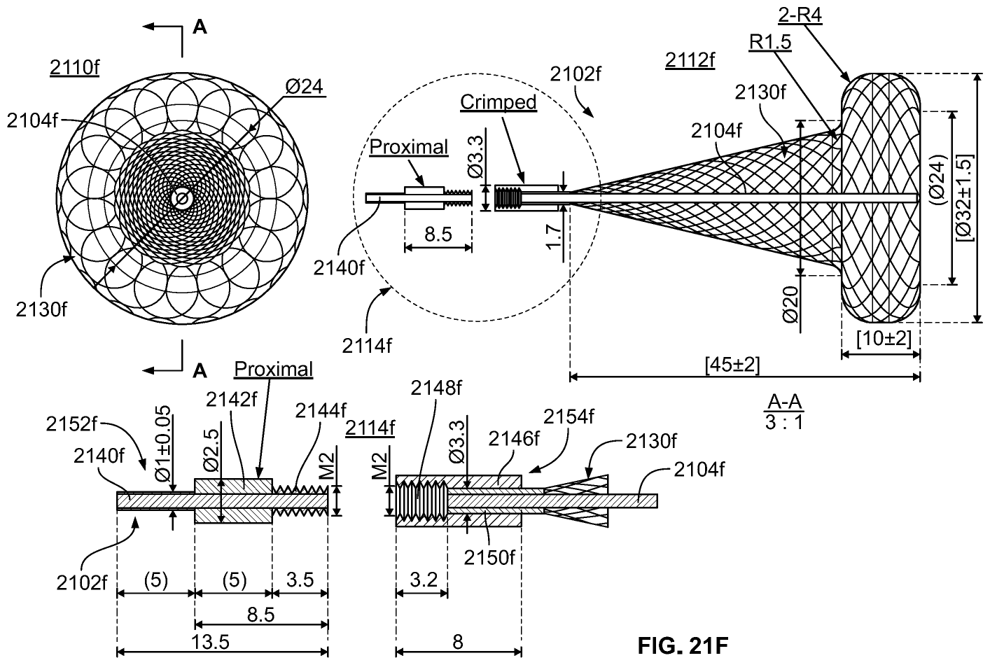

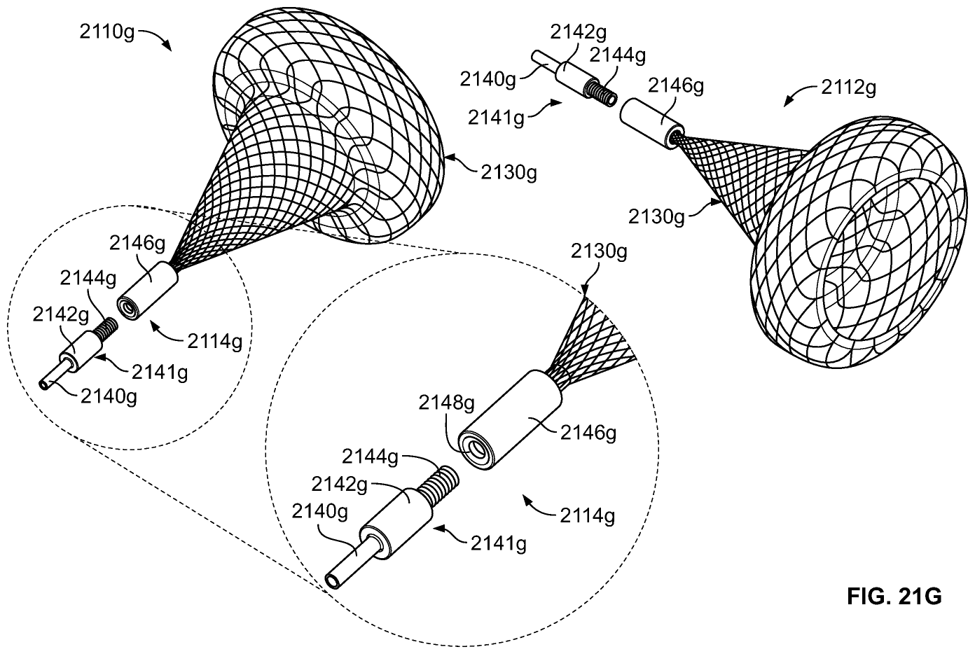

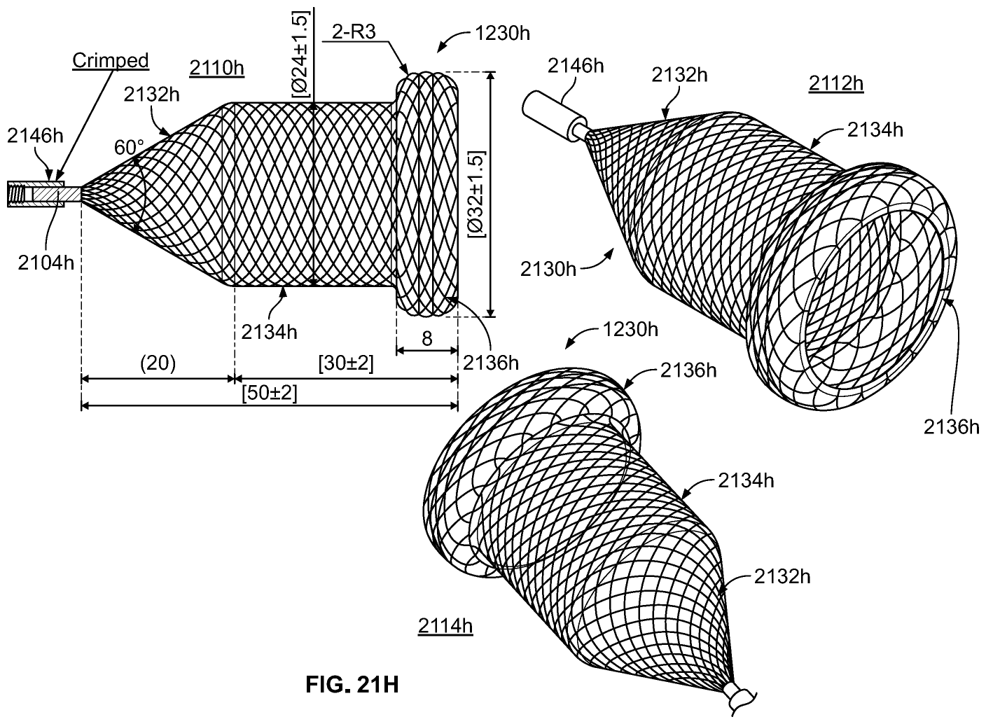

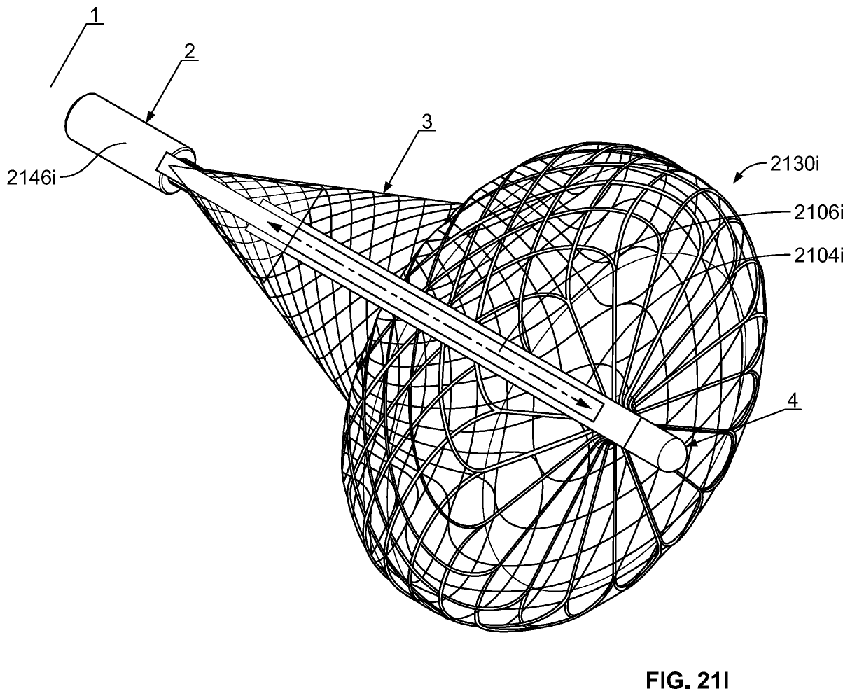

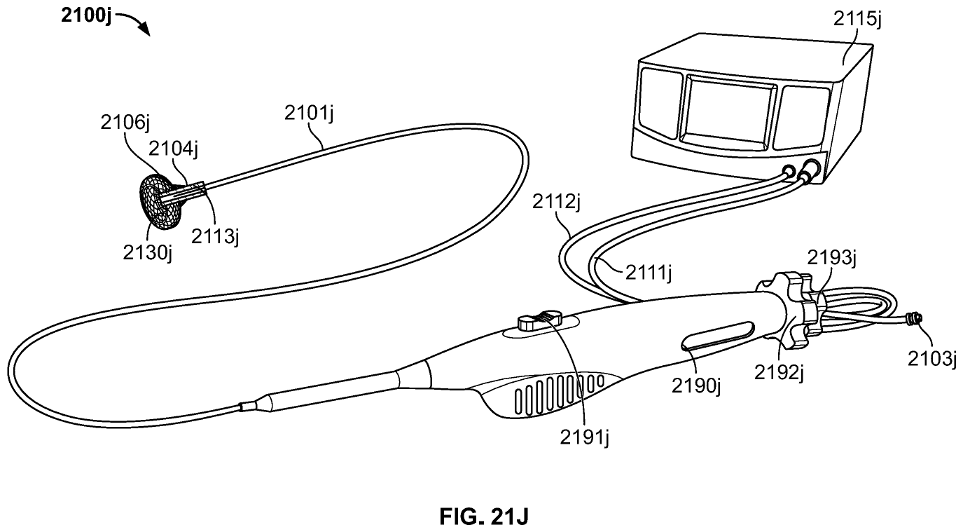

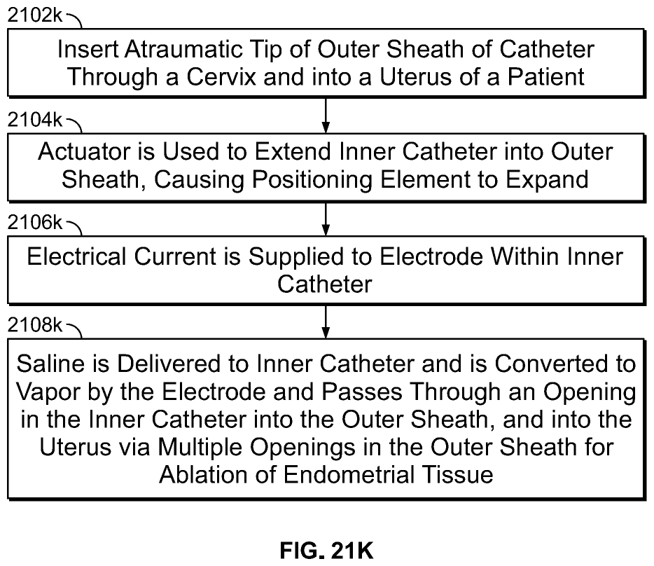

FIG. 21A is a flowchart illustrating a method of ablation of endometrial tissue in accordance with one embodiment of the present specification. Referring to FIG. 21A, the first step 3001 includes inserting a catheter of an ablation device through a cervix and into a uterus of a patient, wherein the catheter includes a hollow shaft through which an ablative agent can travel, at least one first positioning element, at least one second positioning element positioned distal to the at least one first positioning element, and at least one infusion port for delivering the ablative agent. In an embodiment, the ablation device includes a controller comprising a microprocessor for controlling the delivery of the ablative agent. The catheter is passed through the cervix such that a first positioning element is positioned in the cervix and a second positioning element is positioned in the uterine cavity. In one embodiment, the second positioning element is positioned proximate the fundus of the uterus. The two positioning elements are deployed such that the first positioning element contacts the cervix, the second positioning element contacts a portion of the uterine cavity, and the catheter and an infusion port are positioned within a uterine cavity of the patient in step 3002. Finally, in step 3005, an ablative agent is delivered through the infusion port to ablate the endometrial tissue.

Optionally, a sensor is used to measure at least one dimension of the uterine cavity in step 3003 and the measurement is used to determine the amount of ablative agent to be delivered in step 3004.

FIG. 21B is a flowchart illustrating a method of ablating a uterine fibroid. Referring to FIG. 21B, the first step 3011 includes inserting a hysteroscope through the cervix and into a uterus of a patient. Next, in step 3012 a catheter of an ablation device is passed through the hysteroscope, wherein the catheter includes a hollow shaft through which an ablative agent can travel, a puncturing tip at its distal end, at least one positioning element, and a plurality of needles on a distal end of the catheter and configured to deliver ablative agent to said uterine fibroid. In an embodiment, the ablation device includes a controller comprising a microprocessor for controlling the delivery of the ablative agent. The catheter is passed through said hysteroscope such that the puncturing tip of the catheter punctures the uterine fibroid. In the next step 3013, the at least one positioning element is deployed to position the catheter within the uterine fibroid such that the plurality of needles on the distal end of said catheter are positioned within the uterine fibroid. Finally, in step 3014, an ablative agent is delivered through the needles to ablate the fibroid. In some embodiments, the positioning element positions the catheter in the fibroid at approximate″ the average transverse dimension of the fibroid. In other embodiments, the positioning element positions the catheter in the fibroid at approximate 25% to 75% of an average transverse dimension of the fibroid.

FIGS. 21C to 21I illustrate exemplary embodiments of distal end of an endometrial ablation catheter having a single positioning element, in accordance with some embodiments of the present specification. FIG. 21J illustrates a system for endometrial ablation having a catheter with a single positioning element at its distal end, in accordance with various embodiments of the present specification. The embodiments include an inner catheter that contains a coaxial electrode within to convert saline to vapor. The inner coaxial chamber including the electrode is insulated by the inner catheter, and in some embodiments an outer sheath that covers the inner catheter, from external surfaces and tissues. The single positioning element, illustrated in FIGS. 21D to 211, has a proximal surface and a distal surface. In embodiments, proximal surface functions similarly to a proximal disc/positioning element, and distal surface functions similarly to a distal disc/positioning element, where the proximal and distal discs/positioning elements and their functions are described in FIGS. 18A to 18G.

Referring to FIG. 21C, in a first embodiment 2110c, a catheter 2102c includes an outer sheath 2104c and an inner catheter 2106c. Outer sheath 2104c includes a proximal end 2103c and a distal end 2105c with a soft atraumatic tip. Soft tip is configured to be soft and atraumatic to the vaginal canal, external cervical os, and cervix during positioning. Inner catheter 2106c is coaxial to outer sheath 2104c and has a smaller diameter than that of outer sheath 2104c. At least one electrode 2108c is positioned within a lumen 2115c of inner catheter 2106c. In other embodiments, the inner catheter includes at least one microwave antenna in place of the at least one electrode. Electrode 2108c is coaxial to inner catheter 2106c. In some embodiments, electrode 2108c extends along a full length or nearly a full length of inner catheter 2106c, as shown in illustration 2110c. Electrical current is supplied to electrode 2108c to generate heat to convert a fluid flowing by electrode 2108c to a vapor. Referring to illustration 2110c, saline 2101c passed along the length of electrode 2108c converts to vapor 2113c that exits from at least one inner catheter opening 2114c at a distal tip of inner catheter 2106c and spreads within an outer sheath lumen, or space or gap 2107c between an outer surface of inner catheter 2106c and an inner surface of outer sheath 2104c. In some embodiments, the inner catheter 2106c includes further openings to allow vapor 2113c to flow into the space or gap 2107c. Outer sheath 2104c is configured with one or more outer sheath openings 2116c through which the vapor exits catheter 2102c for ablation of endometrial tissue. In some embodiments, the outer sheath 2104c has a length in a range of 0.50 to 3.50 inches. In embodiment 2110c, outer sheath 2104c has a length of 1.75 inches. In some embodiments, outer sheath 2104c is insulated so that temperature of the vapor generated through electrode 2108c is maintained as the vapor exits openings 2114c and 2116c. In some embodiments, the catheter includes a temperature sensor 2109c. In some embodiments, four temperature sensors 2109c are included in the space 2107c between the inner catheter and outer sheath. In some embodiments, a pressure sensor 2111c is included in the fluid pathway, for example, in the space 2107c between the inner catheter and outer sheath.

A second embodiment 2112c a catheter 2122c comprising an inner catheter 2120c that extends only partially into the outer sheath lumen or space or gap 2127c between the inner catheter 2120c and the outer sheath 2124c. At least one electrode 2118c is positioned a lumen 2121c of the inner catheter 2120c. Outer sheath 2124c includes multiple openings 2126c in a portion of its distal length, while inner catheter 2120c and electrode 2118c are positioned in a portion of its proximal length. In some embodiments, openings 2126c are equidistant apart and spread across a surface of the outer sheath 2124c along a length of in a range of 0.25 inches to 2.50 inches of the distal portion of the outer sheath 2124c. In some embodiments, openings 2126c are equidistant apart and spread across a surface of the outer sheath 2124c along a length of 1.25 inches of the distal portion of the outer sheath 2124c. Inner catheter 2120c and electrode 2118c are positioned within a length of 0.5 inches of the proximal portion of the outer sheath 2124c. Saline 2127c passed along electrode 2118c is converted to vapor 2129c that passes through inner sheath opening 2128c into space 2127c and exits through openings 2126c along the distal portion of outer sheath 2124c to ablate endometrial tissue. In some embodiments, the catheter includes a temperature sensor 2139c. In some embodiments, four temperature sensors 2139c are included in the space 2127c between the inner catheter and outer sheath. In some embodiments, a pressure sensor 2131c is included in the fluid pathway, for example, in the space 2107c between the inner catheter and outer sheath.

FIG. 21D illustrates a configuration similar to first embodiment 2110c of FIG. 21C with a positioning element 2130d attached to the catheter 2102d. Positioning element 2130d has an elongated oval shape that extends along the length of outer sheath 2104d. In embodiments, positioning element 2130d is a wire mesh of which a first, proximal end 2131d is attached to a proximal end 2107d of the inner catheter 2106d and a second, distal end 2133d of the positioning element 2130d is attached to a distal end 2105d of the outer sheath 2104d. The wire mesh enables sufficient space between the wires of the mesh for vapor to exit. Positioning element 2130d functions similarly to the first and second positioning elements described with reference to the other endometrial ablation catheters of the present specification. A first surface 2135d of positioning element 2130d, corresponding to the second distal end 2133d, is configured to abut an upper, inner surface of a patient's uterus whiled a second surface 2137d of positioning element 2130d, corresponding to the first proximal end 2131d, is configured to abut an internal cervical os of the patient. In embodiments, the wire mesh is made from Nitinol, or any other shape memory material such that positioning element 2130d expands into a deployed configuration, as shown in FIG. 21D. In embodiments, positioning element 2130d is made from a single wire mesh. In some embodiments, positioning element 2130d is made from a double wire mesh. Fluid or saline is delivered into inner catheter 2106, is converted to vapor as it passes along electrode 2108d. Vapor is then delivered from the outer sheath openings 2116d while positioning element 2130d is in an expanded state and deployed within the uterus. Outer sheath 2104d includes an atraumatic distal tip 2124d that, in embodiments, is also covered externally by, and attached to, the second, distal end 2133d of the positioning element 2130d. FIG. 21D also illustrates an O-ring 2126d that is configured to slidably couple a proximal end 2103d of outer sheath 2104d with an outer surface of inner catheter 2106d. O-ring 2126d is configured to prevent vapor within space 2127d from exiting the catheter proximally back into the cervix. FIGS. 21F and 21G, subsequently described, illustrate details of the connection between inner catheter and outer sheath.

In embodiments, after deployment, electrode 2108d is positioned within inner catheter 2106d above O-ring 2126d and preferably covers the entire length of inner catheter 2106d that is disposed coaxially within outer sheath 2104d. Saline passed through inner catheter 2106d is heated by electrode 2108d and converted to vapor that exits through inner catheter opening 2114d at the distal tip of inner catheter 2106d and further through outer sheath openings 2116d on the surface of outer sheath 2104d. In some embodiments, the openings 2114d and 2116d are circular, slotted, semi-circular, or of any other shape. In some embodiments, 1 to 1000 openings 2116d are distributed over length outer sheath 2104d, where each opening has a length or a diameter in a range of 0.1 to 1 mm. In some embodiments, 64 to 96 openings are distributed over length outer sheath 2104d. In embodiments, the distal tip of outer sheath 2104d is encompassed within positioning element 2130d. In some embodiments, a silicone cover is provided to partially cover a surface of wire mesh of positioning element 2130d. The vapor from openings 2116d escapes through the uncovered portions of the wire mesh of positioning element 2130d.

FIG. 21E shows a deployed configuration 2110e and a compressed configuration 2112e of a distal end of ablation catheter 2102e having a positioning element 2130e, in accordance with some embodiments of the present specification. Catheter 2102e includes an outer sheath 2104e that is slidably coupled with an O-ring 2126e at its proximal end to an outer surface of an inner catheter 2106e. Positioning element 2130e includes two surfaces—a second proximal or bottom surface 2137e attached to inner catheter 2106e, and a first distal or top surface 2135e attached to a distal end of outer sheath 2104e. Initially, before deploying positioning element 2130e (see configuration 2112e), inner catheter 2106e is not yet positioned within outer sheath 2104e and positioning element 2130e is compressed about outer sheath 2130e. When deployed (see configuration 2110e), inner catheter 2106e is pulled coaxially into outer sheath 2104e and positioning element 2130e expands to fill the endometrial space. In some embodiments, positioning element 2130e expands to form a bulbous shape (also termed as bell-shape or teardrop-shape), which has a first distal surface 2135e or ‘disc’ which abuts the inner top surface of the uterus, and a second proximal surface 2137e or ‘disc’ which abuts the inner cervical os. Vapor, delivered through the openings in outer sheath 2104e, travels through the wire mesh of positioning element 2130e, exiting through spaces in the mesh, and contacts the endometrial tissue for ablation.

FIG. 21F illustrates an embodiment of a catheter 2102f with a positioning element 2130f in its deployed configuration, in accordance with some embodiments of the present specification. An outer sheath 2104f is positioned within and along a central longitudinal axis of positioning element 2130f Outer sheath 2104f is configured with openings and is described previously with reference to FIGS. 21C to 21E. Positioning element 2130f is conical in shape with a proximal end that is attached to outer proximal surface of outer sheath 2104f In some embodiments, proximal end of positioning element 2130f is crimped to attach with the outer surface of outer sheath 2104f. A first illustration 2110f shows a top view of positioning element 2130f and outer sheath 2104f located centrally within. A second illustration 2112f shows a cross sectional view of positioning element 2130f and outer sheath 2104f along a central longitudinal axis of outer sheath 2104f. A third illustration 2114f shows an enlarged view of a connection between proximal end of outer sheath 2014f and catheter body 2102f. Referring simultaneously to the three illustrations 2110f, 2112f, and 2114f, positioning element 2130f has its narrower proximal end attached to a proximal end of an inner catheter (not shown), and a distal circular end that has a doughnut shape with a maximum outer diameter in a range of 30.5 to 33.5 mm, an inner diameter of approximately 20 mm, a mean diameter of approximately 24 mm, and a length of approximately 8 to 12 mm. Outer sheath 2104f has a diameter of approximately 1.7 mm at its proximal end. At the distal end, positioning element 2104f has an atraumatic surface or a soft tip that is configured to be soft and atraumatic to the vaginal canal, external cervical os, and cervix during positioning. A proximal surface of the doughnut shaped portion of positioning element 2130f extends proximally in a conical configuration. In some embodiments, a total length of positioning element 2130f in a deployed, expanded configuration is in a range of approximately 43 to 47 mm. In some embodiments, an angle formed by the cone of positioning element 2130f is approximately 26.4°. Positioning element 2130f is made of wire mesh using material such as Nitinol. In some embodiments, a part or whole of the wire mesh is covered using Silicone. Covered portions of positioning element 2130f obstruct flow of vapor through the wire mesh and directs the flow out through the uncovered portions.

Illustration 2114f shows cross-sectional details of a connection between the proximal end of outer sheath 2104f and the distal end of catheter body 2102f. The connection includes a first connector 2152f and a second connector 2154f. The first connector 2152f is positioned on the distal end of the catheter body 2102f. In embodiments, the first connector 2152f has an overall length of 13.5 mm. A central portion has a greater outer thickness about a central lumen 2140f relative to a proximal portion and a distal portion. In some embodiments, the proximal and central portions each have lengths of 5 mm and the distal portion has a length of 3.5 mm. The distal portion includes a thread 2144f with a diameter M2. The catheter body 2102f has a diameter in a range of approximately 0.95 to 1.05 mm in the proximal portion. The thicker central portion of the first connector 2152f has a diameter of approximately 2.8 mm. The second connector 2154f is positioned at the distal end of the outer sheath 2104f and comprises a cylindrical crimp 2146f. In embodiments, the second connector 2154f has a length of approximately 8 mm and an outer diameter of approximately 3.3 mm. In some embodiments, a proximal portion of approximately 3.2 mm length of crimp 2146f is configured centrally within its cylindrical structure in a corrugated hollow 2148f to receive threaded length 2144f of first part of connector 2114f The remaining length 2150f of crimp 2146f is configured to received outer sheath 2104f coaxially within. In embodiments, during operation, an inner catheter (not shown) is pulled through lumen 2140f through hollow 2148f and further within outer sheath 2104f.

FIG. 21G illustrates different three-dimensional views of positioning element 2130g (corresponding to positioning element 21300 in its deployed configuration, and a connection 2114g, comprising first connector 2141g and second connector 2146g, in accordance with some embodiments of the present specification. A first illustration 2110g shows a bottom and side perspective view of positioning element 2130g and connection 2114g. An exploded view depicting the elements of connection 2114g is also shown in the figure. An illustration 2112g shows a top and side perspective view of positioning element 2130g and connection 2114g. First connector 2141g includes a lumen 2140g and comprises three portions. The most proximal portion 2140g is in an elongated cylindrical configuration and provides for passage of an inner catheter, through connector 2114g, into outer sheath (not shown) within positioning element 2130g. A middle portion 2142g has a thicker diameter than the most proximal portion of lumen 2140g. A distal portion 2144g of the first part of connector 2114g has a threaded surface to enable screw-connection with a hollow 2148g inside of second connector 2146g of connection 2114g. Second connector 2146g provides a crimp configured to crimp about and secure a proximal end of the outer sheath.

FIG. 21H illustrates different views of another embodiment of a positioning element 2130h in its deployed configuration, in accordance with some embodiments of the present specification. A first illustration 2110h shows a side view of positioning element 2130h. A second illustration 2112h shows a top and side perspective view of positioning element 2130h. A third illustration 2114h shows another perspective view of positioning element 2130h. Positioning element 2130h includes a proximal first portion 2132h, a middle second portion 2134h, and a distal third portion 2136h. Proximal first portion 2132h has a conical shape with a diameter that increases as the proximal first portion 2132h extends from the proximal end of the positioning element 2130h to the proximal end of the middle second portion 2134h. In embodiments, the proximal first portion 2132 has a length of approximately 20 mm and a conical angle of approximately 60° toward the middle second portion 2134h. Middle second portion 2134h continually extends in a cylindrical form from the distal end of the proximal first portion 2132h for a length, in some embodiments, in a range of 28 to 32 mm. In some embodiments, diameter of the middle second portion 2134h is in a range of 22.5 to 25.5 mm. Distal third portion 2136h includes a donut shape and extends distally from the distal end of the second middle portion 2134h. In some embodiments, distal third portion has a length of approximately 8 mm. Distal third portion 2136h has a diameter greater than a diameter of the middle second portion 2134h. In some embodiments, distal third portion 3136h includes an outer spherical surface covering a maximum diameter in a range of 30.5 to 33.5 mm. An inner circular surface of distal third portion 3136h is planar. In embodiments, the positioning element 2130h comprises a Nitinol mesh. In some embodiments, the mesh is partially covered with a silicone material to prevent the transfer of vapor out of the positioning element at the covered portions. In some embodiments, walls of the entire positioning element 2130h are made from Nitinol wire mesh that are covered with a silicone material, leaving its distal circular end open to enable exit of vapor for ablation. A connector 2146h is included at the proximal end and functions similarly to second connector 2146g of FIG. 21G.

FIG. 21I illustrates a coaxial, telescopic movement of an inner catheter 2106i within an outer sheath 2104i when a positioning element 2130i is deployed to the fully expanded configuration, in accordance with some embodiments of the present specification. Arrows shown inside inner catheter 2106i show the direction of coaxial telescopic movement of inner catheter 2106i inside outer sheath 2104i, which is towards distal end of positioning element 2130i when positioning element 2130i is being deployed to the fully expanded configuration. The direction of coaxial telescopic movement of inner catheter 2106i inside outer sheath 2104i is toward connector 2146i when positioning element 2130i is being compressed and inner catheter 2106i is retracted back to the delivery configuration. Referring to FIGS. 21E and 211 simultaneously, the distal end of the catheter 2102e, while in the compressed configuration shown in illustration 2112e, is advanced into a uterus of a patient. Inner catheter 2106e, 2106i is then advanced coaxially into outer sheath 2104e, 2104i, causing the positioning element to expand and resulting in the fully deployed configuration shown in illustration 2110e of FIG. 21E and in FIG. 21I.

FIG. 21J illustrates a system for use in the ablation of endometrial tissue, in accordance with some embodiments of the present specification. The system is configured to use the a catheter having a distal end with a single positioning element as described with reference to FIGS. 21C through 21I. The ablation system 2100j comprises a catheter 2101j which, in some embodiments, includes a handle 2190j having actuators 2191j, 2192j, 2193j for advancing an inner catheter 2106j within an outer sheath 2104j and deploying a positioning element 2130j at the distal end of the catheter 2101j. At least one electrode 2113j is positioned within the inner catheter. In some embodiments, the electrode is replaced with a microwave antenna. In embodiments, the positioning element 2130j is expandable, positioned at the distal end of the catheter 2101j, and may be compressed within the outer sheath 2104j for delivery. In some embodiments, actuators 2192j and 2193j comprise knobs. In some embodiments, actuator/knob 2192j is used to extend the inner catheter 2106j into the outer sheath 2104j and deploy the positioning element 2130j. For example, in embodiments, actuator/knob 2192j is turned one quarter turn to extend the inner catheter 2106j into the outer sheath 2104j and deploy the positioning element 2130j. In other embodiments, other combinations of actuators/knobs are used to extend/retract the inner catheter 2106j into and out of the outer sheath 2104j and deploy/compress the positioning element 2130j. In some embodiments, the catheter 2101j includes a port 2103j for the delivery of fluid, for example cooling fluid, during ablation. In some embodiments, port 2103j is also configured to provide for fluid collection, provide vacuum, and provide CO₂ for an integrity test. In some embodiments, the port 2103j is positioned on the handle 2190j. The electrode 2113j is configured to receive electrical current, supplied by a connecting wire 2111j extending from the controller 2115j to the catheter 2101j, to heat and convert a fluid, such as saline supplied via tubing 2112j extending from the controller 2115j to the catheter 2101j. Heated fluid or saline is converted to vapor or steam to be delivered by ports along the outer sheath for ablation. In some embodiments, the catheter 2101j is made of or covered with an insulated material to prevent the escape of ablative energy from the catheter body. Delivery of the ablative agent is controlled by the controller 2115j and treatment is controlled by a treating physician via the controller 2115j.

FIG. 21K is a flow chart illustrating the steps involved in using an ablation catheter to ablate an endometrium of a patient, in accordance with embodiments of the present specification. At step 2102k, an atraumatic tip of an outer sheath of the ablation catheter is advanced through a cervix and into a uterus of a patient. At step 2104k, an inner catheter of the ablation catheter is extended into the outer sheath using an actuator, which causes a compressed positioning element to expand and fill the uterus. Electrical current is supplied to at least one electrode positioned within the inner catheter at step 2106k. At step 2108k, saline is delivered into the inner catheter and along the at least one electrode, where it is converted to vapor and passes through an opening in the inner catheter into the outer sheath, where it then exits via multiple openings in the outer sheath to the uterus for ablation of endometrial tissue.

### Urinary Bladder Cancer Ablation and Treating OAB

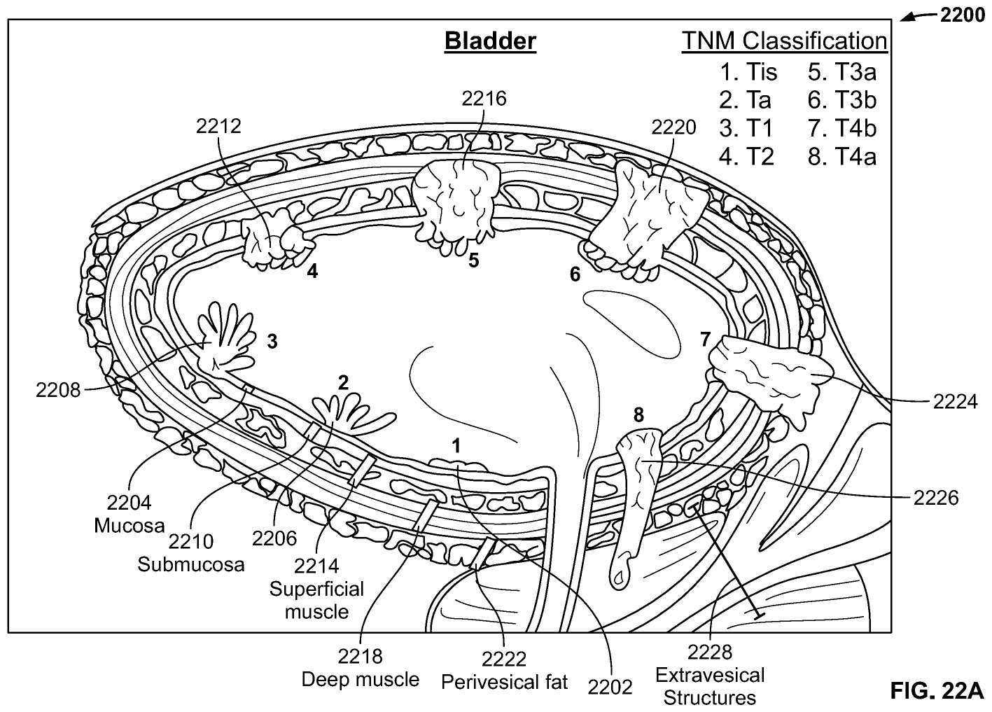

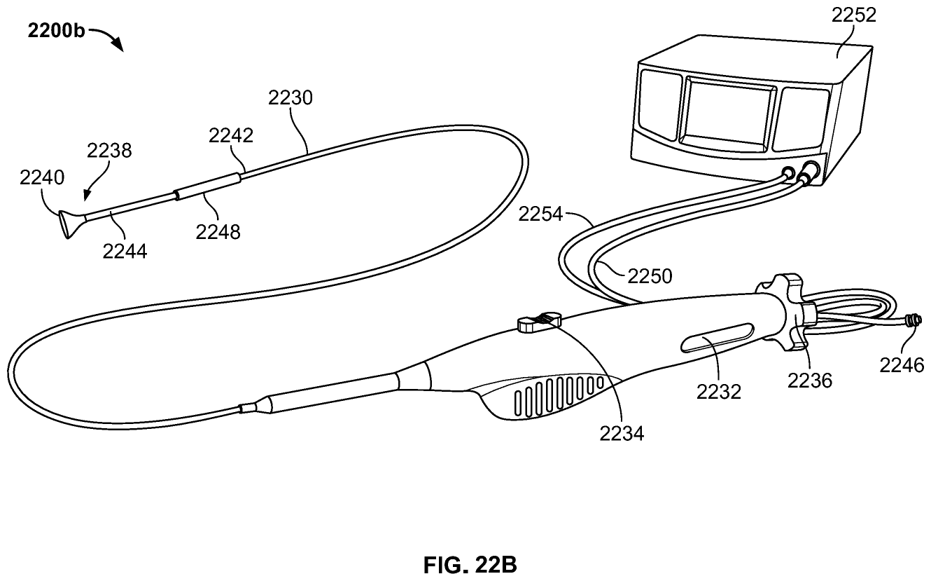

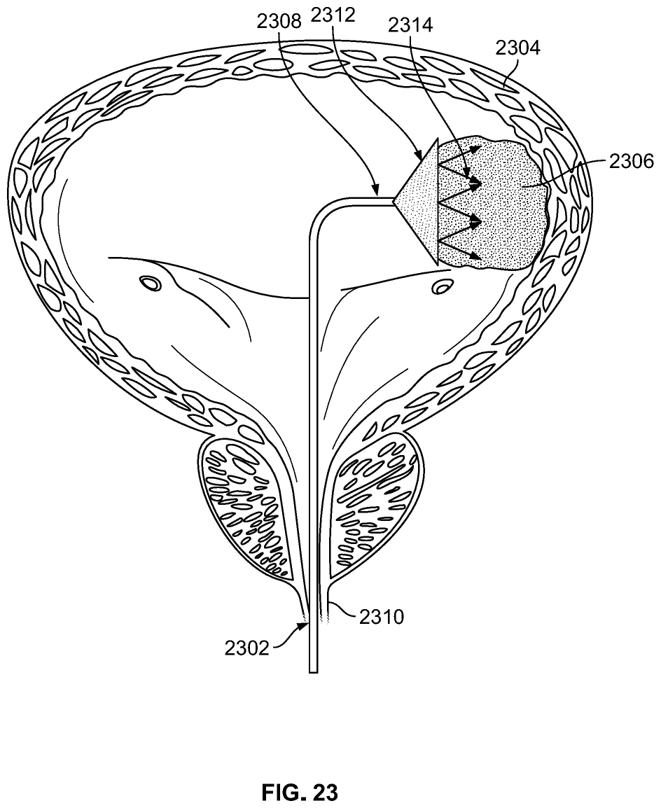

FIG. 22B illustrates a system 2200b for use in the ablation of bladder tissue, in accordance with an embodiment of the present specification. The system 2200b comprises a catheter 2230 which, in some embodiments, includes a handle 2232 having actuators 2234, 2236 for pushing forward a distal tip 2238 of the catheter 2230 and for deploying a distal positioning element 2240 at the distal end of the catheter 2230. In embodiments, the catheter 2230 comprises an outer sheath 2242 and an inner catheter 2244. In embodiments, the distal positioning element 2240 is expandable, positioned at the distal end of the inner catheter 2244, and may be compressed within the outer sheath 2242 for delivery. In some embodiments, actuators 2234 and 2236 comprise knobs. In some embodiments, actuator/knob 2236 is used to deploy the distal positioning element 2240. For example, in embodiments, actuator/knob 2236 is turned one quarter turn to deploy the distal positioning element 2240. In some embodiments, other combinations of actuators/knobs are used to the positioning element 2240. In some embodiments, the catheter 2230 includes a port 2246 for the delivery of fluid, for example cooling fluid, during ablation. In some embodiments, port 2246 is also configured to provide for fluid collection, provide vacuum, and provide CO₂ for an integrity test. In some embodiments, the port 2246 is positioned on the handle 2232. In some embodiments, at least one electrode 2248 is positioned at a distal end of the catheter 2230. The electrode 2248 is configured to receive electrical current, supplied by a connecting wire 2250 extending from a controller 2252 to the catheter 2230, to heat and convert a fluid, such as saline supplied via a tubing 2254 extending from the controller 2252 to the catheter 2230. Heated fluid or saline is converted to vapor or steam to be delivered by ports for ablation. In some embodiments, the catheter 2230 is made of or covered with an insulated material to prevent the escape of ablative energy from the catheter body. A plurality of small delivery ports is positioned on the inner catheter 2244 between the distal positioning element 2240 and the electrode 2248. Ports are used for the infusion of an ablative agent, such as steam. Delivery of the ablative agent is controlled by the controller 2252 and treatment is controlled by a treating physician via the controller 2252. In embodiments, the system 2200b of FIG. 22B is configured to be used for ablation of the urinary bladder, and may be used with catheters, positioning elements, and needles, described subsequently in context of FIGS. 23 to 28.

FIG. 23 illustrates an exemplary catheter 2302 for insertion into a urinary bladder 2304 for ablating bladder cancer 2306, in accordance with some embodiments of the present specification. Exemplary embodiments of distal ends of catheter 2302 are illustrated in context of FIGS. 18V, 18W, and 18X. A distal end 2308 of the catheter 2302 is advanced through a urethra 2310 and into a urinary bladder 2304. A cystoscope may be used for advancement of the catheter or, in some embodiments, visualization capability is provided in the catheter to navigate the catheter. A positioning element 2312 attached to the distal end 2308 of the catheter 2302 is used to position the ablation catheter 2302 inside the bladder 2304. In some embodiments, positioning element 2312 comprises a plurality of wires woven into a pattern, for example a spiral pattern. In embodiments, the wires are composed of a shape memory material to allow for compression of the positioning element 2312 during delivery. In some embodiments, the shape memory material is Nitinol. In various embodiments, the positioning element 2312 has a disc, cone, funnel, bell, spherical, oval, ovoid, or acorn shape and is substantially cylindrical when compressed. The positioning element 2312, when deployed, abuts and rests in the bladder 2304 encircling a portion of the tissue to be ablated.

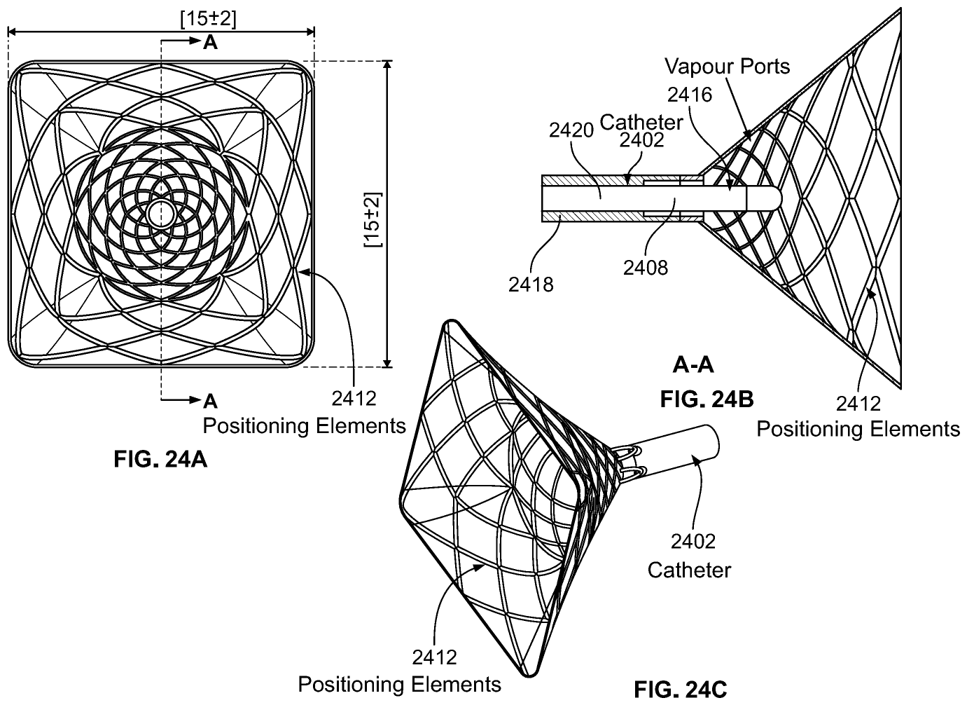

FIGS. 24A, 24B, and 24C illustrate different views of an exemplary configuration of a distal end of a catheter 2402 with a positioning element 2412, in accordance with some embodiments of the present specification. FIG. 24A illustrates a front end view of the positioning element 2412. FIG. 24B illustrates a side view of the catheter 2402 and positioning element 2412. FIG. 24C illustrates a front side perspective view of the catheter 2402 and positioning element 2412. Referring simultaneously to FIGS. 24A, 24B, and 24C, positioning element 2412 is in a shape of a pyramid, with four sides, providing an open square form at its distal end. In some embodiments, the length and width of the positioning element 2412 at its open, distal end is in a range of 13 to 17 mm. Catheter 2402 is attached at its distal end 2408 to the positioning element 2412. Catheter 2402 includes an outer catheter 2418 and an inner catheter 2420. In embodiments, the positioning element 2402 is attached with a connecting mechanism to the distal end 2408 of the outer catheter 2418. The inner catheter 2420 is positioned inside and coaxially with the outer catheter 2418. Vapor ports 2416 are configured on the inner catheter 2420, which provide an outlet for vapor 2314 (FIG. 23) during ablation.

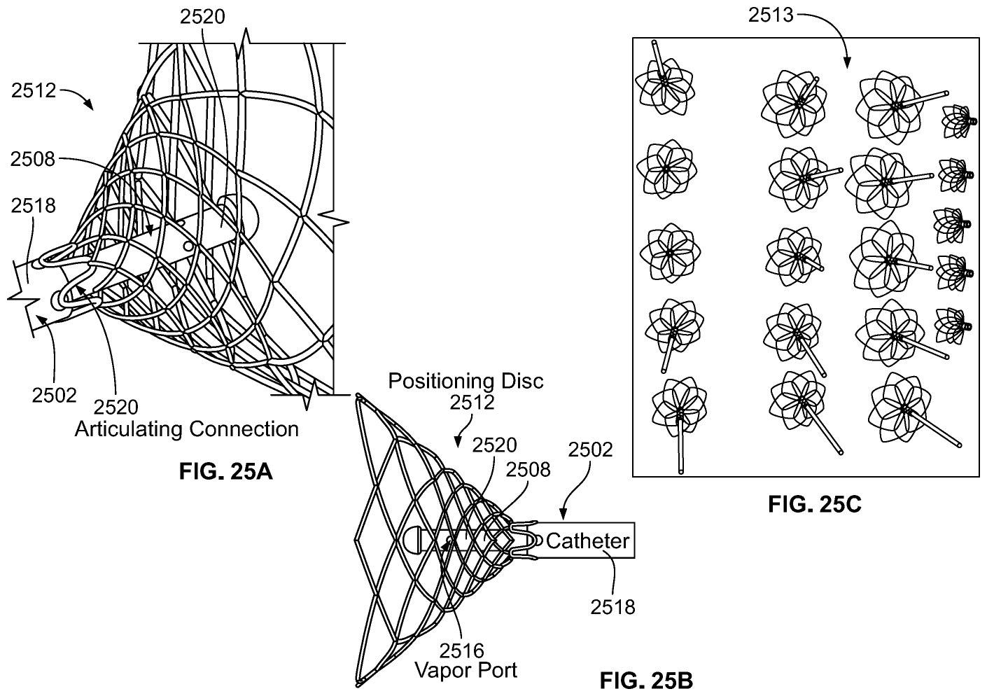

FIGS. 25A, 25B, and 25C illustrate designs of a positioning element 2512, in accordance with some embodiments of the present specification. FIG. 25A illustrates a close-up view of a connection 2520 between positioning element 2512 and a catheter 2502, in accordance with some embodiments of the present specification. In alternative embodiments, the positioning element 2512 is fused with the catheter 2502, is free floating with metal or polymer sutures, is hinged with laser welded Nitinol, where the hinge is cut with a laser, or is attached with a Nitinol sleeve that is welded to it. In some embodiments, the connection 2520 is an over-sleeve or part of a distal end 2508 of the catheter 2502. FIG. 25B illustrates a side view of positioning element 2512 attached to distal end 2508 of the catheter 2502. One or more vapor ports 2516 are configured on an inner catheter 2520 within an outer catheter 2518, at the distal end of the catheter 2502, where the distal portion of the catheter 2502 is located within the funnel-shaped volume of the positioning element 2512. In embodiments, the inner catheter 2520 is movable into and out of the outer catheter 2518 such that the outer catheter 2518 covers the inner catheter 2520 and restrains the positioning element 2512 before insertion into a patient's urethra. The positioning element 2512 is composed of a shape memory material such that it expands into a deployed configuration, as shown in FIG. 25A, once the inner catheter 2520 is extended beyond the distal end of the outer catheter 2518. FIG. 25C illustrates different types of configurations of positioning elements 2513 which may be used in accordance with the embodiments of the present specification. In some embodiments, the positioning elements are conical in shape with diameters varying from 5 mm to 50 mm. In some embodiments, the positioning elements are ovoid cones with a first proximal diameter of the cone less than a second distal diameter of the cone to approximate the shape or dimensions of a urethra. In various embodiments with multiple positioning elements, a first positioning element may have a different shape or size from a second positioning element. One or more positioning elements maybe used to accomplish the therapeutic purpose.

In some embodiments, the positioning elements 2512 are formed with wire made from one, or a combination, of polymers and metal, such as including and not limited to Polyether ether ketone (PEEK) and Nickel Titanium (NiTi). In some embodiments, the wire is covered with elastomers such as PTFE, ePTFE, PU and/or silicone in a variety of patterns. The various cells in the positioning elements 2513 may be covered or uncovered based on hood functionality such as whether it is to be used for sealing, or for venting, or for any other purpose. In embodiments where the positioning elements 2513 are made from Nitinol wire meshes, the wires have a diameter in a range of 0.16 to 0.18 mm. In some embodiments, for the positioning element 2513, the wire mesh is coated with silicone but not the areas between wires in the mesh, therefore allowing steam to escape/vent from these spaces between the wires. In some embodiments, wires and space between wires are covered with silicone.

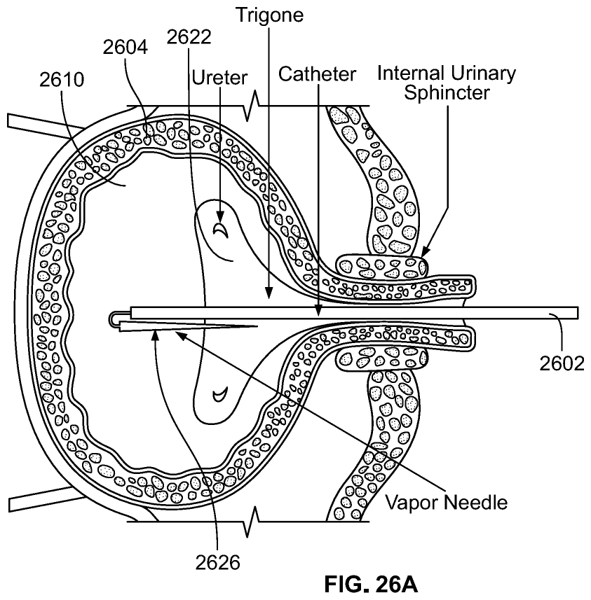

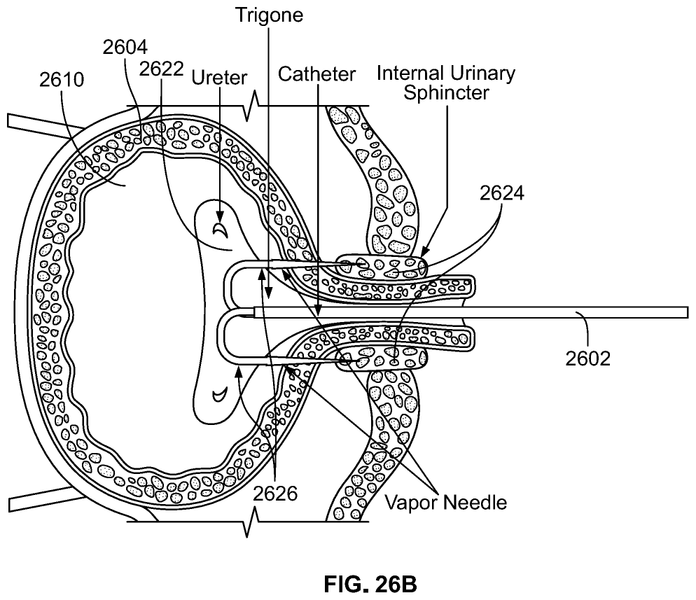

Embodiments of the present specification may also be used in the ablation of bladder neck tissue and/or an internal bladder sphincter for treatment of an OAB, as described with reference to the embodiments of subsequent FIGS. 26A and 26B. An OAB is related to a sudden, uncontrolled need or urge to urinate. OAB is different from stress urinary incontinence (SUI), where people leak urine while sneezing, laughing, or doing other physical activities. OAB may result from improper coordination of the nerve signals between the bladder and the brain. The signals might tell a patient to empty the bladder, even when the bladder is not full. OAB can also be caused when muscles in the bladder are too active. In this case, the bladder muscles contract to pass urine before the bladder is full, causing a sudden urge to urinate. Treating the bladder neck and/or an internal bladder sphincter with the ablation methods of the present specification provide a method of treating OAB. Accordingly, vapor is delivered selectively to ablate nerve-rich layers of deep detrusor and adventitial space beneath trigone. Alternatively, vapor is delivered selectively with the help of the RF generator to ablate the bladder neck, internal urinary sphincter (IUS) and nerves supplying the IUS. The RF generator provides power to electrodes in a heating chamber within the catheter. When fluid flows through spaces in the heating chamber and power is applied to the electrodes causing the electrodes to charge which is conducted through the saline, resistively heating the saline and vaporizing the water in the saline. The thermal energy remodels the tissue, resulting in improved barrier function and fewer random relaxations that cause incontinence due to OAB.

FIG. 26A illustrates positioning of a needle ablation catheter 2602 for delivering vapor to selectively ablate nerve-rich layers of deep detrusor and adventitial space beneath trigone 2622, in accordance with embodiments of the present specification. FIG. 26B illustrates positioning of a needle ablation device for delivering vapor to selectively ablate the bladder neck, IUS and nerves supplying the IUS 2624 and bladder neck, in accordance with embodiments of the present specification. Referring simultaneously to FIGS. 26A and 26B, one or more needles 2626 are used to deliver vapours to a target area 2622 or 2624. In embodiments, sensor probes are used to measure one or more parameters and thereby control the ablation. In one embodiment, a sensor probe may be positioned at the distal end of the heating chambers within the catheter. During vapor generation, the sensor probe communicates a signal to the controller. The controller may use the signal to determine if the fluid has fully developed into vapor before exiting the distal end of the heating chamber. Sensing whether the saline has been fully converted into vapor may be particularly useful for many surgical applications, such as in the ablation of various tissues, where delivering high quality (low water content) steam results in more effective treatment.

The ablation system of FIGS. 26A and 26B comprise a catheter 2602 having an internal heating chamber, disposed within a lumen of the catheter and configured to heat a fluid provided to the catheter 2602 to change the fluid to a vapor for ablation therapy. In some embodiments, the catheter 2602 is made of or covered with an insulated material to prevent the escape of ablative energy from the catheter body. A plurality of openings is located proximate the distal end of the catheter 2602 for enabling a plurality of associated thermally conductive elements, such as needles 2626, to be extended (at an angle from the catheter 2602, wherein the angle ranges between 30 to 180 degrees) and deployed or retracted through the plurality of openings. In accordance with an aspect, the plurality of retractable needles 2626 are hollow and include at least one infusion port to allow delivery of an ablative agent, such as steam or vapor, through the needles 2626 when the needles 2626 are extended and deployed through the plurality of openings on the elongated body of the catheter 2602. In some embodiments, the infusion ports are positioned along a length of the needles 2626. In some embodiments, the infusion ports are positioned at a distal tip of the needles 2626. In various embodiments, a tension wire attached to the needle is used to control the shape and position of the needle to assist in puncturing the bladder wall. In some embodiments, the vapor is applied through the needle to the trigone of the bladder ablating the nerves in trigone of the bladder to prevent or treat OAB.

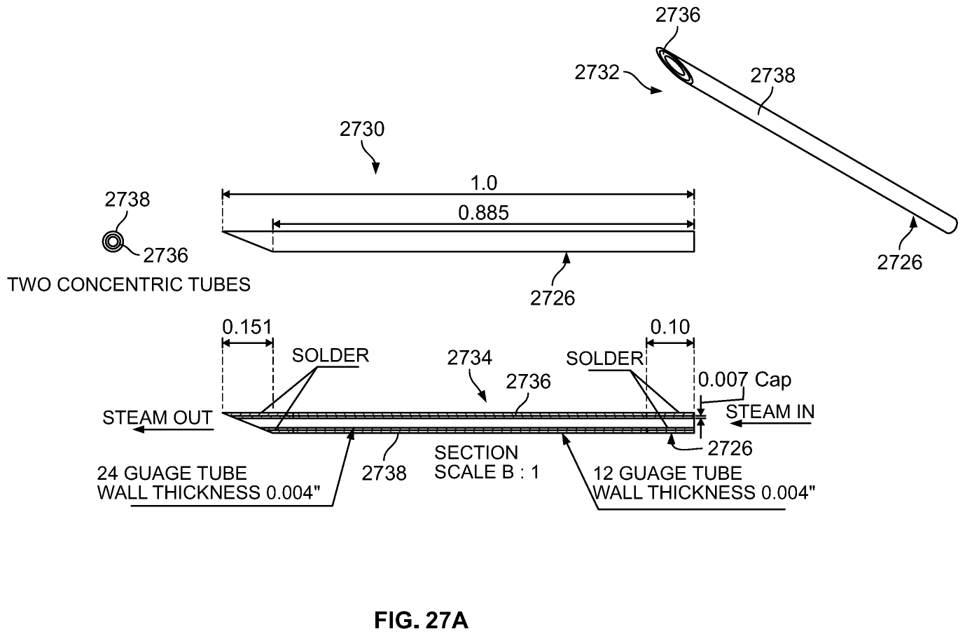

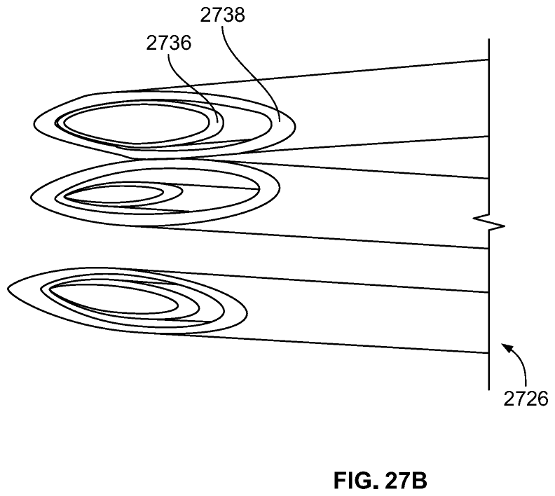

FIG. 27A illustrates different views of a coaxial needle 2726 that may be used for ablation for treatment of OAB, in accordance with some embodiments of the present specification. The figure shows a side view 2730, a front-end side perspective view 2732, and a cross-section of the side view 2734, of the needle 2726. In some embodiments, the needle 2726 comprises two concentric tubes with lumens—an inner tube 2736 within an outer tube 2738. The needle 2726 is diagonally sectioned at its sharp distal end, so that in one embodiment, a length of the needle extending to its sharp pointed distal end is about 1 mm, and a length extending to its proximal distal end is about 0.885 mm. The inner tube 2736 comprises a first lumen to provide for a channel to exit vapours for ablation. In one embodiment, a gap between the inner and outer tubes 2736 and 2738 is of about 0.007 mm. The inner and outer tubes 2736, 2738, are soldered together for about 0.151 mm at the distal end and for about 0.10 mm at the proximal end of the needle 2726. The vapours generated in the catheter travel through one or more openings where the one or more needles 2726 are connected to the catheter, enter the needles 2726 from the hollow of inner tube 2736 at the proximal side of the needle 2726 and exit from the distal side of the needle 2726. FIG. 27B illustrates the distal ends of coaxial needles 2726 comprising inner tubes 2736 with lumens and outer tubes 2738 with lumens, in accordance with some embodiments of the present specification. In some embodiments, a gap between the inner tubes 2736 with lumens and outer tubes 2738 with lumens is filled with air or a fluid for insulation. The gap may be flushed and may be used for aspiration in some embodiments.

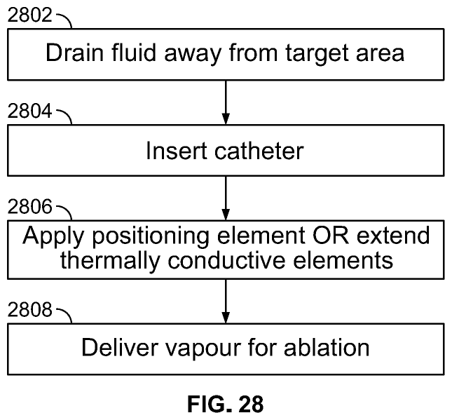

FIG. 28 is a flow chart illustrating an exemplary process of ablating the urinary bladder and/or its peripheral areas, in accordance with some embodiments of the present specification. An ablation system described in context of the various figures above is used for ablating a target area within or proximate the urinary bladder of a patient. The target area may include a tissue, cyst, tumour of stages 1 to 8, within the urinary bladder, so as to treat cancerous growth. The target area may also include nerve-rich layers of the deep detrusor and adventitial space beneath the trigone, and the bladder neck, IUS, and nerves supplying the IUS and bladder neck. In accordance with the present specification, at step 2802, liquid (urine) from the urinary bladder is drained. The bladder is drained to empty the bladder so that there is no anticipation of the urine wetting the target area or pooling on or around the target area. Draining the target area is performed to ensure the removal of bulk urine for effective ablation. In some embodiments, the urine is removed from the bladder. In some embodiments, additionally, air or CO2 is insufflated into the bladder to expand the bladder. The air is used to dry the internal surface of the bladder before ablation. In some embodiments, additionally, the patient is positioned to drain any residual urine away from a targeted part of the bladder using gravity, by making the targeted portion of the bladder non-dependent. At step 2804, a catheter of the ablation system is inserted into the urinary bladder. At step 2806, a positioning element, such as positioning element 2312 of FIG. 23, is deployed proximate the target area, so as to enclose a portion or whole of the target area to be ablated. Alternatively, a thermally conductive element, such as one or more needles 2626 of FIGS. 26A and 26B, are deployed to access peripheral areas of the urinary bladder, including and not limited to, the area beneath the trigone and the IUS of the patient or a prostate of the patient. At step 2808, vapor is delivered to the target area to ablate the target area. In embodiments, pressure within the bladder is maintained to a level below 5 atm during the ablation.

### Imaging Capabilities

Imaging capabilities may be added to the ablation systems used for benign prostatic hyperplasia (BPH), abnormal uterine bleeding (AUB), over-active bladder (OAB), and for any other tissue ablation processes described in the embodiments of the present specification. In embodiments, the imaging capabilities are provided in the form of an integrated optical chip with the ablation system or as a coaxial fiber optical wire with the sheath of the catheter of the ablation system.

FIG. 29 illustrates a system 29100 for use in the ablation and imaging of prostatic tissue, in accordance with an embodiment of the present specification. The system 29100 comprises a catheter 29102 which, in some embodiments, includes a handle 29104 having actuators 29106, 29108 for extending at least one needle or a plurality of needles 29110 from a distal end of the catheter 29112 and expanding a positioning element 29114 at the distal end of the catheter 29112. In some embodiments, actuators 29106 and 29108 may be one of a knob or a slide or any other type of switch or button to enable extending of at least one needle from the plurality of needles 29110. Delivery of vapor via the catheter 29102 is controlled by a controller 29116. In embodiments, the catheter 29102 comprises an outer sheath 29118 and an inner catheter 29120. The needles 29110 extend from the inner catheter 29120 at the distal end of the sheath 29118 or, in some embodiments, through openings proximate the distal end of the sheath 29118. In embodiments, the positioning element 29114 is expandable, positioned at the distal end of the inner catheter 29120, and may be compressed within the outer sheath 29118 for delivery. In some embodiments, actuator 29108 comprises a knob which is turned by a first extent, for example, by a quarter turn, to pull back the outer sheath 29118. As the outer sheath 29118 retracts, the positioning element 29114 is revealed. In embodiments, the positioning element 29114 comprises a disc or cone configured as a bladder anchor. In embodiments, actuator/knob 29108 is turned by a second extend, for example, by a second quarter turn, to pull back the outer sheath 29118 further to deploy the needles 29110. In some embodiments, referring to FIGS. 29, 4C and 4E simultaneously, the needles 29110, 3116a are deployed out of an internal lumen of the inner catheter 29120, 3111a through slots or openings 3115a in the outer sheath 29118, 3110a, which helps control the needle path and insulates the urethra from steam. In some embodiments, the openings are covered with slit covers 3119. In another embodiment, for example, as seen in FIG. 4D, the sleeves 3116b naturally fold outward as the outer sheath 3110b is pulled back.

Referring again to FIG. 29, in some embodiments, the catheter 29102 includes a port 29122 for the delivery of fluid, for example cooling fluid, during ablation. In some embodiments, port 29122 is also configured to provide for fluid collection, provide vacuum, and provide CO₂ for an integrity test. In some embodiments, port 29122 is used for fluid irrigation as well as for steam generation and aspiration. In some embodiments, the port 29122 is positioned on the handle 29104. In some embodiments, at least one electrode 29124 is positioned at a distal end of the catheter 29102 proximal to the needles 29110. The electrode 29124 is configured to receive electrical current, supplied by a connecting wire 29128 extending from the controller 29116 to the catheter 29102, to heat and convert a fluid, such as saline supplied via tubing 29126 extending from the controller 29116 to the catheter 29102. Heated fluid or saline is converted to vapor or steam to be delivered by the needles 29110 for ablation.

In embodiments, a capability for imaging is integrated with the system 29100. In some embodiments, sheath 29118 includes an optical fiber connected to a fiber optic light source 29134 to illuminate the passage of the distal end of catheter 29102. In some embodiments, a sheath 29128 is provided parallel to outer sheath 29118, where the sheath 29128 includes the optical fiber, or includes an optical chip. In some embodiments, the sheath 29128 is coaxial with the outer sheath 29118, parallel to an inner sheath 29120. In some embodiments, the catheter 29102 is a multi-lumen catheter, with one lumen for the camera and electronics (sheath 29128). The sheath 29128 may be made from materials such as polyurethane or thermoplastic polymers. In some embodiments, the system 29100 includes an integrated optical circuit (IC), which is mounted within the system 29100. FIG. 11O illustrates and describes detailed of embodiments of a viewing device that may be integrated with the catheter 29102, in accordance with some embodiments. The IC may be a part of the catheter 29102, or in the generator 29116, or in a third party computing device that is in communication with the system 29100. An eyepiece 29130 is integrated within the handle 29104. The eyepiece 29130 enables a user, such as the physician, to view the passage of the catheter 29102, captured by the optical system (optical fiber, integrated optical circuit). In some embodiments, a video of the images captured by the optical system is transmitted using a video correction cable 29132, to a display, such as a screen of a computer or a phone. A button or an interactive interface or trigger is provided in the generator 29116 or with a third party computing device in communication with the system 29100, which enable controlling the capture of still and video images.

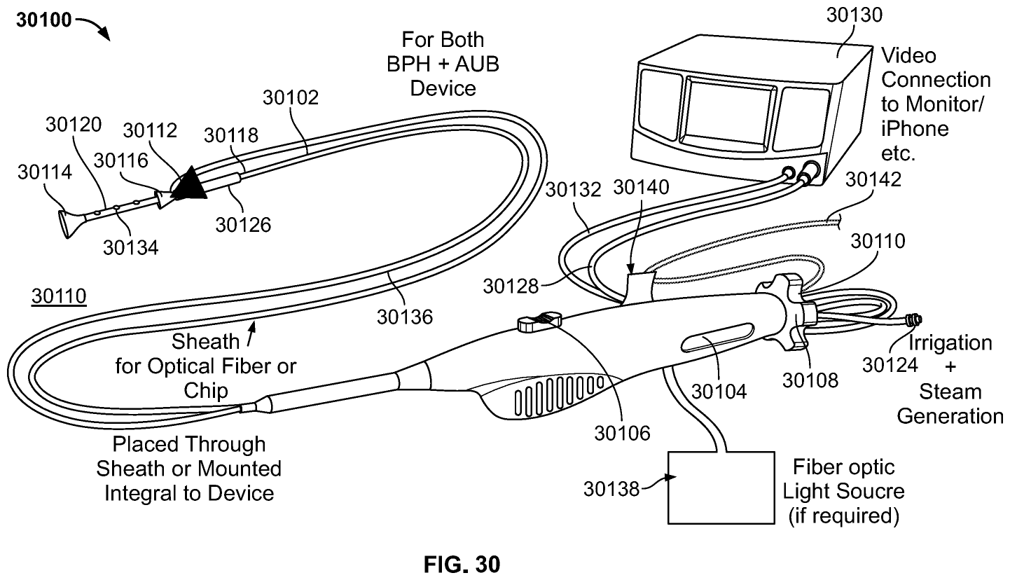

FIG. 30 illustrates a system 30100 for use in the ablation of endometrial tissue, in accordance with an embodiment of the present specification. The ablation system 30100 comprises a catheter 30102 which, in some embodiments, includes a handle 30104 having actuators 30106, 30108, 30110 for pushing forward a distal bulbous tip of the catheter 30102 and for deploying a first distal positioning element 30114 and a second proximal positioning element 30116 at the distal end of the catheter 30102. In embodiments, the catheter 30102 comprises an outer sheath 30118 and an inner catheter 30120. In embodiments, the catheter 30102 includes a cervical collar 30122 configured to rest against an external os once the catheter 30102 has been inserted into a uterus of a patient. In embodiments, the distal first positioning element 30114 and proximal second positioning element 30116 are expandable, positioned at the distal end of the inner catheter 30120, and may be compressed within the outer sheath 30118 for delivery. In some embodiments, actuators 30108 and 30110 comprise knobs. In some embodiments, actuator/knob 30108 is used to deploy the distal first positioning element 30114. For example, in embodiments, actuator/knob 30108 is turned one quarter turn to deploy the distal first positioning element 30114. In some embodiments, actuator/knob 30110 is used to deploy the proximal second positioning element 30116. For example, in embodiments, actuator/knob 30110 is turned one quarter turn to deploy the proximal second positioning element 30116. In some embodiments, the handle 30104 includes only one actuator/knob 30108 which is turned a first quarter turn to deploy the first distal positioning element 30114 and then a second quarter turn to deploy the second proximal positioning element 30116. In other embodiments, other combinations of actuators/knobs are used to deploy one or both of the first distal positioning element 30114 and second proximal positioning element 30116. In some embodiments, the catheter 30102 includes a port 30124 for the delivery of fluid, for example cooling fluid, during ablation. In some embodiments, port 30124 is also configured to provide for fluid collection, provide vacuum, and provide CO2 for an integrity test. In some embodiments, port 30124 is used for fluid irrigation as well as for steam generation or aspiration. In some embodiments, the port 30124 is positioned on the handle 30104. In some embodiments, at least one electrode 30126 is positioned at a distal end of the catheter 30102 proximal to the proximal second positioning element 30116. The electrode 30126 is configured to receive electrical current, supplied by a connecting wire 30128 extending from a controller 30130 to the catheter 30102, to heat and convert a fluid, such as saline supplied via a tubing 30132 extending from the controller 30130 to the catheter 30102. Heated fluid or saline is converted to vapor or steam to be delivered by ports 30134 for ablation. In some embodiments, the catheter 30102 is made of or covered with an insulated material to prevent the escape of ablative energy from the catheter body. A plurality of small delivery ports 30134 is positioned on the inner catheter 30120 between the distal first positioning element 30114 and the second proximal positioning element 30116. Ports 30134 are used for the infusion of an ablative agent, such as steam. Delivery of the ablative agent is controlled by the controller 30130 and treatment is controlled by a treating physician via the controller 30130.

In embodiments, a capability for imaging is integrated with the system 30100. In some embodiments, sheath 30118 includes an optical fiber connected to a fiber optic light source 30138, to illuminate the passage of the distal end of catheter 30102. In some embodiments, a sheath 30136 is provided parallel to outer sheath 30118, where the sheath 30136 includes the optical fiber, or includes an optical chip. In some embodiments the system 30100 includes an integrated optical circuit, which is mounted within the system 30100. FIG. 11O illustrates and describes detailed of embodiments of a viewing device that may be integrated with the catheter 29102, in accordance with some embodiments. An eyepiece 30140 is integrated within the handle 30104. The eyepiece 30140 enables a user, such as the physician, to view the passage of the catheter 30102, captured by the optical system (optical fiber, integrated optical circuit). In some embodiments, a video of the images captured by the optical system is transmitted using a video correction cable 30142, to a display, such as a screen of a computer or a phone.

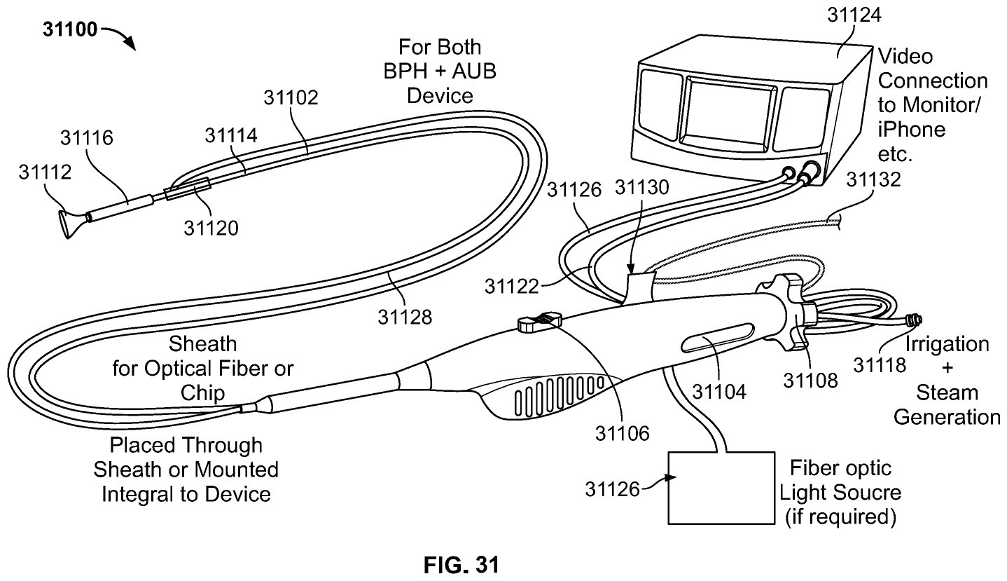

FIG. 31 illustrates a system 31100 for use in the ablation of bladder tissue, in accordance with an embodiment of the present specification. The ablation system 31100 comprises a catheter 31102 which, in some embodiments, includes a handle 31104 having actuators 31106, 31108 for pushing forward a distal tip of the catheter 31102 and for deploying a distal positioning element 31112 at the distal end of the catheter 31102. In embodiments, the catheter 31102 comprises an outer sheath 31114 and an inner catheter 31116. In embodiments, the distal positioning element 31112 is expandable, positioned at the distal end of the inner catheter 31116, and may be compressed within the outer sheath 31114 for delivery. In embodiments, the positioning element 31112 comprises a disc or cone configured as a bladder anchor. In some embodiments, actuators 31106 and 31108 comprise knobs. In some embodiments, actuator/knob 31108 is used to deploy the distal positioning element 31112. For example, in embodiments, actuator/knob 31108 is turned one quarter turn to deploy the distal positioning element 31112. In other embodiments, other combinations of actuators/knobs are used to deploy the first positioning element 31112. In some embodiments, the catheter 31102 includes a port 31118 for the delivery of fluid, for example cooling fluid, during ablation. In some embodiments, port 31118 is also configured to provide for fluid collection, provide vacuum, and provide CO2 for an integrity test. In some embodiments, port 31118 is used for fluid irrigation as well as for steam generation. In some embodiments, the port 31118 is positioned on the handle 31104. In some embodiments, at least one electrode 31120 is positioned at a distal end of the catheter 31102. The electrode 31120 is configured to receive electrical current, supplied by a connecting wire 31122 extending from a controller 31124 to the catheter 31102, to heat and convert a fluid, such as saline supplied via a tubing 31126 extending from the controller 31124 to the catheter 31102. Heated fluid or saline is converted to vapor or steam to be delivered by ports and/or needles for ablation. In some embodiments, the catheter 31102 is made of or covered with an insulated material to prevent the escape of ablative energy from the catheter body. Delivery of the ablative agent is controlled by the controller 31124 and treatment is controlled by a treating physician via the controller 31124.

In embodiments, a capability for imaging is integrated with the system 31100. In some embodiments, sheath 31114 includes an optical fiber connected to a fiber optic light source 31126, to illuminate the passage of the distal end of catheter 31102. In some embodiments, a sheath 31128 is provided parallel to outer sheath 31114, where the sheath 31128 includes the optical fiber, or includes an optical chip. FIG. 11O illustrates and describes detailed of embodiments of a viewing device that may be integrated with the catheter 29102, in accordance with some embodiments. In some embodiments the system 31100 includes an integrated optical circuit, which is mounted within the system 31100. An eyepiece 31130 is integrated within the handle 31104. The eyepiece 31130 enables a user, such as the physician, to view the passage of the catheter 31102, captured by the optical system (optical fiber, integrated optical circuit). In some embodiments, a video of the images captured by the optical system is transmitted using a video correction cable 31132, to a display, such as a screen of a computer or a phone.

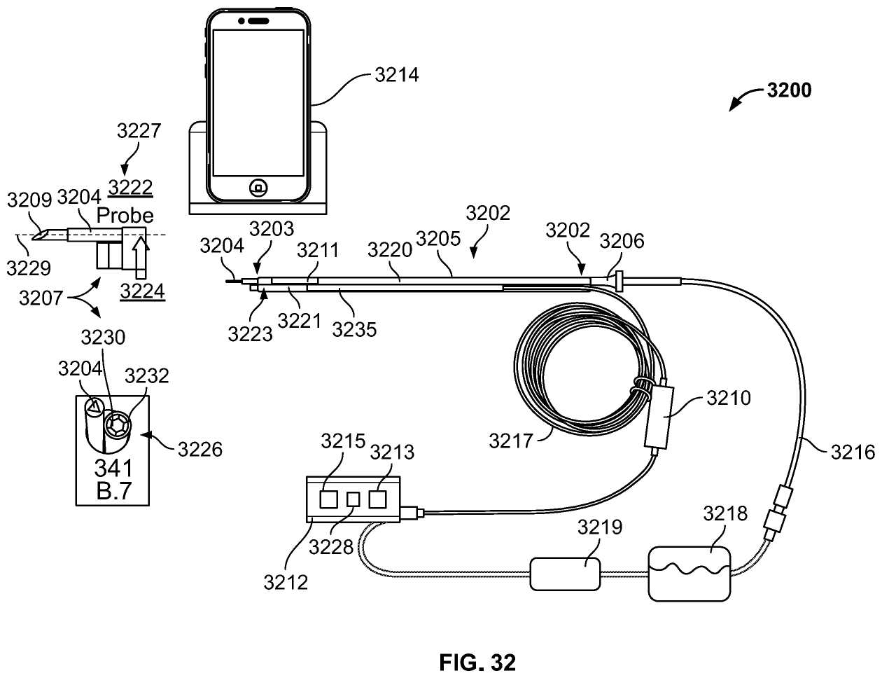

FIG. 32 illustrates various components of an optical/viewing system 3200 for direct visualization that can be used in accordance with the embodiments of the present specification. In embodiments, the system 3200 includes an ablation catheter 3202 configured to deliver an ablation fluid to a volume of prostate tissue or a volume of fibroid tissue. The ablation catheter 3202 provides for direct visualization that enables an optical capture of a movement and/or location of a needle for prostate or fibroid treatment. The direct visualization is achieved using an optical system, comprising a camera and light source, integrated into the same catheter that includes the needle and heating component for vapor generation. In embodiments, the catheter comprises ablation components and viewing and illumination components, in the form of a camera and a light source, to enable viewing of a target area during ablation. The catheter 3202 comprises a channel or a sheath 3205 having at least one first lumen 3220 configured to receive a volume of fluid, such as saline, from a fluid reservoir or source 3128. At least one needle 3204 is positioned at a distal end 3203 of the catheter 3202 and is configured to be deployed from a surface of a distal tip 3207 of the catheter 3202. The at least one needle 3204 includes at least one port 3209 for the delivery of an ablative agent. In embodiments, the at least one needle 3204 is configured to be deployed at an angle relative to a longitudinal axis 3229 defining a direction of the distal tip 3207. In embodiments, the angle is in a range of 10 degrees to 90 degrees relative to the longitudinal axis 3229 defining the direction of the distal tip 3207. At least one heating component 3211 is positioned within the first lumen 3220 proximate the distal tip 3207. In some embodiments, the at least one heating component 3211 comprises an electrode. In some embodiments, the electrode is a flat electrode. In some embodiments, the electrode has a tapered structure so that a distal tip of the electrode is thinner than a proximal portion of the electrode. A handle 3206 is coupled to the proximal end 3201 of the sheath 3205. In embodiments, the handle 3206 is configured to enable an operator to deploy and retract the at least one needle 3204 from and into the distal tip 3207 of the catheter 3202.

The catheter further comprises a camera 3230 at the distal tip 3207. In embodiments, the camera 3230 is configured to visually capture a movement and location of the at least one needle 3204 when the needle 3204 is extended out from the surface of the distal tip 3207. A light source 3232 is positioned proximate the camera 3230 and is configured to illuminate a target tissue area and assist in visualization via the camera 3230. In embodiments, the camera 3230 and light source 3232 are physically coupled into the sheath 3205. In some embodiments, a second lumen 3221 is positioned within the sheath 3205 and extends parallel to the first lumen 3220. In some embodiments, the camera 3230 and light source 3232 are positioned within a distal end 3223 of the second lumen 3221. The catheter 3204 further includes optical data transmission circuitry 3235 coupled to the camera 3230. In embodiments, the optical data transmission circuitry 3235 is configured to transmit visual data captured by the camera 3230 to a controller 3212, wherein the controller 3212 comprises a processor 3213 configured to process the visual data and present a visual image to the operator on a display device 3214. In some embodiments, the optical data transmission circuitry 3235 is positioned within the second lumen 3221. In some embodiments, the second lumen 3221 has a diameter that is equal to or less than 4 mm and the first lumen 3220 has a diameter that is equal to or less than 4 mm. A probe 3222 is formed at the distal tip 3207 of the catheter 3204 by the combined presence of the needle 3204, camera 3230, and light source 3232. In embodiments, ablation catheter 3202 has a diameter of 8 mm or less, preferably 7 mm or less, preferably 6 mm or less, and most preferably in a range of 4 mm to 5 mm.

Ablation catheter 3202 is steered by a physician with controls that are provided on the 3206 that is coupled to the proximal end 3202 of sheath 3205. In embodiments, handle 3206 is a multi-function handle that connects the first lumen 3220 of the ablation catheter 3202 with a fluid source 3218 via a connection tube 3216 that provides the fluid or saline to be converted to vapor for ablation. Connection tube 3216 is in fluid communication with a fluid source or reservoir 3218 to provide fluid to the first lumen 3220. In embodiments, the fluid is saline. Fluid source or reservoir 3218 is in pressure communication with a pump 3219 which is coupled to the controller 3212. In embodiments, the controller 3212 is in electrical communication with the at least one heating component 3211 and is programmed to deliver an electrical current to the heating component 3211 and to cause, by controlling the pump, a volume of fluid to pass into the first lumen 3220 from the fluid reservoir 3218 when activated. Fluid passing by the heating component 3211 is converted to vapor and delivered via the at least one port 3209 of the needle 3204 to ablate a target tissue. In embodiments, the controller 3212 is programmed to deliver the electrical current to the at least one heating component 3211 for a continuous period of time that is equal to, or less than, one minute. In embodiments, the controller 3212 further comprises a power source 3215 positioned therewithin and coupled to the camera 3230 and light source 3232. The power source 3215 is configured to provide power to the camera 3230 and light source 3232.

The distal tip 3207 of the catheter 3202 comprises a ‘probe’ 3222, which comprises the needle 3204 and distal end of the first lumen 3220, the camera 3230, and the light source 3232. In some embodiments, the light source 3232 comprises at least one LED light. The camera 3230 captures images of movement and location of the needle 3204 when it is deployed from the distal tip 3207 of catheter 3202. The ability to directly capture images of movement and location of the needle during an ablation procedure is especially useful for prostate and fibroid treatments. Conventional approaches require the use of an additional scope, separate and distinct from the ablation catheter, to achieve visualization. However, the use of a separate scope creates greater complexity to a procedure, makes it difficult for a single operator to perform the procedure, and increases overall costs. Embodiments of the present specification avoid the need for a separate scope, such as an endoscope, for visualization of the treatment. A close-up view 3227 and a front-end view 3226 of probe 3222 or distal tip 3207 of the catheter 3202 illustrate the at least one needle 3204 and camera 3230 surrounded by a light source 3232, positioned together. Referring to the close-up view 3227, an arrow 3224 points to a location where the at least one heating component is positioned for fluid or saline to vapor generation. The optical data transmission circuitry 3235 is in electrical communication with the controller 3212 via an electrical wire 3217. In some embodiments, the electrical wire includes a button, a switch, or any other type of interface 3210 which enables the user to control an intensity of the light emitted by the light source 3232. In other embodiments, intensity of the light source 3232 is controlled using the handle 3206. In some embodiments, the controller 3212 includes a wireless transmitter 3228 for communicating the images taken by the camera 3230 to a peripheral display device 3214 for viewing. In some embodiments, the peripheral display device 3214 is a television or a computer screen, a mobile or portable display device, or a mobile phone. In some embodiments, communication between the controller 3212 and peripheral display device 3214 is wired. In the embodiments of the present specification, catheter 3202, including the attached camera 3230 and light source 3232, is disposable. In embodiments, the entirety of the catheter 3202, from its distal end 3203 and including the distal tip 3207 with needle 3204, camera 3230, and light source 3232, to a proximal end of connection tube 3216, where it connects to fluid reservoir 3218, and a proximal end of electrical wire 3217, where it connects to controller 3212, and all components therebetween, is disposable.

FIG. 33 illustrates components of a distal end 3350 of an ablation system that may be used in treatment of abnormal uterine bleeding (AUB), for use in accordance with the embodiments of the present specification. The distal end 3350 comprises a distal hood or positioning element 3352, inner catheter shaft 3353, and a proximal hood or positioning element 3354 extending from an ablation catheter 3355, and a viewing device 3356 with a light source and a camera of an optical/electrical catheter 3342. The viewing device 3356 is positioned so that the proximal hood 3354 is located distal to the viewing device along the length of the distal end 3350. In operation, a physician may view, using the viewing device 3356, the distal end of the catheter 3355, along with the distal hood 3352, inner catheter shaft 3353, and proximal hood, 3352, to ensure proper positioning of these elements prior to the initiation of vapor delivery. In embodiments, the size, stiffness, and position of each hood 3352 and 3354 is adjustable (see FIGS. 18S, 18T, 19A-C, for details). In embodiments, a length of the distal end 3350 that is between the distal 3352 and proximal 3354 hoods is also adjustable. The length, once adjusted, may be locked to position and hold the hoods 3352 and 3354 in place.

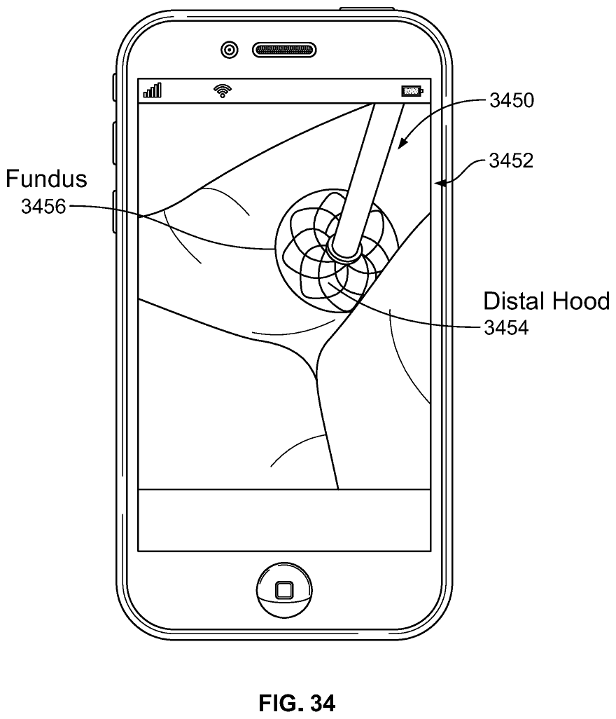

FIG. 34 illustrates an image 3450 viewed on a display device 3452, such as an iPhone, in accordance with some embodiments of the present specification. The exemplary image shows a distal hood 3454 (similar to distal hood 3352) reaching a surface that could be a fundus 3456 of a uterus during an ablation procedure. The image 3450 is captured by a viewing device such as the device 3356 of FIG. 33.

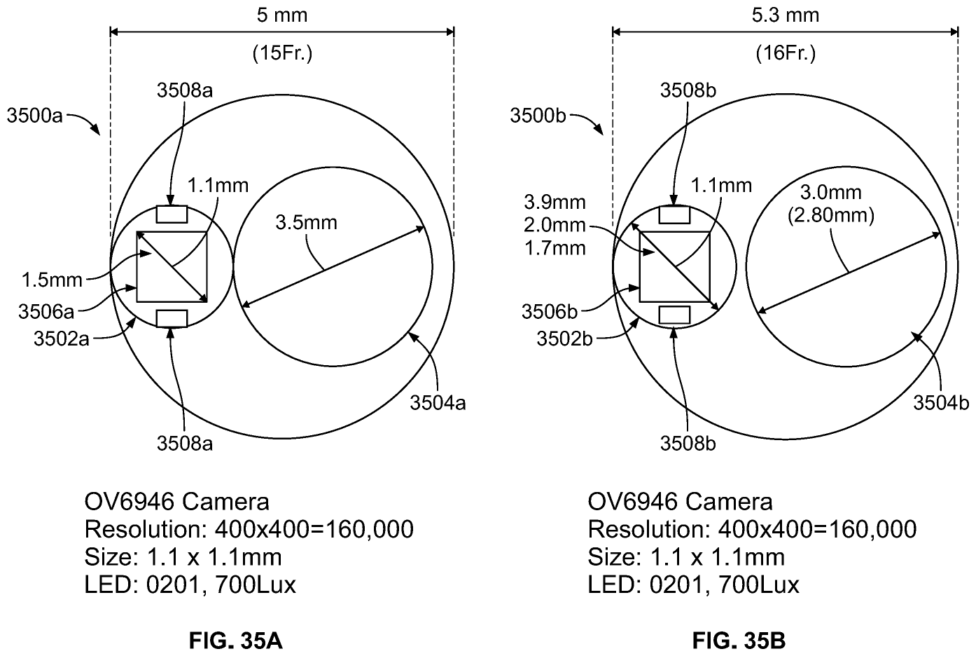

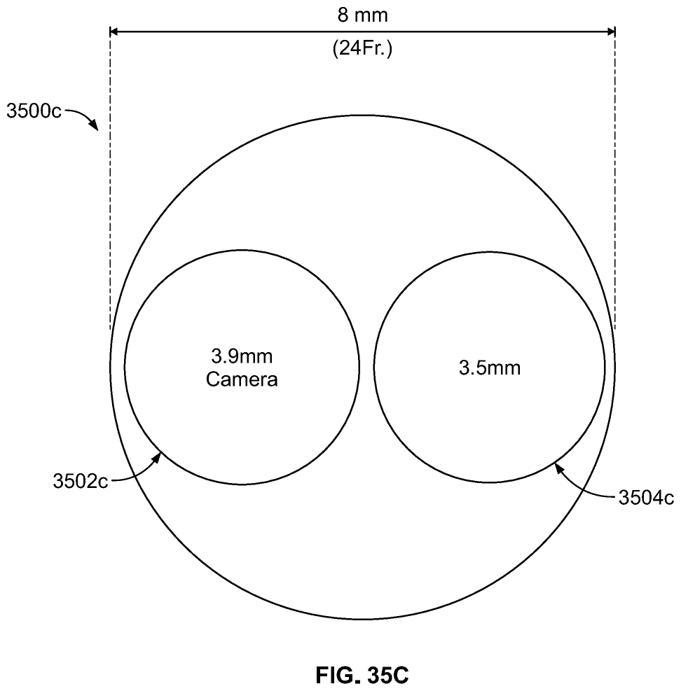

FIG. 35A depicts a cross-sectional view of an embodiment of a combination catheter 3500a comprising lumens 3502a for an optical/electrical catheter alongside a lumen 3504a for an ablation catheter, in accordance with some embodiments of the present specification. Referring simultaneously to FIG. 33, the ablation catheter lumen 3504a is configured to receive the ablation catheter shaft 3355. In some embodiments, the ablation catheter lumen 3504a has a diameter of approximately 3.5 mm. Similarly, the lumens 3502a for the optical/electrical catheter are configured to receive the optical/electrical catheter 3342 components that may include the viewing device 3356 with a light source and a camera. The lumens 3502a for the optical/electrical catheter include a camera lumen 3506a for the electronics for the viewing device 3356. The camera lumen 3506a may be square in shape, with a diagonal distance extending to 1.5 mm and sides of 1.1 mm, in an embodiment. In some embodiments, the camera lumen 3506a is configured to receive the electronics for an OV6946 camera with a resolution of 160,000 (400×400). Additionally, lumens 3502a for the optical/electrical catheter include lumens 3508a above and below lumen 3506a for holding the electronics for the light sources. The lumens 3508a may be configured to receive the electronics for LEDs with an illuminance of approximately 700 Lux. In some embodiments, the lumens 3508a are rectangular in shape. The combination catheter 3500a may be circular with a diameter of approximately 5 mm, so as to accommodate the optical/electrical catheter 3342 alongside the ablation catheter 3355.

FIG. 35B depicts a cross-sectional view of another embodiment of a combination catheter 3500b comprising lumens 3502b an optical/electrical catheter alongside a lumen 3504b for an ablation catheter, in accordance with some embodiments of the present specification. Referring simultaneously to FIG. 33, the ablation catheter lumen 3504b is configured to receive the ablation catheter shaft 3355. In some embodiments, the ablation catheter lumen 3504b has a diameter in a range of approximately 2.8 to 3.0 mm. Similarly, the lumens 3502b for the optical/electrical catheter are configured to receive the optical/electrical catheter 3342 components that may include the viewing device 3356 with a light source and a camera. In some embodiments, the lumens 3502b for the optical/electrical catheter comprise an area of the combination catheter 3500b having a diameter that may range from 1.7 mm to 3.9 mm. In an embodiment, the diameter of the area of the combination catheter housing the lumens 3502b for the optical/electrical catheter is approximately 2.0 mm. The lumens 3502b for the optical/electrical catheter include a camera lumen 3506b for the electronics for the viewing device 3356. The camera lumen 3506b may be square in shape, with a diagonal distance extending to 1.5 mm and sides of 1.1 mm, in an embodiment. In some embodiments, the camera lumen 3506b is configured to hold an OV6946 camera with a resolution of 160,000 (400×400). Additionally, lumens 3502b for the optical/electrical catheter include lumens 3508b above and below lumen 3506b for holding the electronics for the light sources. The lumens 3508b may be configured to receive the electronics for LEDs with an illuminance of 700 Lux. In some embodiments, the lumens 3508b are rectangular in shape. The combination catheter 3500b may be circular with a diameter of approximately 5.3 mm, so as to accommodate the optical/electrical catheter 3342 alongside the ablation catheter 3355.

FIG. 35C depicts a cross-sectional view of yet another embodiment of a combination catheter 3500c comprising a lumen 3502c for an optical/electrical catheter alongside a lumen 3504c for an ablation catheter 3504c, in accordance with some embodiments of the present specification. In embodiments, the catheter 3500c may have a diameter of approximately 8 mm. In embodiments, the optical/electrical catheter lumen 3502c has a diameter 3.9 mm and is configured to receive the optical/electrical catheter 3342 of FIG. 33, including the electronics for both the camera and LEDs. In embodiments, the ablation catheter lumen 3504c has a diameter 3.5 mm and is configured to receive the ablation catheter 3355 of FIG. 33. Therefore, in different embodiments, different sizes of combination catheter, optical/electrical catheter, and ablation catheter are possible.

Handle Mechanisms

Multiple embodiments of handle mechanism that may be used with ablation devices for prostate ablation are now described. While these embodiments are used for prostate ablation, they may also be used with other systems of the present specification. The multiple embodiments of handle mechanism include systems for needle deployment and retraction, which can be achieved in different ways such as and not limited to buttons, push/pull on distal or proximal end, slide button in a track, rotation with push/pull (plunger embodiment with wishbone paddle). The internal components of the embodiments of the handle mechanism are made generally using stainless steel. The external components of the embodiments of the handle mechanism are made generally using a combination of ABS, plastics, rigid polymers, and elastomeric polymers, among other material. The various embodiments also provide for strain relief for the back end for the fluid tube and the electrical cable, and strain relief on the front end to provide support for the catheter segment. In various embodiments, the handles described in the present specification have lengths ranging from 3 inches to 24 inches and diameters ranging from ¼ inch to 5 inches.

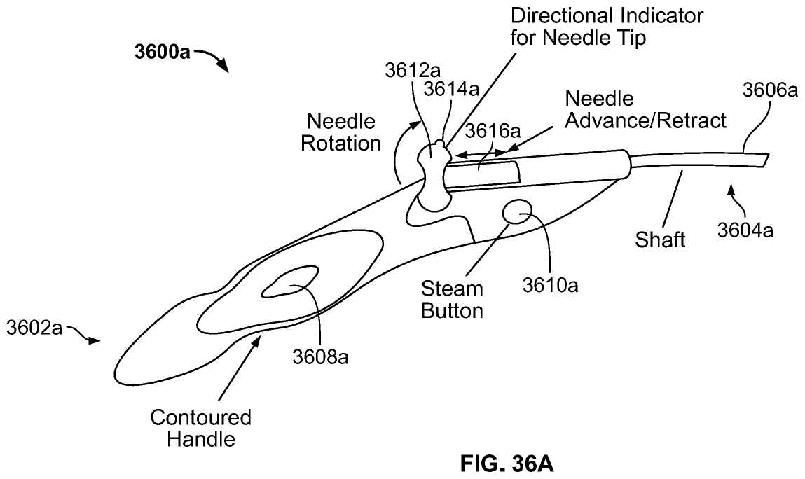

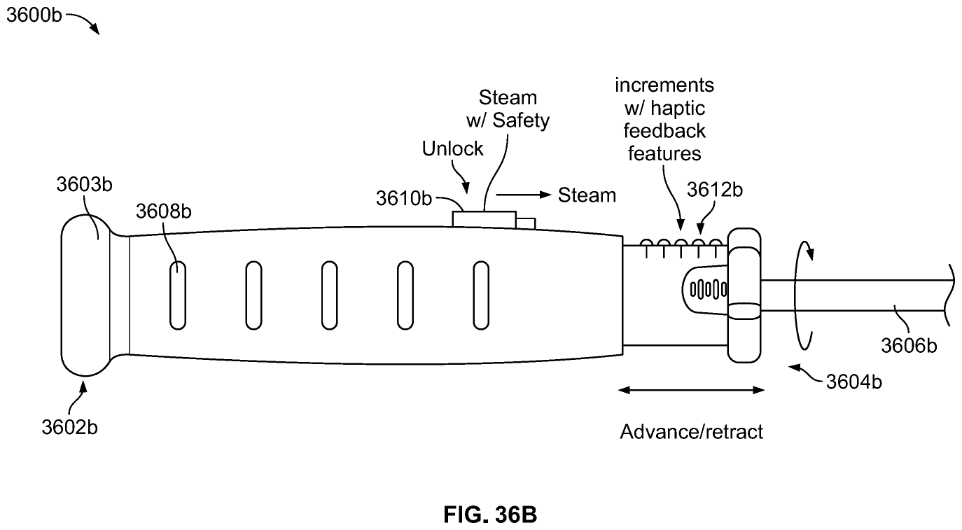

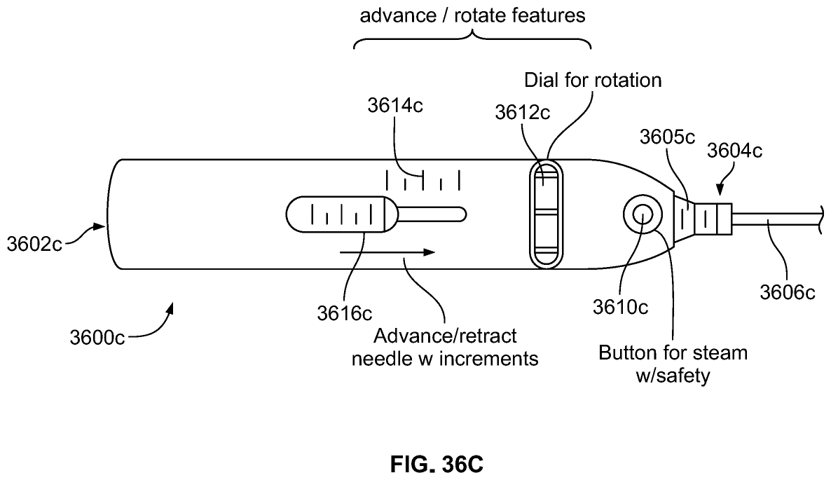

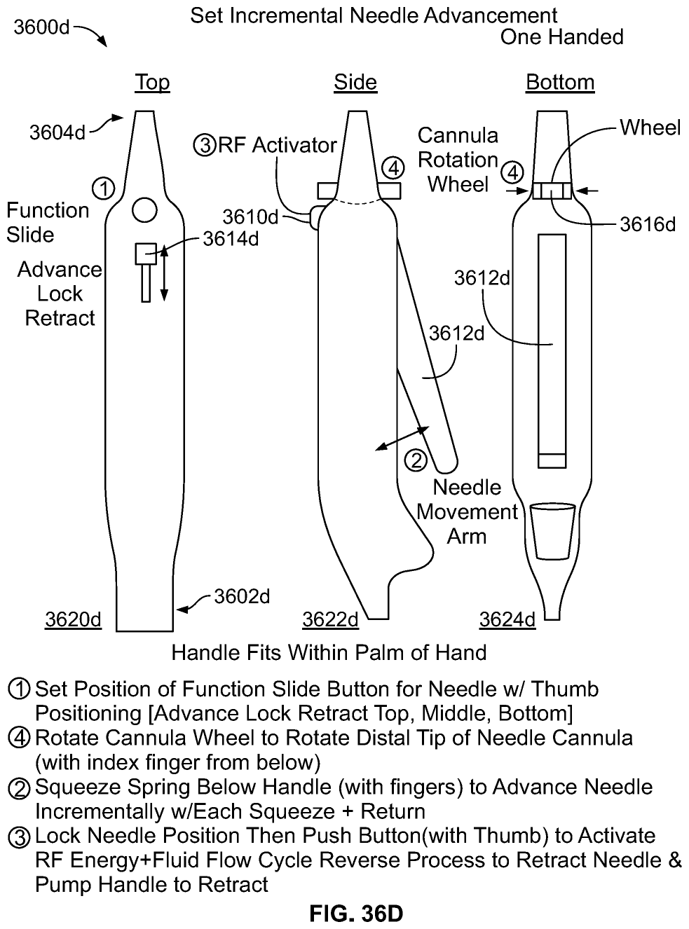

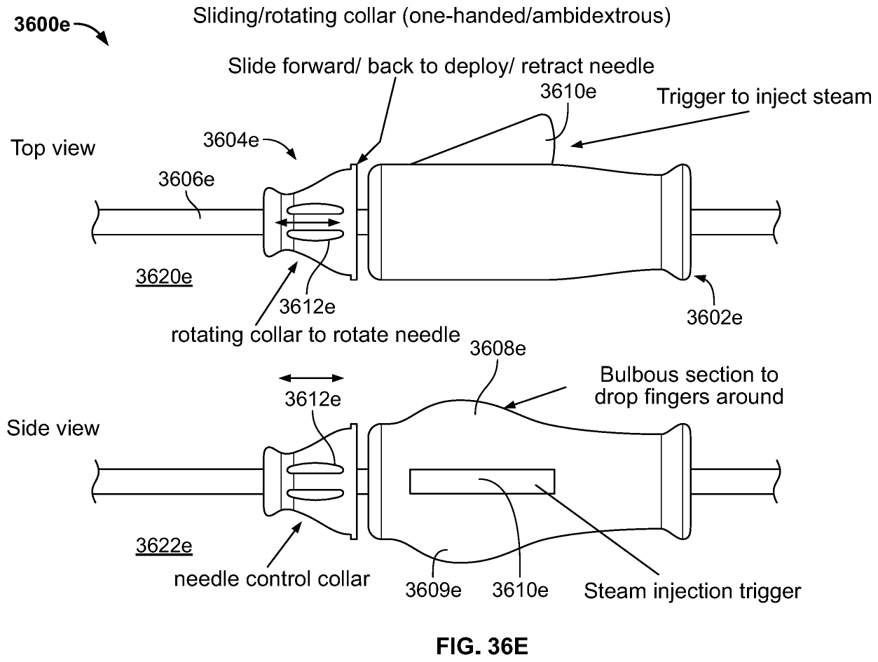

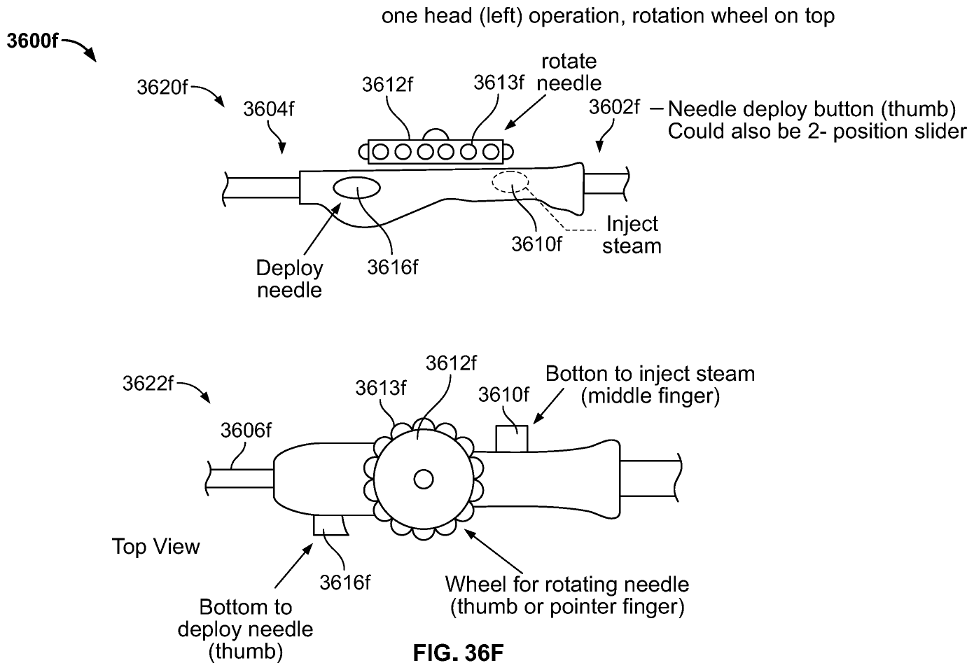

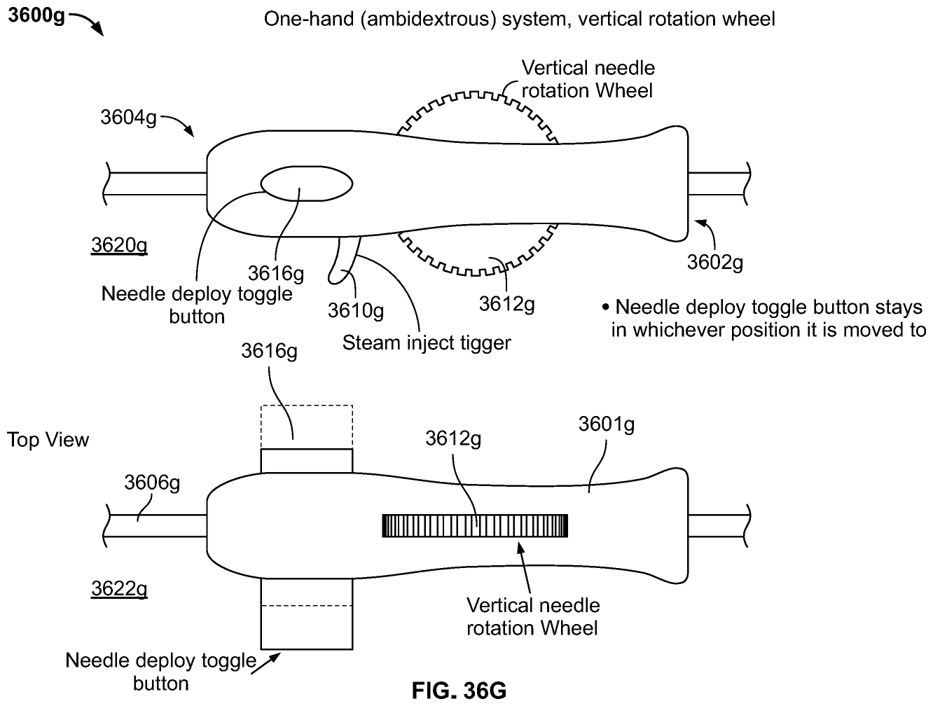

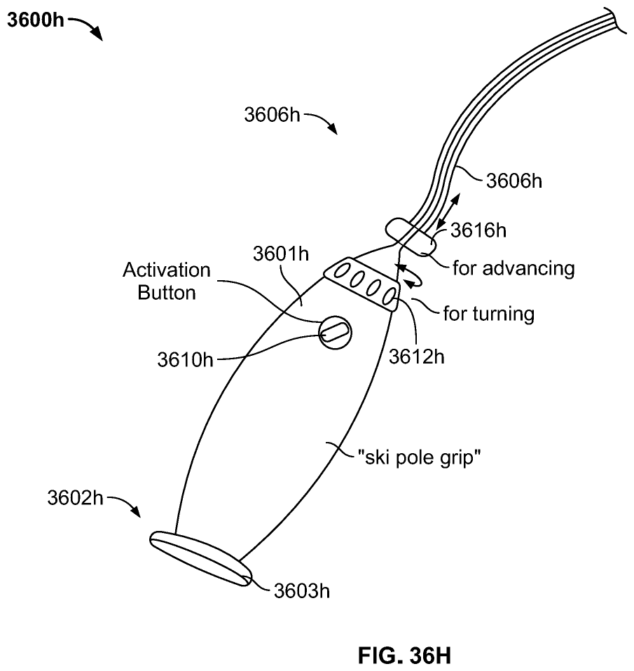

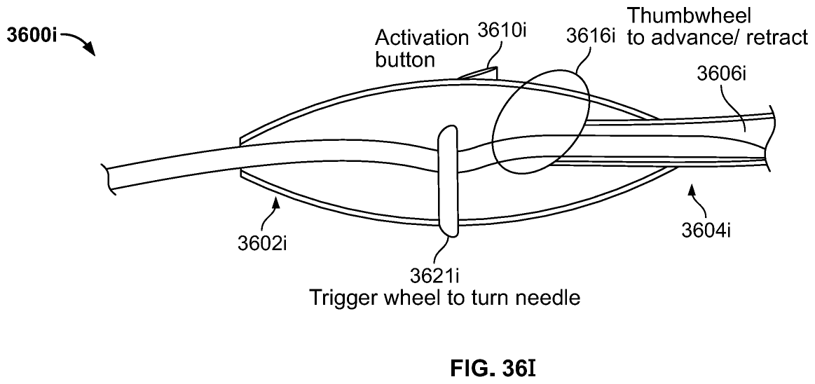

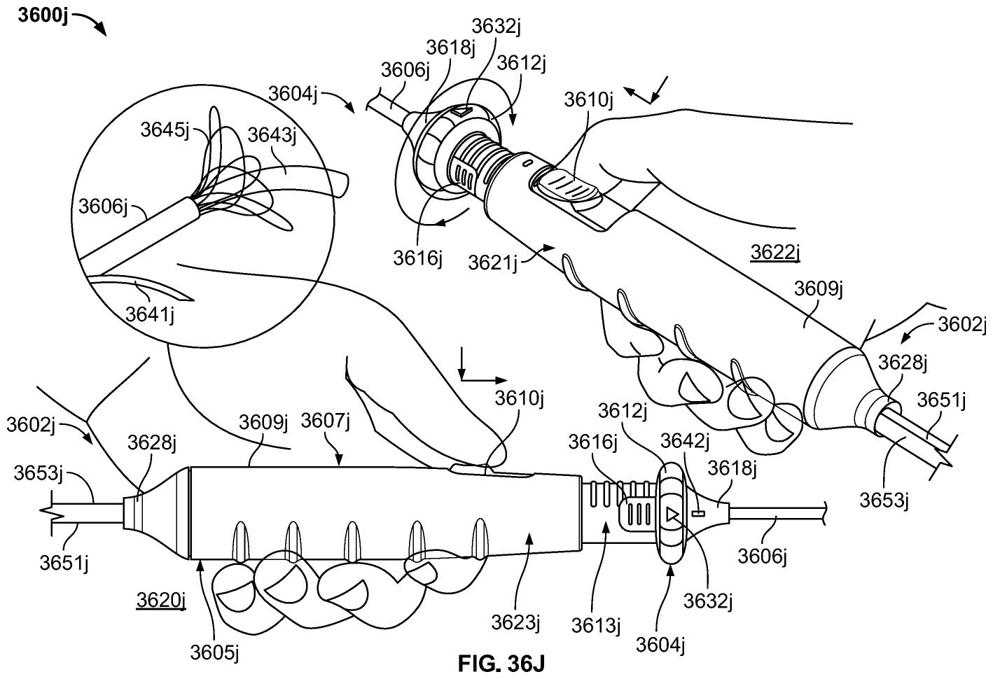

FIGS. 36A through 36J illustrate embodiments of handles to be used with the ablation systems of the present specification wherein the shape of the handle approximates that of a fishing rod, having an elongate, cylindrical length.

FIG. 36A illustrates an embodiment of a catheter handle mechanism 3600a in accordance with some embodiments of the present specification. The handle 3600a has an elongated tubular structure with a proximal end 3602a and a distal end 3604a, where the distal end corresponds to the end of the handle 3600a to which a catheter shaft 3606a is attached. Proximate the proximal end 3602a, the tubular body of the handle 3600a is slightly depressed and contoured 3608a in shape. In some embodiments, the contoured shape 3608a is provided on two opposite sides of the handle 3600a proximate its proximal end 3602a. The contours provide an ergonomic means to grip the handle 3600a during its use. The extent of the contours along a length of the handle is sufficient for a user to encompass the handle with their fingers curved around the contoured shape 3608a while holding the handle 3600a. A thumb of the user is free to operate other functions configured in the handle 3600a, such as and not limited to, a button 3610a configured to deliver steam for ablation, upon its pressing.

Additionally, the handle 3600a may include a rotating knob 3612a. An extent of rotation of the knob 3612a by the user may correspond to an equal amount of rotation of one or more needles at a distal end of the catheter shaft 3606a. The knob 3612a may also include a directional indicator 3614a that indicates a direction of the needle tip. In an embodiment, the indicator 3614a is a narrow, horizontally protruding portion on a small part of the circumference of the knob 3612a. In another embodiment, the indicator 3614a is a mark, such as an arrow that is printed on a top surface of the knob 3612a, so that the mark is visible to the user when the knob 3612A is rotated.

In embodiments, a button or a dial 3616a is provided along a length of the handle 3600a, to enable the user to control the advancement and retraction of the needle(s) at the distal end of the catheter shaft 3606a. The dial 3616a may be rotated in a forward direction to advance movement of the needle out from the distal end of catheter shaft 3606a, and an in a reverse direction to retract the needle into the shaft 3606a.

FIG. 36B illustrates another embodiment of a catheter handle mechanism 3600b in accordance with some embodiments of the present specification. The handle 3600b has an elongated tubular structure with a proximal end 3602b and a distal end 3604b, where the distal end corresponds to the end of the handle 3600b to which a catheter shaft 3606b is attached. At the proximal end 3602b, the tubular body of the handle 3600b has a slightly larger diameter than the remaining body of the handle 3600b, providing a disc-shaped structure 3603b at the proximal end 3602b. In some embodiments, the elongated tubular body of handle 3600b is provided with friction grips 3608b. The friction grips 3608b may be made of material such as rubber to provide an ergonomic grip to the user.

A button 3610b is provided on the handle 3600b, preferably proximate the distal end 3604b. The button 3610b enable the user to control the generation of steam for ablation during deployment of needles at a distal end of the catheter shaft 3606b. The button 3610b is moved forward by the user for generating steam. The steam is generated for as long as the user keeps the button 3610b in the forward direction. Steam generation is stopped or disabled as soon as the button 3610b is released such that it passively returns to its original position. In embodiments, button 3610b is configured to be press-locked at its original position and/or its forward position, for safety.

Additionally, the handle 3600a may include a sliding portion 3612b that is attached at one of the proximal end 3602b or distal end 3604b of the handle 3600b. In operation, sliding the sliding portion 3612b forward triggers a forward advancement of the one or more needles, for deployment, at the distal end of the catheter shaft 3606b. Similarly, sliding the sliding portion 3612b in a reverse direction, towards the proximal end 3602b of the handle 3600b, may trigger retraction of the needle(s). A surface of the sliding portion 3612b is marked with measurements to indicate the extent of movement of the needle(s). In some embodiments, the marks are supported with a haptic feedback feature in the form of small protrusions that indicate a unit of forward or reverse movement of the needle(s). In embodiments, the sliding portion 3612b may also be rotated to control rotation of the needle(s). An additional set of markings on the surface of the sliding portion 3612b may indicate the extent of rotation of the needle(s).

FIG. 36C illustrates another embodiment of a catheter handle mechanism 3600c in accordance with some embodiments of the present specification. The handle 3600c has an elongated tubular structure with a proximal end 3602c and a distal end 3604c, where the distal end corresponds to the end of the handle 3600c to which a catheter shaft 3606c is attached. In embodiments, a strain relief 3605c is included at the distal end 3604c of the handle 3600c to provide support to the catheter shaft 3606c. The handle 3600c is shaped like a pen, with a narrow opening at its distal end 3604c from where the shaft 3606c emerges. A button 3610c is configured proximate the distal end 3604c, to control the generation of steam. The button 3610c may be a press button, and may include a safety feature so that the button must be unlocked for operation to generate steam. A dial 3612c is configured on a side of the handle 3600c, proximate the distal end 3604c. The user may rotate dial 3612c to rotate needle(s) positioned at a distal end of shaft 3606c, during deployment. Further, a button 3616c is configured preferably in the middle of the length of the handle 3600c, which enables the user to control the forward and reverse movements of the needle(s). In embodiments, the button 3616c is a sliding button, and the extent of sliding the button 3616c in the forward or reverse directions determines the extent of forward and reverse movements of the needle(s). Markings 3614c are provided along the slidable length of the button 3616c which indicate the measurement of a distance that the needle(s) is extended from the distal end of the shaft 3606c.

FIG. 36D illustrates another embodiment of a catheter handle mechanism 3600d in accordance with some embodiments of the present specification. The figure illustrates three views—a top view 3620d, a side view 3622d, and a bottom view 3624d—of the handle 3600d. The handle 3600d has an elongated tubular structure with a proximal end 3602d and a distal end 3604d, where the distal end corresponds to the end of the handle 3600d to which a catheter shaft (not shown) is attached. At its distal end 3604d, the handle 3600c is tapered and shaped like a pen, with a narrow opening from which the shaft emerges. The proximal side of the handle 3600d is slightly bent downward to provide an ergonomic shape and enable a user to hold and operate the handle 3600d with a single hand. The handle 3600d is shaped similar in form and function to a caulking gun. A press button 3610d is provided proximal to the distal end 3604d to control the generation of steam for ablation. In embodiments, button 3610d is configured to control the system to deliver an RF signal to an electrode in the catheter to convert fluid to vapor for ablation. A lever 3612d, similar to the lever of a caulking gun, is provided along a length of one side of the handle. The user may squeeze the lever 3612d to advance the needle.

A function slide button 3614d is provided on the handle 3600d, preferably proximate its distal end. The button 3614d is configured to be slidable by the user to different positions along a length of the handle 3600d which results in positioning of one or more needles located at a distal end of the catheter shaft. In an embodiment, a first position of the button 3614d corresponds to advancing the position of the needle, a second (middle) position corresponds to locking the position of the needle, and a third position corresponds to retracting the needle from its position. A user may use the thumb of the hand that holds the handle 3600d to operate the button 3614d. A rotating wheel button proximate the distal end 3604d is configured to be operated by an index finger of the same hand of the user to manage rotation of a distal tip of a needle cannula.

FIG. 36E illustrates another embodiment of a catheter handle mechanism 3600e in accordance with some embodiments of the present specification. The figure illustrates two views—a top view 3620e, and a side view 3622e—of the handle 3600e. The handle 3600e has an elongated tubular structure with a proximal end 3602e and a distal end 3604e, where the distal end corresponds to the end of the handle 3600e to which a catheter shaft 3606e is attached. A middle section 3609e along a circular length of the handle 3600e is configured in a smooth bulbous shape to allow the user to hold and manage the handle's function with a single hand. A triangular button 3610e protrudes horizontally outwards from a side of the circular surface of the handle 36100e, which may be pressed by the user to activate steam generation. A distal section 3612e of the handle 3600e is configured as a needle control collar that may be rotated to rotate the needle, and slid forward and backward to control the advancement and retraction respectively, of the needle, from a distal end of the catheter shaft 3606e.

FIG. 36F illustrates another embodiment of a catheter handle mechanism 3600f in accordance with some embodiments of the present specification. The figure illustrates two views—a side view 3620f, and a top view 3622f—of the handle 3600f. The handle 3600f has an elongated tubular structure with a proximal end 3602f and a distal end 3604f, where the distal end corresponds to the end of the handle 3600f to which a catheter shaft 3606f is attached. A first button 3610f on a first side of the handle 3600f is provided to enable a user, in embodiments, to use their middle finger to operate the button to activate the generation of steam. In embodiments, the first button 3610f is positioned proximate the proximal end 3602f of the handle 3600f A second or top side of the handle 3600f, in some embodiments, 90 degrees rotated from the first side, is provided with a rotating wheel 3612f which may be controlled by the user with a thumb and pointer finger of the same hand that holds the handle 3600f, to rotate a needle positioned at a distal end of the shaft 3606f A flat surface of the rotating wheel 3612f is positioned horizontally relative to the surface of the handle 3600f In embodiments, the rotating wheel 3612f includes a plurality of tactile members 3613f that facilitate manipulation of the rotating wheel 3612f by the user. A second button 3616f is provided to control the deployment of the needle. The user's thumb may be used to manage button 3616f In different embodiments the button 3616f may be a press button or a two-position slide button. In embodiments, the second button 3616f may be pressed to deploy the needle from a distal end of the shaft 3606f and released to retract the needle. In other embodiments, the second button 3616f may be slid forward to extend the needle and slid back to retract the needle. In embodiments, the second button 3616f is positioned on a third side of the handle, opposite the first side, and at a position proximate the distal end 3604f of the handle 3600f.

FIG. 36G illustrates another embodiment of a catheter handle mechanism 3600g in accordance with some embodiments of the present specification. The figure illustrates two views—a side view 3620g, and a top view 3622g—of the handle 3600g. The handle 3600g has an elongated tubular structure with a proximal end 3602g and a distal end 3604g, where the distal end corresponds to the end of the handle 3600g to which a catheter shaft 3606g is attached. A trigger 3610g, in some embodiments positioned on a first side of the handle 3600g proximate the distal end 3604g, is provided to enable a user to use their index finger to operate the trigger so as to activate the generation of steam. A central portion of the handle 3600g is provided with a rotating wheel 3612g. In embodiments, the rotating wheel 3612g is positioned within, and extends from a top and bottom side of, a handle body 3601g of the handle 3600g. The rotating wheel 3612g is configured to by freely rotated within the handle body 3601g by a user. A circumference of the rotating wheel 3612g emerges from two opposite sides of the handle body 3601g of the handle. The rotating wheel 3612g allows the user to use a thumb or one of the fingers to rotate a needle positioned at a distal end of the shaft 3606g. Additionally, a button 3616g, in some embodiments positioned on a second side of the handle 90 degrees rotated from the first side of the handle and proximate the distal end 3604g, is provided to be used to deploy the needle from the catheter shaft 3606g.

FIG. 36H illustrates another embodiment of a catheter handle mechanism 3600h in accordance with some embodiments of the present specification. The handle 3600h has an elongated tubular structure with a proximal end 3602h and a distal end 3604h, where the distal end corresponds to the end of the handle 3600h to which a catheter shaft 3606h is attached. The tubular body of the handle 3600h has a varying diameter along its length such that a diameter of the handle 3600h at a center along its length is greater than a diameter of the handle 3600h at its distal and proximal ends, providing a ‘ski pole’ grip to the user. In embodiments, a disc shaped member 3603h is included at the proximal end 3602h of the handle 3600h to assist with securing a user's grip. A button 3610h on a side of the handle 3600h is provided to enable a user to use their index finger to operate the button 3610h so as to activate the generation of steam. A distal portion of the handle 3600h is provided with a first rotating wheel 3612h. A circumference of the first rotating wheel 3612h is greater than a circumference of the handle body 3601h at the distal portion. The first rotating wheel 3612h allows the user to use a thumb or one of the fingers to rotate a needle positioned at a distal end of the shaft 3606h. Additionally, a finger grip 3616h is provided proximate the distal end 3606h of the handle 3600h. In embodiments, the finger grip 3616h has a suture-wings shape, similar to a butterfly suture. The finger grip 3616h may be gripped by the used and moved in a longitudinal direction to move the catheter shaft 3606h back and forth to advance or retract the needle.

FIG. 36I illustrates yet another embodiment of a catheter handle mechanism 3600i in accordance with some embodiments of the present specification. The handle 3600i has an elongated tubular structure with a proximal end 3602i and a distal end 3604i, where the distal end corresponds to the end of the handle 3600i to which a catheter shaft 3606i is attached. The tubular body of the handle 3600i has a varying diameter along its length such that a diameter of the handle 3600h at a center along its length is greater than a diameter of the handle 3600h at its distal and proximal ends, providing a ‘ski pole’ grip to the user. A button 3610i, in embodiments positioned on a side of the handle 3610i and proximate a center of the handle 3600i along its length, is provided to enable a user to use their index finger to operate the button 3610i so as to activate the generation of steam. A first rotating wheel 3612i is positioned, in some embodiments, proximate the center of the handle 3600i along its length. The first rotating wheel 3612i allows the user to use a finger to rotate a needle positioned at a distal end of the shaft 3606i. Additionally, a second rotating wheel 3616i, in some embodiments positioned distal to the button 3610i and first rotating wheel 3612i along a length of the handle 3600i, is provided to be used to deploy the needle from the catheter shaft 3606i.

FIG. 36J illustrates another embodiment of a catheter handle mechanism 3600j in accordance with some embodiments of the present specification. The embodiments depicted in FIG. 36J include a combination of functions and features preferred in a fishing rod themed handle for controlling the ablation systems of the present specification. The figure illustrates two views of the handle 3600j—a side horizontal view 3620j and a top perspective view 3622j. The handle 3600j has a tubular structure with a proximal end 3602j and a distal end 3604j, and is smoothly curved to provide an ergonomic grip to the user. A catheter shaft 3606j extends from the distal end 3604j. In various embodiments, the catheter shaft 3606j extends from the distal end 3604j along a longitudinal axis of the handle 3600j. In some embodiments, the handle 3600j includes a first button 3616j on a portion proximate the distal end 3604j and positioned on a lateral first side 3621j or a lateral second side 3623j of the handle 3600j. The first button 3616j is configured such that the user's finger can press the button while also gripping the handle with the remaining fingers of the same hand. In some embodiments, the button 3616j is configured, when pressed on a first side 3621j, to incrementally or instantly retract a needle 3641j positioned at a distal end of a catheter shaft 3606j. In some embodiments, the button 3616j is configured, when pressed on a second side 3623j, to incrementally extend the needle 3641j positioned at a distal end of a catheter shaft 3606j. In some embodiments, the function of the button 3616j, when pressed, is reversed. In some embodiments, the handle 3600j includes a distal portion 3613j at its distal end 3604j which extends along the longitudinal axis of the handle 3600j and includes first button 3616j. The catheter shaft 3606j extends from the distal portion 3613j, in a same longitudinal axis of the distal portion 3613j. In embodiments, a first strain relief 3618j is positioned at a distal end of the distal portion 3613j and is configured to provide support to the catheter shaft 3606j as it exits from the distal portion 3613dj.

A second button 3610j is positioned on a top or third side 3607j proximate the distal end 3604j, and on the body 3609j of the handle 3600j, to activate steam generation. In other embodiments, the second button 3610j is positioned on a bottom or fourth side 3605j of the handle. In some embodiments, the second button 3610j is a press button with a safety feature that enables the user to lock the button when it does not need to be operated. In operation, the third button 3610j must be first slid forward and then can be pushed down to activate steam generation. In embodiments, the third button 3610j is a push and slide button that is configured to be pushed in and slid forward to activate steam generation. In embodiments, a rotating dial 3612j is included in the distal end 3604j of the handle, at a position distal to button 3616j, between first strain relief 3618j and distal portion 3613j, and is configured to be rotated by the user to cause the needle 3641j at the distal end of the shaft 3606j to rotate. In some embodiments, the rotating dial 3612j comprises debossed arrows 3632j and the handle 3600j includes degree indicator markings 3642j proximate the rotating dial 3612j to indicate to the user the degree of rotation of the needle 3641j when the arrow 3632j and specific marking 3642j align.

In some embodiments, the catheter shaft 3606j includes, at its distal end, the needle 3641j, a soft coude tip 3643j, and a positioning element 3645j configured to stabilize the catheter shaft 3606j in a bladder of a patient. The soft coude tip 3643j is configured to be atraumatic to body tissues during advancement of the catheter shaft 3606j. The handle further comprises a fluid line 3651j extending from its proximal end 3602j and through the body 3609j of the handle into the catheter shaft 3606j, configured to receive fluid for conversion into vapor. The handle 3600j further comprises a power line 3653j extending from its proximal end 3602j, through the body 3609j of the handle and into a proximal portion of the catheter shaft 3606j, configured to receive an electrical current to heat an electrode positioned within the proximal portion of the catheter shaft to convert the fluid to vapor for ablation. In some embodiments, a second strain relief 3628j is positioned at the proximal end 3602j of the handle to provide support to the fluid line 3651j and power line 3653j.

It should be noted that the various embodiments described in context of FIGS. 36A to 36J may use features and configurations from each other. In some embodiments, a structure and shape of a handle may be any of those described in the figures. Similarly, a combination of the type of control (button, slider, wheel, lever, or toggle) used to activate the generation of steam, the rotation and position of needle, can be selected from the different embodiments.

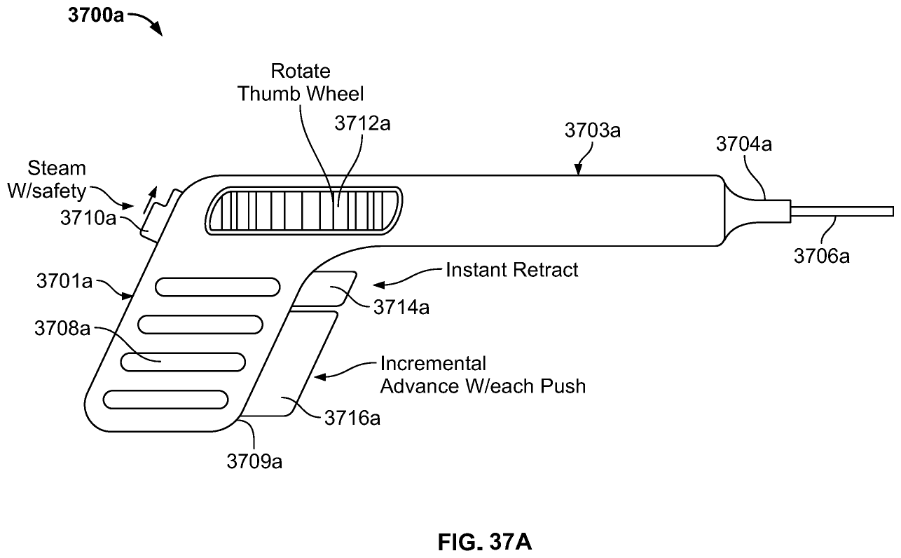

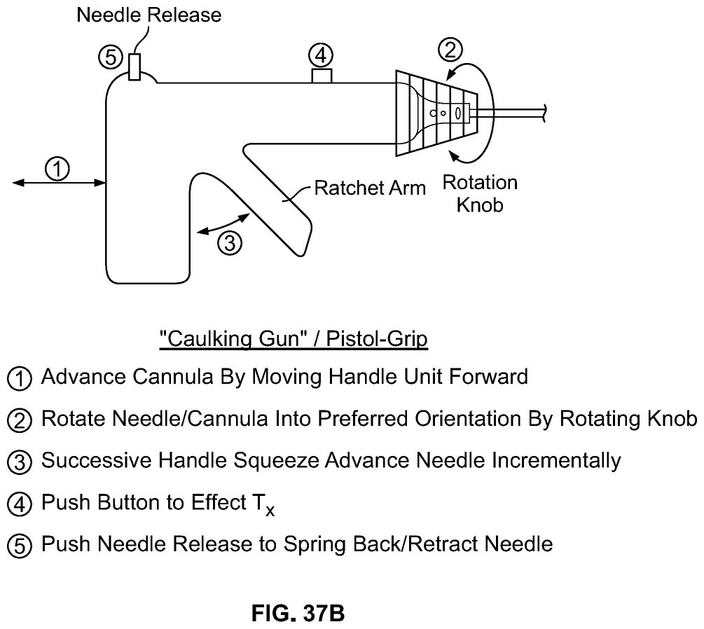

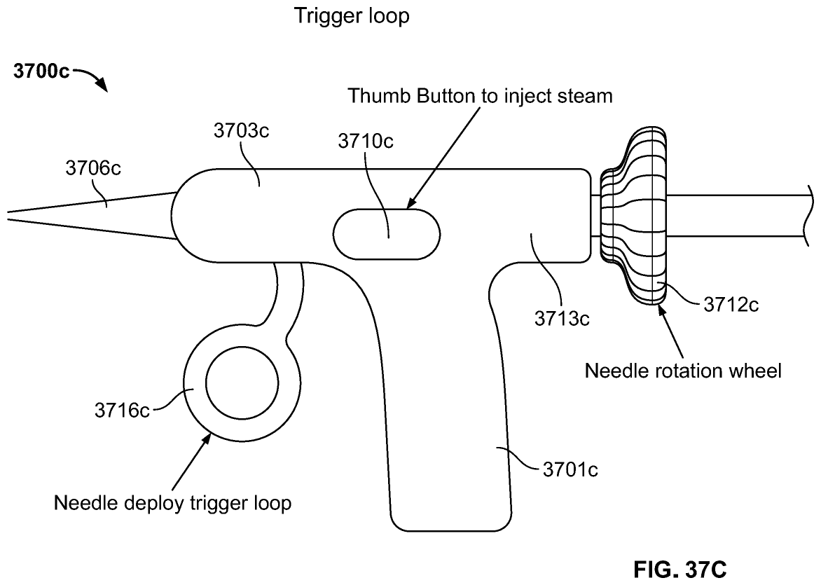

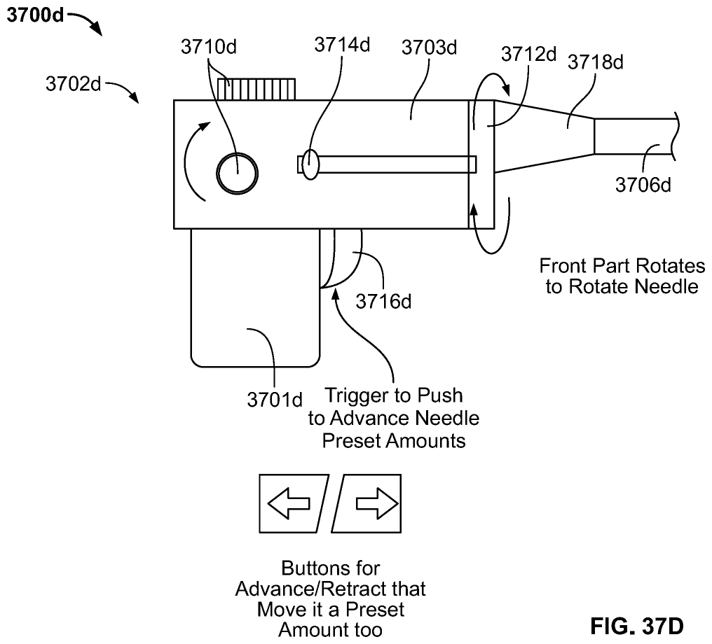

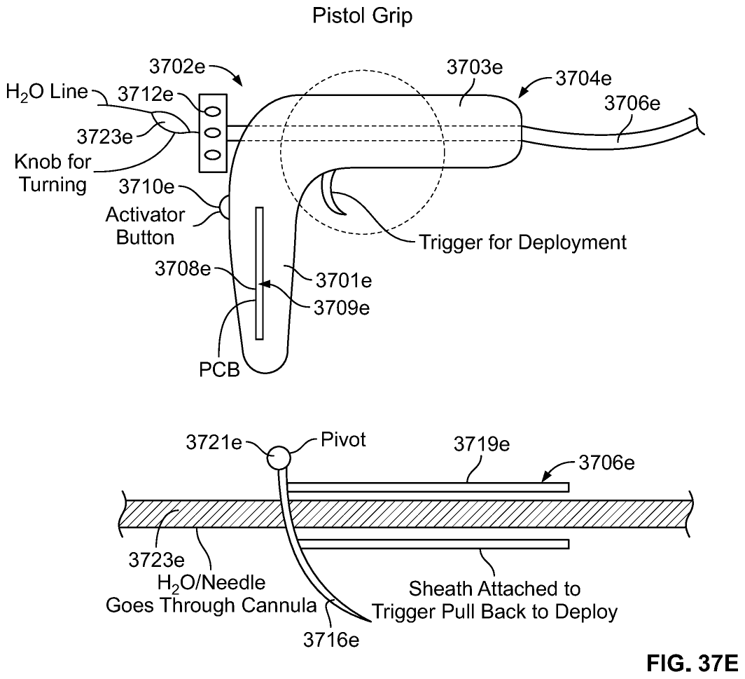

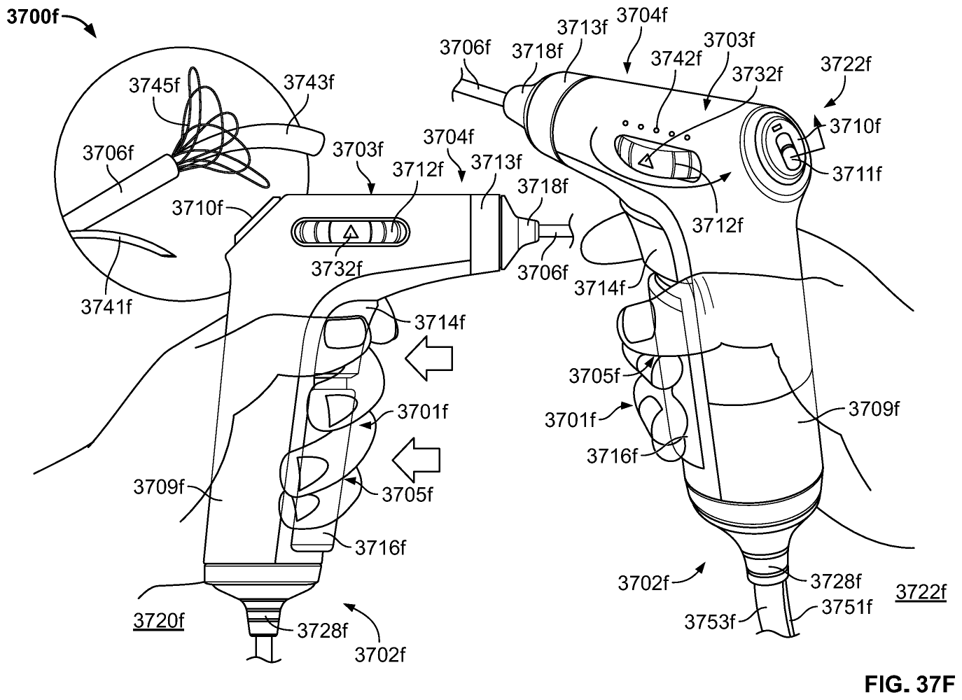

FIGS. 37A through 37F illustrate embodiments of handles to be used with the ablation systems of the present specification wherein the shape of the handle approximates that of a pistol grip, having first and second portions configured at angles in a range of 0 to 180 degrees to one another, such that the first portion is configured to be held in a user's hand and the second portion extends from an end of the first portion and includes a catheter extending therefrom.

FIG. 37A illustrates an embodiment of a catheter handle mechanism 3700a in accordance with some embodiments of the present specification. The handle 3700a is shaped like a handheld gun or a pistol, which allows it to be conveniently operated by a physician for the ablation treatment. The handle 3700a includes a first portion 3701a and a second portion 3703a, coupled together in a range of 0 to 180 degrees relative to one another, such that the first portion 3701a is configured to be held in a user's hand and the second portion 3703a extends from an end of the first portion 3701a and includes a catheter shaft 3706a extending therefrom. A strain relief 3704a, configured to provide support to the catheter shaft 3706a, is positioned at a distal end of the second portion 3702a. Ablation needles are coupled to the catheter shaft 3706a, as explained in the embodiments above, and are used to deliver steam vapor to a target tissue. In embodiments, the first portion 3701a is provided with friction grips 3708a to secure a grip of the user. The friction grips 3708a may be made of material such as rubber to provide an ergonomic grip to the user. A first button 3710a, in embodiments, positioned on a proximal top edge where the first portion 3701a and second portion 3703a connect, is provided to activate steam generation. In some embodiments, the first button 3710a is a press button with a safety feature that enables the user to lock the button when it does not need to be operated. In embodiments, the second portion 3703a of the handle 3700a includes a rotating wheel 3712a that rotates along a longitudinal length of the second portion 3702a. The rotating wheel 3712a allows the user to use a finger to rotate the wheel 3712a, which results in rotation of a needle(s) positioned at the distal end of the catheter shaft 3706a. Additionally, in some embodiments, a second button 3714a and a third button 3716a are provided along a distal facing surface 3709a of the first portion 3701a. In embodiments, the second button 3714a is configured to enable instant retracting of the needles when pressed, while the third button 3716a is configured to incremental advance the needle(s) by a preset distance, such as, for example, 5 mm, with each time that the third button 3716a is pressed.

FIG. 37b illustrates another embodiment of a catheter handle mechanism 3700b in accordance with some embodiments of the present specification. The handle 3700b is shaped like a handheld gun or a pistol, which allows it to be conveniently operated by a physician for the ablation treatment. The handle 3700b includes a first portion 3701b and a second portion 3703b, coupled together in a range of 0 to 180 degrees relative to one another, such that the first portion 3701b is configured to be held in a user's hand and the second portion 3703b extends from an end of the first portion 3701b and includes a catheter shaft 3706b extending therefrom. Ablation needles are coupled to the catheter shaft 3706b, as explained in the embodiments above, and are used to deliver steam vapor to a target tissue. In embodiments, the first portion 3701b may be provided with friction grips to provide an ergonomic grip to the user. A first button 3710b, in embodiments positioned on a top surface of the second portion 3703b of the handle 3700b, is provided to activate steam generation. In some embodiments, the first button 3710b is a press button with a safety feature that enables the user to lock the first button when it does not need to be operated. In embodiments, a rotating knob 3712b is included at a distal end of the second portion 3703b that may be rotated by the user to rotate a needle positioned at the distal end of the catheter shaft 3706b. A second button 3714b, in embodiments positioned on a proximal top edge where the first portion 3701b and second portion 3703b connect, is provided to enable the user to instantly retract the needle when pressed. In some embodiments, a ratchet arm 3716b is provided at a distal bottom surface where the first portion 3701b and second portion 3703b connect. The user may successively squeeze the ratchet arm 3716b for incrementally advancing the needle by a preset distance, such as, for example, 5 mm.

FIG. 37C illustrates another embodiment of a catheter handle mechanism 3700c in accordance with some embodiments of the present specification. The handle 3700c is shaped like a handheld gun or a pistol, which allows it to be conveniently operated by a physician for the ablation treatment. The handle 3700c includes a first portion 3701c and a second portion 3703c, coupled together in a range of 0 to 180 degrees relative to one another, such that the first portion 3701c is configured to be held in a user's hand and the second portion 3703c extends from an end of the first portion 3701c and includes a catheter shaft 3706c extending therefrom. In some embodiments, a proximal section 3713c of the second portion 3703c extends proximally beyond the intersection of the first portion 3701c and second portion 3703c. Ablation needles are coupled to the catheter shaft 3706c, as explained in the embodiments above, and are used to deliver steam vapor to a target tissue. The first portion 3701c may be provided with friction grips to provide an ergonomic grip to the user. A button 3710c on a side of the second portion 3702c of the handle 3700c is provided to activate steam generation. In some embodiments, the button 3710c is a press button with a safety feature that enables the user to lock the button when it does not need to be operated. In some embodiments, the proximal section 3713c of the second portion 3703c includes, at its proximal end, a rotation knob 3712c that may be rotated by the user to rotate a needle positioned at the distal end of the shaft 3706c. In embodiments, a circular trigger loop 3716c extends from a bottom surface of the second portion 3703c of the handle 3700c proximal the distal end of the handle 3700c. The user may successively squeeze the trigger loop 3716c for incrementally advancing the needle by a preset distance, such as, for example, 5 mm. In some embodiments, the trigger loop 3716c may be fully squeezed to retract the needle. In other embodiments, the rotation knob 3712c assists with needle retraction.

FIG. 37D illustrates another embodiment of a catheter handle mechanism 3700d in accordance with some embodiments of the present specification. The handle 3700d is shaped like a handheld gun or a pistol, which allows it to be conveniently operated by a physician for the ablation treatment. The handle 3700d includes a first portion 3701d and a second portion 3703d, coupled together in a range of 0 to 180 degrees relative to one another, such that the first portion 3701d is configured to be held in a user's hand and the second portion 3703d extends from an end of the first portion 3701d and includes a catheter shaft 3706d extending therefrom. Ablation needles are coupled to the catheter shaft 3706d, as explained in the embodiments above, and are used to deliver steam vapor to a target tissue. In some embodiments, the handle 3700d includes a strain relief 3718d at the distal end of the second portion 3703d to provide support to the catheter shaft 3706d. The first portion 3701d may be provided with friction grips to provide an ergonomic grip to the user. A button 3710d is provided to activate steam generation. In some embodiments, the button 3710b is positioned on a side of the second portion 3703d proximate a proximal end 3702d of the handle 3700d. In other embodiments, the button 3710b is positioned on atop surface of the second portion 3703d proximate a proximal end 3702d of the handle 3700d. In some embodiments, the button 3710b is a press or a rotate button with a safety feature that enables the user to lock the button when it does not need to be operated. A rotating knob 3712d is positioned at a distal end of the second portion 3712d and is configured to be rotated by the user to rotate a needle positioned at the distal end of the shaft 3706d. A slide button 3714d is provided on one or both sides of the second portion 3703d and is configured to be slid forward by a user to extend needles from a distal end of the catheter shaft 3706 and slid backward to retract the needles. In some embodiments, a trigger arm 3716d is provided at a distal edge where the first portion 3701d and second portion 3703d connect. The user may successively squeeze the trigger arm 3716d for incrementally advancing the needle by a preset distance, such as, for example, 5 mm. In other embodiments, the handle 3700d includes direction buttons 3715d which may be pushed by a user to incrementally advance or retract the needles a preset distance.

FIG. 37E illustrates another embodiment of a catheter handle mechanism 3700e in accordance with some embodiments of the present specification. The handle 3700e is shaped like a handheld gun or a pistol, which allows it to be conveniently operated by a physician for the ablation treatment. The handle 3700e includes a first portion 3701e and a second portion 3703e, coupled together in a range of 0 to 180 degrees relative to one another, such that the first portion 3701e is configured to be held in a user's hand and the second portion 3703e extends from an end of the first portion 3701e and includes a catheter shaft 3706e extending therefrom. Ablation needles are coupled to the catheter shaft 3706e, as explained in the embodiments above, and are used to deliver steam vapor to a target tissue. In embodiments, the first portion 3701e includes a slot 3709e for receiving a printed circuit board (PCB) 3708e, configured to removably place the PCB for controlling the handle 3700e. The first portion 3701e may be provided with friction grips to provide an ergonomic grip to the user. A button 3710e on a proximal surface of the first portion 3701e is provided to activate steam generation. In some embodiments, the button 3710e is a press or a rotate button with a safety feature that enables the user to lock the button when it does not need to be operated. A rotating knob 3712e, in embodiments positioned at a proximal end of the second portion 3703e, is configured to be rotated by the user to rotate a needle positioned at the distal end of the shaft 3706e. A trigger arm 3716e is provided on a bottom surface along a length of the section portion 3703e, extending downwards at an angle with the second portion 3703e of the handle 3700e. The trigger arm 3716e, in some embodiments, is shaped like an arc. A portion 3717e of the trigger arm 3716e extends inside the second portion 3703e of the handle 3700e and is attached to a sheath 3719e of the catheter shaft 3706e. The trigger arm 3716e is movable about a pivot point 3721e and, when the trigger arm 3716e is squeezed, the trigger arm 3716e is configured to pull the sheath 3719e back so as to deploy the needle from the catheter shaft 3706e. The user may successively squeeze the trigger arm 3716e for incrementally advancing the needle by a preset distance, such as, for example, 5 mm. A length of tubing 3723e is shown extending through the rotating knob 3712e, second portion 3703e, and catheter shaft 3706e and is configured to receive a fluid to be heated and converted to vapor for ablation.

FIG. 37F illustrates another embodiment of a catheter handle mechanism 3700f in accordance with some embodiments of the present specification. The embodiments depicted in FIG. 37F include a combination of functions and features preferred in a gun or pistol themed handle for controlling the ablation systems of the present specification. The figure illustrates two views of the handle 3700f—a side vertical view 3720f and a side vertical perspective view 3722f The handle 3700f includes a first portion 3701f and a second portion 3703f, coupled together in a range of 0 to 180 degrees relative to one another, such that the first portion 3701f is configured to be held in a user's hand and the second portion 3703f extends from an end of the first portion 3701f and includes a catheter shaft 3706f extending therefrom. The first portion 3701f of the handle 3700f has a tubular structure and is smoothly curved to provide an ergonomic grip to the user. A catheter shaft 3706f extends from a distal end 3704f of the second portion 3703f In various embodiments, the catheter shaft 3706f extends from the distal end 3704f along a longitudinal axis of the second portion 3703f of the handle 3700f In some embodiments, the handle 3700f includes a first button 3714f and a second button 3716f along a length of a distal side or first side 3705f of the first portion 3701f The first button 3714f and second button 3716f are configured such that the user's fingers can press the buttons while also gripping the handle with the same fingers. In some embodiments, the first button 3714f is configured, when pressed, to incrementally or instantly retract a needle 3741f positioned at a distal end of a catheter shaft 3706f, while the second button 3716f is configured, when pressed, to incrementally advance the needle 3741f. In other embodiments, the functions of the buttons 3714f, 3716f is reversed. The buttons 3714f, 3716f may be shaped so as to provide an ergonomic grip of the handle's 3700f first portion 3701f in the user's hand. In some embodiments, the handle 3700f includes a distal portion 3713f at its distal end 3704f which extends along the longitudinal axis of the second portion 3703f the handle 3700f. In embodiments, the catheter shaft 3706f extends from the distal portion 3713f in a same longitudinal axis of the distal portion 3713f. In embodiments, a first strain relief 3718f is positioned at a distal end of the distal portion 3713f and is configured to provide support to the catheter shaft 3706f as it exits from the distal portion 3713f.

A third button 3710f is positioned on a proximal end 3722f of the second portion 3703f where the first portion 3701f and the second portion 3703f meet, to activate steam generation. In some embodiments, the third button 3710f is a press button with a safety feature that enables the user to lock the button when it does not need to be operated. In embodiments, the third button 3710f includes a slide 3711f that must be first pushed forward or up to unlock the button 3710f. The entire button 3710f may then be pressed to activate steam generation. Sliding the slide 3711f back down locks the button 3710f and prevents inadvertent activation. In embodiments, a rotating dial 3712f is included in, and extends through, the second portion 3703f of the handle, such that portions of the rotating dial 3712f extend from the sides of the second portion and are accessible by a user. In embodiments, the rotating dial 3712f is configured to be rotated by the user to cause the needle 3741f at the distal end of the shaft 3706f to rotate. In some embodiments, the rotating dial 3712f comprises debossed arrows 3732f and the handle 3700f includes degree indicator markings 3742f proximate the rotating dial 3712f to indicate to the user the degree of rotation of the needle 3741f when the arrow 3732f and specific marking 3742f align.

In some embodiments, the catheter shaft 3706f includes, at its distal end, the needle 3741f, a soft coude tip 3743f, and a positioning element 3745f configured to stabilize the catheter shaft 3706f in a bladder of a patient. The soft coude tip 3743f is configured to be atraumatic to body tissues during advancement of the catheter shaft 3706f. The handle further comprises a fluid line 3751f extending from a proximal end 3702f of the first portion 3701f and through the body 3709f of the handle into the catheter shaft 3706f, configured to receive fluid for conversion into vapor. The handle 3700f further comprises a power line 3753f extending from the proximal end 3702f of the first portion 3701f, through the body 3709f of the handle and into a proximal portion of the catheter shaft 3706f, configured to receive an electrical current to heat an electrode positioned within the proximal portion of the catheter shaft to convert the fluid to vapor for ablation. In some embodiments, a second strain relief 3728f is positioned at the proximal end 3702f of the first portion 3701f to provide support to the fluid line 3751f and power line 3753f.

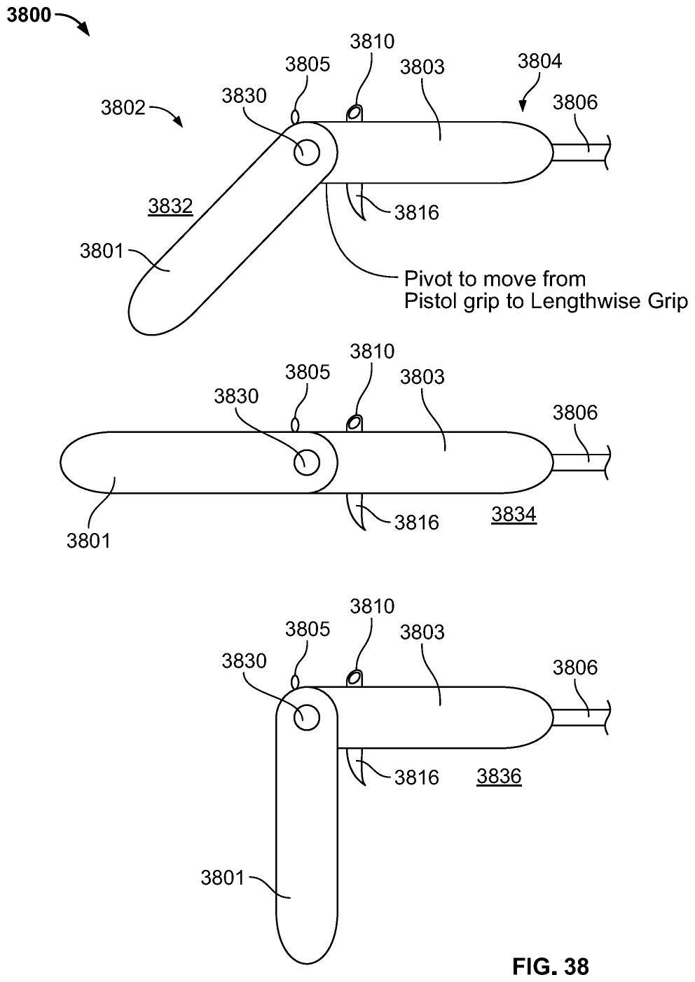

FIG. 38 illustrates another embodiment of a catheter handle mechanism 3800 in accordance with some embodiments of the present specification. A shape of the handle 3800 is configured to be adjusted around a pivot 3830, between a handheld gun or a pistol similar to the shapes illustrated in FIGS. 37A to 37E, and linear or tubular fishing rod similar to the shapes illustrated in FIGS. 36A to 361. The transformation of the shape of handle 3800 allows a user to adjust the handle 3800 to suit the grip that is convenient to the user for the ablation treatment, and is particularly useful in real-time imaging and ablation. The handle 3800 includes a first portion 3801 and a second portion 3803e coupled together at the pivot 3830 and movable in a range of 0 to 180 degrees relative to one another about the pivot 3830, such that the first portion 3801 is configured to be held in a user's hand and the second portion 3803 extends from an end of the first portion 3801 and includes a catheter shaft 3806 extending therefrom. The figure illustrates three side views of the handle 3800—view 3832 illustrates the structure of the handle 3800 when a first portion 3801 of the handle 3800 is at an angle between 90 and 180 degrees relative to second portion 3803 of the handle 3800; view 3834 illustrates the structure of the handle 3800 when the first portion 3801 is rotated around the pivot 3800 180 degrees relative to the second portion 3803, aligning the first portion 3801 horizontally with the second portion 3803, thereby providing a linear structure approximating a fishing rod shape and offering a lengthwise grip to the user; and, view 3836 illustrates the structure of the handle 3800 when the first portion 3801 of the handle 3800 is rotated around the pivot 3830 at an angle of 90 degrees relative to the second portion 3803 of the handle, creating a pistol grip for the user. In embodiments, the first portion 3801 may be rotated to any angle between 0 and 180 degrees relative to the second portion 3803, and may be fixed by the user at a selected angle by enabling a lock 3805. The lock 3805 may be disabled to allow rotation around the pivot 3830. In embodiments, a trigger arm 3816 may be provided at a bottom surface of the second portion 3803, which may be pressed to advance the needles in the catheter shaft 3806 for a preset increment of distance. In one embodiment, once the needles are advanced to the maximum distance by repeatedly pressing the trigger 3816, further pressing of the trigger results in retraction of the needles. The retraction may be in an instant, or one increment of distance at a time with each press of the trigger arm 3816. The handle 3800 may also include a press button 3810, in embodiments positioned on a top surface of the second portion 3803, to enable the user to activate or deactivate generation of steam for ablation.

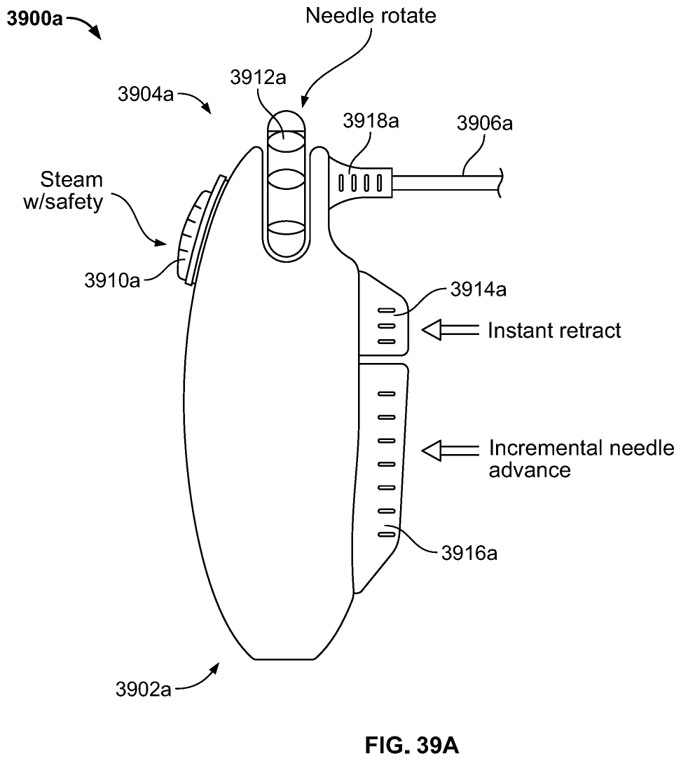

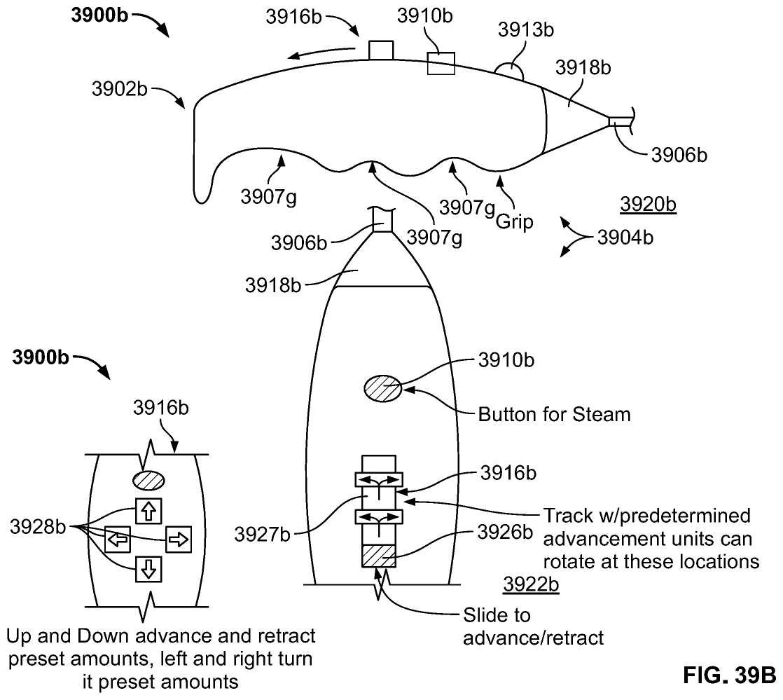

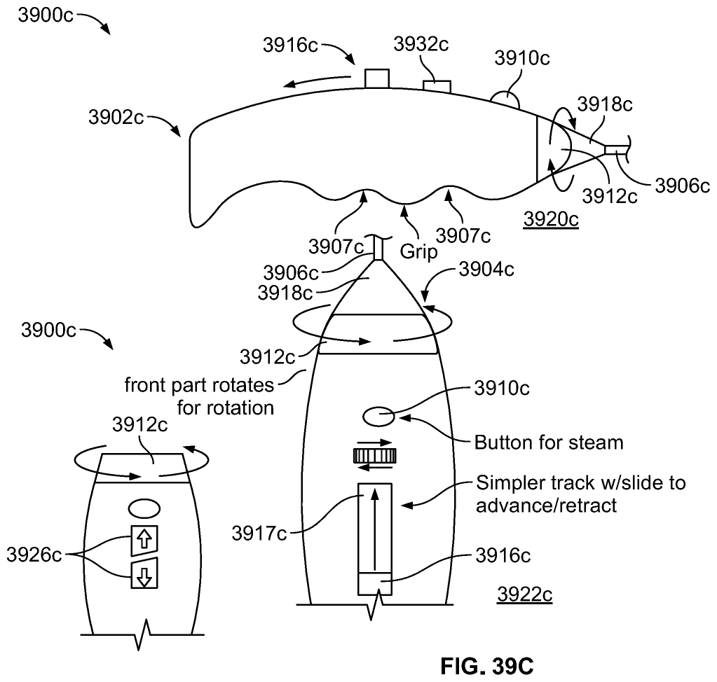

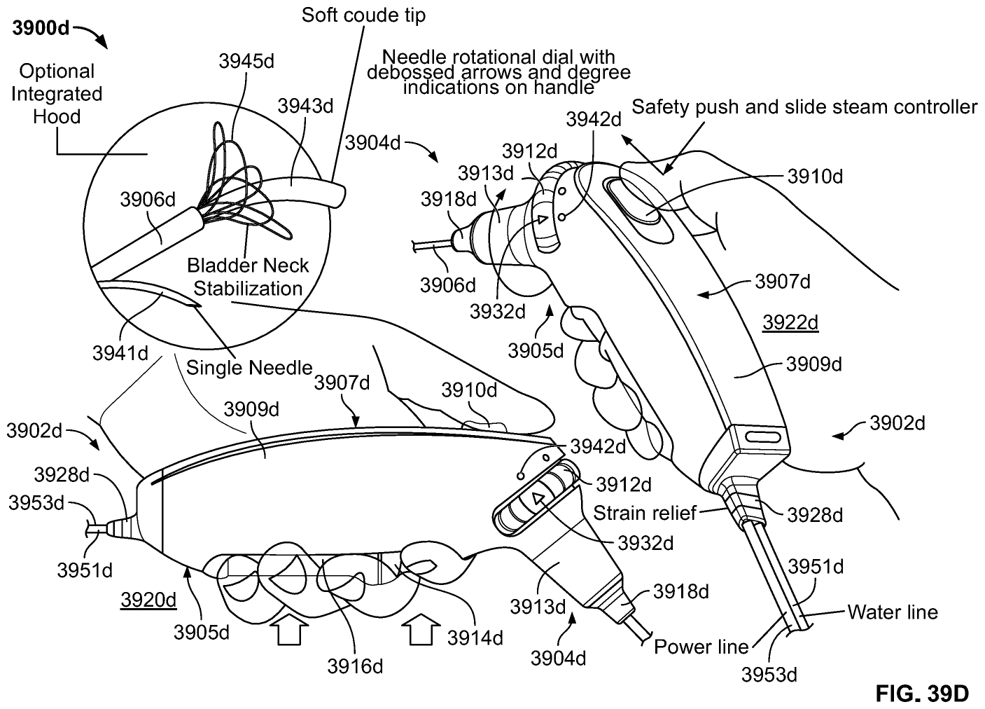

FIGS. 39A through 39D illustrate embodiments of handles to be used with the ablation systems of the present specification wherein the shape of the handle approximates that of a video game controller, comprising a plurality of actuators in the form of buttons, knobs, and slides and including a catheter shaft extending from a portion of the handle.

FIG. 39A illustrates another embodiment of a catheter handle mechanism 3900a in accordance with some embodiments of the present specification. The handle 3900a has a linear structure with a non-uniform diameter along its length. In embodiments, the handle 3900a has a diameter at a center point along its length that is greater than a diameter at its proximal end 3902a and distal end 3904a, and is smoothly curved to provide an ergonomic grip to the user. A catheter shaft 3906a is attached proximate a distal end 3904a of the handle 3900a. In an embodiment, the catheter shaft 3906a extends perpendicularly from the elongated linear structure of the handle 3900a. In some embodiments, a strain relief 3918a is included at the distal end 3904a of the handle 3900a to provide support to the catheter shaft 3906a as it exits from the handle's 3900a body. In embodiments, a rotating knob 3912a is positioned at the distal end 3904a, proximate the exit of the catheter shaft 3906a from the handle 3900a, and is configured to enable the user to rotate a needle positioned at the distal end of the shaft 3906a. In embodiments, the handle 3900a includes a first button 3916a and a second button 3914a on a first side of the handle 3900a. The needle is incrementally advanced by pressing the first button 3916a and retracted, either instantly or incrementally depending on the strength of the press, by pressing the second button 3914a. Buttons 3914a and 3916a may be positioned along the vertical length of the handle 3900a, aligned with and below the exit of the catheter shaft 3906a. A third button 3910a on a second side opposite the first side and positioned proximate the distal end 3904a of the handle, is provided to activate steam generation. In some embodiments, the third button 3910a is a press or a rotate button with a safety feature that enables the user to lock the third button 3910a when it does not need to be operated.

FIG. 39B illustrates another embodiment of a catheter handle mechanism 3900b in accordance with some embodiments of the present specification. The figure illustrates two views of the handle 3900b—a side horizontal view 3920b and a top horizontal view 3922b. The handle 3900b has a tubular structure with a proximal end 3902b and a distal end 3904b, and is smoothly curved to provide an ergonomic grip to the user. In some embodiments, the handle 3900b is shaped to provide smooth grooves 3907g around a partial circumference of the tubular body to place fingers for ergonomically gripping the handle 3900b in a user's hand. A catheter shaft 3906b extends from the distal end 3904b of the handle 3900b. The catheter shaft 3906b extends linearly along a longitudinal axis of the handle 3900b. In some embodiments, a strain relief 3918b is positioned at the distal end 3904b of the handle 3900b to provide support to the catheter shaft 3906b as it exits from the handle's 3900b body. A button 3910b is provided on a surface of the handle 3900b proximate the distal end 3904b, on a second side opposite a first side comprising the grooves 3907g for holding, to activate steam generation. In some embodiments, the button 3910b is a press or a rotate button with a safety feature that enables the user to lock the button when it does not need to be operated. In embodiments, a rotating knob 3913b may be provided on the surface of the handle, which enables the user to rotate a needle positioned at the distal end of the shaft 3906b. Alternatively, a combination of buttons 3916b may be provided to manipulate the movement of the needles for rotating as well as advancing and retracting. In an embodiment, the combination of buttons 3916b includes a slider button 3926b that is moved towards the distal end 3904b for advancing and towards the proximal end 3902b for retracting the needle. The same button may be slid or switched sideways to effectuate incremental rotation of the needle. The track 3927b for sliding may be marked with predetermined amounts of distance that the needle may be advanced or retracted. The needle may also be incrementally advanced and retracted by sliding button 3926b in the required direction. Combination buttons 3916b may be positioned along the longitudinal length of the handle 3900b, aligned with the button 3910b for activating generation of steam. In some alternate embodiments, the combination of button 3916b include a set of four directional buttons 3928b with arrows indicating the direction of movement (advance, retract, left rotation, right rotation), to effectuate an incremental movement of needles in that direction.

FIG. 39C illustrates another embodiment of a catheter handle mechanism 3900c in accordance with some embodiments of the present specification. The figure illustrates two views of the handle 3900c—a side horizontal view 3920c and a top horizontal view 3922c. The handle 3900c has a tubular structure with a proximal end 3902c and a distal end 3904c, and is smoothly curved to provide an ergonomic grip to the user. In some embodiments, the handle 3900c is shaped to provide smooth grooves 3907c around a partial circumference of the tubular body to place fingers for ergonomically gripping the handle 3900c in a user's hand. A catheter shaft 3906c extends from the distal end 3904c of the handle 3900c. The catheter shaft 3906c extends linearly along a longitudinal axis of the handle 3900c. In some embodiments, a strain relief 3918c is positioned at the distal end 3904c of the handle 3900c to provide support to the catheter shaft 3906c as it exits from the handle's 3900c body. In embodiments, a first rotating knob 3912c is positioned at a distal end 3904c of the handle 3900c, proximal to the strain relief 3918c, and is configured to be rotated by the user to rotate a needle positioned at the distal end of the shaft 3906c. In other embodiments, a rotating knob 3932c is positioned on a second side of the handle 3900c, opposite to a first side configured with the grooves 3907c, with or without the inclusion of the first rotating knob 3912c, and serves the same function of the first rotating knob 3912c. Positioning the rotating knob 3932c on the side of the handle 3900c may be preferred for single-hand use such that the user, while gripping the handle 3900c, is able to use a finger of the same hand to operate the rotating knob 3932c. In embodiments, a sliding button 3916c is positioned on the second side of the handle 3900c and is configured to be slid within a track 3917c towards the distal end 3904c so that the needle is incrementally advanced within extended from the catheter shaft 3906c. Similarly, the button 3916c is slid towards the proximal end to retract the needle. Alternatively, a combination of directional buttons 3926c may be provided to manipulate the movement of the needles for advancing and retracting. In an embodiment, the combination of buttons 3926c includes a press button pointing towards the distal end 3904c for advancing and another press button pointing towards the proximal end 3902c for retracting the needle. Combination buttons 3926c may be positioned along the longitudinal length of the handle 3900c, aligned with the button 3910c for activating generation of steam.

FIG. 39D illustrates another embodiment of a catheter handle mechanism 3900d in accordance with some embodiments of the present specification. The embodiments depicted in FIG. 39D include a combination of functions and features preferred in a video game controller themed handle for controlling the ablation systems of the present specification. The figure illustrates two views of the handle 3900d—a side horizontal view 3920d and a top perspective view 3922d. The handle 3900d has a tubular structure with a proximal end 3902d and a distal end 3904d, and is smoothly curved to provide an ergonomic grip to the user. A catheter shaft 3906d extends from the distal end 3904d. In various embodiments, the catheter shaft 3906d extends from the distal end 3904d at an angle ranging from 0 to 180 degrees from a longitudinal axis of the handle 3900d. In some embodiments, the handle 3900d includes a first button 3914d and a second button 3916d along a length of a bottom or first side 3905d of the handle 3900d. The first button 3914d and second button 3916d are configured such that the user's fingers can press the buttons while also gripping the handle with the same fingers. In some embodiments, the first button 3914d is configured, when pressed, to incrementally or instantly retract a needle 3941d positioned at a distal end of a catheter shaft 3906d, while the second button 3916d is configured, when pressed, to incrementally advance the needle 3941d. In other embodiments, the functions of the buttons 3914d, 3916d is reversed. The buttons 3914d, 3916d may be shaped to as to provide an ergonomic grip of the handle 3900d in the user's hand. In some embodiments, the handle 3900d includes a distal portion 3913d at its distal end 3904d which extends at an angle relative to the longitudinal axis of the handle 3900d. In embodiments, the angle ranges between 0 and 180 degrees. The catheter shaft 3906d extends from the distal portion 3913d, in a same longitudinal axis of the distal portion 3913d. In embodiments, a first strain relief 3918d is positioned at a distal end of the distal portion 3913d and is configured to provide support to the catheter shaft 3906d as it exits from the distal portion 3913d.

A third button 3910d is positioned on a top or second side 3907d proximate the distal end 3904d, and opposite the buttons 3914d and 3916d on the first side 3905d, to activate steam generation. In some embodiments, the third button 3910d is a press or a rotate button with a safety feature that enables the user to lock the button when it does not need to be operated. In embodiments, the third button 3910d is a push and slide button that is configured to be pushed in and slid forward to activate steam generation. In embodiments, a rotating dial 3912d is included in the distal end 3904d of the handle, at a position where the distal end 3904d and distal portion 3913d meet, and is configured to be rotated by the user to cause the needle 3941d at the distal end of the shaft 3906d to rotate. In some embodiments, the rotating dial 3912d comprises debossed arrows 3932d and the handle 3900d includes degree indicator markings 3942d proximate the rotating dial 3912d to indicate to the user the degree of rotation of the needle 3941d when the arrow 3932d and specific marking 3942d align.

In some embodiments, the catheter shaft 3906d includes, at its distal end, the needle 3941d, a soft coude tip 3943d, and a positioning element 3945 configured to stabilize the catheter shaft 3906d in a bladder of a patient. The soft coude tip 3943d is configured to be atraumatic to body tissues during advancement of the catheter shaft 3906d. The handle further comprises a fluid line 3951 extending from its proximal end 3902d and through the body 3909d of the handle into the catheter shaft 3906d, configured to receive fluid for conversion into vapor. The handle 3900d further comprises a power line 3953 extending from its proximal end 3902d, through the body 3909d of the handle and into a proximal portion of the catheter shaft 3906d, configured to receive an electrical current to heat an electrode positioned within the proximal portion of the catheter shaft to convert the fluid to vapor for ablation. In some embodiments, a second strain relief 3928d is positioned at the proximal end 3902d of the handle to provide support to the fluid line 3951d and power line 3953d.

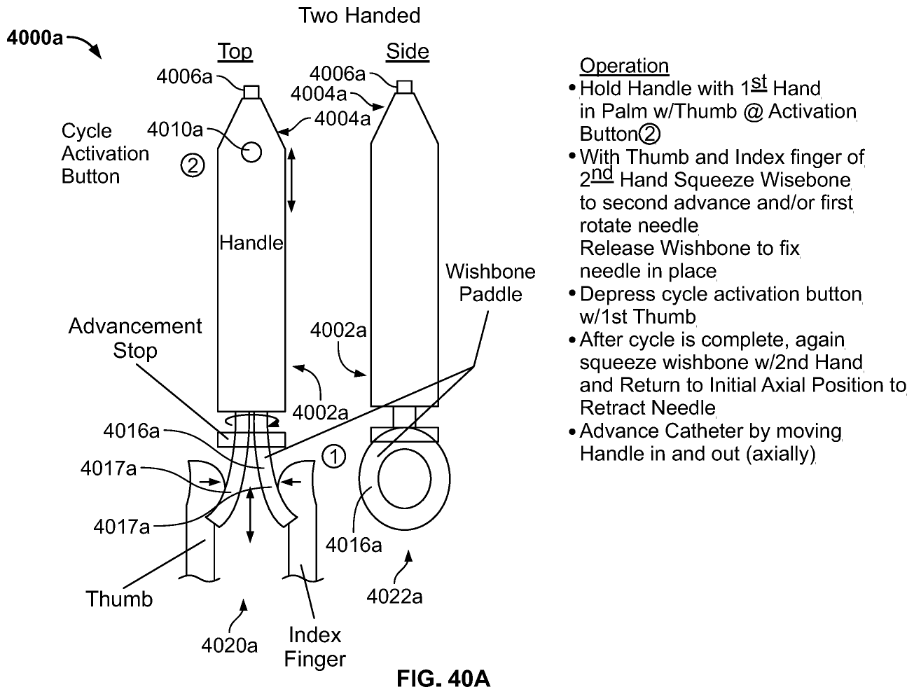

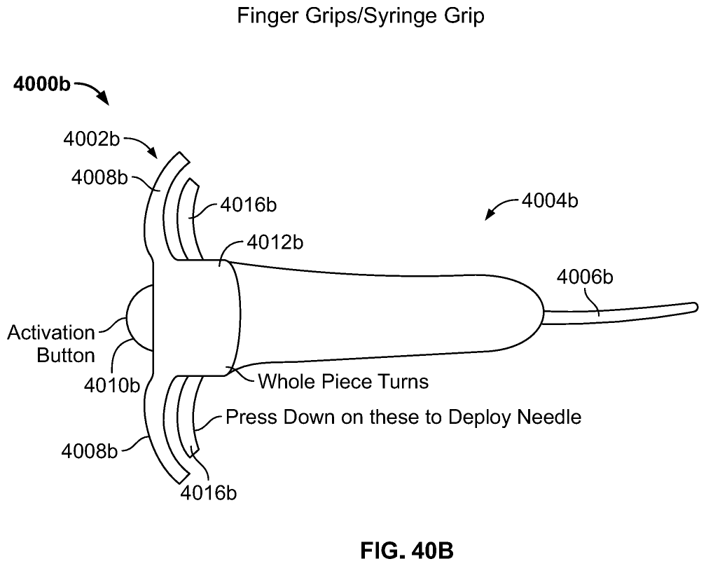

FIGS. 40A through 41 illustrate embodiments of handles to be used with the ablation systems of the present specification wherein the shape of the handle approximates that of a syringe, comprising an actuation mechanism at a proximal end of the handle and a catheter shaft extending from a distal end of the handle.

FIG. 40A illustrates another embodiment of a catheter handle mechanism 4000a in accordance with some embodiments of the present specification. The figure illustrates two views of the handle 4000a—a top view 4020a and a side view 4022a. The handle 4000a is configured to operate similar to a syringe. The handle 4000a is shaped in the form of an elongated tubular structure, with a distal end 4004a that is tapered to have a conical shape and a diameter that decreases at it extend distally to provide an exit for a catheter shaft 4006a. A proximal end 4002a is provided with an actuation mechanism 4016a, in an embodiment, comprising a wishbone-shaped paddle with two actuation members 4017a, that is configured to rotate and move axially in and out of the handle 4000a. The actuation mechanism 4016a is rotated by the user to achieve the desired rotational position of a needle at a distal end of the catheter shaft. In an embodiment, the user holds the handle 4000a in the palm of a first hand. The user may use the thumb of the first hand to operate a button 4010a on a surface near the distal end 4004a to activate steam generation. In some embodiments, the button 4010a is a press or a rotate button with a safety feature that enables the user to lock the button when it does not need to be operated. Using the thumb and index finger of the second hand, the user rotates the actuation mechanism 4016a to the desired rotary position for the needle, and then advances or retracts the needle by squeezing the actuation members 4017a of the actuation mechanism 4016a toward each other. The actuation mechanism 4016a may be released by the user to fix the position of the needle. The distal end of the catheter shaft 4006a may be advanced and retracted by moving the actuation mechanism 4016a forward and backward at the proximal end 4002a, axially into and out of the handle 4000a.

FIG. 40B illustrates another embodiment of a catheter handle mechanism 4000b in accordance with some embodiments of the present specification. The handle 4000b is configured to operate similar to a syringe. The handle 4000b is shaped in the form of an elongated tubular structure, with a rounded distal end 4004b from which a catheter shaft 4006b extends. A proximal end 4002b of the handle is provided with an actuation mechanism 4012b which comprises a finger grip 4008b and triggers 4016b. In some embodiments, the finger grip 4008b comprises a disc at the proximal end of the actuation mechanism 401bb. In other embodiments, the finger grip 4008b comprises at least two arms extending from opposite sides of the actuation mechanism 4012b. The triggers 4016b extend from the body of the actuation mechanism distal to the finger grip 4008b and are configured to be pressed in a proximal direction to cause a needle to be deployed from a distal end of the catheter shaft 4006b. The actuation mechanism 4012b further includes a button 4010b on its proximal end to activate steam generation. In some embodiments, the button 4010b is a press or a rotate button with a safety feature that enables the user to lock the button when it does not need to be operated. The actuation mechanism is further configured to be rotatable around a longitudinal axis of the handle 4000b. The actuation mechanism 4012b is rotated by the user to cause the needle at the distal end of the catheter shaft to rotate. The triggers 4016b extending outwards from the actuation mechanism 4012b may be pressed down by the user to advance the needle at the distal end of the catheter shaft 4006b.

FIG. 41 illustrates another embodiment of a catheter handle mechanism 4100 in accordance with some embodiments of the present specification. The embodiments depicted in FIG. 41 include a combination of functions and features preferred in a syringe themed handle for controlling the ablation systems of the present specification. The figure illustrates two views of the handle 4100—a side horizontal view 4120 and a top perspective view 4122. The handle 4100 has a tubular structure with a proximal end 4102 and a distal end 4104, and is smoothly curved to provide an ergonomic grip to the user. A catheter shaft 4106 extends from the distal end 4104. In various embodiments, the catheter shaft 4106 extends along a longitudinal axis of the handle 4100. In some embodiments, the handle 4100 includes a plunger mechanism 4116 comprising an elongate plunger body 4117 connected at its proximal end to a paddle handle 4112, wherein the distal end of the plunger body 4117 is configured to be movable coaxially into and out of the proximal end 4102 of the handle 4100. A user may grip the paddle handle 4112 to push and pull the plunger body 4117 into and out of the distal end 4102 of the handle body 4109 so as to incrementally advance or retract the needle 4141. The paddle handle 4112 may include ridges 4113 to provide a textured grip for the user. The plunger body 4117 includes a plurality of markings 4119 to indicate to the user the distance the needle 4141 has been advanced beyond the distal end of the catheter shaft 4106. In embodiments, the plunger mechanism 4116 is configured to be rotated by the user, rotating the plunger body 4117 within the handle body 4109, to cause the needle 4141 at the distal end of the shaft 4106 to rotate. In some embodiments, the paddle handle 4112 comprises debossed arrows 4132 and the handle body 4109 includes degree indicator markings 4142 proximate its proximal end 4102 to indicate to the user the degree of rotation of the needle 4141 when the arrow 4132 and specific marking 4142 align. In embodiments, a first strain relief 4118 is positioned at the distal end 4103 of the handle 4100 and is configured to provide support to the catheter shaft 4106 as it exits from the distal end 4104.

A first button 4110 is positioned on a top or first side 4107 of the handle 4100, proximate the distal end 4104, to activate steam generation. In other embodiments, the first button 4110 is positioned on a bottom or second side 4105 of the handle 4100, opposite the first side 4107. In some embodiments, the first button 4110 is a press button with a safety feature that enables the user to lock the button when it does not need to be operated. In operation, the first button 4110 must be first slid forward and then can be pushed down to activate steam generation. In embodiments, the first button 4110 is a push and slide button that is configured to be pushed in and slid forward to activate steam generation.

In some embodiments, the catheter shaft 4106 includes, at its distal end, the needle 4141, a soft coude tip 4143, and a positioning element 4145 configured to stabilize the catheter shaft 4106 in a bladder of a patient. The soft coude tip 4143 is configured to be atraumatic to body tissues during advancement of the catheter shaft 4106. In some embodiments, the bottom or second side 4105 of the handle 4100 comprises a finger grip 4115 which is contoured with a plurality of grooves 4125 and is configured to provide an ergonomic grip to the user. In embodiments, the finger grip 4115 is composed of a less rigid material relative to the material of the handle body 4109 to provide the user a comfortable grip. The handle 4100 further comprises a fluid line 4151 extending from a proximal end of the finger grip 4115 and through the body 4109 of the handle into the catheter shaft 4106, configured to receive fluid for conversion into vapor. The handle 4100 further comprises a power line 4153 extending from the proximal end of the finger grip 4115, through the body 4109 of the handle and into a proximal portion of the catheter shaft 4106, configured to receive an electrical current to heat an electrode positioned within the proximal portion of the catheter shaft to convert the fluid to vapor for ablation. In some embodiments, a second strain relief 4128 is positioned at the proximal end of the finger grip 4115 to provide support to the fluid line 4151 and power line 4153.

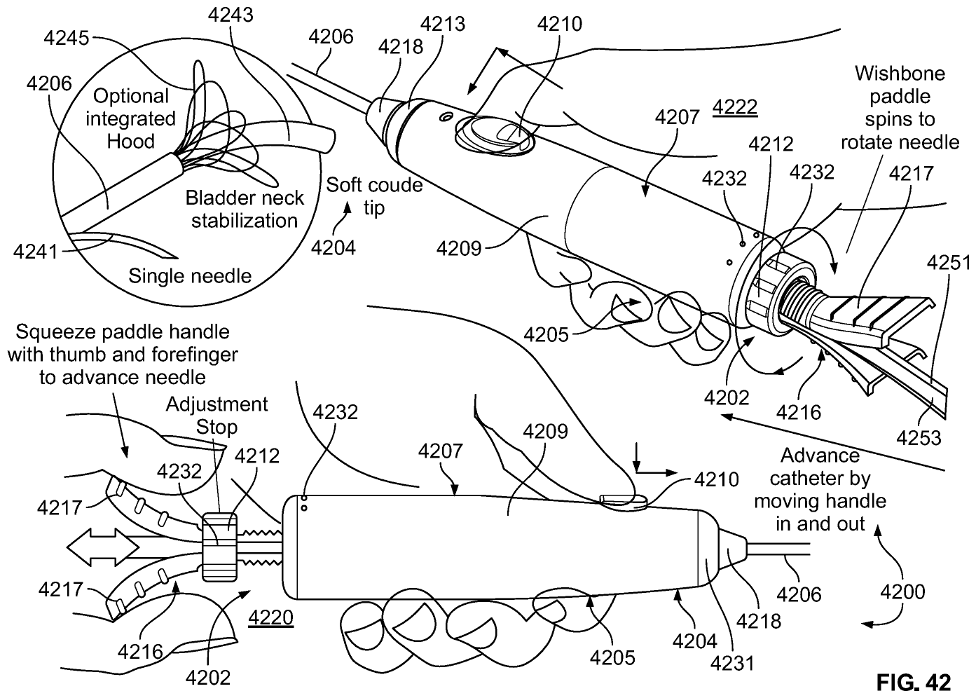

FIG. 42 illustrates another embodiment of a catheter handle mechanism 4200 in accordance with some embodiments of the present specification. The embodiments depicted in FIG. 42 include a combination of functions and features preferred in a handle having a wishbone-shaped paddle actuation mechanism for controlling the ablation systems of the present specification. The figure illustrates two views of the handle 4200—a side horizontal view 4220 and a top perspective view 4222. The handle 4200 has a tubular structure with a proximal end 4202 and a distal end 4204, and is smoothly curved to provide an ergonomic grip to the user. A catheter shaft 4206 extends from the distal end 4204. In various embodiments, the catheter shaft 4206 extends along a longitudinal axis of the handle 4200. In some embodiments, the handle 4200 includes an actuation mechanism 4216 comprising two actuation mechanism arms 4217 and an adjustment stop 4212 at the proximal end 4202. In embodiments, the actuation mechanism comprises a wishbone-shaped paddle 4216, the actuation mechanism arms comprise two paddle-shaped arms 4217, and the adjustment stop 4212 comprises a rotating wheel. A user uses a thumb and a forefinger of the same hand to squeeze the pair of paddle arms 4217 so as to incrementally advance or retract the needle 4241. The needle 4241 may be retracted by fully pressing the paddle arms 4217 together. The paddles 4217 may be shaped to as to provide an ergonomic grip between the user's fingers. The wishbone-shaped paddle 4216 is configured to be moved longitudinally into and out of the proximal end 4202 of the handle body 4209 to advance and retract the catheter shaft 4206. The adjustment stop 4212 is configured to be rotated to move the adjustment stop 4212 longitudinally along a length of the actuation mechanism or wishbone-shaped paddle 4216 to but up against the proximal end 4202, preventing further longitudinal movement of the wishbone-shaped paddle 4216 and thereby locking the catheter shaft 4206 at a desired distance from the distal end 4204 of the handle 4200. In embodiments, the user rotates the wishbone-shaped paddle 4216 to cause the needle 4241 at the distal end of the shaft 4206 to rotate. In some embodiments, the wishbone-shaped paddle 4212 comprises debossed arrows 4232 and the handle 4200 includes degree indicator markings 4242 proximate the wishbone-shaped paddle to indicate to the user the degree of rotation of the needle 4241 when the arrow 4232 and specific marking 4242 align. In some embodiments, the handle 4200 includes a distal portion 4213 at its distal end 4204 which extends along the longitudinal axis of the handle 4200. The catheter shaft 4206 extends from the distal portion 4213, in a same longitudinal axis of the distal portion 4213. In embodiments, a first strain relief 4218 is positioned at a distal end of the distal portion 4213 and is configured to provide support to the catheter shaft 4206 as it exits from the distal portion 4213.

A first button 4210 is positioned on a top or first side 4207 of the handle 4200, proximate the distal end 4204, to activate steam generation. In other embodiments, the first button 4210 is positioned on a bottom or second side 4205 of the handle 4200, opposite the first side 4207. In some embodiments, the first button 4210 is a press button with a safety feature that enables the user to lock the button when it does not need to be operated. In operation, the first button 4210 must be first slid forward and then can be pushed down to activate steam generation. In embodiments, the first button 4210 is a push and slide button that is configured to be pushed in and slid forward to activate steam generation.

In some embodiments, the catheter shaft 4206 includes, at its distal end, the needle 4241, a soft coude tip 4243, and a positioning element 4245 configured to stabilize the catheter shaft 4206 in a bladder of a patient. The soft coude tip 4243 is configured to be atraumatic to body tissues during advancement of the catheter shaft 4206. The handle further comprises a fluid line 4251 extending from its proximal end 4202 and through the body 4209 of the handle into the catheter shaft 4206, configured to receive fluid for conversion into vapor. The handle 4200 further comprises a power line 4253 extending from its proximal end 4202, through the body 4209 of the handle and into a proximal portion of the catheter shaft 4206, configured to receive an electrical current to heat an electrode positioned within the proximal portion of the catheter shaft to convert the fluid to vapor for ablation.

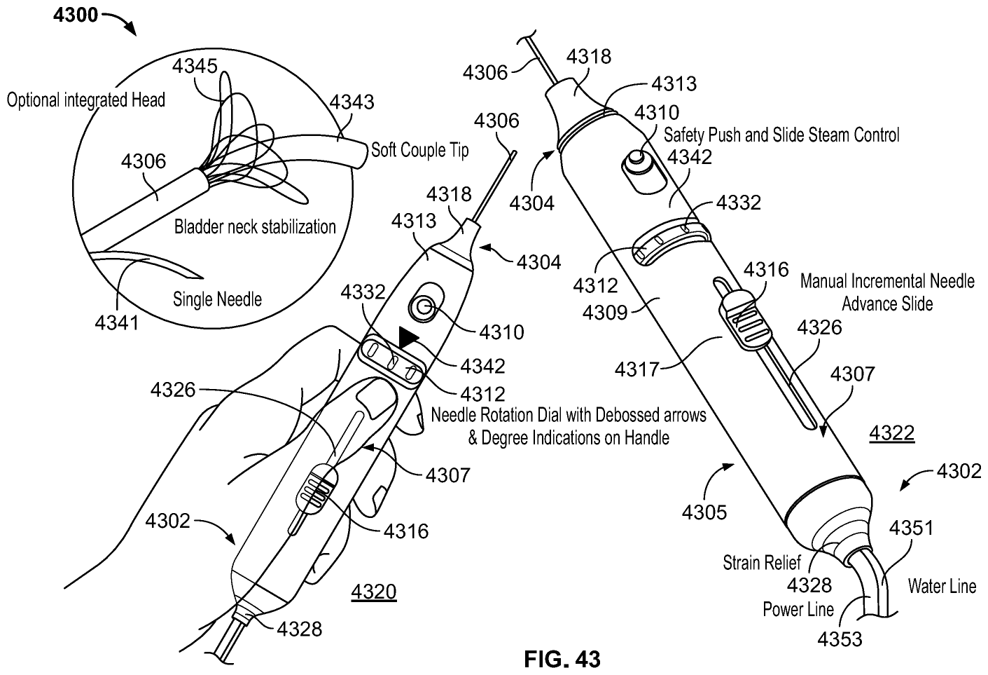

FIG. 43 illustrates another embodiment of a catheter handle mechanism 4300 in accordance with some embodiments of the present specification. The embodiments depicted in FIG. 43 include a combination of functions and features preferred in a pen themed handle for controlling the ablation systems of the present specification. The figure illustrates two views of the handle 4300—a top view 4320 and a top perspective view 4322. The handle 4300 has a tubular structure with a proximal end 4302 and a distal end 4304, and is smoothly curved to provide an ergonomic grip to the user. A catheter shaft 4306 extends from the distal end 4304. In various embodiments, the catheter shaft 4306 extends from the distal end 4304 along a longitudinal axis of the handle 4300. In some embodiments, the handle 4300 includes a first sliding button 4316 within a track 4326 along a length of a top or first side 4307 of the handle 4300. In other embodiments, the sliding button and track are positioned along a length of a bottom or second side 4305 of the handle 4300, opposite the first side 4307. The sliding button 4316 is configured for manual operation by the user to slide the button 4316 incrementally backward to retract a needle 4341 positioned at a distal end of a catheter shaft 4306, and incrementally forward to advance the needle 4341. The handle 4300 may include markings 4317 on its body 4309, proximate the track 4326, to indicate units of distance advanced or retracted by the needle 4341 by manual movement of the button 4316. The sliding button 4316 may be shaped to as to provide an ergonomic grip under the thumb of the user while the user holds the handle 4100 in a single hand. In some embodiments, a diameter of the handle 4300 at a distal portion 4313 is less than a diameter of the handle proximally along the length of its body 4309 and the handle has a tapered distal end 4304. In embodiments, a first strain relief 4318 is positioned at a distal end of the distal portion 4313 and is configured to provide support to the catheter shaft 4306 as it exits from the distal portion 4313.

A second button 4310 is positioned on the top side 4307 proximate the distal end 4304, and aligned with the button 4316, to activate steam generation. In other embodiments, the second button 4310 is positioned on the bottom or second side 4305 of the handle 4300, opposite the first side 4307. In some embodiments, the second button 4310 is a press button with a safety feature that enables the user to lock the button when it does not need to be operated. In operation, the second button 4310 must be first slid forward and then can be pushed down to activate steam generation. In embodiments, the second button 4310 is a push and slide button that is configured to be pushed in and slid forward to activate steam generation. In embodiments, a rotating dial 4312 is included within the handle body 4309 and a portion of the rotating dial 4312 extends from the first side 4307 of the handle 4300 and is accessible by a user. In some embodiments, the rotating dial is positioned between the first sliding button 4316 and the second button 4310. In other embodiments, the rotating dial 4312 extends on the bottom or second side 4305 of the handle 4300. The rotating dial 4312 is configured to be rotated by the user to cause the needle 4341 at the distal end of the shaft 4306 to rotate. In some embodiments, the rotating dial 4312 comprises debossed arrows 4332 and the handle 4300 may include degree indicator markings 4342 proximate the rotating dial 4312 to indicate to the user the degree of rotation of the needle 4341 when the arrow 4332 and specific marking 4342 align.

In some embodiments, the catheter shaft 4306 includes, at its distal end, the needle 4341, a soft coude tip 4343, and a positioning element 4345 configured to stabilize the catheter shaft 4306 in a bladder of a patient. The soft coude tip 4343 is configured to be atraumatic to body tissues during advancement of the catheter shaft 4306. The handle further comprises a fluid line 4351 extending from its proximal end 4302 and through the body 4309 of the handle into the catheter shaft 4306, configured to receive fluid for conversion into vapor. The handle 4300 further comprises a power line 4353 extending from its proximal end 4302, through the body 4309 of the handle and into a proximal portion of the catheter shaft 4306, configured to receive an electrical current to heat an electrode positioned within the proximal portion of the catheter shaft to convert the fluid to vapor for ablation. In some embodiments, a second strain relief 4328 is positioned at the proximal end 4302 of the handle to provide support to the fluid line 4351 and power line 4353.

The various handle mechanisms described in context of FIGS. 36A to 43 may be used with any of the systems of the present specification, such as those shown in FIGS. 1A, 1M, 1P, 1R, 22B, 29, 30, and 31. In the different illustrated embodiments, different types of buttons or controls may be used in place of the types of buttons or controls that are described. For example, the types of buttons or control used may be selected from a press button with or without safety, a rotating wheel type of control for controlling linear or circular movements, slider buttons, toggle buttons, or any other types of buttons that may be suitable for the purposes of operating the handle in accordance with the embodiments of the present specification. Additionally, the buttons may be placed on either side (left or right) of the handle, to suit a left or a right handed user, or may be centrally placed so as to suit both right and left handed users.

In all the above embodiments described in context of FIGS. 36A to 43, the catheters of the handle mechanisms also comprises a heating chamber, which is used to generate steam or vapor for supplying to the catheter. The heating chamber is activated by operating the button 3610/3710/3810/3910/4010. In some embodiments, the heating chamber is operated with RF. In some embodiments, the heating chamber comprises an electrode within the catheter shaft. The chamber is filled with water via a water inlet port located at a proximal end of the handle mechanism. In embodiments, sterile water or saline is supplied from a fluid source into the handle for conversion into vapor. The handle is also equipped with an electrical connection to supply the coil with electrical current from a current generator. Alternating current is provided to the electrode, thereby heating the electrode in the chamber and causing the fluid within to vaporize. The resulting steam or vapor, generated in the chamber, is delivered through the needles placed at the appropriate location to ablate target tissue. A start/stop button is provided on the handle to initiate or stop ablation therapy as required. While some embodiments have separate buttons or controls for advancing and for retracting the needles, all the embodiments may have separate buttons for these purposes, or once the needles are advanced to the maximum distance by repeatedly pressing the trigger for advancing, further pressing of the trigger results in retraction of the needles. In all of the above embodiments, the retraction may be in an instant, or one increment of distance at a time. Additionally, in all of the embodiments of the handle mechanism, markers may be placed on the handle, which indicate the depth of insertion of the needles. The markers may be placed by printing, etching, painting, engraving, or by using any other means known in the art suitable for the purpose. The ablation needles may be inserted or retracted in increments of a fixed distance—such as 5 mm, and therefore markers are placed correspondingly to reflect the increments. Similar markers may also be provided for buttons, dials, or rotating wheels, that are used to rotate the needles. The same functionality can be achieved by other handle form-factors known in the art and also described in this application.

Handle Mechanisms for Endometrial Ablation

As described previously, FIGS. 17A and 17B illustrate a typical anatomy 1700 of the uterus 1706 and uterine tubes of a human female. FIGS. 18A to 18X illustrate various embodiments of ablation catheter arrangements for ablating the uterus 1706, in accordance with the present specification. Referring simultaneously to FIGS. 17A, 17B and 18A to 18X, in embodiments, a coaxial catheter is used to insert into vagina of a patient and advanced toward the cervix. The catheter comprises an outer catheter and an inner catheter. The inner catheter is concentric with and has a smaller radius than the outer catheter. An electrode for heating the catheter tip is located between two positioning elements. In some embodiments, the electrode is proximal to a proximal positioning element. In some embodiments, the two positioning elements are discs—a proximal disc and a distal disc, which may also be referred to as hoods or baskets. The hoods may be made from wires with different wire stiffness. The distal hood is configured to contact fundus of the uterus, and acts like a scaffolding to push two halves of uterus away from each other. The proximal hood is configured to occlude an internal cervical os. Further, FIGS. 19A to 19P illustrate different embodiments of the positioning elements and their deployment at the distal end of the catheter.

Multiple embodiments of handle mechanism that may be used with the endometrial ablation devices are now described. While these embodiments are used for endometrial ablation, they may also be used with other systems of the present specification, such as those shown in FIGS. 1A, 1M, 1P, 1R, 22B, 29, 30, and 31. The multiple embodiments of handle mechanism include systems for deployment and retraction of positioning elements, which can be achieved in different ways such as and not limited to buttons, push/pull on distal or proximal end, slide button in a track, rotation with push/pull. The internal components of the embodiments of the handle mechanism are made generally using stainless steel. The external components of the embodiments of the handle mechanism are made generally using a combination of ABS, plastics, rigid polymers, and elastomeric polymers, among other material. The various embodiments also provide for strain relief for the back end for the fluid tube and the electrical cable, and strain relief on the front end to provide support for the catheter segment. In various embodiments, the handles described in the present specification have lengths ranging from 3 inches to 24 inches and diameters or widths ranging from ¼ inch to 5 inches.

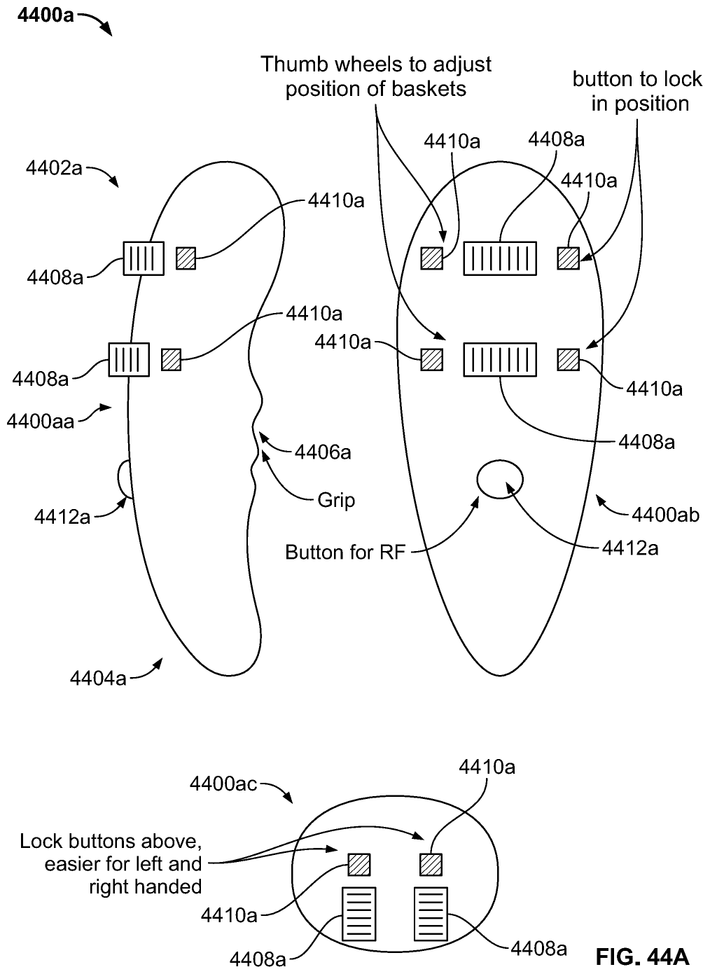

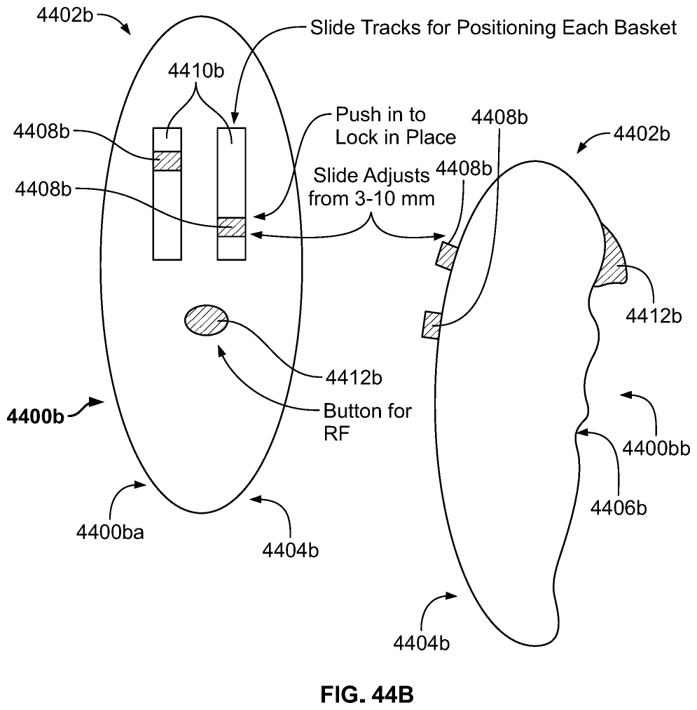

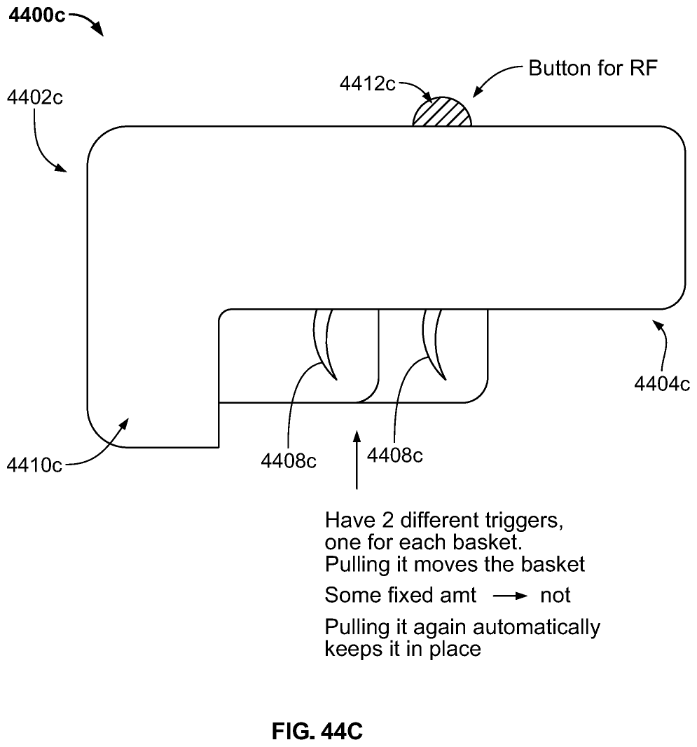

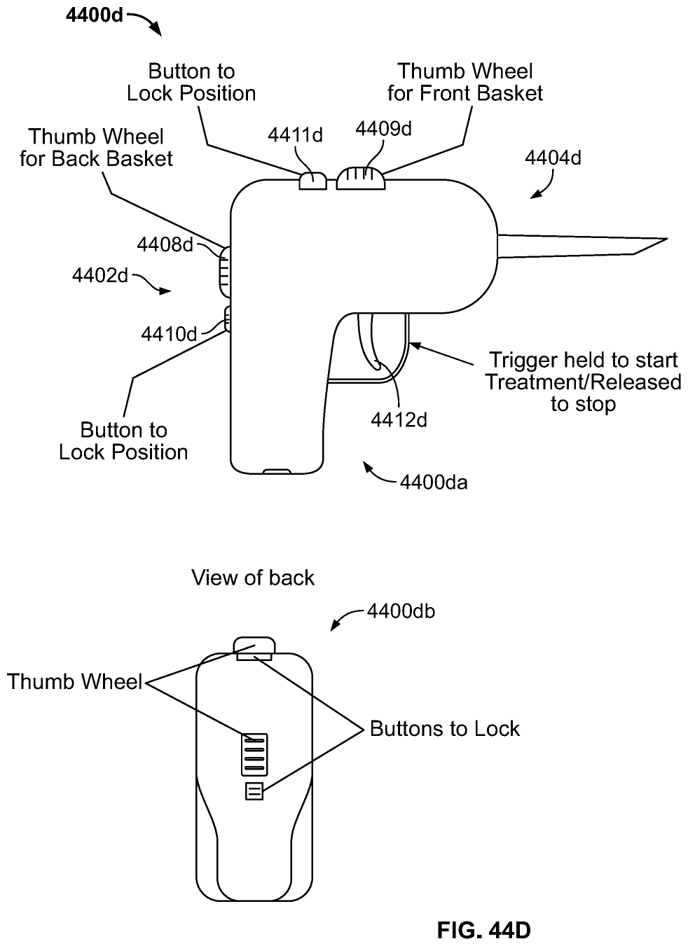

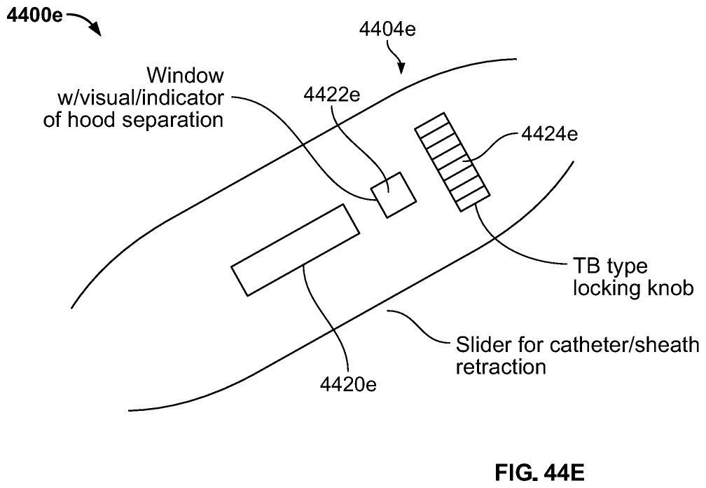

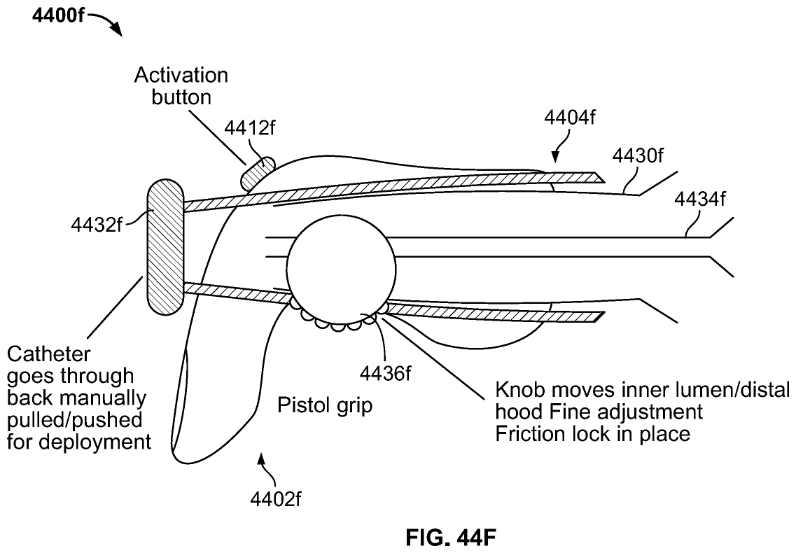

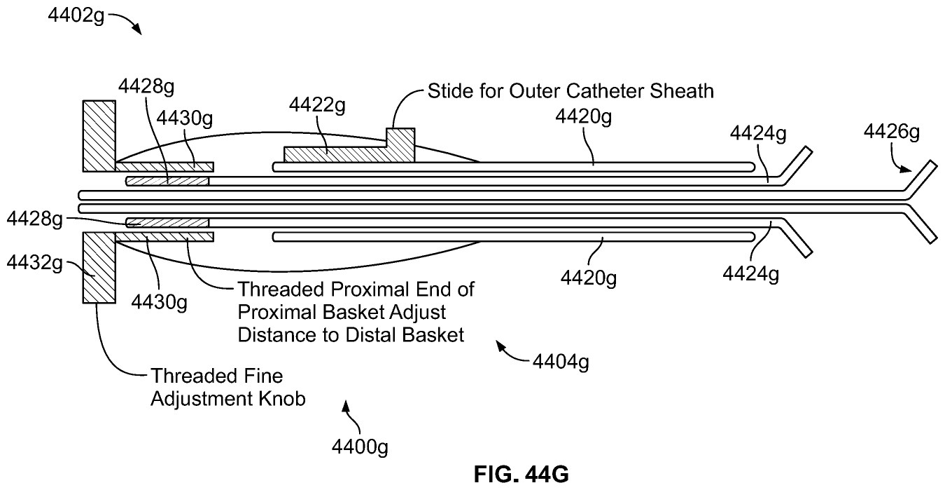

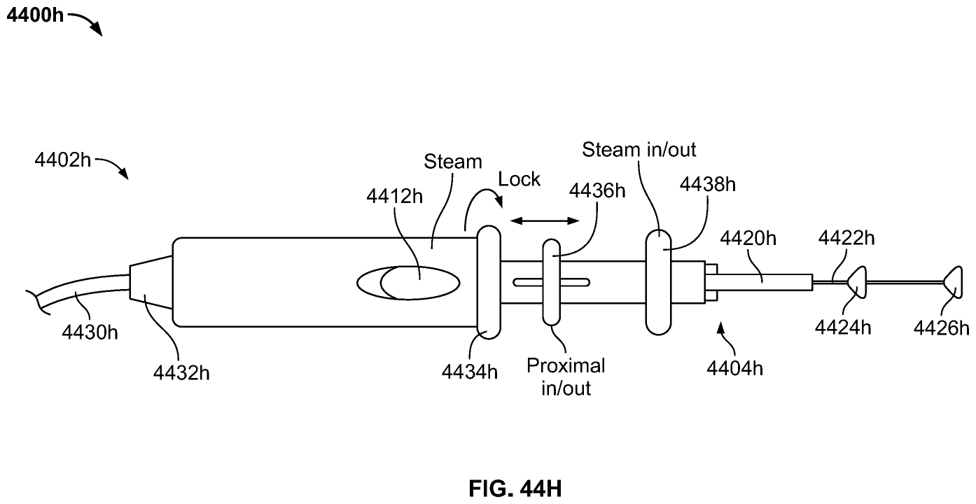

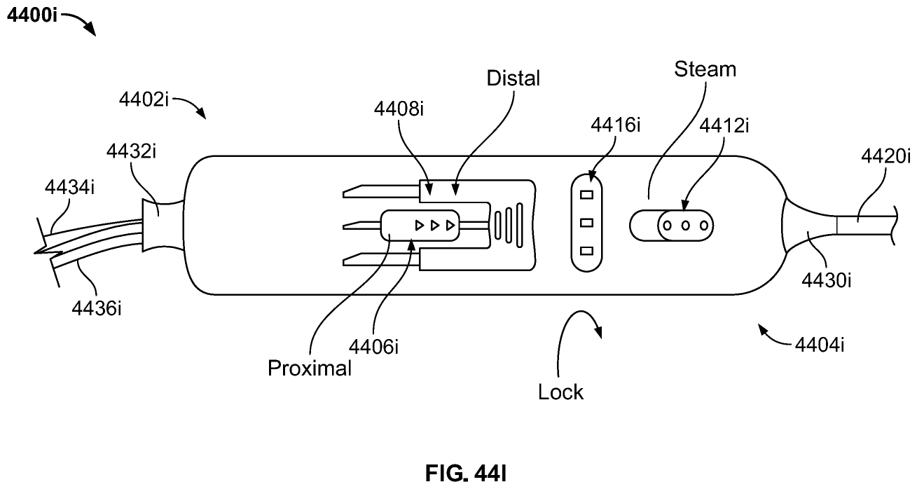

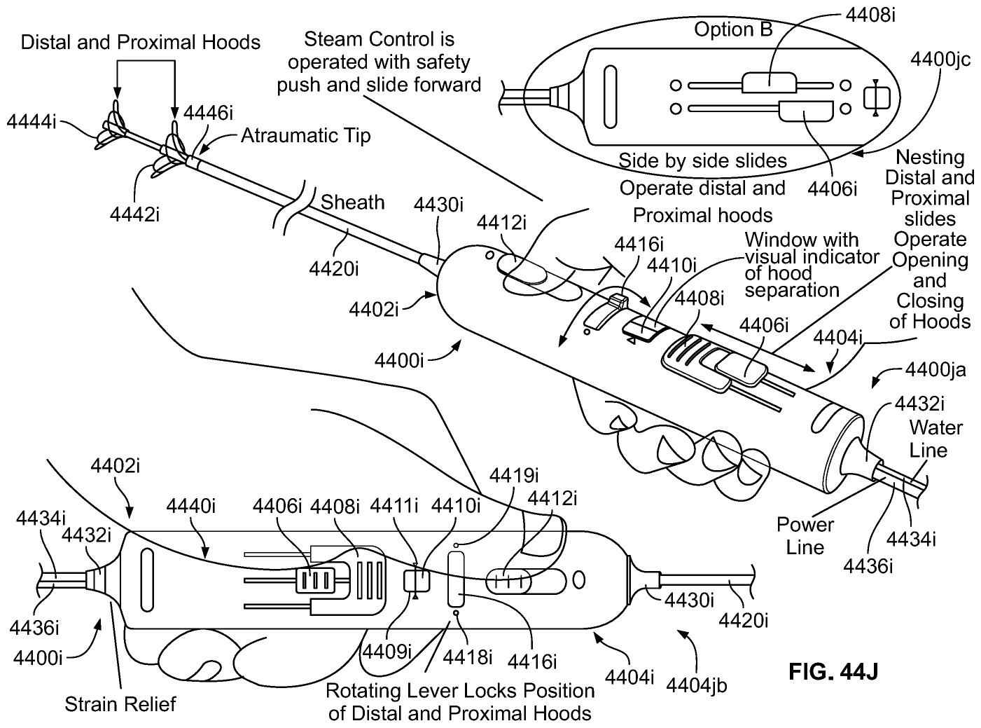

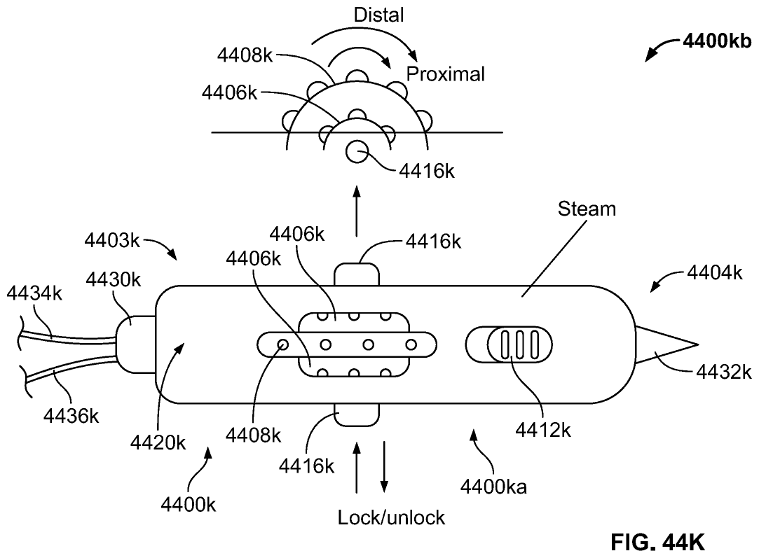

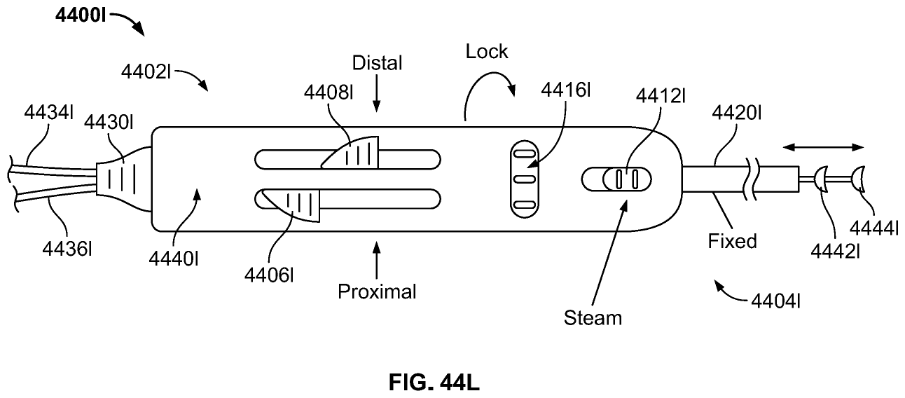

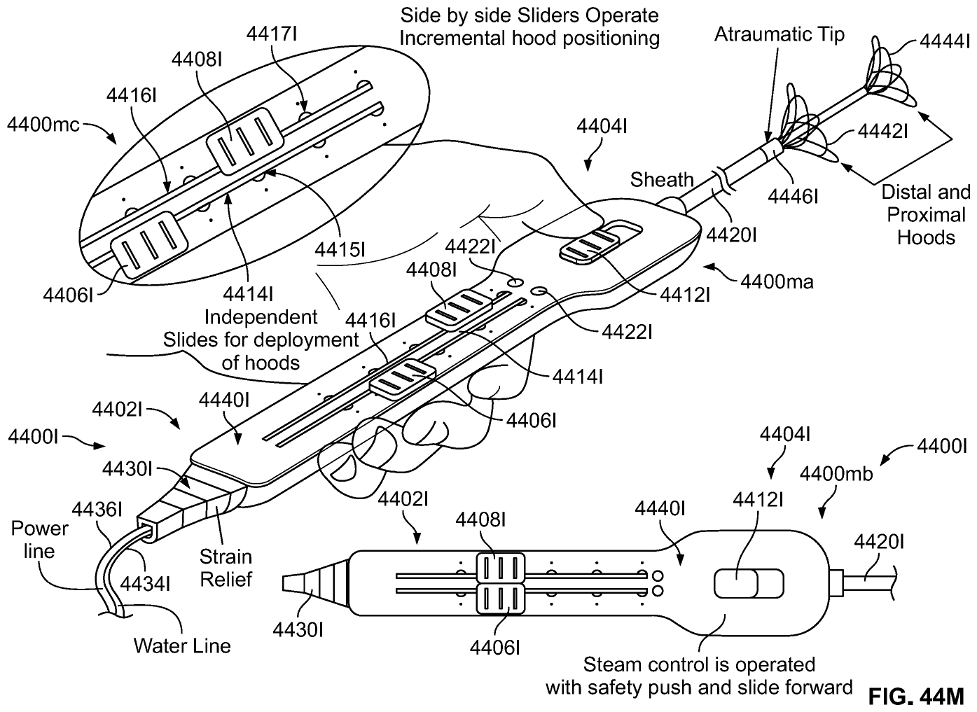

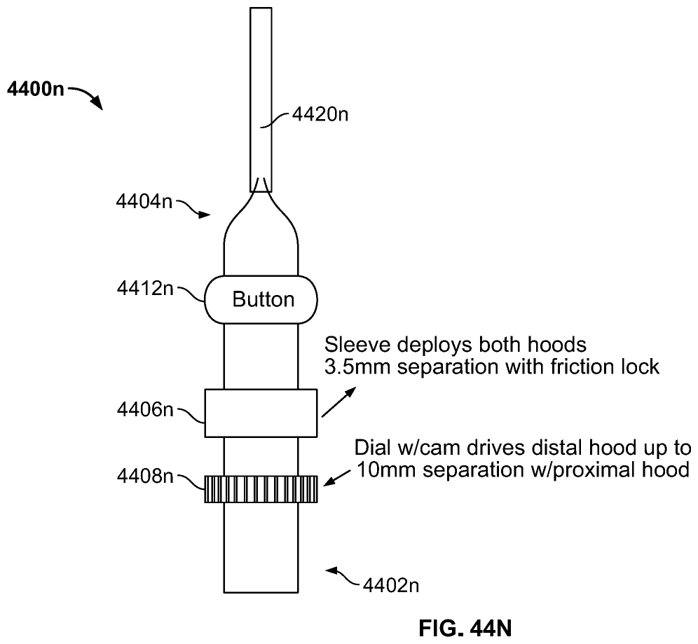

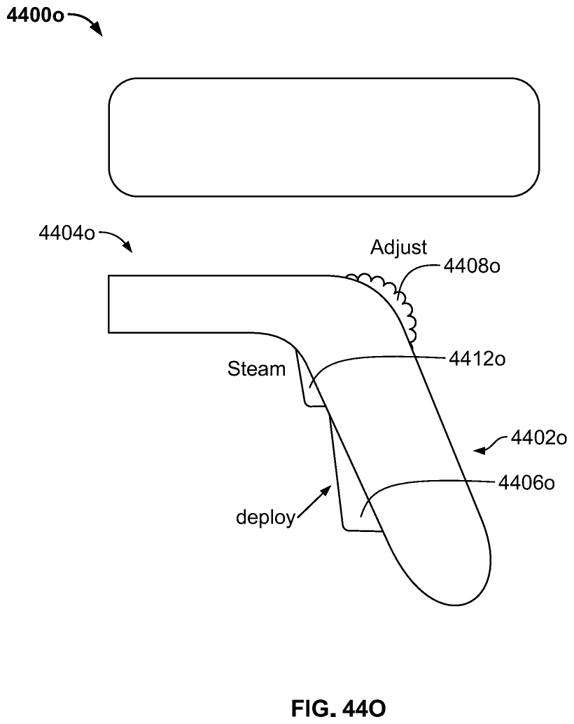

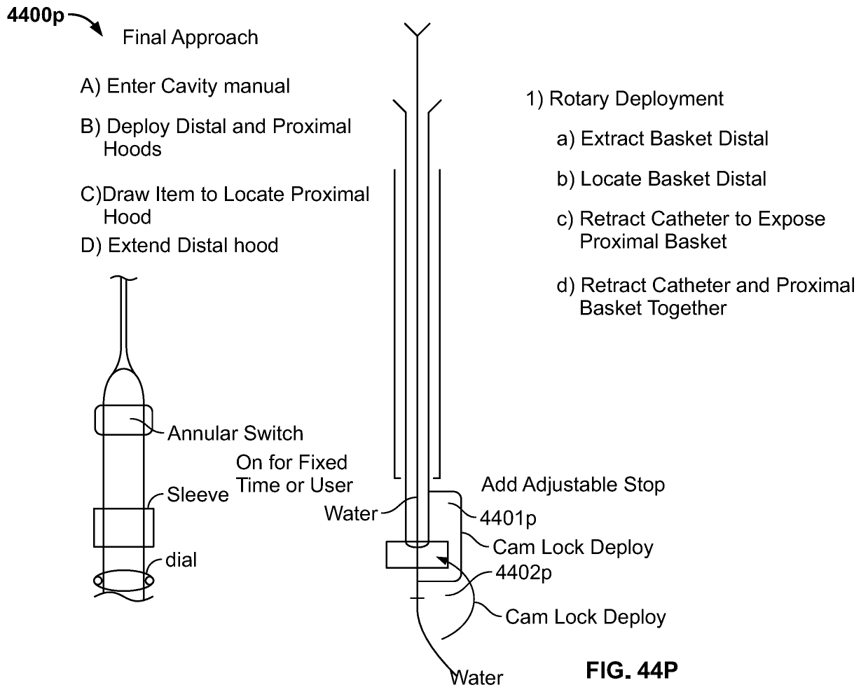

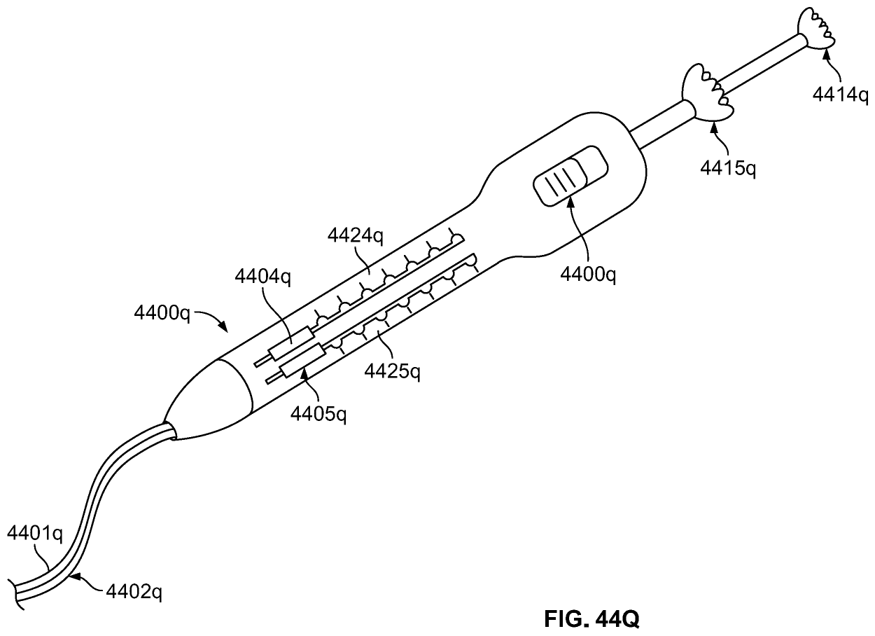

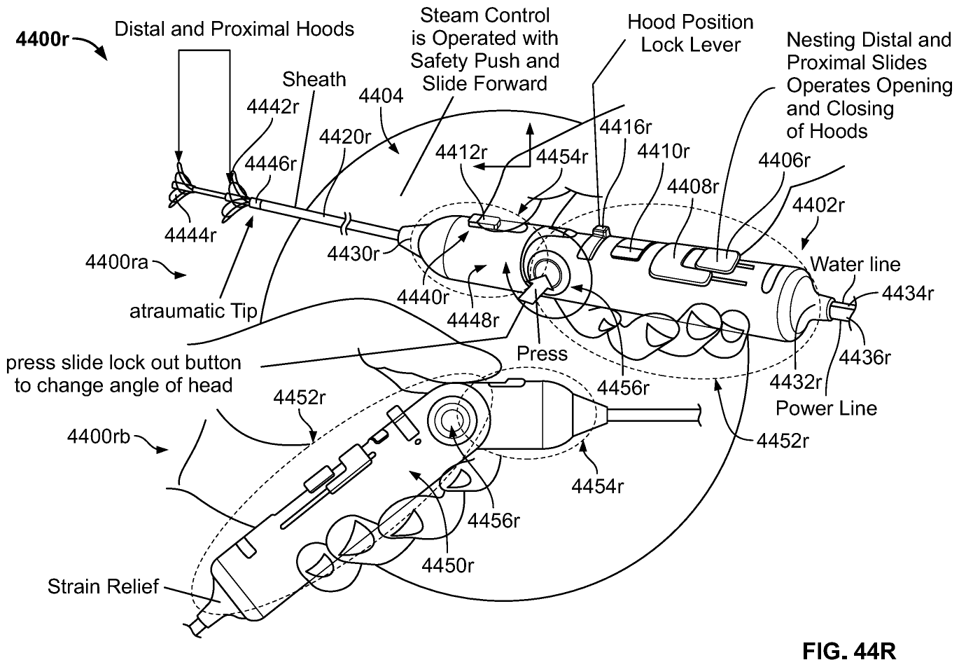

FIG. 44A illustrates an embodiment of a handle 4400a to be used with the endometrial ablation systems of the present specification wherein the shape of the handle 4400a approximates that of having an elongate cylindrical length. The figure shows a side elongated view 4400aa and a front elongated view 4400ab of the handle 4400a, in accordance with an embodiment. The handle 4400a has an elongated tubular structure with a proximal end 4402a and a distal end 4404a, where the distal end 4404a corresponds to the end of the handle 4400a to which a catheter shaft is attached. The tubular body of the handle 4400a has a varying diameter along its length such that a diameter at proximal end 4402a along its length is greater than a diameter at its distal end 4404a. In embodiments, the tubular body is slightly elliptical so that a broader curved surface is available on a front side and a back side of the handle 4400a. At least two, or more, grooves 4406a are provided at the back side of the handle 4400a to enable a user to ergonomically grip the handle 4400a by resting at least two fingers in the at least two groves. A pair of thumbwheels 4408a are located on the front portion of the handle, close to the proximal side 4402a. Each thumbwheel 4408a corresponds to a positioning element. Thumbwheels 4408a are located along a length of on the handle 4400a so as to enable the user to place a thumb on either wheel and rotate the wheel to adjust position of the corresponding positioning element. At least one button 4410a for each wheel 4408a is located near the corresponding wheel. The buttons 4410a are used to lock the positioning element operated by the corresponding wheel, in a position. Button 4410a could be on one side (left or right) of the corresponding wheel 4408a, or could be on both (left and right) sides of the corresponding wheel 4408a, so that the handle 4400a is operable using both the left hand and the right hand. In some embodiments, as shown in 4400ac, lock buttons 4410a could be positioned above or below the corresponding wheels 4408a, where the wheels 4408a are located next to each other along a width of the handle 4400a. A push button 4412a near the central-distal end on the front side of handle 4400a is provided to enable a user to use their thumb or index finger to operate the button 4412a so as to activate the generation of steam.

FIG. 44B illustrates an embodiment of a handle 4400b to be used with the endometrial ablation systems of the present specification wherein the shape of the handle 4400b approximates that of having an elongate cylindrical length. The figure shows a front elongated view 4400ba of one embodiment and a side elongated view 4400bb of another embodiment, of the handle 4400b. The handle 4400b has an elongated tubular structure with a proximal end 4402b and a distal end 4404b, where the distal end 4404b corresponds to the end of the handle 4400b to which a catheter shaft is attached. The tubular body of the handle 4400b has a varying diameter along its length such that a diameter at proximal end 4402b along its length is greater than a diameter at its distal end 4404b. In embodiments, the tubular body is slightly elliptical so that a broader curved surface is available on a front side and a back side of the handle 4400b. At least two, or more, grooves 4406b are provided at the back side of the handle 4400b to enable a user to ergonomically grip the handle 4400b by resting at least two fingers in the at least two groves. A pair of sliding buttons 4408b are located on the front portion of the handle, close to the proximal side 4402b. Each sliding button can be slid by the user's thumb to move along a sliding track 4410b. A pair of sliding tracks 4410b provide a track for each sliding button 4408b. Buttons 4408b can be slid within a distance of 3 to 10 mm within their corresponding tracks 4410b, so as to position a positioning element that is connected to each button 4408b. Further, after positioning the corresponding positioning elements, buttons 4408b can be pressed within their tracks to lock the positioning elements in their place. In some embodiments, tracks 4410b are parallel and located along a length of the handle 4400b. As shown in view 4400ba, a button 4412b near the central-distal end on the front side of handle 4400a is provided to enable a user to use their thumb or index finger to operate the button 4412b so as to activate the generation of steam. In an alternative embodiment, shown in view 4400bb, the button 4412b is positioned at the back side, near the proximal side 4402b of the handle 4400b. The button 4412 may be a round push button, may be a triangular trigger button, or may have any other shape or structure that provides an ergonomic control for the user to operate the heating of electrode(s).

FIG. 44C illustrates an embodiment of a catheter handle mechanism 4400c in accordance with some embodiments of the present specification. The handle 4400c is shaped like a handheld gun or a pistol, which allows it to be conveniently operated by a physician for the ablation treatment. The handle 4400c includes a first portion 4402c and a second portion 4404c, coupled together at an angle in a range of 0 to 180 degrees relative to one another, such that the first portion 4402c is configured to be held in a user's hand and the second portion 4404c extends from an end of the first portion 4402c, and includes a catheter shaft extending therefrom. A strain relief may be configured to provide support to the catheter shaft positioned at a distal end of the second portion 4404c. Positioning elements are coupled to the catheter shaft, as explained in the embodiments above, and one or more vapor ports between the positioning elements are used to deliver steam or vapor to a target tissue. In embodiments, the first portion 4402c may be provided with friction grips to secure a grip of the user. A push button 4412c, in embodiments, positioned on a top edge of the second portion 4404c, is provided to activate steam generation. In some embodiments, the button 4412c is a press button with a safety feature that enables the user to lock the button when it does not need to be operated. A pair of triggers 4408c are provided at the bottom edge of second portion 4404c. Each trigger 4408c corresponds to a positioning element in the catheter shaft. Pulling the trigger moves the corresponding positioning element over a distance. The positioning elements are held in their place when the corresponding trigger is not operated.

FIG. 44D illustrates an embodiment of a catheter handle mechanism 4400d in accordance with some embodiments of the present specification. The figure shows a side view 4400da and a back view 4400db of the handle 4400d, in accordance with some embodiments. The handle 4400d is shaped like a handheld gun or a pistol, which allows it to be conveniently operated by a physician for the ablation treatment. The handle 4400d includes a first portion 4402d and a second portion 4404d, coupled together at an angle in a range of 0 to 180 degrees relative to one another, such that the first portion 4402d is configured to be held in a user's hand and the second portion 4404d extends from an end of the first portion 4402d, and includes a catheter shaft extending therefrom. A strain relief may be configured to provide support to the catheter shaft positioned at a distal end of the second portion 4404d. Positioning elements are coupled to the catheter shaft, as explained in the embodiments above, and one or more vapor ports between the positioning elements are used to deliver steam or vapor to a target tissue. In embodiments, the first portion 4402d may be provided with friction grips to secure a grip of the user. A pair of thumbwheels 4408d and 4409d are provided to control movement and placing of a positioning element each. In some embodiments, a first thumbwheel 4408d is provided on a back side along the length of the first portion 4402d. In one embodiment, the thumbwheel 4408d controls the proximal positioning element and a second thumbwheel 4409d controls the distal positioning element. In alternative embodiments, thumbwheel 4408d controls the distal positioning element and thumbwheel 4409d controls the proximal positioning element. Thumbwheel 4409d is positioned along a top edge of the second portion 4404d. A press button 4410d accompanies the thumbwheel 4408d and is configured to lock position of the corresponding positioning element, when operated. The lock button 4410d may be positioned adjacent to thumbwheel 4408d, such as below the thumbwheel 4408d as shown. Similarly, a press button 4411d accompanies the thumbwheel 4409d and is configured to lock position of the corresponding positioning element, when operated. The lock button 4411d may be positioned adjacent to thumbwheel 4409d, such as behind the thumbwheel 4409d as shown. A trigger pull 4412d, in embodiments, positioned along a bottom edge of the second portion 4404d, is provided to activate steam generation. In some embodiments, the button 4412d is pulled to start the ablation treatment with generation of steam/vapor, and is released to stop the treatment.

FIG. 44E illustrates a portion 4404e of a handle mechanism 4400e, in accordance with some embodiments of the present specification. The handle 4400e may shaped like a handheld gun or a pistol, or may be of a tubular elongated length, which allows it to be conveniently operated by a physician for the ablation treatment. The handle 4400e includes a first portion (not shown) and a second portion 4404e, coupled together at an angle in a range of 0 to 180 degrees relative to one another, such that the first portion is configured to be held in a user's hand and the second portion 4404e extends from an end of the first portion, and includes a catheter shaft extending therefrom. The figure illustrates a top view of the second portion 4404e. In some alternative embodiments, the components illustrated in the figure are configured along an edge of the first portion 4402e. A top edge of the second portion includes a sliding button configured within a sliding track 4420e. The sliding button may be slid forward or backward by the user for a corresponding retraction of the catheter shaft. A visual indicator 4422e is placed in front of the track 4420e, along the top edge of the second portion 4404e. The indicator 4422e is configured to provide a color, line, or any other visual indicator to show the extent of separation between the positioning elements on the catheter shaft. A locking knob 4424e is also provided along the top edge of the second portion 4404e, so as to lock the catheter shaft in its position. In some embodiments, the locking knob 4424e is a T-B type locking knob.

FIG. 44F illustrates another embodiment of a handle mechanism 4400f for an endometrial ablation system, in accordance with some embodiments of the present specification. The handle 4400f is shaped like a handheld gun or a pistol, which allows it to be conveniently operated by a physician for the ablation treatment. The handle 4400f includes a first portion 4402f and a second portion 4404f, coupled together at an angle in a range of 0 to 180 degrees relative to one another, such that the first portion 4402f is configured to be held in a user's hand and the second portion 4404f extends from an end of the first portion 4402f, and includes a catheter shaft 4430f extending therefrom. Shaft 4430f is configured to go through an opening 4432f at a proximal edge of the second portion 4404f and emerge through the distal end of the second portion 4404f The user may manually push or pull the catheter shaft 4430f just before the opening 4432f, so as to push or pull the catheter shaft 4430f for deployment. An inner catheter sheath 4434f is positioning coaxially inside the catheter shaft. Positioning elements are attached to the inner catheter sheath 4434f, as described in the previous embodiments. A rotating knob 4436f is provided along a bottom edge of the second portion 4404f A portion of the knob 4436f is within the body of second portion 4404f and is in communication with the inner catheter sheath 4434f Another portion of the rotating knob is outside the body of second portion 4404f, which can be rotated by the user to move the inner catheter 4434f and therefore the positioning element(s) attached to the inner catheter 4434f In embodiments, a friction lock is configured with the rotating knob 4436f, such that is locks the inner catheter sheath 4434f in its place when it is not operated. A press button 4412f, in embodiments, positioned along a proximal corner edge of the second portion 4404f, is provided to activate steam generation. In some embodiments, the button 4412f is pressed to start the ablation treatment with generation of steam/vapor, and is released to stop the treatment.

FIG. 44G illustrates a cross-sectional view of another embodiment of a handle mechanism 4400g for an endometrial ablation system, in accordance with some embodiments of the present specification. The handle mechanism 4400g has a proximal side 4402g and a distal side 4404g that form a linear, tubular structure that is bulbous at the center, for the user to grip the handle during use. The proximal side 4402g is configured to be held in the user's hand and the distal side 4404g linearly extends from the proximal side 4402g, and includes a catheter shaft 4420g extending therefrom. As slider button 4422g is configured along a top surface of the handle 4400g. The button 4422g is used to retract the outer catheter shaft 4420g so that an inner catheter lumen comprising at least two positioning elements—a proximal positioning element 4424g and a distal positioning element 4426g are deployed at the distal end of the shaft 4420g. At the proximal edge of proximal side 4402g of handle 4400g, the lumen comprising the proximal positioning element 4424g has an external threaded surface 4428g. Surface 4428g further interfaces with an internal threaded surface 4430g within the handle 4400g. The internal threaded surface 4430g is further attached at its proximal side, to a threaded knob 4432g external to the proximal side 4402g of handle 4400g. Therefore, as a user rotates the knob, the surface 4428g interfacing with the knob 4432g enables the proximal positioning element 4424g to move relative to the distal positioning element 4426g. The knob 4432g provides a tool for fine adjustment of the distance between the proximal and distal positioning elements 4424g and 4426g.

FIG. 44H illustrates another embodiment of a handle mechanism 4400h, for use with an endometrial ablation system, in accordance with some embodiments of the present specification. The handle 4400h includes two portions—a proximal portion 4402h, and a distal portion 4404h, which are linearly connected to each other. In some embodiments, the proximal portion 4402h is cylindrical in shape, and is smoothly curved to provide an ergonomic grip to the user. A cable 4430h containing one or more wires for connecting the handle 4400h to at least one of power and saline or fluid for ablation, is configured to enter the handle 4400h at the proximal end of the proximal portion 4402h. A strain relief 4432h may be configured to provide support to the catheter shaft 4430h positioned at the proximal end of the proximal portion 4402h. Further, the surface of the cylindrical body of proximal portion 4402h comprises a button 4412h, provided to activate steam generation. In some embodiments, the button 4412h is a press button, a push button, or a slide button, which is operated by the user to start the ablation treatment with generation of steam/vapor. A rotating ring 4434h is provided at the distal circular edge of the proximal portion 4402h. The ring 4434h is configured to be rotated by the user to lock. The distal portion 4404h extends along the same central horizontal axis as that of proximal portion 4402h. Distal portion 4404h is also cylindrical in shape, and has a diameter that is less than that of the proximal portion. A catheter sheath 4420h extends from the distal end of the distal portion 4404h. The sheath 4420h contains a coaxial inner catheter lumen 4422h within. The inner lumen 4422h further has a proximal positioning element 4424h and a distal positioning element 4426h attached towards its distal end. Distal portion 4404h includes at least two circular dials configured on its external surface. A first dial 4436h attaches to the proximal positioning element 4424h and can be either rotated, or pushed/pulled in a direction along the horizontal axis of the distal portion 4404h, so as to position the proximal positioning element 4424h at a distance relative to the distal positioning element 4426h. A second dial 4438h, may be positioned at a distal side relative to, and at a distance from, the first dial 4436h. The second dial 4438h is configured to be rotated by the user to displace the catheter 4420h so as to deploy the one or more positioning elements 4424h/4426h on the inner lumen 4422h.

FIGS. 44I and 44J illustrate another embodiment of a handle mechanism 4400h, for use with an endometrial ablation system, in accordance with some embodiments of the present specification. The embodiments depicted in FIGS. 441 and 44J include a combination of features preferred in a linear handle 4400i for controlling the endometrial ablation systems of the present specification. FIG. 44J illustrates two views of the handle 4400i—a top perspective view 4400ja and a top view 4400jb of a lateral first side 4440i. Referring simultaneously to FIGS. 441 and 44J, the handle 4400i has a tubular structure with a proximal end 4402i and a distal end 4404i, and is smoothly curved to provide an ergonomic grip to the user. A catheter shaft 4420i extends from the distal end 4404i. In various embodiments, the catheter shaft 4420i extends from the distal end 4404i along a longitudinal axis of the handle 4400i. In some embodiments, the handle 4400i includes a first button 4412i on a portion proximate the distal end 4404i and positioned on lateral first side 4440i of the handle 4400i. The first button 4412i is configured such that the user's finger can slide the button while also gripping the handle with the remaining fingers of the same hand. In embodiments, the button 4412i is used to control steam generation. In some embodiments, the button 4412i is a press button with a safety feature that enables the user to lock the button when it does not need to be operated. In operation, the button 4412i must be first slid forward and then can be pushed down to activate steam generation. In embodiments, the button 4412i is a push and slide button that is configured to be pushed in and slid forward to activate steam generation. In embodiments, a first strain relief 4430i is positioned at a distal end of the distal portion 4404i and is configured to provide support to the catheter shaft 4420i as it exits from the distal portion 4404i.

In embodiments, the handle 4400i includes a second button 4406i on a portion proximate the proximal end 4402i and positioned on lateral first side 4440i. The second button 4406i is configured for the user to slide the button while also gripping the handle 4400i with the remaining fingers of the same hand. Button 4406i is used to operate opening and closing of a proximal positioning element 4442i located at a distal end of the catheter shaft 4420i, proximal to an atraumatic tip 4446i. The soft atraumatic tip 4446i is configured to be atraumatic to body tissues during advancement of the catheter shaft 4420i. In embodiments, the handle 4400i further includes a third button 4408i on a portion proximate the proximal end 4402i and positioned on lateral first side 4440i. The third button 4408i is configured for the user to slide the button while also gripping the handle 4400i with the remaining fingers of the same hand. Button 4408i is used to operate opening and closing of a distal positioning element 4444i located distal to the proximal positioning element 4442i, at a distal end of the catheter shaft 4420i. In some embodiments, as shown in FIG. 44I and views 4400ja and 4400jb of FIG. 44J, the second button 4406i is nested within the third button 4408i. In an alternative embodiment, as shown in view 4400j c, the second and third button 4406i and 4408i, are parallel to each other on the lateral first side 4440i. A window 4410i is positioned on the lateral first side 4440i on a distal side to second and third buttons 4406i/4408i, and on a proximal side to first button 4412i. Window 4410i may be a square, circular, or any other shaped window that provides a visual indicator of an extent of separation between the proximal and distal positioning elements 4442i/4444i. In some embodiments, a marked scale 4409i within the window 4410i shows the extent of the separation. In some embodiments, the window 4410i includes degree indicator markings 4409i and comprises debossed arrows 4411i on the handle 4400i proximate the window 4410i to indicate to the user the degree of separation between the positioning elements 4442i and 4444i, when the arrows 4411i and specific marking 4409i align.

In embodiments, a rotating lever 4416i is included on the lateral first side 4440i, on a proximal side of the first button 4412i and distal side of the window 4410i. The rotating lever 4416i is configured to be rotated from one side, where the positioning elements 4442i/4444i are locked in their positions, to the other side, where the positioning elements 4442i/4444i are unlocked. The rotating lever 4416i is configured to rotate along the circumference of the tubular handle 4400i. In some embodiments, the rotating lever 4416i is accompanied with graphic indicators 4418i and 4419i that are printed on either side of the path of rotation of the lever 4416i, which guide the user to a lock and an unlock position, respectively.

The handle 4400i further comprises a fluid line 4434i extending from its proximal end 4402i and through the body of the handle 4400i into the catheter shaft 4420i, configured to receive fluid for conversion into vapor. The handle 4400i further comprises a power line 4436i extending from its proximal end 4402i, through the body of the handle 4400i and into a proximal portion of the catheter shaft 4420i, configured to receive an electrical current to heat an electrode positioned within the catheter shaft 4420i to convert the fluid to vapor for ablation. In some embodiments, a second strain relief 4432i is positioned at the proximal end 4402i of the handle 4400i to provide support to the fluid line 4434i and power line 4436i.

FIG. 44K illustrates yet another embodiment of a handle mechanism 4400k for use with an endometrial ablation system, in accordance with some embodiments of the present specification. The handle 4400k is linearly shaped and comprises a proximal portion 4402k and a distal portion 4404k. A catheter shaft extends from the distal end of the distal portion 4404k. The handle 4400k further comprises a fluid line 4434k extending from its proximal end 4402k and through the body of the handle 4400k into the catheter shaft, configured to receive fluid for conversion into vapor. The handle 4400k further comprises a power line 4436k extending from its proximal end 4402k, through the body of the handle 4400k and into a proximal portion of the catheter shaft, configured to receive an electrical current to heat an electrode positioned within the catheter shaft to convert the fluid to vapor for ablation. In some embodiments, a first strain relief 4430k is positioned at the proximal end 4402k of the handle 4400k to provide support to the fluid line 4434k and power line 4436k. A second strain relief 4432k is provided at the distal end of the distal portion 4404k to provide support to the catheter shaft.

The handle 4400k is largely tubular in shape with a first lateral side 4420. Second and third lateral sides are adjacent to the first lateral side at an angle of 90 degrees each to the first lateral side, and are opposite to each other. The figure shows a top view 4400ka illustrating the first lateral side 4420k of handle 4400k, and a side view 4400kb illustrating a portion of second/third lateral side of handle 4400k. A first rotating dial 4406k is configured on the first lateral side, approximate a central portion of handle 4400k. The first rotating dial 4406k is used to operate opening and closing of a proximal positioning element. A second rotating dial 4408k is also configured on the first lateral side, adjacent to the first rotating dial 4406k, approximate a central portion of handle 4400k. The second rotating dial 4408k is used to operate opening and closing of a distal positioning element. In some embodiments, a circumference of the first rotating dial 4406k is smaller than circumference of second rotating dial 4408k. In embodiments, the second rotating dial 4408k is nestled between first rotating dial 4406k. A toggle button 4416k toggles between second lateral side and third lateral side, on either sides of the rotating dials 4406k and 4408k. The toggle button 4416k is used to lock and unlock the position of the positioning elements. Further, the first lateral side 4420k on the surface of the handle 4400k, between the rotating dials 4406k/4408k and the distal end 4404k, comprises a button 4412k, provided to activate steam generation. In some embodiments, the button 4412k is a press button, a push button, or a slide button, which is operated by the user to start the ablation treatment with generation of steam/vapor.

FIGS. 44L and 44M illustrate another embodiment of a handle mechanism 4400l, for use with an endometrial ablation system, in accordance with some embodiments of the present specification. The embodiments depicted in FIGS. 44L and 44M include a combination of features preferred in a flat linear handle 4400l for controlling the endometrial ablation systems of the present specification. FIG. 44M illustrates two views of the handle 4400l—a top perspective view 4400ma and a top view 4400mb of a lateral first side 4440l. Referring simultaneously to FIGS. 44L and 44M, the handle 4400l has a flat spatula-like structure. The first lateral side 4440l has a plain surface, whereas the bottom side opposite to the first lateral side 4440l is mostly planar with smooth rounded edges that join the first planar side 4440l to provide a smooth and ergonomic grip to the user. The handle 4400l has a proximal end 4402l and a distal end 4404l. The distal end 4404l has a greater second width relative to a lesser first width of the proximal end 4402l. The first width from the proximal end 4402l extending towards the distal end 4404l is the same for most length of the handle, and then it increases to the second width, when the length of the handle 4400l is proximal to the distal end 4404l. The change is the width of the handle 4400l along its length, provides it a shape similar to that of a spoon or a spatula. A catheter shaft 4420l extends from the distal end 4404l. In various embodiments, the catheter shaft 4420l extends from the distal end 4404l along a longitudinal axis of the handle 4400l. In some embodiments, the handle 4400l includes a first button 4412l on the relatively wider portion proximate the distal end 4404l and positioned on lateral first side 4440l of the handle 4400l. The first button 4412l is configured such that the user's thumb can slide the button while also gripping the handle with the remaining fingers of the same hand. In embodiments, the button 4412l is used to control steam generation. In some embodiments, the button 4412l is a press button with a safety feature that enables the user to lock the button when it does not need to be operated. In operation, the button 4412l must be first slid forward and then can be pushed down to activate steam generation. In embodiments, the button 4412l is a push and slide button that is configured to be pushed in and slid forward to activate steam generation. In embodiments, the catheter shaft 4420l is fixedly attached to the distal end 4404l.

In embodiments, the handle 4400l includes a second button 4406l and a third button 4408l, which can be slid along their respective sliding tracks 4414l and 4416l. A view 4400mc shows a larger view of the sliding tracks 4414l/4416l and buttons 4406l/4408l. The tracks 4414l/4416l extend along the length of the handle 4400l, over a portion of the first width. Sliding buttons 4406l/4408l are configured to be slid of their respective track 4414l/4416l, while the user also grips the handle 4400l with remaining fingers of the same hand. Buttons 4406l and 4408l are used to deploy and retract a proximal positioning element 4442l and a distal positioning element 4444l, respectively. The proximal and distal positioning elements 4442l and 4444l are located at a distal end of the catheter shaft 4420l. The proximal positioning element 4442l is located proximal to an atraumatic tip 4446l. The soft atraumatic tip 4446l is configured to be atraumatic to body tissues during advancement of the catheter shaft 4420l. In embodiments, the two tracks 4414l and 4416l extend parallel to each other along the same length on the first lateral side 4440l. Each track 4414l and 4416l includes an equal number of recesses. Track 4414l includes recesses 4415l, and track 4416l includes recesses 4417l. Each recess in a track is equally spaced. In one embodiment, both tracks 4414l and 4416l each have five recesses 4415l and 4417l, respectively. In operation, when the user slides the buttons 4406l and 4408l, the buttons rest within a recess in their track, thereby providing incremental positioning of the positioning elements 4442l and 4444l. At rest, the button 4406; and 4408l lock the position of the corresponding positioning element. The recesses 4415l and 4417l also indicate the extent of deployment of the corresponding positioning element. The positions of buttons 4406l and 4408l, indicate the relative distance between the two positioning elements. In embodiments, graphic or text symbols 4422l are printed or embossed at the end of each track, to show whether the track corresponds to the distal or the proximal positioning element. Therefore, embodiments of handle mechanism 4400l provide independent slides for the deployment of the two positioning elements.

The handle 4400l further comprises a fluid line 4434l extending from its proximal end 4402l and through the body of the handle 4400l into the catheter shaft 4420l, configured to receive fluid for conversion into vapor. The handle 4400l further comprises a power line 4436l extending from its proximal end 4402l, through the body of the handle 4400l and into a proximal portion of the catheter shaft 4420l, configured to receive an electrical current to heat an electrode positioned within the catheter shaft 4420l to convert the fluid to vapor for ablation. In some embodiments, a strain relief 4430l is positioned at the proximal end 4402l of the handle 4400l to provide support to the fluid line 4434l and power line 4436l.

FIG. 44N illustrates another embodiment of a handle mechanism 4400n for use with an endometrial ablation system, in accordance with some embodiments of the present specification. The handle 4400n is linearly shaped and comprises a proximal end 4402n and a distal end 4404n. A catheter shaft 4420n extends from the distal end 4404n. The handle 4400n comprises a fluid and a power line extending from its proximal end 4402n and through the body of the handle 4400n into the catheter shaft 4420n, configured to receive fluid and power to heat electrodes for conversion into vapor. In some embodiments, a strain relief is provided on the proximal end 4402 to support the fluid and power lines. Similarly, a strain relief may be provided at the distal end 4404n to provide support to the catheter shaft 4420n.

The handle 4400n is largely cylindrical in shape. A circular sleeve 4406n is configured coaxially on an external surface around the handle 4400n, approximate a central portion along the length of handle 4400n. The circular sleeve 4406n is used to operate deployment of both—a proximal and a distal—positioning elements located at the distal end of the catheter shaft 4420n. In operation, the user slides the sleeve towards the distal end 4404n to deploy the two positioning elements, and slides in an opposite direction to retract the positioning elements. In embodiments, the two positioning elements are separated by a distance of approximately 3.5 mm, which is maintained during their deployment using sleeve 4406n. The deployed position is locked when the sleeve is stationary. A friction lock is in effect during deployment. Further, a rotating dial 4408n is also configured coaxially on the external surface around the handle 4400n, proximal to the proximal end 4402n, and between the proximal end 4402n and sleeve 4406n. The rotating dial 4408n can be rotated by the user in one direction to incrementally drive the distal positioning element farther from the proximal positioning element. In an embodiment, the distal positioning element can be driven up to a distance of 10 mm from the proximal positioning element. Rotating the dial 4408n may retract the distal positioning element to its original distance (such as for example, 3.5 mm, as stated above) from the proximal positioning element. Moreover, a button 4412n is configured on the external surface of the handle 4400n, distal to the sleeve 4406n and proximate the distal end 4404n. The button 4412n is provided to activate steam generation. In some embodiments, the button 4412n is a press button, a push button, a rotating dial, a circular sleeve, or a slide button, which is operated by the user to start the ablation treatment with generation of steam/vapor.

FIG. 44O illustrates an embodiment of a catheter handle mechanism 4400o in accordance with some embodiments of the present specification. The handle 4400o is shaped like a handheld gun or a pistol, which allows it to be conveniently operated by a physician for the ablation treatment. The handle 4400o includes a first portion 4402o and a second portion 4404o, coupled together at an angle in a range of 0 to 180 degrees relative to one another, such that the first portion 4402o is configured to be held in a user's hand and the second portion 4404o extends from an end of the first portion 4402o, and includes a catheter shaft extending therefrom. A strain relief may be configured to provide support to the catheter shaft positioned at a distal end of the second portion 4404o. Positioning elements are coupled to the catheter shaft, as explained in the embodiments above, and one or more vapor ports between the positioning elements are used to deliver steam or vapor to a target tissue. In embodiments, the first portion 4402o is provided a first trigger 4406o. The first trigger 4406o is positioned along an internal length of the first portion 4402o, where the internal length is the length of the first portion 4402o that forms an angle between 0 to 180 degrees with the second portion 4404o. In operation, the user pulls the first trigger 4406o to deploy at least two positioning elements that are located at the distal end of the catheter shaft. In some embodiments, the two positioning elements are deployed at a fixed distance from each other. A rotating dial 4408o is configured at an external corner of the joint between the first portion 4402o and the second portion 4404o. The rotating dial 4408o is operated by the user to adjust a distance between the two positioning elements deployed at the distal end of the catheter. Rotating the dial 4408o may drive the distal positioning element farther away from the proximal positioning element up to a specific length. In one embodiment, the distal positioning element is driven up to a distance of 10 mm from the proximal positioning element. A second trigger 4412o, in embodiments, positioned proximal an internal corner of the joint between the first portion 4402o and the second portion 4404o, and along the internal length of the first portion 4402o, is provided to activate steam generation. In some embodiments, the button 4412o is a press button with a safety feature that enables the user to lock the button when it does not need to be operated.

FIG. 44P illustrates an embodiment of a catheter handle mechanism 4400p in accordance with some embodiments of the present specification. The handle mechanism 4400p includes cam locks 4401p, 4402p for deploying, retracting, and locking distal and proximal positioning elements of an ablation catheter.

FIGS. 44Q and 44R illustrate another embodiment of a handle mechanism 4400q, for use with an endometrial ablation system, in accordance with some embodiments of the present specification. The embodiments depicted in FIG. 44Q are mostly similar to those depicted and described in FIGS. 441 and 44J. Descriptions of these embodiments are not repeated here for the sake of brevity. FIG. 44Q illustrates a single view of handle 4400q. Handle 4400q includes steam line 4401q and power line 4402q. A thumb slide 4403q is included to control the delivery of vapor or steam. A first independent slide 4404q controls the deployment, retraction, and positioning of a first, distal positioning element or hood 4414q and a second independent slide 4405q controls the deployment, retraction, and positioning of a second, proximal positioning element 4415q. A first set of incremental distal positioning element or hood indicators 4424q indicates the distance the first distal positioning element or hood 4414q has been extended. A second set of incremental proximal positioning element or hood indicators 4425q indicates the distance the second proximal positioning element or hood 4415q has been extended. FIG. 44R illustrates two views of the handle 4400r. A first view 4400ra is a perspective view of handle 4400r in a first arrangement; and a second view 4400rb is a perspective view of handle 4400r in a second arrangement. The handle 4400r is broadly divided in to two sections—a first proximal section 4452r comprising the proximal end 4402r, and a second distal section 4454r comprising the distal end 4404r. In embodiments, the button 4412r is also comprised on first lateral side 4440r in the second distal section 4454r. Whereas, the remaining interfaces on the first lateral side 4440r of the handle 4400r, such as including the rotating lever 4416r, window 4410r, and buttons 4406r and 4408r, are comprised in the first proximal section 4452r. The handle 4400r further shows a second lateral side 4448r and a third lateral side 4450r, which are each adjacent to the first lateral side 4440r, and parallel and opposite to each other. A side lock out button 4456r is configured at the distal end of the first proximal section 4452r, distal to rotating lever 4416r. The button 4456r is configured as a press button that is accessible to the user to press with a thumb or a finger while holding the handle 4400r with the remaining fingers of the same hand. The button 4456r is accessible on both—the second lateral side 4448r and the third lateral side 4450r. In operation, the user presses the button 4456r to change the angle between the first proximal section 4452r and the second proximal section 4454r. The first and the second sections 4452r and 4454r are attached to each other and are rotatable relative to each other. First view 4400ra illustrates the arrangement between first and second section 4452r and 4454r when they are linear, at an angle of 180 degrees, relative to each other. Second arrangement 4400rb shows the two sections 4452r and 4454r at a certain angle, relative to each other. The angled position is attained by the user by pressing the button 4456r to disengage a lock that attaches the two sections 4452r and 4454r in a locked position. The user can then manually rotate the second section 4454r to bring it at an angle relative to the first section 4452r. Button 4456r can be pressed again to lock the final angled arrangement.

It should be noted that the various embodiments described in context of FIGS. 44A to 44R may use features and configurations from each other. In some embodiments, a structure and shape of a handle may be any of those described in the figures. Similarly, a combination of the type of control (button, slider, wheel, lever, or toggle) used to activate the generation of steam, the operation of the positioning elements, can be selected from the different embodiments.

The various handle mechanisms described in context of FIGS. 44A to 44R may be used with any of the systems of the present specification, such as those shown in FIGS. 1A, 1M, 1P, 1R, 22B, 29, 30, and 31. In the different illustrated embodiments, different types of buttons or controls may be used in place of the types of buttons or controls that are described. For example, the types of buttons or control used may be selected from a press button with or without safety, a rotating wheel type of control for controlling linear or circular movements, slider buttons, toggle buttons, or any other types of buttons that may be suitable for the purposes of operating the handle in accordance with the embodiments of the present specification. Additionally, the buttons may be placed on either side (left or right) of the handle, to suit a left or a right handed user, or may be centrally placed so as to suit both right and left handed users.

In all the above embodiments described in context of FIGS. 44A to 44R, the catheters of the handle mechanisms also comprises a heating chamber, which is used to generate steam or vapor for supplying to the catheter. The heating chamber is activated by operating the button 4412. In some embodiments, the heating chamber is operated with RF. In some embodiments, the heating chamber comprises an electrode within the catheter shaft. The chamber is filled with water via a water/fluid inlet port located at a proximal end of the handle mechanism. In embodiments, sterile water or saline is supplied from a fluid source into the handle for conversion into vapor. The handle is also equipped with an electrical connection to supply the coil with electrical current from a current generator. Alternating current is provided to the electrode, thereby heating the electrode in the chamber and causing the fluid within to vaporize. The resulting steam or vapor, generated in the chamber, is delivered through the vapor ports or the at least one hole configured between the two positioning elements. A start/stop button is provided on the handle to initiate or stop ablation therapy as required. While some embodiments have separate buttons or controls for advancing and for retracting each positioning element, all the embodiments may have separate buttons for these purposes. In all of the above embodiments, the retraction may be in an instant, or one increment of distance at a time. Additionally, in all of the embodiments of the handle mechanism, markers may be placed on the handle, which indicate the extent of advancement of the positioning elements and the distance between the two positioning elements. The markers may be placed by printing, etching, painting, engraving, or by using any other means known in the art suitable for the purpose. Similar markers may also be provided for buttons, dials, or rotating wheels, that are used to rotate the needles. The same functionality can be achieved by other handle form-factors known in the art and also described in this application.

In embodiments, microwave-based ablation systems and methods are used. Accordingly, microwave is used in place of electrodes to deliver energy to create steam. In the embodiments described below, the ablation systems include catheters with a heating element comprising at least one microwave antenna. The microwave antenna is configured to receive a current and generate heat to convert a fluid passing the antenna into a vapor for ablation.

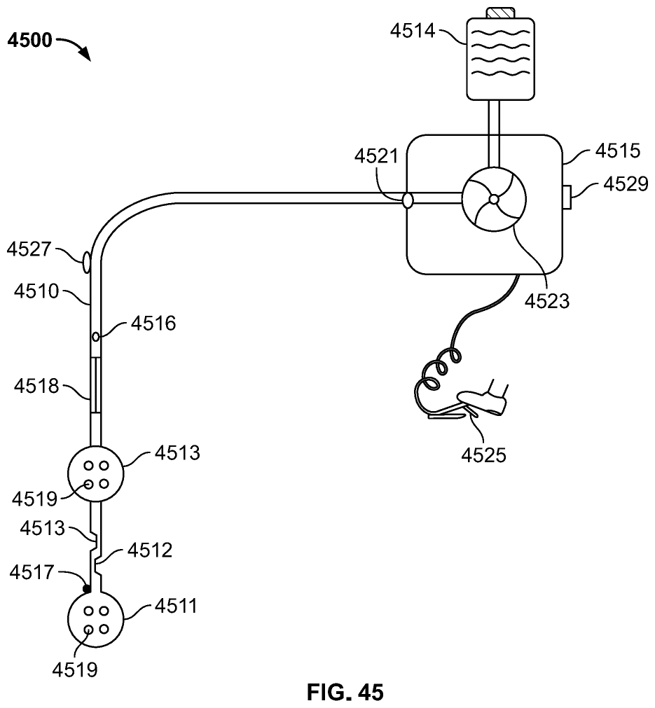

FIG. 45 illustrates an ablation system 4500, in accordance with embodiments of the present specification. The ablation system comprises a catheter 4510 having at least one first distal attachment or positioning element 4511 and an internal heating chamber 4518, disposed within a lumen of the catheter 4510 and configured to heat a fluid provided to the catheter 4510 to change said fluid to a vapor for ablation therapy. In some embodiments, the internal heating chamber 4518 comprises at least one microwave antenna that is separated from thermally conductive element by a segment of the catheter 4510 which is electrically non-conductive. In some embodiments, the catheter 4510 is made of or covered with an insulated material to prevent the escape of ablative energy from the catheter body. The catheter 4510 comprises one or more infusion ports 4512 for the infusion of ablative agent, such as steam. In some embodiments, the one or more infusion ports 4512 comprises a single infusion port at the distal end of a needle. In some embodiments, the catheter includes a second positioning element 4513 proximal to the infusion ports 4512. In various embodiments, the first distal attachment or positioning element 4511 and second positioning element 4513 may be any one of a disc, hood, cap, or inflatable balloon. In some embodiments the distal attachment or positioning element has a wire mesh structure with or without a covering membrane. In some embodiments, the first distal attachment or positioning element 4511 and second positioning element 4513 include pores 4519 for the escape of air or ablative agent. A fluid, such as saline, is stored in a reservoir, such as a saline pump 4514, connected to the catheter 4510. Delivery of the ablative agent is controlled by a controller 4515 and treatment is controlled by a treating physician via the controller 4515. The controller 4515 includes at least one processor 4523 in data communication with the saline pump 4514 and a catheter connection port 4521 in fluid communication with the saline pump 4514. In some embodiments, at least one optional sensor 4517 monitors changes in an ablation area to guide flow of ablative agent. In some embodiments, optional sensor 4517 comprises at least one of a temperature sensor or pressure sensor. In some embodiments, the catheter 4510 includes a filter 4516 with micro-pores which provides back pressure to the delivered steam, thereby pressurizing the steam. The predetermined size of micro-pores in filter 4516 determine the backpressure and hence the temperature of the steam being generated. In some embodiments, the system further comprises a foot pedal 4525 in data communication with the controller 4515, a switch 4527 on the catheter 4510, or a switch 4529 on the controller 4515, for controlling vapor flow. In various embodiments, the switch 4529 is positioned on the generator or the catheter handle.

In one embodiment, a user interface included with the controller 4515 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

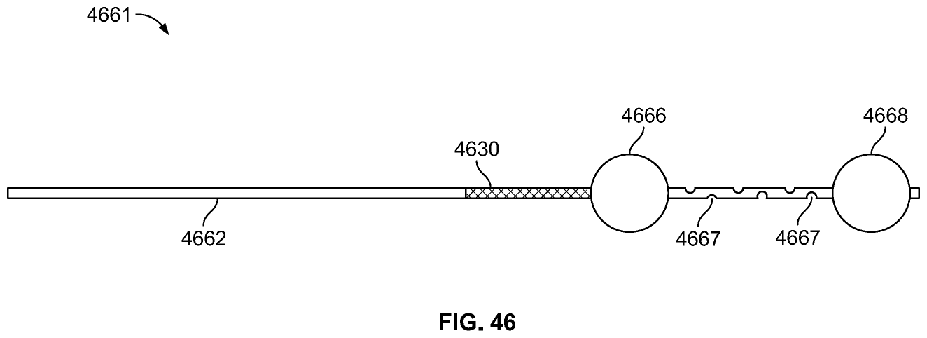

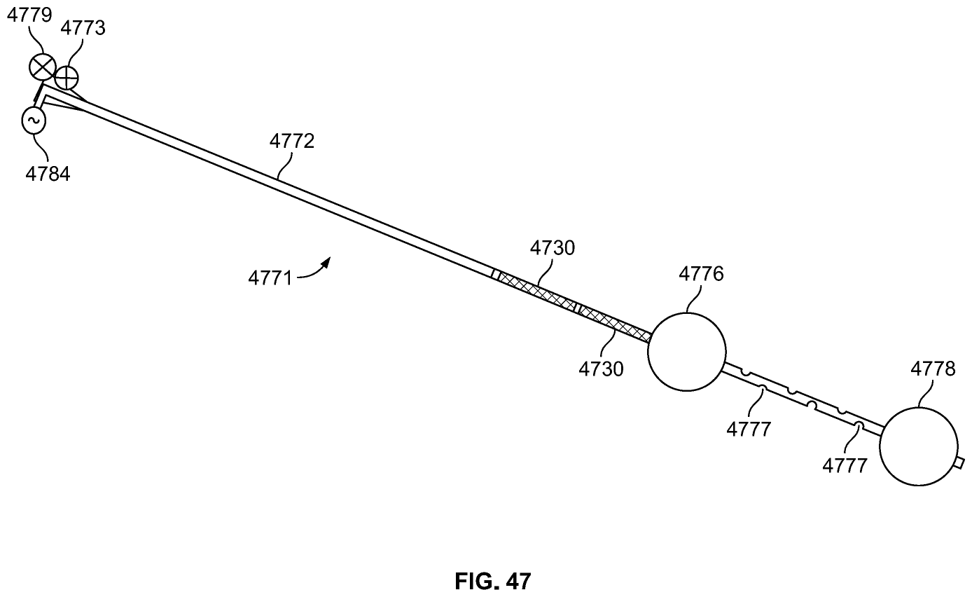

FIG. 46 and FIG. 47 illustrate multiple lumen balloon catheters 4661 and 4671 respectively, in accordance with embodiments of the present specification. The catheters 4661, 4771 each include an elongate body 4662, 4772 with a proximal end and a distal end. The catheters 4661, 4771 include at least one positioning element proximate their distal ends. In various embodiments, the positioning element is a balloon. In some embodiments, the catheters include more than one positioning element.

In the embodiments depicted in FIGS. 46 and 47, the catheters 4661, 4771 each include a proximal balloon 4666, 4776 and a distal balloon 4668, 4778 positioned proximate the distal end of the body 4662, 4772 with a plurality of infusion ports 4667, 4777 located on the body 4662, 4772 between the two balloons 4666, 4776, and 4668, 4778. The body 4662, 4772 also includes at least one heating chamber 4630/4730 proximate and just proximal to the proximal balloon 4666, 4776. Each heating chamber 4630/4730 includes at least one microwave antenna for generating heat. The embodiment of FIG. 46 illustrates one heating chamber 4630 included in the body 4665 proximate and just proximal to the proximal balloon 4666. In some embodiments, multiple heating chambers are arranged in series in the body of the catheter.

In the embodiment of FIG. 47, two heating chambers 4730 are arranged in the body 4772 proximate and just proximal to the proximal balloon 4776. Referring to FIG. 47, for inflating the balloons 4776, 4778 and providing electrical current and liquid to the catheter 4771, a fluid pump 4779, an air pump 4773 and an RF generator 4784 are coupled to the proximal end of the body 4772. The air pump 4773 pumps air via a first port through a first lumen (extending along a length of the body 4772) to inflate the balloons 4776, 4778 so that the catheter 4771 is held in position for an ablation treatment. In another embodiment, the catheter 4771 includes an additional air-port and an additional air lumen so that the balloons 4776, 4778 may be inflated individually. The fluid pump 4779 pumps the fluid through a second lumen (extending along the length of the body 4772) to the heating chambers 4730. The RF generator 4784 supplies electrical current to the at least one microwave antenna, causing the at least one microwave antenna to generate heat and thereby converting the fluid flowing through the heating chambers 4730 into vapor. The generated vapor flows through the second lumen and exits the ports 4777. The flexible heating chambers 4730 impart improved flexibility and maneuverability to the catheters 4661, 4771, allowing a physician to better position the catheters 4661, 4771 when performing ablation procedures, such as ablating Barrett's esophagus tissue in an esophagus of a patient.

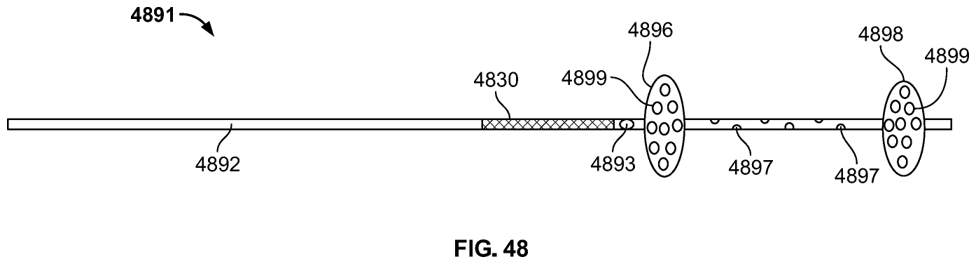

FIG. 48 illustrates a catheter 4891 with proximal and distal positioning elements 4896, 4898 and one or more microwave antenna-based heating chamber 4830, in accordance with embodiments of the present specification. The catheter 4891 includes an elongate body 4892 with a proximal end and a distal end. The catheter 4891 includes a proximal positioning element 4896 and a distal positioning element 4898 positioned proximate the distal end of the body 4892 with a plurality of infusion ports 4897 located on the body 4892 between the two positioning elements 4896, 4898. The body 4892 also includes at least one heating chamber 4830 within a central lumen. In some embodiments, the proximal positioning element 4896 and distal positioning element 4898 comprises compressible discs which expand on deployment. In some embodiments, the proximal positioning element 4896 and distal positioning element 4898 are comprised of a shape memory metal and are transformable from a first, compressed configuration for delivery through a lumen of an endoscope and a second, expanded configuration for treatment. In embodiments, the discs include a plurality of pores 4899 to allow for the escape of air at the start of an ablation procedure and for the escape of steam once the pressure and/or temperature within an enclosed treatment volume created between the two positioning elements 4896, 4898 reaches a predefined limit, as described above. In some embodiments, the catheter 4891 includes a filter 4893 with micro-pores which provides back pressure to the delivered steam, thereby pressurizing the steam. The predetermined size of micro-pores in the filter determine the backpressure and hence the temperature of the steam being generated.

It should be appreciated that the filter 4893 may be any structure that permits the flow of vapor out of a port and restricts the flow of vapor back into, or upstream within, the catheter. Preferably, the filter is a thin porous metal or plastic structure, positioned in the catheter lumen and proximate one or more ports. Alternatively, a one-way valve may be used which permits vapor to flow out of a port but not back into the catheter. In one embodiment, this structure 4893, which may be a filter, valve or porous structure, is positioned within 5 cm of a port, preferably in a range of 0.1 cm to 5 cm from a port, and more preferably within less than 1 cm from the port, which is defined as the actual opening through which vapor may flow out of the catheter and into the patient.

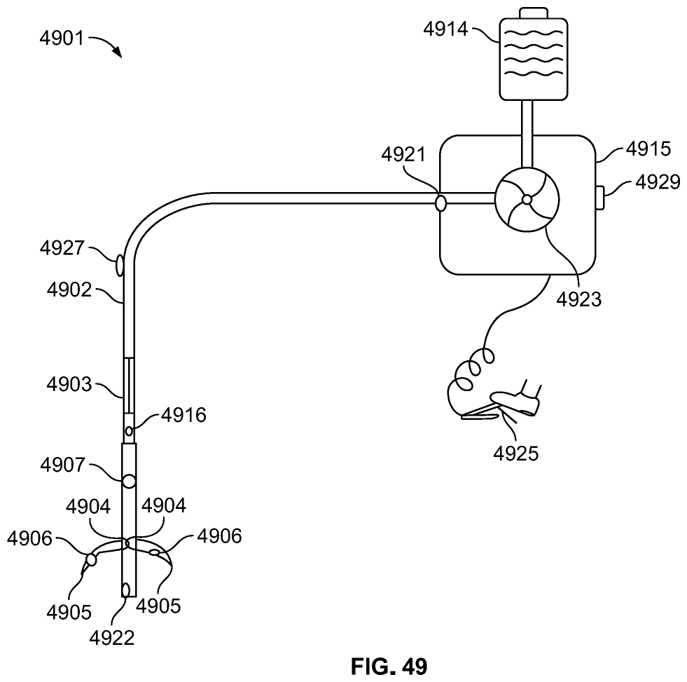

FIG. 49 illustrates an ablation system 4901 suitable for use in ablating prostate tissue, in accordance with some embodiments of the present specification. The ablation system 4901 comprises a catheter 4902 having an internal heating chamber 4903, disposed within a lumen of the catheter 4902 and configured to heat a fluid provided to the catheter 4902 to change said fluid to a vapor for ablation therapy. In one embodiment the fluid is electrically conductive saline and is converted into electrically non-conductive or poorly conductive vapor. In one embodiment, there is at least a 25% decrease in the conductivity, preferably a 50% decrease and more preferably a 90% decrease in the conductivity, of the fluid as determined by comparing the conductivity of the fluid, such as saline, prior to passing through the heating chamber to the conductivity of the ablative agent, such as steam, after passing through the heating chamber. It should further be appreciated that, for each of the embodiments disclosed in this specification, the term ablative agent preferably refers solely to the heated vapor, or steam, and the inherent heat energy stored therein, without any augmentation from any other energy source, including a radio frequency, electrical, ultrasonic, optical, or other energy modality.

In some embodiments, the catheter 4902 is made of or covered with an insulated material to prevent the escape of ablative energy from the catheter body. A plurality of openings 4904 are located proximate the distal end of the catheter 4902 for enabling a plurality of associated thermally conductive elements, such as needles 4905, to be extended (at an angle from the catheter 4902, wherein the angle ranges between 30 to 90 degrees) and deployed or retracted through the plurality of openings 4904. In accordance with an aspect, the plurality of retractable needles 4905 are hollow and include at least one infusion port 4906 to allow delivery of an ablative agent, such as steam or vapor, through the needles 4905 when the needles 4905 are extended and deployed through the plurality of openings 4904 on the elongated body of the catheter 4902. In some embodiments, the infusion ports are positioned along a length of the needles 4905. In some embodiments, the infusion ports 4906 are positioned at a distal tip of the needles 4905. During use, cooling fluid such as water, air, or CO₂ is circulated through an optional port 4907 to cool the catheter 4902. Vapor for ablation and cooling fluid for cooling are supplied to the catheter 4902 at its proximal end. A fluid, such as saline, is stored in a reservoir, such as a saline pump 4914, connected to the catheter 4902. Delivery of the ablative agent is controlled by a controller 4915 and treatment is controlled by a treating physician via the controller 4915. The controller 4915 includes at least one processor 4923 in data communication with the saline pump 4914 and a catheter connection port 4921 in fluid communication with the saline pump 4914. In some embodiments, at least one optional sensor 4922 monitors changes in an ablation area to guide flow of ablative agent. In some embodiments, the optional sensor comprises at least one of a temperature sensor or pressure sensor. In some embodiments, the catheter 4902 includes a filter 4916 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 4925 in data communication with the controller 4915, a switch 4927 on the catheter 4902, or a switch 4929 on the controller 4915, for controlling vapor flow. In some embodiment, the needles have attached mechanism to change their direction from being relatively parallel to the catheter to being at an angle between 30°-90° to the catheter. In one embodiment, the aforementioned mechanism is a pull wire. In some embodiments, the openings in the catheter are shaped to change the direction of the needle from being relatively parallel to the catheter to being at an angle between 30°-90° to the catheter.

In one embodiment, a user interface included with the microprocessor 4915 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

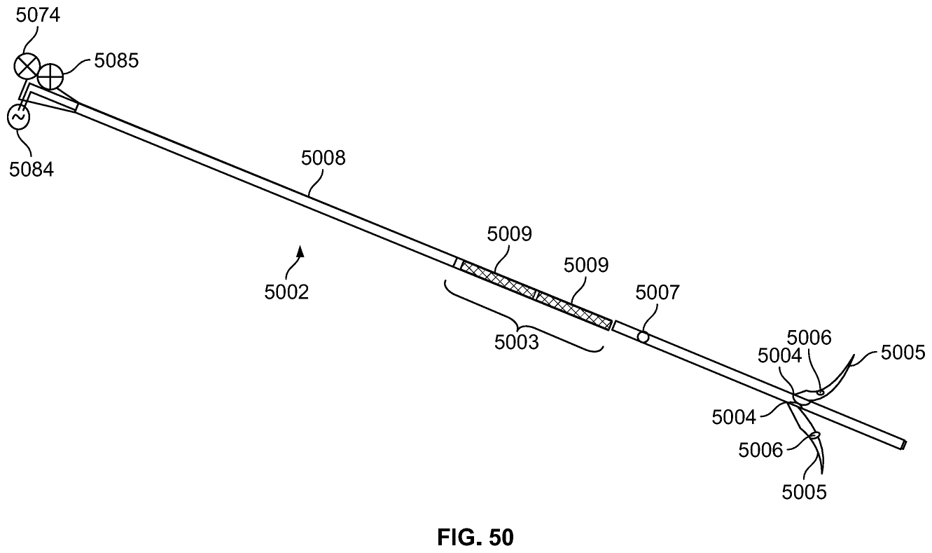

FIG. 50 illustrates another view of a catheter 5002 of FIG. 49, in accordance with some embodiments of the present specification. The catheter 5002 includes an elongate body 5008 with a proximal end and a distal end. A plurality of openings 5004 are located proximate the distal end of the catheter 5002 for enabling a plurality of associated thermally conductive elements, such as needles 5005, to be extended (at an angle from the catheter 5002, wherein the angle ranges between 10 to 90 degrees) and deployed or retracted through the plurality of openings 5004. In accordance with an aspect, the plurality of retractable needles 5005 are hollow and include at least one infusion port 5006 to allow delivery of an ablative agent, such as steam or vapor, through the needles 5005 when the needles 5005 are extended and deployed through the plurality of openings 5004 on the elongated body of the catheter 5002. In some embodiments, the infusion ports are positioned along a length of the needles 5005. In some embodiments, the infusion ports 5006 are positioned at a distal tip of the needles 5005. Optionally, during use, cooling fluid, such as water, air, or CO₂ is circulated through an optional port 5007 to cool the catheter 5002. The body 5008 includes at least one heating chamber 5003 proximate and just proximal to the optional port 5007 or openings 5004. In embodiments, the heating chamber 5003 comprises at least two microwave antenna or microwave antenna arrays 5009 configured to receive RF current, heat, and convert supplied fluid, such as saline, to vapor or steam, for ablation.

Referring to FIG. 50, for providing electrical current, fluid for ablation, and optional cooling fluid to the catheter 5002, an RF generator 5084, a first fluid pump 5074, and a second fluid pump 5085 are coupled to the proximal end of the body 5008. The first fluid pump 5074 pumps a first fluid, such as saline, through a first lumen (extending along the length of the body 5008) to the heating chamber 5003. The RF generator 5084 supplies electrical current to the microwave antenna array 5009, causing the microwave antenna array 5009 to generate heat and thereby converting the fluid flowing through the heating chamber 5003 into vapor. The generated vapor flows through the first lumen, openings 5004, needles 5005, and exits the infusion ports 5006 to ablate prostatic tissue. Optionally, in some embodiments, a second fluid pump 5085 pumps a second fluid, such as water, through a second lumen (extending along a length of the body 5008) to optional port 5007, where the second fluid exits the catheter 5002 to circulate in and cool the area of ablation. The flexible heating chamber 5003 imparts improved flexibility and maneuverability to the catheter 5002, allowing a physician to better position the catheter 5002 when performing ablation procedures, such as ablating prostate tissue of a patient.

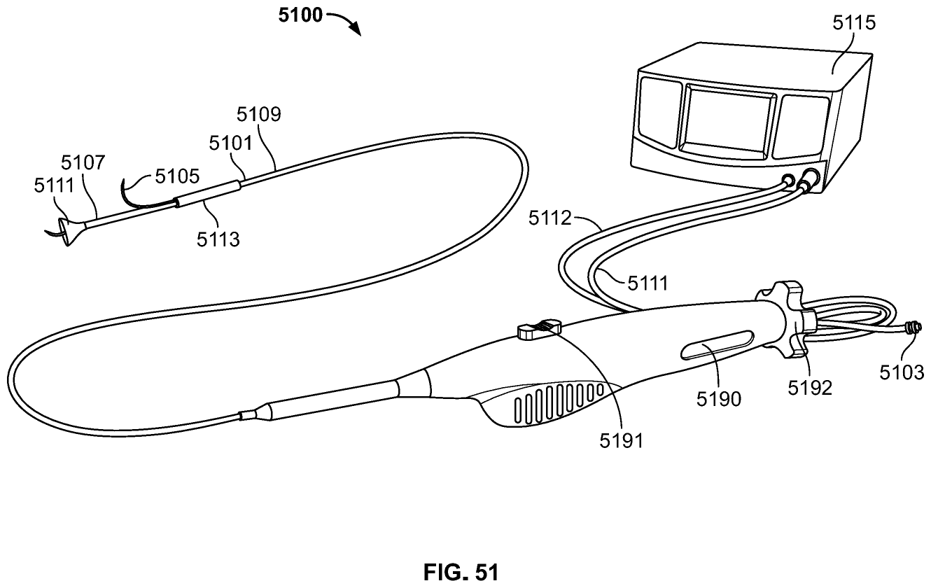

FIG. 51 illustrates a system 5100 for use in the ablation of prostatic tissue, in accordance with another embodiment of the present specification. The system 5100 comprises a catheter 5101 which, in some embodiments, includes a handle 5190 having actuators 5191, 5192 for extending at least one needle 5105 or a plurality of needles from a distal end of the catheter 5101 and expanding a positioning element 5111 at a distal end of the catheter 5101. In some embodiments, actuators 5191 and 5192 may be one of a knob or a slide or any other type of switch or button to enable extending of the at least one needle 5105 or plurality of needles. Delivery of vapor via the catheter 5101 is controlled by a controller 5115. In embodiments, the catheter 5101 comprises an outer sheath 5109 and an inner catheter 5107. The needle 5105 extends from the inner catheter 5107 at the distal end of the sheath 5109 or, in some embodiments, through openings proximate the distal end of the sheath 5109. In embodiments, the positioning element 5111 is expandable, positioned at the distal end of the inner catheter 5107, and may be compressed within the outer sheath 5109 for delivery. In some embodiments, actuator 5191 comprises a knob which is turned by a first extent, for example, by a quarter turn, to pull back the outer sheath 5109. As the outer sheath 5109 retracts, the positioning element 5111 is revealed. In embodiments, the positioning element 5111 comprises a disc or cone configured as a bladder anchor. In embodiments, actuator/knob is turned by a second extend, for example, by a second quarter turn, to pull back the outer sheath further 5109 to deploy the needle 5105. In some embodiments, the number of needles that is deployed is two or more than two. In some embodiments, referring to FIGS. 51, 4C and 4E simultaneously, the needle or needles 5105, 3116a are deployed out of an internal lumen of the inner catheter 5107, 3111a through slots or openings 3115a in the outer sheath 5109, 3110a, which helps control the needle path and insulates the urethra from steam. In some embodiments, the openings are covered with slit covers 3119. In another embodiment, for example, as seen in FIG. 4D, the sleeves 3116b naturally fold outward as the outer sheath 3110b is pulled back.

Referring again to FIG. 51, in some embodiments, the catheter 5101 includes a port 5103 for the delivery of fluid, for example cooling fluid, during ablation. In some embodiments, port 5103 is also configured to provide for fluid collection, provide vacuum, and provide CO₂ for an integrity test. In some embodiments, the port 5103 is positioned on the handle 5190. In some embodiments, at least one microwave antenna 5113 is positioned at a distal end of the catheter 5101 proximal to the needles 5105. The microwave antenna 5113 is configured to receive electrical current, supplied by a connecting wire 5111 extending from the controller 5115 to the catheter 5101, to heat and convert a fluid, such as saline supplied via tubing 5112 extending from the controller 5115 to the catheter 5101. Heated fluid or saline is converted to vapor or steam to be delivered by needle 5105 for ablation.

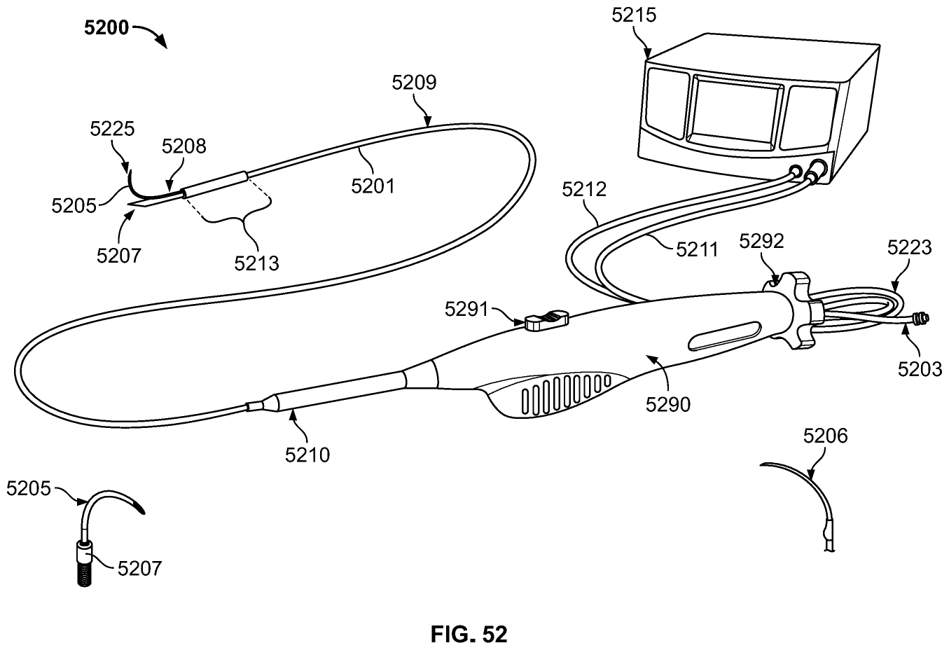

FIG. 52 illustrates a system 5200 for use in the ablation of prostatic tissue, in accordance with another embodiment of the present specification. The system 5200 comprises a catheter 5201 which, in some embodiments, includes a handle 5290 having actuators 5291, 5292 for extending at least one needle 5205 or a plurality of needles from a distal end of the catheter 5201. A drive mechanism configured within the handle 5290 deploys and retracts the needle 5205 in and out of a tip of the catheter shaft 5201. In some embodiments, actuators 5291 and 5292 may be one of a knob or a slide or any other type of switch or button to enable extending of the at least one needle 5205 or plurality of needles. In some embodiments, actuator 5291 is a button or a switch that allows a physician to activate treatment using system 5200 from the handle 5290 as well as a foot pedal (not shown). In some embodiments, a strain relief mechanism 5210 is configured at a distal end of the handle 5290 that connects the handle 5290 to the catheter 5201. The strain relief mechanism 5210 provides support to the catheter shaft 5201. Delivery of vapor via the catheter 5201 is controlled by a controller 5215. A cable sub-assembly 5223 including an electrical cable, in the handle 5290, connects the catheter 5201 to the controller 5215. In embodiments, the catheter 5201 comprises an outer sheath 5209 and an inner catheter (not shown).

In various embodiments, the controller 5215r (and controllers 4515, 5115, 5515p of FIGS. 45, 51, and 55 respectively) of the systems of the present specification comprises a computing device having one or more processors or central processing units, one or more computer-readable storage media such as RAM, hard disk, or any other optical or magnetic media, a controller such as an input/output controller, at least one communication interface and a system memory. The system memory includes at least one random access memory (RAM) and at least one read-only memory (ROM). In embodiments, the memory includes a database for storing raw data, images, and data related to these images. The plurality of functional and operational elements is in communication with the central processing unit (CPU) to enable operation of the computing device. In various embodiments, the computing device may be a conventional standalone computer or alternatively, the functions of the computing device may be distributed across a network of multiple computer systems and architectures and/or a cloud computing system. In some embodiments, execution of a plurality of sequences of programmatic instructions or code, which are stored in one or more non-volatile memories, enable or cause the CPU of the computing device to perform various functions and processes as described in the present specification. In alternate embodiments, hard-wired circuitry may be used in place of, or in combination with, software instructions for implementation of the processes of systems and methods described in this application. Thus, the systems and methods described are not limited to any specific combination of hardware and software.

A needle tip assembly 5225 is positioned within a needle chamber 5208, within the outer sheath 5209. The needle chamber 5208 may be a metal or plastic sleeve that is configured to house the needle 5205 during delivery to assist in needle deployment and retraction. The needle tip assembly 5225, including the needle 5205, extends from the inner catheter when pushed out of its chamber 5208, at the distal end of the sheath 5209 or, in some embodiments, through openings proximate the distal end of the sheath 5209. In embodiments, a positioning element is also provided at the distal end of the inner catheter. The positioning element may be expandable, and may be compressed within the outer sheath 5209 for delivery. In some embodiments, actuator 5292 comprises a knob which is turned by a first extent, for example, by a quarter turn, to pull back the outer sheath 5209. As the outer sheath 5209 retracts, the positioning element is revealed. In embodiments, actuator/knob 5292 is turned by a second extend, for example, by a second quarter turn, to pull back the outer sheath 5209 further to deploy the needle 5205. In some embodiments, the number of needles that is deployed is two or more than two.

FIG. 52 illustrates an expanded view of a needle tip assembly 5225, which includes a needle 5205 attached to a needle attachment component 5207 which, in some embodiments, comprises a metal threaded fitting. The needle attachment component, or threaded fitting, 5207 connects the needle 5205 to the catheter 5201. In embodiments, the needle attachment component 5207 comprises a threaded surface fixedly attached to a tip of the catheter 5201 and configured to have a needle 5205 screwed thereto. In some embodiments, the needle 5205 is a 22 to 25 G needle. In some embodiments, needle 5205 has a gradient of coating for insulation or echogenicity. An insulation coating 5206 may be ceramic, polymer, or any other material suitable for coating the needle 5205 and providing insulation and/or echogenicity to the needle 5205. The coatings are provided at the base of the needle 5205 to varying lengths to the needle tip.

Referring again to FIG. 52, in some embodiments, the catheter 5201 includes a tubing and connector sub-assembly (port) 5203 for the delivery of fluid, for example cooling fluid, during ablation. In some embodiments, port 5203 is also configured to provide for fluid collection, provide vacuum, and provide CO2 for an integrity test. In some embodiments, the port 5203 is positioned on the handle 5290. In some embodiments, one or more microwave antenna 5213 is positioned at a distal end of the catheter 5201 proximal to the one or more needles 5205. The one or more microwave antenna 5213 is configured to receive electrical current, supplied by a connecting wire 5211 extending from the controller 5215 to the catheter 5201, to heat and convert a fluid, such as saline, supplied via tubing 5212 extending from the controller 5215 to the catheter 5201. Heated fluid or saline is converted to vapor or steam to be delivered by needle 5205 for ablation.

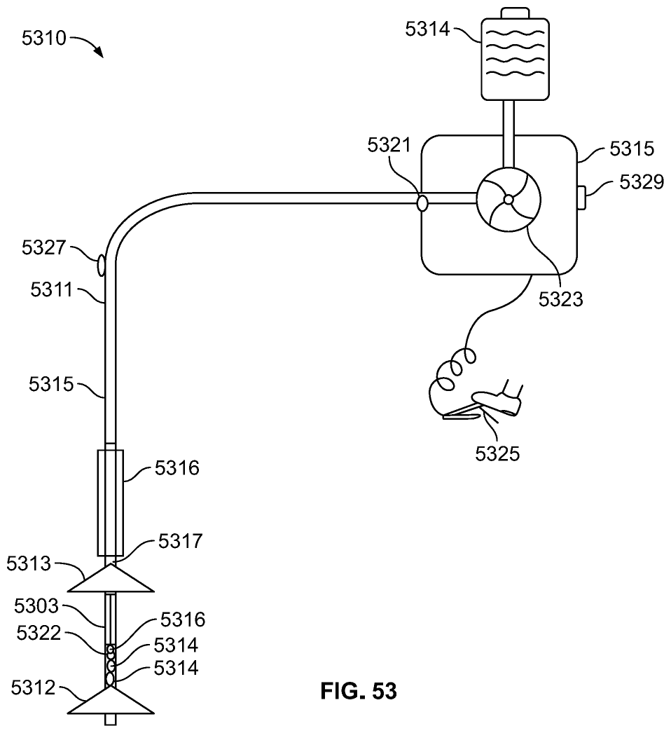

FIG. 53 illustrates an ablation system 5310 suitable for use in ablating an endometrial tissue, in accordance with embodiments of the present specification. The ablation system 5310 comprises a catheter 5311 having a catheter body 5315 comprising an outer catheter 5316 with an inner catheter 5317 concentrically positioned inside and extendable outside from a distal end of the outer catheter 5316. The inner catheter 5317 includes at least one first distal attachment or positioning element 5312 and a second proximal attachment or positioning element 5313. The inner catheter 5317 is positioned within the outer catheter 5316 during positioning of the catheter 5311 within a cervix or uterus of a patient. The first and second positioning elements 5312, 5313, in first, compressed configurations, are constrained by, and positioned within, the outer catheter 5316 during positioning of the catheter 5311. Once the distal end of the outer catheter 5311 has been positioned within a cervix of a patient, the inner catheter 5317 is extended distally from the distal end of the outer catheter 5316 and into a uterus of the patient. The first and second positioning elements 5312, 5313 expand and become deployed within the uterus. In embodiments, the first and second positioning elements 5312, 5313 comprise shape memory properties, allowing them to expand once deployed. In some embodiments, the first and second positioning elements 5312, 5313 are comprised of Nitinol. In some embodiments, once deployed, the first, distal positioning element 5312 is configured to contact a uterine wall, positioning the inner catheter 5317 within the uterus, and the second, proximal positioning element 5313, once deployed, is configured to abut a distal portion of the cervix just within the uterus, blocking passage of ablative vapor back into the cervical os. An internal heating chamber 5303 is disposed within a lumen of the inner catheter 5317 and configured to heat a fluid provided to the catheter 5311 to change said fluid to a vapor for ablation therapy. In some embodiments, the internal heating chamber is positioned just distal to the second positioning element 5313. In some embodiments, the catheter 5311 is made of or covered with an insulated material to prevent the escape of ablative energy from the catheter body. The inner catheter 5317 comprises one or more infusion ports 5314 for the infusion of an ablative agent, such as steam. In some embodiments, the one or more infusion ports 5314 are positioned on the catheter 5311 between the first and second positioning elements 5312 and 5313. In various embodiments, the first distal attachment or positioning element 5312 and second positioning element 5313 comprise discs. A fluid, such as saline, is stored in a reservoir, such as a saline pump 5314, connected to the catheter 5311. Delivery of the ablative agent is controlled by a controller 5315 and treatment is controlled by a treating physician via the controller 5315. The controller 5315 includes at least one processor 5323 in data communication with the saline pump 5314 and a catheter connection port 5321 in fluid communication with the saline pump 5314. In some embodiments, at least one optional sensor 5322 monitors changes in an ablation area to guide flow of ablative agent. In some embodiments, the optional sensor comprises at least one of a temperature sensor or pressure sensor. In some embodiments, the catheter 5311 includes a filter 5316 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 5325 in data communication with the controller 5315, a switch 5327 on the catheter 5311, or a switch 5329 on the controller 5315, for controlling vapor flow.

In one embodiment, a user interface included with the microprocessor 5315 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

In another embodiment, the outer catheter 5316 abuts a cervical canal mucosa without blocking the cervix and the outflow from the uterine cavity. A space between the outer catheter 5316 and inner catheter 5317 allows for venting via a channel for heated air, vapor or fluid to escape out of the uterine cavity without contacting and damaging the cervical canal.

The ablation device also includes safety mechanisms to prevent users from being burned while manipulating the catheter, including insulation, and optionally, cool air flush, cool water flush, and alarms/tones to indicate start and stop of treatment.

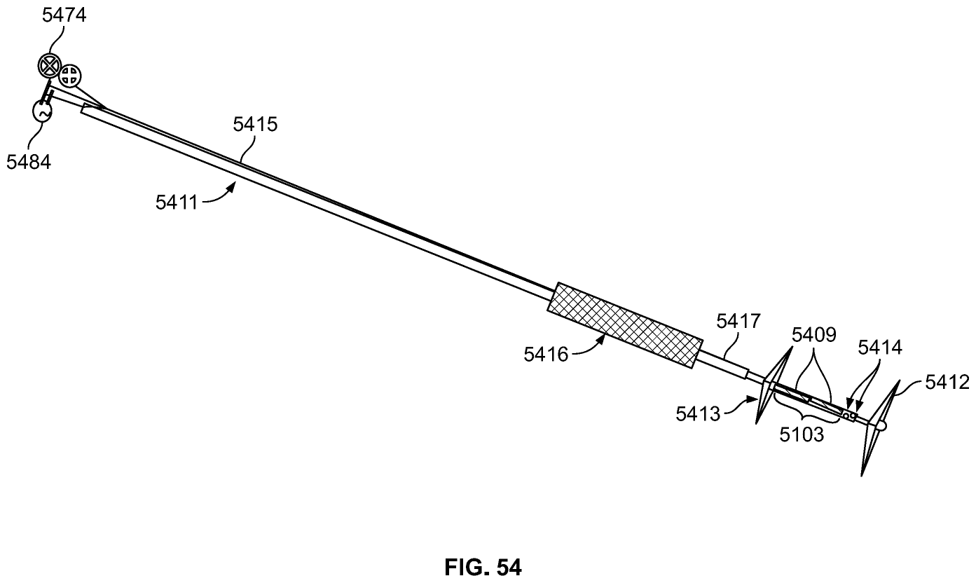

FIG. 54 illustrates another view of a catheter 5311 (herein referred to catheter 5411) of FIG. 53, in accordance with some embodiments of the present specification. The catheter 5411 includes an elongate body 5415 with a proximal end and a distal end. At the distal end, the catheter body 5415 includes an outer catheter 5416 with an inner catheter 5417 concentrically positioned inside and extendable outside from a distal end of the outer catheter 5416. The inner catheter 5417 includes a distal positioning element 5412, proximate its distal end, and a proximal positioning element 5413 proximal to the distal positioning element 5412. In various embodiments, the positioning elements are discs. The outer catheter 5416 is configured to receive the inner catheter 5417 and constrain the positioning elements 5412, 5413 before positioning, as described above. A plurality of infusion ports 5414 are located on the inner catheter 5417 between the two positioning elements 5412, 5413. The inner catheter 5417 also includes at least one heating chamber 5403 just distal to the proximal disc 5413. In some embodiments, the heating chamber 5403 includes two or more microwave antenna 5409 configured to receive RF current, heat, and convert supplied fluid, such as saline, to vapor, or steam, for ablation.

Referring to FIG. 54, for providing electrical current and liquid to the catheter 5411, a fluid pump 5474 and an RF generator 5484 are coupled to the proximal end of the body 5415. The fluid pump 5474 pumps the fluid, such as saline, through a first lumen (extending along the length of the body 5415) to the heating chamber 5403. The RF generator 5484 supplies electrical current to the microwave antenna 5409, causing the microwave antenna 5409 to generate heat and thereby converting the fluid flowing through the heating chamber 5403 into vapor. The generated vapor flows through the first lumen and exits the ports 5414 to ablate endometrial tissue. The flexible heating chamber 5403 imparts improved flexibility and maneuverability to the catheter 5411, allowing a physician to better position the catheter 5411 when performing ablation procedures, such as ablating endometrial tissue of a patient.

In various embodiments, the heating microwave antenna 5409 is proximal to the proximal positioning element 5413, extends beyond the distal end of the proximal positioning element 5413, or is completely distal to the distal end of the proximal positioning element 5413 but does not substantially extend beyond a proximal end of the distal positioning element 5412.

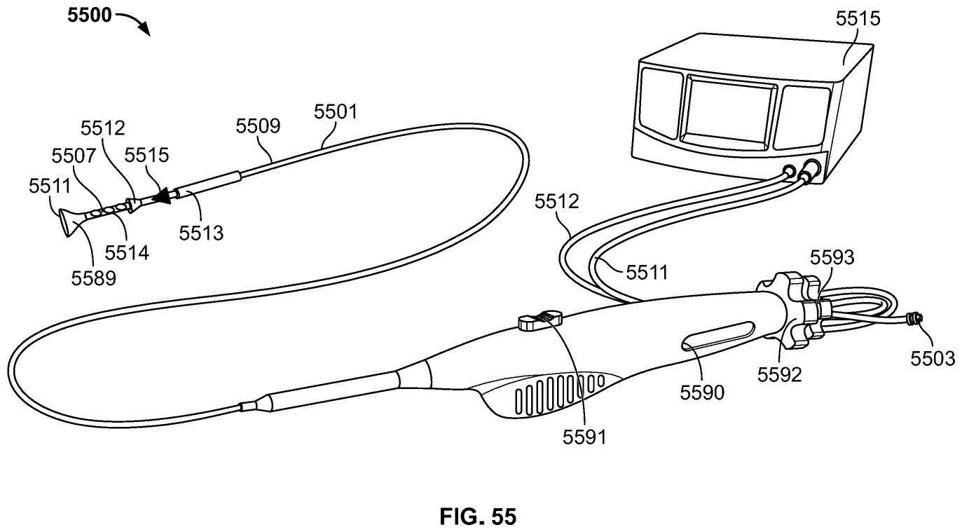

FIG. 55 illustrates a system 5500 for use in the ablation of endometrial tissue, in accordance with another embodiment of the present specification. The ablation system 5500 comprises a catheter 5501 which, in some embodiments, includes a handle 5590 having actuators 5591, 5592, 5593 for pushing forward a distal bulbous tip 5589 of the catheter 5501 and for deploying a first distal positioning element 5511 and a second proximal positioning element 5512 at the distal end of the catheter 550. In embodiments, the catheter 5501 comprises an outer sheath 5509 and an inner catheter 5507. In embodiments, the catheter 5501 includes a cervical collar 5515 configured to rest against an external os once the catheter 5501 has been inserted into a uterus of a patient. In embodiments, the distal first positioning element 5511 and proximal second positioning element 5512 are expandable, positioned at the distal end of the inner catheter 5507, and may be compressed within the outer sheath 5509 for delivery. In some embodiments, actuators 5592 and 5593 comprise knobs. In some embodiments, actuator/knob 5592 is used to deploy the distal first positioning element 5511. For example, in embodiments, actuator/knob 5592 is turned one quarter turn to deploy the distal first positioning element 5511. In some embodiments, actuator/knob 5593 is used to deploy the proximal second positioning element 5512. For example, in embodiments, actuator/knob 5593 is turned one quarter turn to deploy the proximal second positioning element 5512. In some embodiments, the handle 5590 includes only one actuator/knob 5592 which is turned a first quarter turn to deploy the first distal positioning element 5511 and then a second quarter turn to deploy the second proximal positioning element 5512. In other embodiments, other combinations of actuators/knobs are used to deploy one or both of the first distal positioning element 5511 and second proximal positioning element 5512. In some embodiments, the catheter 5501 includes a port 5503 for the delivery of fluid, for example cooling fluid, during ablation. In some embodiments, port 5503 is also configured to provide for fluid collection, provide vacuum, and provide CO2 for an integrity test. In some embodiments, the port 5503 is positioned on the handle 5590. In some embodiments, at least one microwave antenna 5513 is positioned at a distal end of the catheter 5501 proximal to the proximal second positioning element 5512. The microwave antenna 5513 is configured to receive electrical current, supplied by a connecting wire 5511 extending from the controller 5515 to the catheter 5501, to heat and convert a fluid, such as saline supplied via tubing 5512 extending from the controller 5515 to the catheter 5501. Heated fluid or saline is converted to vapor or steam to be delivered by ports 5514 ablation. In some embodiments, the catheter 5501 is made of or covered with an insulated material to prevent the escape of ablative energy from the catheter body. A plurality of small delivery ports 5514 is positioned on the inner catheter 5507 between the distal first positioning element 5511 and the second proximal positioning element 5512. Ports 5514 are used for the infusion of an ablative agent, such as steam. Delivery of the ablative agent is controlled by the controller 5515 and treatment is controlled by a treating physician via the controller 5515.

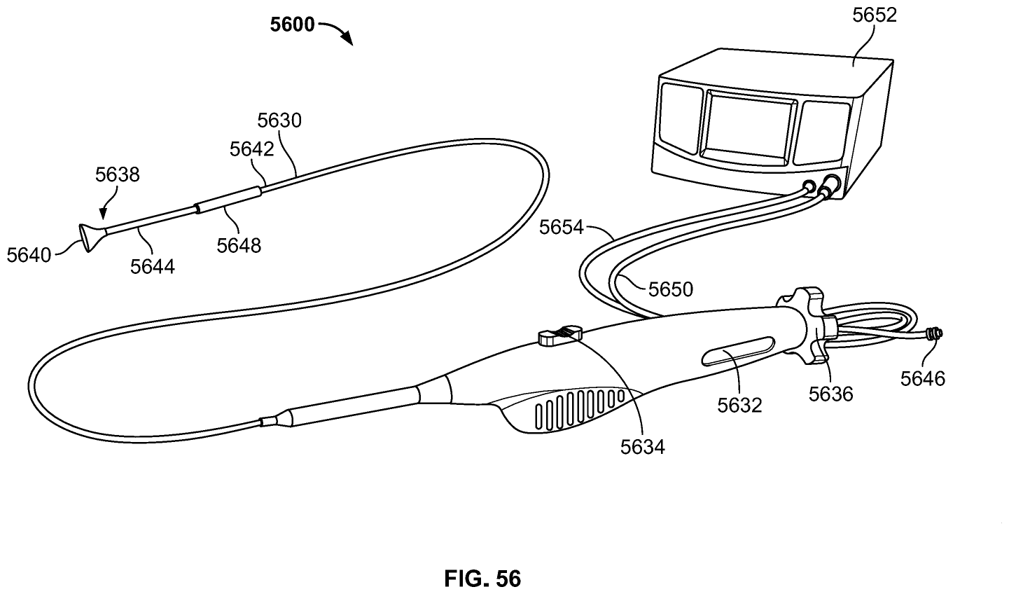

FIG. 56 illustrates a system 5600 for use in the ablation of bladder tissue, in accordance with an embodiment of the present specification. The system 5600 comprises a catheter 5630 which, in some embodiments, includes a handle 5632 having actuators 5634, 5636 for pushing forward a distal tip 5638 of the catheter 5630 and for deploying a distal positioning element 5640 at the distal end of the catheter 5630. In embodiments, the catheter 5630 comprises an outer sheath 5642 and an inner catheter 5644. In embodiments, the distal positioning element 5640 is expandable, positioned at the distal end of the inner catheter 5644, and may be compressed within the outer sheath 5642 for delivery. In some embodiments, actuators 5634 and 5636 comprise knobs. In some embodiments, actuator/knob 5636 is used to deploy the distal positioning element 5640. For example, in embodiments, actuator/knob 5636 is turned one quarter turn to deploy the distal positioning element 5640. In some embodiments, other combinations of actuators/knobs are used to the positioning element 5640. In some embodiments, the catheter 5630 includes a port 5646 for the delivery of fluid, for example cooling fluid, during ablation. In some embodiments, port 5646 is also configured to provide for fluid collection, provide vacuum, and provide CO2 for an integrity test. In some embodiments, the port 5646 is positioned on the handle 5632. In some embodiments, at least one microwave antenna 5648 is positioned at a distal end of the catheter 5630. The microwave antenna 5648 is configured to receive electrical current, supplied by a connecting wire 5650 extending from a controller 5652 to the catheter 5630, to heat and convert a fluid, such as saline supplied via a tubing 5654 extending from the controller 5652 to the catheter 5630. Heated fluid or saline is converted to vapor or steam to be delivered by ports for ablation. In some embodiments, the catheter 5630 is made of or covered with an insulated material to prevent the escape of ablative energy from the catheter body. A plurality of small delivery ports are positioned on the inner catheter 5644 between the distal positioning element 5640 and the microwave antenna 5648. Ports are used for the infusion of an ablative agent, such as steam. Delivery of the ablative agent is controlled by the controller 5652 and treatment is controlled by a treating physician via the controller 5652.

In embodiments, the microwave-based ablation systems described herein allow for smaller diameters and greater flexibility, since microwave antennas are more flexible than electrodes. It is possible to achieve greater than 80% vapor efficiency, because microwave antennas are more efficient than electrodes. The vapor efficiency achieved by microwave-based ablation systems is greater by more than 10% than that achieved by electrode-based ablation systems. Additionally, the microwave-based ablation systems can ablate a relatively bigger lesion in 10 seconds as a result of its efficient steam generation.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. An ablation catheter configured to deliver an ablation fluid to at least one of a volume of prostate tissue or a volume of fibroid tissue, comprising:
a sheath having at least one lumen, wherein the lumen is configured to receive a volume of fluid;
at least one needle positioned within a distal tip of the catheter and configured to be deployed from a surface of the distal tip;
at least one port positioned in the at least one needle;
at least one heating component positioned within the lumen and proximate the distal tip, wherein the at least one heating component is configured to receive the volume of fluid;
a handle coupled to a proximal end of the sheath;
a camera positioned proximate the distal tip and configured to visually capture a movement and location of the at least one needle when the at least one needle is extended out from the surface of the distal tip;
a light source positioned proximate the camera, wherein the camera and the light source are physically coupled into the sheath; and
optical data transmission circuitry coupled to the camera, wherein a value defining a maximum diameter of the catheter is equal to, or less than, 8 mm.

2. The ablation catheter of claim 1, wherein the at least one heating component is a flat electrode.

3. The ablation catheter of claim 1, wherein the at least one needle is configured to be deployed at an angle relative to a longitudinal axis defining a direction of the distal tip.

4. The ablation catheter of claim 3, wherein the angle is in a range of 10 degrees to 90 degrees relative to the longitudinal axis defining the direction of the distal tip.

5. The ablation catheter of claim 1, wherein the camera and the light source are not positioned in a scope physically separate from the sheath.

6. The ablation catheter of claim 1, wherein the maximum diameter of the catheter is in a range of 4 mm to 5 mm.

7. The ablation catheter of claim 1, wherein the at least one heating component comprises an electrode and wherein the electrode is tapered such that a distal tip of the electrode is thinner than a proximal portion of the electrode.

8. The ablation catheter of claim 1, wherein the fluid is saline.

9. The ablation catheter of claim 1, wherein the sheath comprises a second lumen running parallel to the at least one lumen and wherein the optical data transmission circuitry is positioned within the second lumen.

10. An ablation system configured to deliver an ablation fluid to at least one of a volume of prostate tissue or a volume of fibroid tissue, comprising:
a catheter having:
a sheath having at least one lumen, wherein the lumen is configured to receive a volume of fluid;
at least one needle positioned within a distal tip of the catheter and configured to be deployed from a surface of the distal tip;
at least one port positioned in the at least one needle;
at least one heating component positioned within the lumen and proximate the distal tip, wherein the at least one heating component is configured to receive the volume of fluid;
a handle coupled to a proximal end of the sheath;
a camera positioned proximate the distal tip and configured to visually capture a movement and location of the at least one needle when the at least one needle is extended out from the surface of the distal tip;
a light source positioned proximate the camera, wherein the camera and the light source are physically coupled to the sheath; and
optical data transmission circuitry coupled to the camera, wherein a value defining a maximum diameter of the catheter is equal to, or less than, 8 mm;
a fluid reservoir configured to contain the volume of fluid and coupled to the at least one lumen;
a pump in pressure communication with the fluid reservoir; and
a controller coupled to the pump, wherein the controller is in electrical communication with the at least one heating component and programmed to deliver an electrical current to the at least one heating component and to cause the volume of fluid to pass into the lumen from the fluid reservoir when activated.

11. The ablation system of claim 10, further comprising a power source positioned in the controller and coupled to the light source and the camera.

12. The ablation system of claim 10, wherein the at least one heating component is a flat electrode.

13. The ablation system of claim 10, wherein the at least one needle is configured to be deployed at an angle relative to a longitudinal axis defining a direction of the distal tip.

14. The ablation system of claim 13, wherein the angle is in a range of 10 degrees to 90 degrees relative to the longitudinal axis defining the direction of the distal tip.

15. The ablation system of claim 10, wherein the camera and the light source are not positioned in a scope physically separate from the sheath.

16. The ablation system of claim 10, wherein the maximum diameter of the catheter is in a range of 4 mm to 5 mm.

17. The ablation system of claim 10, wherein the at least one heating component is an electrode and wherein the electrode is tapered such that a distal tip of the electrode is thinner than a proximal portion of the electrode.

18. The ablation system of claim 10, wherein the controller is programmed to deliver the electrical current to the at least one heating component for a continuous period of time that is equal to, or less than, one minute.

19. The ablation system of claim 10, wherein the sheath comprises a second lumen running parallel to the at least one lumen and wherein the optical data transmission circuitry is positioned within the second lumen.

20. The ablation system of claim 19, wherein the second lumen has a diameter that is equal to or less than 4 mm and the at least one lumen has a diameter that is equal to or less than 4 mm.
